(19) United States
(12) Patent Application Publication
(10) Pub. No.: US 2026/0265376 A1
(43) Pub. Date: Sep. 10, 2026

(54) MULTISPECIFIC ANTIGEN BINDING PROTEINS AND USES THEREOF

(71) Applicant: ITABMED CO., LTD., Grand Cayman (KY)

(72) Inventors: Guiyun TU, Shanghai (CN); Wuzhong SHEN, Shanghai (CN); Hanyang CHEN, Shanghai (CN); Shengjie XUE, Shanghai (CN); Zheng YANG, Shanghai (CN); Bingbin XIE, Shanghai (CN); Ke WANG, Shanghai (CN); Zuowen DUAN, Shanghai (CN); Xiaoqiang YAN, Shanghai (CN)

(21) Appl. No.: 19/165,465

(22) PCT Filed: Mar. 18, 2024
(86) PCT No.: PCT/CN2024/082246

§ 371 Date: Sep. 15, 2025

(30) Foreign Application Priority Data
Mar. 16, 2023 (WO) PCT/CN2023/082015
Oct. 30, 2023 (WO) PCT/CN2023/127757

(51) Int. Cl. C07K 16/28; A61K 39/00

(52) U.S. Cl. CPC C07K 16/2809; C07K 16/2803; C07K 16/2815; A61K 2039/505; C07K 2317/31; C07K 2317/51; C07K 2317/515; C07K 2317/522; C07K 2317/55; C07K 2317/565; C07K 2317/622

(57) ABSTRACT

The invention provides novel heterodimeric proteins including heterodimeric antibodies. The present invention describes novel immunoglobulin compositions that simultaneously co-engage antigens, where both of the antigens are bound monovalently. The novel immunoglobulins described preferably utilize heterodimeric Fe regions. Methods of using the novel immunoglobulin compositions, particularly for therapeutic purposes, are also described herein.

### CROSS-REFERENCE TO RELATED APPLICATIONS

This application claims the priority benefit of International Patent Application No. PCT/CN2023/082015, filed Mar. 16, 2023, and International Patent Application No. PCT/CN2023/127757, filed Oct. 30, 2023, the contents of each of which are incorporated herein by reference in their entirety.

### REFERENCE TO AN ELECTRONIC SEQUENCE LISTING

The contents of the electronic sequence listing (221992000742SEQLIST.xml; Size: 190,159 bytes; and Date of Creation: Mar. 13, 2024) is herein incorporated by reference in its entirety.

### FIELD OF THE INVENTION

The present invention relates to multispecific antigen binding proteins (MSAPs, e.g., trispecific antigen binding proteins (TSAPs)) that specifically bind to CD3, CD8, and a target antigen (e.g., tumor-specific antigen (TSA) or tumor-associated antigen (TAA)). Further provided herein are antibodies or antigen binding fragments thereof targeting CD3 or CD8, pharmaceutical compositions comprising the MSAPs (e.g., TSAPs), methods of treating cancer using the MSAPs (e.g., TSAPs), methods of producing thereof, and kits comprising the MSAPs (e.g., TSAPs).

### BACKGROUND OF THE INVENTION

Many abnormal cells and tissues as well as many diseases display unique antigens that can be leveraged for immune cell-mediated clearance. Exemplary diseases include, but are not limited to, cancers (e.g., solid tumors or liquid cancers), autoimmune disorders, bacterial diseases, viral diseases, or fungal diseases that can each display one or more different target antigens or one or more different epitopes of the same target antigen. For example, some antigens are over-expressed, mutagenized, or selectively mutagenized in tumor tissues. Therefore, antibodies targeting specific antigens on the surface of cancer cells can be used as cancer therapeutics.

CD3, comprising three different polypeptide chains (8, 8, and γ chains), is an antigen expressed by T cells. The three CD3 polypeptide chains associate with the T-cell receptor (TCR) and the ζ-chain to form the TCR complex, which has the function of activating signaling cascades in T cells. Currently, many therapeutic strategies target the TCR signal transduction to treat diseases using anti-human CD3 monoclonal antibodies. The CD3 specific antibody OKT3 is the first monoclonal antibody approved for human therapeutic use and is clinically used as an immunomodulator for the treatment of allogenic transplant rejections.

Although bispecific antibodies targeting CD3 and a target antigen (e.g., tumor antigen) have been shown to have potential in effectively killing cancer cells, severe adverse effects, including systemic immune activation, immunogenicity (anti-drug antibody effect), and the generally poor manufacturability of these molecules, have greatly limited the widespread application of this type of drugs.

The drawbacks of current formats of bispecific antibodies remain great challenges for their widespread application in the treatment of cancer patients with good efficacy and safety.

### BRIEF SUMMARY OF THE INVENTION

The present invention provides multispecific antigen binding proteins (“MSAPs”, such as trispecific antigen binding proteins, “TSAPs”) that specifically bind to CD3 and CD8 and a target antigen (e.g., a tumor-specific antigen (TSA) or tumor-associated antigen (TAA)), pharmaceutical compositions comprising the MSAPs, and methods of using the MSAPs (e.g., treating cancer). The present invention further provides antibodies and antigen binding fragments thereof targeting CD8 or CD3.

In one aspect of the present invention, there is provided an MSAP comprising: i) an anti-CD3 moiety that specifically binds to CD3; ii) an anti-CD8 moiety that specifically binds to CD8; and iii) an anti-target epitope moiety that specifically binds to a target epitope (e.g., TSA or TAA, such as CD19, EpCAM, or WT1), wherein a first moiety of the three moieties is a Fab fragment, wherein the Fab fragment comprises a first polypeptide comprising a heavy chain variable region (VH) and a heavy chain constant region (CH1), and a second polypeptide comprising a light chain variable region (VL) and a light chain constant region (CL); wherein a second moiety of the three moieties is a first antigen binding fragment (e.g., scFv); wherein a third moiety of the three moieties is a second antigen binding fragment (e.g., scFv); and wherein (a) the first antigen binding fragment is fused to the N-terminus of the VH of the Fab fragment, and the second antigen binding fragment is fused to the N-terminus of the VL of the Fab fragment; (b) the first antigen binding fragment is fused to the C-terminus of the CH1 of the Fab fragment, and the second antigen binding fragment is fused to the N-terminus of the VL of the Fab fragment; (c) the first antigen binding fragment is fused to the N-terminus of the VH of the Fab fragment, and the second antigen binding fragment is fused to the C-terminus of the CL of the Fab fragment; (d) the first antigen binding fragment is fused to the N-terminus of the VH of the Fab fragment, and the second antigen binding fragment is fused to the C-terminus of the CH1 of the Fab fragment; or (e) the first antigen binding fragment is fused to the N-terminus of the VL of the Fab fragment, and the second antigen binding fragment is fused to the C-terminus of the CL of the Fab fragment.

In some embodiments according to any of the MSAPs described above, (a) the anti-CD3 moiety is the Fab fragment (“anti-CD3 Fab fragment”), the anti-CD8 moiety is the first antigen binding fragment (“anti-CD8 first antigen binding fragment”), and the anti-target epitope moiety is the second antigen binding fragment (“anti-target epitope second antigen binding fragment”); (b) the anti-CD3 moiety is the Fab fragment, the anti-target epitope moiety is the first antigen binding fragment (“anti-target epitope first antigen binding fragment”), and the anti-CD8 moiety is the second antigen binding fragment (“anti-CD8 second antigen binding fragment”); (c) the anti-CD8 moiety is the Fab fragment (“anti-CD8 Fab fragment”), the anti-CD3 moiety is the first antigen binding fragment (“anti-CD3 first antigen binding fragment”), and the anti-target epitope moiety is the second antigen binding fragment; (d) the anti-CD8 moiety is the Fab fragment, the anti-target epitope moiety is the first antigen binding fragment, and the anti-CD3 moiety is the second antigen binding fragment (“anti-CD3 second antigen binding fragment”); (e) the anti-target epitope (e.g., TSA or TAA, such as CD19, EpCAM, or WT1) moiety is the Fab fragment (“anti-target epitope Fab fragment”), the anti-CD3 moiety is the first antigen binding fragment, and the anti-CD8 moiety is the second antigen binding fragment; or (f) the anti-target epitope moiety is the Fab fragment, the anti-CD8 moiety is the first antigen binding fragment, and the anti-CD3 moiety is the second antigen binding fragment.

In some embodiments according to any of the MSAPs described above, the first antigen binding fragment and the second antigen binding fragment are independently selected from the group consisting of Fab, Fab′, Fab′-SH, F(ab′)₂, Fv, sdAb, scFv, and any combination thereof. In some embodiments, the first antigen binding fragment and the second antigen binding fragment are both scFv.

In some embodiments according to any of the MSAPs described above, the anti-CD8 moiety specifically binds to CD8α. In some embodiments, the anti-CD8 moiety comprises: i) a heavy chain hypervariable region-1 (HVR-H1) comprising the amino acid sequence of SEQ ID NO: 13, an HVR-H2 comprising the amino acid sequence of SEQ ID NO: 14, an HVR-H3 comprising the amino acid sequence of SEQ ID NO: 15, a light chain hypervariable region-1 (HVR-L1) comprising the amino acid sequence of SEQ ID NO: 4, an HVR-L2 comprising the amino acid sequence of SEQ ID NO: 5, and an HVR-L3 comprising the amino acid sequence of SEQ ID NO: 6; ii) an HVR-H1 comprising the amino acid sequence of SEQ ID NO: 1, an HVR-H2 comprising the amino acid sequence of SEQ ID NO: 16, an HVR-H3 comprising the amino acid sequence of SEQ ID NO: 3, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iii) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iv) an HVR-H1 comprising the amino acid sequence of SEQ ID NO: 1, an HVR-H2 comprising the amino acid sequence of SEQ ID NO: 18, an HVR-H3 comprising the amino acid sequence of SEQ ID NO: 19, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; v) an HVR-H1 comprising the amino acid sequence of SEQ ID NO: 7, an HVR-H2 comprising the amino acid sequence of SEQ ID NO: 8, an HVR-H3 comprising the amino acid sequence of SEQ ID NO: 9, an HVR-L1 comprising the amino acid sequence of SEQ ID NO: 10, an HVR-L2 comprising the amino acid sequence of SEQ ID NO: 11, and an HVR-L3 comprising the amino acid sequence of SEQ ID NO: 12; or vi) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23. In some embodiments, the anti-CD8 moiety comprises: i) a VH comprising the amino acid sequence of SEQ ID NO: 24, and a VL comprising the amino acid sequence of SEQ ID NO: 25; ii) a VH comprising the amino acid sequence of SEQ ID NO: 26, and a VL comprising the amino acid sequence of SEQ ID NO: 27; iii) a VH comprising the amino acid sequence of SEQ ID NO: 28, and a VL comprising the amino acid sequence of SEQ ID NO: 29; iv) a VH comprising the amino acid sequence of SEQ ID NO: 30, and a VL comprising the amino acid sequence of SEQ ID NO: 25; v) a VH comprising the amino acid sequence of SEQ ID NO: 32, and a VL comprising the amino acid sequence of SEQ ID NO: 33; or vi) a VH comprising the amino acid sequence of SEQ ID NO: 34, and a VL comprising the amino acid sequence of SEQ ID NO: 35. In some embodiments, the anti-CD8 moiety is an anti-CD8 scFv, and the anti-CD8 scFv comprises the amino acid sequence of any one of SEQ ID NOs: 21, 44-49, and 51-55.

In some embodiments according to any of the MSAPs described above, the anti-CD8 moiety comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23. In some embodiments, the anti-CD8 moiety comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD8 moiety is an anti-CD8 scFv, and the anti-CD8 scFv comprises the amino acid sequence of any one of SEQ ID NOs: 46, 49, 52, and 55.

In some embodiments according to any of the MSAPs described above, the anti-CD3 moiety comprises: (a) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (b) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (c) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the anti-CD3 moiety comprises: (a) a VH comprising the amino acid sequence of SEQ ID NO: 42, and a VL comprising the amino acid sequence of SEQ ID NO: 43; (b) a VH comprising the amino acid sequence of SEQ ID NO: 159, and a VL comprising the amino acid sequence of SEQ ID NO: 43; or (c) a VH comprising the amino acid sequence of SEQ ID NO: 167, and a VL comprising the amino acid sequence of SEQ ID NO: 43. In some embodiments, the anti-CD3 moiety is an anti-CD3 scFv, and the anti-CD3 scFv comprises the amino acid sequence of any of SEQ ID NOs: 50, 56, 163, 164, 183, and 184.

In some embodiments according to any of the MSAPs described above, the anti-target epitope moiety specifically recognizes a target epitope of a tumor-associated antigen (TAA) or a tumor-specific antigen (TSA). In some embodiments, the TAA is CD19 or EpCAM. In some embodiments, the TSA is WT1.

In some embodiments according to any of the MSAPs described above, the anti-target epitope moiety specifically recognizes CD19 (anti-CD19 moiety). In some embodiments, the anti-CD19 moiety comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD19 moiety comprises a VH comprising the amino acid sequence of SEQ ID NO: 63, and a VL comprising the amino acid sequence of SEQ ID NO: 64. In some embodiments, the anti-CD19 moiety is an anti-CD19 scFv, and the anti-CD19 scFv comprises the amino acid sequence of SEQ ID NO: 65 or 66. In some embodiments, the anti-CD19 moiety is an anti-CD19 Fab, and wherein the anti-CD19 Fab fragment comprises a first polypeptide comprising the amino acid sequence of SEQ ID NO: 137, and a second polypeptide comprising the amino acid sequence of SEQ ID NO: 139 or 169.

In some embodiments according to any of the MSAPs described above, the anti-target epitope moiety specifically recognizes EpCAM (anti-EpCAM moiety). In some embodiments, the anti-EpCAM moiety comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, the anti-EpCAM moiety comprises a VH comprising the amino acid sequence of SEQ ID NO: 73, and a VL comprising the amino acid sequence of SEQ ID NO: 74. In some embodiments, the anti-EpCAM moiety is an anti-EpCAM scFv, and the anti-EpCAM scFv comprises the amino acid sequence of SEQ ID NO: 75 or 78.

In some embodiments according to any of the MSAPs described above, the anti-target epitope moiety specifically recognizes WT1 (anti-WT1 moiety). In some embodiments, the anti-WT1 moiety comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the anti-WT1 moiety comprises a VH comprising the amino acid sequence of SEQ ID NO: 85, and a VL comprising the amino acid sequence of SEQ ID NO: 86. In some embodiments, the anti-WT1 moiety is an anti-WT1 scFv, and the anti-WT1 scFv comprises the amino acid sequence of SEQ ID NO: 87 or 88.

In some embodiments according to any of the MSAPs described above, the CH1 comprises the amino acid sequence of SEQ ID NO: 93, and the CL comprises the amino acid sequence of SEQ ID NO: 94 or 165.

In some embodiments according to any of the MSAPs described above, the first antigen binding fragment is fused to the Fab fragment via a first linker, and/or the second antigen binding fragment is fused to the Fab fragment via a second linker. In some embodiments, the first linker and/or the second linker is about 2 to about 30 amino acid residues in length. In some embodiments, the first linker and/or the second linker comprises amino acid residues selected from the group consisting of glycine, serine, arginine, and alanine. In some embodiments, the first linker and/or the second linker comprises an amino acid sequence independently selected from the group consisting of GG and SEQ ID NOs: 108-110.

In some embodiments according to any of the MSAPs described above, the MSAP comprises: i) an anti-CD3 Fab fragment, an anti-CD8 first scFv, and an anti-WT1 second scFv; or ii) an anti-CD3 Fab fragment, an anti-WT1 first scFv, and an anti-CD8 second scFv. In some embodiments, the anti-CD3 Fab fragment comprises: (i) a first polypeptide comprising the amino acid sequence of SEQ ID NO: 89, and a second polypeptide comprising the amino acid sequence of SEQ ID NO: 90 or 179; (ii) a first polypeptide comprising the amino acid sequence of SEQ ID NO: 160, and a second polypeptide comprising the amino acid sequence of SEQ ID NO: 90 or 179; or (iii) a first polypeptide comprising the amino acid sequence of SEQ ID NO: 168, and a second polypeptide comprising the amino acid sequence of SEQ ID NO: 90 or 179. In some embodiments, the MSAP comprises a first fusion polypeptide comprising an anti-WT1 first scFv fused to the N-terminus of the VH of the anti-CD3 Fab fragment via a first linker, and a second fusion polypeptide comprising an anti-CD8 second scFv fused to the N-terminus of the VL of the anti-CD3 Fab fragment via a second linker; and wherein the first fusion polypeptide comprises the amino acid sequence of SEQ ID NO: 95, and the second fusion polypeptide comprises the amino acid sequence of SEQ ID NO: 96.

In some embodiments according to any of the MSAPs described above, the MSAP comprises: i) an anti-EpCAM Fab fragment, an anti-CD3 first scFv, and an anti-CD8 second scFv; or ii) an anti-EpCAM Fab fragment, an anti-CD8 first scFv, and an anti-CD3 second scFv. In some embodiments, the anti-EpCAM Fab fragment comprises a first polypeptide comprising the amino acid sequence of SEQ ID NO: 76, and a second polypeptide comprising the amino acid sequence of SEQ ID NO: 77. In some embodiments, the multispecific antigen binding protein comprises a first fusion polypeptide comprising an anti-CD3 first scFv fused to the N-terminus of the VH of the anti-EpCAM Fab fragment via a first linker, and a second fusion polypeptide comprising an anti-CD8 second scFv fused to the N-terminus of the VL of the anti-EpCAM Fab fragment via a second linker; and wherein the first fusion polypeptide comprises the amino acid sequence of SEQ ID NO: 97, and the second fusion polypeptide comprises the amino acid sequence of SEQ ID NO: 98.

In some embodiments according to any of the MSAPs described above, the MSAP comprises: i) an anti-CD8 Fab fragment, an anti-EpCAM first scFv, and an anti-CD3 second scFv; or ii) an anti-CD8 Fab fragment, an anti-CD3 first scFv, and an anti-EpCAM second scFv. In some embodiments, the anti-CD8 Fab fragment comprises: i) a first polypeptide comprising the amino acid sequence of SEQ ID NO: 147, and a second polypeptide comprising the amino acid sequence of SEQ ID NO: 148; ii) a first polypeptide comprising the amino acid sequence of SEQ ID NO: 149, and a second polypeptide comprising the amino acid sequence of SEQ ID NO: 150; iii) a first polypeptide comprising the amino acid sequence of SEQ ID NO: 91, and a second polypeptide comprising the amino acid sequence of SEQ ID NO: 92; iv) a first polypeptide comprising the amino acid sequence of SEQ ID NO: 151, and a second polypeptide comprising the amino acid sequence of SEQ ID NO: 148; v) a first polypeptide comprising the amino acid sequence of SEQ ID NO: 153, and a second polypeptide comprising the amino acid sequence of SEQ ID NO: 154; or vi) a first polypeptide comprising the amino acid sequence of SEQ ID NO: 155, and a second polypeptide comprising the amino acid sequence of SEQ ID NO: 156. In some embodiments, the multispecific antigen binding protein comprises a first fusion polypeptide comprising an anti-EpCAM first scFv fused to the N-terminus of the VH of the anti-CD8 Fab fragment via a first linker, and a second fusion polypeptide comprising an anti-CD3 second scFv fused to the N-terminus of the VL of the anti-CD8 Fab fragment via a second linker; and wherein the first fusion polypeptide comprises the amino acid sequence of SEQ ID NO: 99, and the second fusion polypeptide comprises the amino acid sequence of SEQ ID NO: 100.

In some embodiments according to any of the MSAPs described above, the MSAP comprises: i) an anti-CD3 Fab fragment, an anti-EpCAM first scFv, and an anti-CD8 second scFv; or ii) an anti-CD3 Fab fragment, an anti-CD8 first scFv, and an anti-EpCAM second scFv. In some embodiments, the anti-CD3 Fab fragment comprises: (i) a first polypeptide comprising the amino acid sequence of SEQ ID NO: 89, and a second polypeptide comprising the amino acid sequence of SEQ ID NO: 90 or 179; (ii) a first polypeptide comprising the amino acid sequence of SEQ ID NO: 160, and a second polypeptide comprising the amino acid sequence of SEQ ID NO: 90 or 179; or (iii) a first polypeptide comprising the amino acid sequence of SEQ ID NO: 168, and a second polypeptide comprising the amino acid sequence of SEQ ID NO: 90 or 179. In some embodiments, the multispecific antigen binding protein comprises a first fusion polypeptide comprising an anti-EpCAM first scFv fused to the N-terminus of the VH of the anti-CD3 Fab fragment via a first linker, and a second fusion polypeptide comprising an anti-CD8 second scFv fused to the N-terminus of the VL of the anti-CD3 Fab fragment via a second linker; and wherein the first fusion polypeptide comprises the amino acid sequence of SEQ ID NO: 101, and the second fusion polypeptide comprises the amino acid sequence of SEQ ID NO: 102.

In some embodiments according to any of the MSAPs described above, the MSAP comprises: i) an anti-CD3 Fab fragment, an anti-CD19 first scFv, and an anti-CD8 second scFv; or ii) an anti-CD3 Fab fragment, an anti-CD8 first scFv, and an anti-CD19 second scFv. In some embodiments, the anti-CD3 Fab fragment comprises: (i) a first polypeptide comprising the amino acid sequence of SEQ ID NO: 89, and a second polypeptide comprising the amino acid sequence of SEQ ID NO: 90 or 179; ii) a first polypeptide comprising the amino acid sequence of SEQ ID NO: 160, and a second polypeptide comprising the amino acid sequence of SEQ ID NO: 90 or 179; or (iii) a first polypeptide comprising the amino acid sequence of SEQ ID NO: 168, and a second polypeptide comprising the amino acid sequence of SEQ ID NO: 90 or 179. In some embodiments, the multispecific antigen binding protein comprises a first fusion polypeptide comprising an anti-CD19 first scFv fused to the N-terminus of the VH of the anti-CD3 Fab fragment via a first linker, and a second fusion polypeptide comprising an anti-CD8 second scFv fused to the N-terminus of the VL of the anti-CD3 Fab fragment via a second linker; and wherein i) the first fusion polypeptide comprises the amino acid sequence of SEQ ID NO: 103, and the second fusion polypeptide comprises the amino acid sequence of SEQ ID NO: 104; ii) the first fusion polypeptide comprises the amino acid sequence of SEQ ID NO: 103, and the second fusion polypeptide comprises the amino acid sequence of SEQ ID NO: 106; or iii) the first fusion polypeptide comprises the amino acid sequence of SEQ ID NO: 161, and the second fusion polypeptide comprises the amino acid sequence of SEQ ID NO: 106.

In some embodiments according to any of the MSAPs described above, the MSAP comprises: i) an anti-CD19 Fab fragment, an anti-CD8 first scFv, and an anti-CD3 second scFv; or ii) an anti-CD19 Fab fragment, an anti-CD3 first scFv, and an anti-CD8 second scFv. In some embodiments, the MSAP comprises a first fusion polypeptide comprising an anti-CD3 first scFv fused to the C-terminus of the CH1 of the anti-CD19 Fab fragment via a first linker, and a second fusion polypeptide comprising an anti-CD8 second scFv fused to the N-terminus of the VL of the anti-CD19 Fab fragment via a second linker; and wherein the first fusion polypeptide comprises the amino acid sequence of SEQ ID NO: 170, and the second fusion polypeptide comprises the amino acid sequence of SEQ ID NO: 171. In some embodiments, the MSAP comprises a first fusion polypeptide comprising an anti-CD8 first scFv fused to the C-terminus of the CH1 of the anti-CD19 Fab fragment via a first linker, and a second fusion polypeptide comprising an anti-CD3 second scFv fused to the N-terminus of the VL of the anti-CD19 Fab fragment via a second linker; and wherein: i) the first fusion polypeptide comprises the amino acid sequence of SEQ ID NO: 172, and the second fusion polypeptide comprises the amino acid sequence of SEQ ID NO: 173; ii) the first fusion polypeptide comprises the amino acid sequence of SEQ ID NO: 174, and the second fusion polypeptide comprises the amino acid sequence of SEQ ID NO: 175; iii) the first fusion polypeptide comprises the amino acid sequence of SEQ ID NO: 172, and the second fusion polypeptide comprises the amino acid sequence of SEQ ID NO: 176; or iv) the first fusion polypeptide comprises the amino acid sequence of SEQ ID NO: 172, and the second fusion polypeptide comprises the amino acid sequence of SEQ ID NO: 182. In some embodiments, the MSAP comprises a first fusion polypeptide comprising an anti-CD3 first scFv fused to the N-terminus of the VH of the anti-CD19 Fab fragment via a first linker, and a second fusion polypeptide comprising an anti-CD8 second scFv fused to the C-terminus of the CL of the anti-CD19 Fab fragment via a second linker; and wherein the first fusion polypeptide comprises the amino acid sequence of SEQ ID NO: 177, and the second fusion polypeptide comprises the amino acid sequence of SEQ ID NO: 178. In some embodiments, the MSAP comprises: i) a fusion polypeptide comprising an anti-CD3 first scFv fused to the N-terminus of the VH of the anti-CD19 Fab fragment via a first linker, and an anti-CD8 second scFv fused to the C-terminus of the CH1 of the anti-CD19 Fab fragment via a second linker, and ii) the second polypeptide of the anti-CD19 Fab fragment; and wherein the fusion polypeptide comprises the amino acid sequence of SEQ ID NO: 180, and the second polypeptide of the anti-CD19 Fab fragment comprises the amino acid sequence of SEQ ID NO: 169. In some embodiments, the MSAP comprises: i) the first polypeptide of the anti-CD19 Fab fragment; and ii) a fusion polypeptide comprising an anti-CD3 first scFv fused to the N-terminus of the VL of the anti-CD19 Fab fragment via a first linker, and an anti-CD8 second scFv fused to the C-terminus of the CL of the anti-CD19 Fab fragment via a second linker; and wherein the first polypeptide of the anti-CD19 Fab fragment comprises the amino acid sequence of SEQ ID NO: 137, and the fusion polypeptide comprises the amino acid sequence of SEQ ID NO: 181.

Also provided are isolated nucleic acids encoding any of the MSAPs described herein, vectors (e.g., viral vector, such as lentiviral vector or AAV) comprising such nucleic acids, and host cells comprising such nucleic acids or vectors.

Also provided are methods of making any of the MSAPs described above, comprising: i) culturing a host cell comprising any of the isolated nucleic acids or the vectors described above, or any of the host cells described above, under a condition suitable for the expression of the MSAP; and ii) obtaining the expressed MSAP from said host cell.

Also provided are pharmaceutical compositions comprising: i) any of the MSAPs described herein, or isolated nucleic acids or vectors (e.g., viral vector, such as lentiviral vector or AAV) encoding thereof; and optionally ii) a pharmaceutically acceptable carrier.

Also provided are methods of treating a disease associated with a target epitope in an individual, comprising administering to the individual an effective amount of any of the MSAPs described above or any of the pharmaceutical compositions described above (e.g., viral vector pharmaceutical composition). In some embodiments, the disease associated with the target epitope is a cancer expressing the target epitope (e.g., CD19+ cancer, EpCAM+ cancer, or WT1+ cancer). In some embodiments, the cancer is a solid cancer or a liquid cancer. In some embodiments, the cancer is Burkitt lymphoma, Wilms' tumor, or colorectal cancer. In some embodiments, the MSAP or the pharmaceutical composition is administered subcutaneously or intravenously. In some embodiments, the individual is a human.

The present invention in another aspect provides anti-CD8 antibodies or antigen binding fragments thereof that specifically bind to CD8, wherein the anti-CD8 antibody or antigen binding fragment thereof comprises: an HVR-H1 comprising the amino acid sequence of any of SEQ ID NOs: 1, 7, 13, and 20, or a variant thereof comprising up to about 5 amino acid variations; an HVR-H2 comprising the amino acid sequence of any of SEQ ID NOs: 2, 8, 14, 16, and 18, or a variant thereof comprising up to about 5 amino acid variations; an HVR-H3 comprising the amino acid sequence of any of SEQ ID NOs: 3, 9, 15, 17, 19, and 22, or a variant thereof comprising up to about 5 amino acid variations; an HVR-L1 comprising the amino acid sequence of SEQ ID NO: 4 or 10, or a variant thereof comprising up to about 5 amino acid variations; an HVR-L2 comprising the amino acid sequence of SEQ ID NO: 5 or 11, or a variant thereof comprising up to about 5 amino acid variations; and an HVR-L3 comprising the amino acid sequence of any of SEQ ID NOs: 6, 12, and 23, or a variant thereof comprising up to about 5 amino acid variations. In some embodiments, the anti-CD8 antibody or antigen binding fragment thereof comprises: i) an HVR-H1 comprising the amino acid sequence of SEQ ID NO: 13, an HVR-H2 comprising the amino acid sequence of SEQ ID NO: 14, an HVR-H3 comprising the amino acid sequence of SEQ ID NO: 15, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; ii) an HVR-H1 comprising the amino acid sequence of SEQ ID NO: 1, an HVR-H2 comprising the amino acid sequence of SEQ ID NO: 16, an HVR-H3 comprising the amino acid sequence of SEQ ID NO: 3, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iii) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iv) an HVR-H1 comprising the amino acid sequence of SEQ ID NO: 1, an HVR-H2 comprising the amino acid sequence of SEQ ID NO: 18, an HVR-H3 comprising the amino acid sequence of SEQ ID NO: 19, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; v) an HVR-H1 comprising the amino acid sequence of SEQ ID NO: 7, an HVR-H2 comprising the amino acid sequence of SEQ ID NO: 8, an HVR-H3 comprising the amino acid sequence of SEQ ID NO: 9, an HVR-L1 comprising the amino acid sequence of SEQ ID NO: 10, an HVR-L2 comprising the amino acid sequence of SEQ ID NO: 11, and an HVR-L3 comprising the amino acid sequence of SEQ ID NO: 12; or vi) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23.

In some embodiments according to any of the anti-CD8 antibodies or antigen binding fragments thereof described above, the anti-CD8 antibody or antigen binding fragment thereof comprises: i) a VH comprising the amino acid sequence of SEQ ID NO: 24, or a variant thereof having at least about 80% sequence identity to the amino acid sequence of SEQ ID NO: 24, and a VL comprising the amino acid sequence of SEQ ID NO: 25, or a variant thereof having at least about 80% sequence identity to the amino acid sequence of SEQ ID NO: 25; ii) a VH comprising the amino acid sequence of SEQ ID NO: 26, or a variant thereof having at least about 80% sequence identity to the amino acid sequence of SEQ ID NO: 26, and a VL comprising the amino acid sequence of SEQ ID NO: 27, or a variant thereof having at least about 80% sequence identity to the amino acid sequence of SEQ ID NO: 27; iii) a VH comprising the amino acid sequence of SEQ ID NO: 28, or a variant thereof having at least about 80% sequence identity to the amino acid sequence of SEQ ID NO: 28, and a VL comprising the amino acid sequence of SEQ ID NO: 29, or a variant thereof having at least about 80% sequence identity to the amino acid sequence of SEQ ID NO: 29; iv) a VH comprising the amino acid sequence of SEQ ID NO: 30, or a variant thereof having at least about 80% sequence identity to the amino acid sequence of SEQ ID NO: 30, and a VL comprising the amino acid sequence of SEQ ID NO: 25, or a variant thereof having at least about 80% sequence identity to the amino acid sequence of SEQ ID NO: 25; v) a VH comprising the amino acid sequence of SEQ ID NO: 32, or a variant thereof having at least about 80% sequence identity to the amino acid sequence of SEQ ID NO: 32, and a VL comprising the amino acid sequence of SEQ ID NO: 33, or a variant thereof having at least about 80% sequence identity to the amino acid sequence of SEQ ID NO: 33; or vi) a VH comprising the amino acid sequence of SEQ ID NO: 34, or a variant thereof having at least about 80% sequence identity to the amino acid sequence of SEQ ID NO: 34, and a VL comprising the amino acid sequence of SEQ ID NO: 35, or a variant thereof having at least about 80% sequence identity to the amino acid sequence of SEQ ID NO: 35. In some embodiments, the anti-CD8 antibody or antigen binding fragment thereof comprises: i) a VH comprising the amino acid sequence of SEQ ID NO: 24, and a VL comprising the amino acid sequence of SEQ ID NO: 25; ii) a VH comprising the amino acid sequence of SEQ ID NO: 26, and a VL comprising the amino acid sequence of SEQ ID NO: 27; iii) a VH comprising the amino acid sequence of SEQ ID NO: 28, and a VL comprising the amino acid sequence of SEQ ID NO: 29; iv) a VH comprising the amino acid sequence of SEQ ID NO: 30, and a VL comprising the amino acid sequence of SEQ ID NO: 25; v) a VH comprising the amino acid sequence of SEQ ID NO: 32, and a VL comprising the amino acid sequence of SEQ ID NO: 33; or vi) a VH comprising the amino acid sequence of SEQ ID NO: 34, and a VL comprising the amino acid sequence of SEQ ID NO: 35.

In some embodiments according to any of the anti-CD8 antibodies or antigen binding fragments thereof described above, the anti-CD8 antibody or antigen binding fragment thereof is selected from the group consisting of a full-length antibody, a Fab, a Fab′, a Fab′-SH, a F(ab′)₂, an Fv, and an scFv. In some embodiments, the anti-CD8 antigen binding fragment is an anti-CD8 scFv. In some embodiments, the anti-CD8 scFv comprises the amino acid sequence of any of SEQ ID NOs: 21, 44-49, and 51-55. In some embodiments, the anti-CD8 antigen binding fragment is an anti-CD8 Fab fragment. In some embodiments, the anti-CD8 Fab fragment comprises: i) a first polypeptide comprising the amino acid sequence of SEQ ID NO: 147, and a second polypeptide comprising the amino acid sequence of SEQ ID NO: 148; ii) a first polypeptide comprising the amino acid sequence of SEQ ID NO: 149, and a second polypeptide comprising the amino acid sequence of SEQ ID NO: 150; iii) a first polypeptide comprising the amino acid sequence of SEQ ID NO: 91, and a second polypeptide comprising the amino acid sequence of SEQ ID NO: 92; iv) a first polypeptide comprising the amino acid sequence of SEQ ID NO: 151, and a second polypeptide comprising the amino acid sequence of SEQ ID NO: 148; v) a first polypeptide comprising the amino acid sequence of SEQ ID NO: 153, and a second polypeptide comprising the amino acid sequence of SEQ ID NO: 154; or vi) a first polypeptide comprising the amino acid sequence of SEQ ID NO: 155, and a second polypeptide comprising the amino acid sequence of SEQ ID NO: 156.

Also provided are MSAPs (e.g., bispecific antigen binding protein (BSAP), TSAP) comprising any of the anti-CD8 antibodies or antigen binding fragments thereof described above.

Also provided are isolated nucleic acids encoding any of the anti-CD8 antibodies or antigen binding fragments thereof or the MSAPs described above, vectors (e.g., viral vector, such as lentiviral vector or AAV) comprising such nucleic acids, and host cells comprising such nucleic acids or vectors.

Also provided are methods of making an anti-CD8 antibody or antigen binding fragment thereof, or an MSAP thereof, comprising: i) culturing a host cell comprising any of the isolated nucleic acids or vectors encoding anti-CD8 antibodies, antigen binding fragments thereof, or MSAPs described above, or any of the host cells encoding anti-CD8 antibodies, antigen binding fragments thereof, or MSAPs described above, under a condition suitable for the expression of the anti-CD8 antibody or antigen binding fragment thereof, or the MSAP thereof; and ii) obtaining the expressed anti-CD8 antibody or antigen binding fragment thereof, or the MSAP thereof, from said host cell.

Also provided are pharmaceutical compositions comprising: i) any of the anti-CD8 antibodies or antigen binding fragments thereof described above, or any of the MSAPs described above, or isolated nucleic acids or vectors (e.g., viral vector, such as lentiviral vector or AAV) encoding thereof; and optionally ii) a pharmaceutically acceptable carrier. Also provided are methods of treating a disease in an individual, comprising administering to the individual an effective amount of any of the MSAPs described above or any of the pharmaceutical compositions described above (e.g., viral vector pharmaceutical composition). In some embodiments, the disease (e.g., cancer) expresses a target epitope (e.g., CD19, EpCAM, or WT1) recognized by the MSAP.

The present invention in another aspect provides anti-CD3 antibodies or antigen binding fragments thereof that specifically bind to CD3, wherein the anti-CD3 antibody or antigen binding fragment thereof comprises: an HVR-H1 comprising the amino acid sequence of SEQ ID NO: 36, or a variant thereof comprising up to about 5 amino acid variations; an HVR-H2 comprising the amino acid sequence of any of SEQ ID NOs: 37, 157, and 166, or a variant thereof comprising up to about 5 amino acid variations; an HVR-H3 comprising the amino acid sequence of SEQ ID NO: 38 or 158, or a variant thereof comprising up to about 5 amino acid variations; an HVR-L1 comprising the amino acid sequence of SEQ ID NO: 39, or a variant thereof comprising up to about 5 amino acid variations; an HVR-L2 comprising the amino acid sequence of SEQ ID NO: 40, or a variant thereof comprising up to about 5 amino acid variations; and an HVR-L3 comprising the amino acid sequence of SEQ ID NO: 41, or a variant thereof comprising up to about 5 amino acid variations.

In some embodiments according to any of the anti-CD3 antibodies or antigen binding fragments thereof described above, the anti-CD3 antibody or antigen binding fragment thereof comprises: (a) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (b) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (c) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the anti-CD3 antibody or antigen binding fragment thereof comprises: (a) a VH comprising the amino acid sequence of SEQ ID NO: 42, or a variant thereof having at least about 80% sequence identity to the amino acid sequence of SEQ ID NO: 42, and a VL comprising the amino acid sequence of SEQ ID NO: 43, or a variant thereof having at least about 80% sequence identity to the amino acid sequence of SEQ ID NO: 43; (b) a VH comprising the amino acid sequence of SEQ ID NO: 159, or a variant thereof having at least about 80% sequence identity to the amino acid sequence of SEQ ID NO: 159, and a VL comprising the amino acid sequence of SEQ ID NO: 43, or a variant thereof having at least about 80% sequence identity to the amino acid sequence of SEQ ID NO: 43; or (c) a VH comprising the amino acid sequence of SEQ ID NO: 167, or a variant thereof having at least about 80% sequence identity to the amino acid sequence of SEQ ID NO: 167, and a VL comprising the amino acid sequence of SEQ ID NO: 43, or a variant thereof having at least about 80% sequence identity to the amino acid sequence of SEQ ID NO: 43. In some embodiments, the anti-CD3 antibody or antigen binding fragment thereof comprises: (a) a VH comprising the amino acid sequence of SEQ ID NO: 42, and a VL comprising the amino acid sequence of SEQ ID NO: 43; (b) a VH comprising the amino acid sequence of SEQ ID NO: 159, and a VL comprising the amino acid sequence of SEQ ID NO: 43; or (c) a VH comprising the amino acid sequence of SEQ ID NO: 167, and a VL comprising the amino acid sequence of SEQ ID NO: 43.

In some embodiments according to any of the anti-CD3 antibodies or antigen binding fragments thereof described above, the anti-CD3 antibody or antigen binding fragment thereof is selected from the group consisting of a full-length antibody, a Fab, a Fab′, a Fab′-SH, a F(ab′)₂, an Fv, and an scFv. In some embodiments, the anti-CD3 antigen binding fragment is an anti-CD3 scFv. In some embodiments, the anti-CD3 scFv comprises the amino acid sequence of any of SEQ ID NOs: 50, 56, 163, 164, 183, and 184. In some embodiments, the anti-CD3 antigen binding fragment is an anti-CD3 Fab fragment. In some embodiments, the anti-CD3 Fab fragment comprises: (a) a first polypeptide comprising the amino acid sequence of SEQ ID NO: 89, and a second polypeptide comprising the amino acid sequence of SEQ ID NO: 90 or 179; (b) a first polypeptide comprising the amino acid sequence of SEQ ID NO: 160, and a second polypeptide comprising the amino acid sequence of SEQ ID NO: 90 or 179; or (c) a first polypeptide comprising the amino acid sequence of SEQ ID NO: 168, and a second polypeptide comprising the amino acid sequence of SEQ ID NO: 90 or 179.

Also provided are MSAPs (e.g., BSAP, TSAP) comprising any of the anti-CD3 antibodies or antigen binding fragments thereof described above.

Also provided are isolated nucleic acids encoding any of the anti-CD3 antibodies or antigen binding fragments thereof, or the MSAPs described above, vectors (e.g., viral vector, such as lentiviral vector or AAV) comprising such nucleic acids, and host cells comprising such nucleic acids or vectors.

Also provided are methods of making any of the anti-CD3 antibodies or antigen binding fragments thereof, or MSAPs thereof, comprising: i) culturing a host cell comprising any of the isolated nucleic acids or vectors encoding anti-CD3 antibodies, antigen binding fragments thereof, or MSAPs described above, or any of the host cells encoding anti-CD3 antibodies, antigen binding fragments thereof, or MSAPs described above, under a condition suitable for the expression of the anti-CD3 antibody or antigen binding fragment thereof, or the MSAP thereof; and ii) obtaining the expressed anti-CD3 antibody or antigen binding fragment thereof, or the MSAP thereof, from said host cell.

Also provided are pharmaceutical compositions comprising: i) any of the anti-CD3 antibodies or antigen binding fragments thereof or the MSAPs described above, or isolated nucleic acids or vectors (e.g., viral vector, such as lentiviral vector or AAV) encoding thereof; and optionally ii) a pharmaceutically acceptable carrier. Also provided are methods of treating a disease in an individual, comprising administering to the individual an effective amount of any of the MSAPs described above or any of the pharmaceutical compositions described above (e.g., viral vector pharmaceutical composition). In some embodiments, the disease (e.g., cancer) expresses a target epitope (e.g., CD19, EpCAM, or WT1) recognized by the MSAP.

These and other aspects and advantages of the present invention will become apparent from the subsequent detailed description and the appended claims. It is to be understood that one, some, or all of the properties of the various embodiments described herein may be combined to form other embodiments of the present invention.

The disclosures of all publications, patents, patent applications and published patent applications referred to herein are hereby incorporated herein by reference in their entirety.

### BRIEF DESCRIPTION OF THE DRAWINGS

FIG. 1A depicts the structure of a first exemplary MSAP, e.g., TSAP construct 002. FIG. 1B depicts the structure of a second exemplary MSAP, e.g., TSAP construct 003. FIG. 1C depicts the structure of a third exemplary MSAP, e.g., TSAP construct 004.

FIGS. 2A-2B depict the EC₅₀ for exemplary EpCAM×CD8 BSAP constructs 007 through 012 (anti-EpCAM scFv fused to the N-terminus of anti-CD8 Fab) binding to human CD8α (FIG. 2A) or cynomolgus monkey CD8α (FIG. 2B) in an ELISA binding assay.

FIG. 3 depicts a representative panel of flow cytometry data of the binding of an exemplary WT1×CD8×CD3 TSAP construct 001 (FIG. 1A structure) to CD8+ and to CD4+ T cells. FSC, forward scatter; SSC, side scatter.

FIG. 4 depicts the binding curve of the exemplary WT1×CD8×CD3 TSAP construct 001 (see FIG. 1A structure) to CD8+ and to CD4+ T cells. Conc., concentration.

FIG. 5 depicts a representative panel of flow cytometry data of the binding of the exemplary CD19×CD8×CD3 TSAP constructs 005 and 006 (FIG. 1A structure) to CD8+ and to CD4+ T cells compared to the corresponding BSAP control construct 014 (anti-CD3 scFv fused to the N-terminus of anti-CD19 Fab). FSC, forward scatter; SSC, side scatter.

FIG. 6 depicts the binding curves of BSAP control construct 014 and of exemplary CD19×CD8×CD3 TSAP constructs 005 and 006 (FIG. 1A structure) to CD8+ and to CD4+ T cells. Conc., concentration.

FIG. 7 depicts the specific cytotoxicity of target SW620 cancer cells by T cells that were incubated with either the exemplary WT1×CD8×CD3 TSAP construct 001 (FIG. 1A structure) or the corresponding BSAP control construct 013 (anti-WT1 scFv fused to the N-terminus of anti-CD3 Fab). Conc., concentration.

FIG. 8 depicts the specific cytotoxicity of target Raji cancer cells by T cells that were incubated with either the exemplary CD19×CD8×CD3 TSAP construct 006 (FIG. 1A structure) or the corresponding BSAP control construct 014 (anti-CD3 scFv fused to the N-terminus of anti-CD19 Fab). Conc., concentration.

FIGS. 9A-9B show the CD4+ T cell activation level (FIG. 9A) and the CD8+ T cell activation level (FIG. 9B) of T cells co-cultured with Raji cells by the degree of CD69+ staining via flow cytometry for CD4+ T cells that were incubated with either the exemplary CD19×CD8×CD3 TSAP construct 006 (FIG. 1A structure) or the corresponding BSAP control construct 014 (anti-CD3 scFv fused to the N-terminus of anti-CD19 Fab). Conc., concentration.

FIGS. 9C-9F compare the T cell activation levels of CD4+ T cells versus CD8+ T cells treated with either BSAP control construct 014 or TSAP construct 006. FIGS. 9C-9D show the CD4+ T cell activation level (FIG. 9C) and the CD8+ T cell activation level (FIG. 9D) of human PBMCs by the degree of CD69+ staining via flow cytometry for CD4+ T cells or CD8+ T cells that were incubated with either the exemplary CD19×CD8×CD3 TSAP construct 006 (FIG. 1A structure) or the corresponding BSAP control construct 014 (anti-CD3 scFv fused to the N-terminus of anti-CD19 Fab). FIGS. 9E-9F show the T cell activation of CD4+ T cells versus CD8+ T cells as indicated by levels of the T cell activation marker CD69 when the T cells have been incubated with either BSAP control construct 014 (FIG. 9E) or TSAP construct 006 (FIG. 9F).Conc., concentration; hPBMCs, human peripheral blood mononuclear cells.

FIGS. 10A-10B show the degree of TNFα production in CD4+ T cells (FIG. 10A) and CD8+ T cells (FIG. 10B) by flow cytometry for CD4+ T cells that were incubated with either the exemplary CD19×CD8×CD3 TSAP construct 006 (FIG. 1A structure) or the corresponding BSAP control construct 014 (anti-CD3 scFv fused to the N-terminus of anti-CD19 Fab). SSC, side scatter.

FIGS. 11A-11B show the degree of IFNγ production in CD4+ T cells (FIG. 11A) and CD8+ T cells (FIG. 11B) by flow cytometry for CD4+ T cells that were incubated with either the exemplary CD19×CD8×CD3 TSAP construct 006 (FIG. 1A structure) or the corresponding BSAP control construct 014 (anti-CD3 scFv fused to the N-terminus of anti-CD19 Fab). SSC, side scatter.

FIGS. 12A-12B show the degree of IL-2 production in CD4+ T cells (FIG. 12A) and CD8+ T cells (FIG. 12B) by flow cytometry for CD4+ T cells that were incubated with either the exemplary CD19×CD8×CD3 TSAP construct 006 (FIG. 1A structure) or the corresponding BSAP control construct 014 (anti-CD3 scFv fused to the N-terminus of anti-CD19 Fab). SSC, side scatter.

FIGS. 13A-13C show the EC₅₀ curves for TNFα production in CD4+ T cells (FIG. 13A) and CD8+ T cells (FIG. 13B) that were incubated with either the exemplary CD19×CD8×CD3 Fab TSAP constructs 005 and 006 (FIG. 1A structure) or the corresponding BSAP control construct 014 (anti-CD3 scFv fused to the N-terminus of anti-CD19 Fab), or a comparison of TNFα production in CD4+ T cells versus CD8+ T cells treated with either the exemplary CD19×CD8×CD3 Fab TSAP construct 006 or the corresponding CD19 Fab×CD3 BSAP control construct 014 (FIG. 13C).

FIGS. 14A-14C show the EC₅₀ curves for IFNγ production in CD4+ T cells (FIG. 14A) and CD8+ T cells (FIG. 14B) that were incubated with either the exemplary CD19×CD8×CD3 Fab TSAP con constructs 005 and 006 (FIG. 1A structure) or the corresponding BSAP control construct 014 (anti-CD3 scFv fused to the N-terminus of anti-CD19 Fab), or a comparison of IFNγ production in CD4+ T cells versus CD8+ T cells treated with either the exemplary CD19×CD8×CD3 Fab TSAP construct 006 or the corresponding CD19 Fab×CD3 BSAP control construct 014 (FIG. 14C).

FIGS. 15A-15C show the EC₅₀ curves for IL-2 production in CD4+ T cells (FIG. 15A) and CD8+ T cells (FIG. 15B) that were incubated with either the exemplary CD19×CD8×CD3 Fab TSAP constructs 005 and 006 (FIG. 1A structure) or the corresponding BSAP control construct 014 (anti-CD3 scFv fused to the N-terminus of anti-CD19 Fab), or a comparison of IL-2 production in CD4+ T cells versus CD8+ T cells treated with either the exemplary CD19×CD8×CD3 Fab TSAP construct 006 or the corresponding CD19 Fab×CD3 BSAP control construct 014 (FIG. 15C).

FIGS. 16A-16C show the EC₅₀ curves for the secretion of pro-inflammatory cytokines IL-6 (FIG. 16A), IL-2 (FIG. 16B), and IFNγ (FIG. 16C) in human PBMCs that were incubated with either the exemplary CD19×CD8×CD3 Fab TSAP construct 015 (FIG. 1A structure) or the corresponding CD19 Fab×CD3 BSAP control construct 014.

FIGS. 17A-17B show the levels of B cells circulating in the peripheral blood of cynomolgus monkeys after treatment with the exemplary CD19×CD8×CD3 Fab TSAP construct 015 (FIG. 1A structure), or the corresponding CD19 Fab×CD3 BSAP control construct 014 by either a single dose of 2-hr intravenous infusion (FIG. 17A) or multiple doses of subcutaneous injections (S.C.) given at hours 0, 48, and 96 (FIG. 17B).

FIGS. 18A-18D show the levels of CD4+ T cell (FIGS. 18A and 18C) or CD8+ T cell (FIGS. 18B and 18D) redistribution and recovery from the peripheral blood of cynomolgus monkeys after treatment with the exemplary CD19×CD8×CD3 Fab TSAP construct 015 (FIG. 1A structure) or the corresponding CD19 Fab×CD3 BSAP control construct 014 by either a single I.V. dose (FIGS. 18A-18B) or multiple S.C. doses given at hours 0, 48, and 96 (FIGS. 18C-18D).

FIGS. 19A-19E show the levels of secreted pro-inflammatory cytokines circulating in the peripheral blood of cynomolgus monkeys after treatment with vehicle (PBS), the exemplary CD19×CD8×CD3 Fab TSAP construct 015 (FIG. 1A structure) or the corresponding CD19 Fab×CD3 BSAP control construct 014. FIGS. 19A-19B compare the levels of IL-6 production in cynomolgus monkeys treated with CD19 Fab×CD3 BSAP 014 by 30 μg/kg I.V. (n=3) or 150 μg/kg S.C. (n=3) (FIG. 19A) or with CD19×CD8×CD3 Fab TSAP 015 by 40 μg/kg I.V. (n=3) or 400 μg/kg S.C. (n=6) (FIG. 19B). FIGS. 19C-19E show the levels of IL-2 production (FIG. 19C), IFNγ production (FIG. 19D), or TNFα production (FIG. 19E) in cynomolgus monkeys treated with CD19×CD8×CD3 Fab TSAP 015 by 40 μg/kg I.V. (n=3) or 400 μg/kg S.C. (n=6).

FIG. 20 shows the serum concentration of CD19×CD8×CD3 Fab TSAP 015 over time when administered by S.C. of 400 μg/kg TSAP 015 (N=5) or I.V. of 40 μg/kg TSAP 015 (N=3) in cynomolgus monkeys.

FIGS. 21A-21E depict additional trispecific T cell engager formats. FIG. 21A shows the structure of a fourth exemplary MSAP, e.g., TSAP construct 017. FIG. 21B shows the structure of a fifth exemplary MSAP, e.g., TSAP constructs 018, 019, 020, and 022. FIG. 21C shows the structure of a sixth exemplary MSAP, e.g., TSAP construct 021. FIG. 21D shows the structure of a seventh exemplary MSAP, e.g., TSAP construct 023. FIG. 21E shows the structure of an eighth exemplary MSAP, e.g., TSAP construct 024.

FIGS. 22A-22G show the binding of exemplary CD19×CD8×CD3 TSAP constructs (formats depicted in FIGS. 21A-21E) to CD4+ T cells and CD8+ T cells: TSAP construct 017 (FIG. 22A), TSAP construct 018 (FIG. 22B), TSAP construct 019 (FIG. 22C), TSAP construct 020 (FIG. 22D), TSAP construct 021 (FIG. 22E), TSAP construct 023 (FIG. 22F), and TSAP construct 024 (FIG. 22G). Conc., concentration.

FIGS. 23A-23D show the specific cytotoxicity against target CD20+ B cells and CD8+ T cells by T cells that were incubated with the exemplary CD19×CD8×CD3 TSAP constructs (formats depicted in FIGS. 21A-21B): TSAP construct 017 (FIG. 23A), TSAP construct 018 (FIG. 23B), TSAP construct 019 (FIG. 23C), and TSAP construct 020 (FIG. 23D).

FIGS. 24A-24I show the level of T cell activation of CD4+ T cells and of CD8+ T cells as indicated by levels of the T cell activation marker CD69 when the T cells have been incubated with either BSAP control construct 014 (FIG. 24A) or exemplary CD19×CD8×CD3 TSAP constructs (formats depicted in FIGS. 21A-21E): TSAP construct 017 (FIG. 24B), TSAP construct 018 (FIG. 24C), TSAP construct 019 (FIG. 24D), TSAP construct 020 (FIG. 24E), TSAP construct 021 (FIG. 24F), TSAP construct 022 (FIG. 24G), TSAP construct 023 (FIG. 24H), and TSAP construct 024 (FIG. 24I). Conc., concentration.

FIGS. 25A-25B show the level of CD4+ T cell proliferation and CD8+ T cell proliferation as assessed by the level of Ki-67, a nuclear protein associated with cell cycle, after incubation with TSAP construct 020. FIG. 25A depicts a representative panel of flow cytometry data of human PBMCs stained for CD8+ T cells, CD4+ T cells, and Ki-67 as a proliferation marker. FIG. 25B shows the percentage of CD8+ T cells and CD4+ T cells that are Ki-67+. Graphpad Prism software was used to fit the dose-response curve and calculate the corresponding EC₅₀ values. SSC, side scatter; FSC, forward scatter.

FIG. 26 shows the tumor growth inhibitory effect of the CD19×CD8×CD3 TSAP construct 020 against Raji xenograft tumors that were subcutaneously implanted into immune-reconstructed NOG mice inoculated with human PBMCs. Vehicle injection (PBST) served as negative control. SC, subcutaneous; N, number of mice.

FIGS. 27A-27B show the average number per mg tissue of human CD4+ T cells (FIG. 27A) and human CD8+ T cells (FIG. 27B) that were present within the spleens of the immune-reconstructed NOG mice inoculated with human PBMCs and subcutaneously implanted with Raji xenograft tumors on Day 28 post-Raji implantation. TSAP construct 020 was administered at the indicated dosage. Vehicle injection (PBST) served as negative control.

### DETAILED DESCRIPTION OF THE INVENTION

After extensive investigation, inventors of the present application discovered that the targetxCD8×CD3 MSAP (such as TSAP) format described herein has several unexpected advantages compared to other multispecific proteins. First, the targetxCD8×CD3 MSAPs described herein have enhanced cytotoxic activities against target (e.g., cancer) cells, for example, CD19-positive Raji Burkitt lymphoma cancer cells and WT1-positive SW620 colorectal cancer cells. Second, the targetxCD8×CD3 MSAPs described herein, by employing a Fab fragment, may have extended half-life that enables lower dosing frequency and shorter infusion time, providing more convenience to the patients. Third, the targetxCD8×CD3 MSAPs described herein significantly increased CD8+ T cell activation and target-antigen directed binding, did not induce cytotoxicity against CD8+ T cells, and minimized side effects such as cytokine storm induced by CD4+ T cell activation, such as compared to a target×CD3×BSAP or TSAP (not comprising an anti-CD8 moiety) not differentiating between CD4+ and CD8+ T cell binding. Fourth, the target×CD8×CD3 MSAPs described herein have cross-reactivity to both human and cynomolgus monkey CD8, which can facilitate extrapolation of toxicity and efficacy study results from cynomolgus monkeys to human clinical studies. Fifth, the target×CD8×CD3 MSAPs described herein showed in vivo clearance of target cells (e.g., CD19+ B cells) in cynomolgus monkeys as well as rapid CD8+ T cell redistribution and recovery that is indicative of CD8+ T cell activation in vivo. Sixth, the target×CD8×CD3 MSAPs described herein showed stable pharmacokinetic properties upon both subcutaneous (S.C.) and intravenous (I.V.) administration to cynomolgus monkeys. Seventh, the target×CD8×CD3 MSAPs described herein showed comparable in vivo efficacy upon S.C. or I.V. administration, and further surprisingly demonstrated that S.C. administration had improved safety compared to BSAP control based on the in vivo production and systemic circulation of pro-inflammatory cytokines (e.g., IL-6, TNFα) that can induce cytokine release syndrome or cytokine storm. These surprising effects were demonstrated for MSAP of various structures, sequences, and target antigens tested. Taken together, these findings demonstrate the unique pharmacokinetic and pharmacodynamic properties of the target×CD8×CD3 MSAPs described herein and their improved therapeutic efficacy compared to bispecific antibodies (i.e., BSAPs).

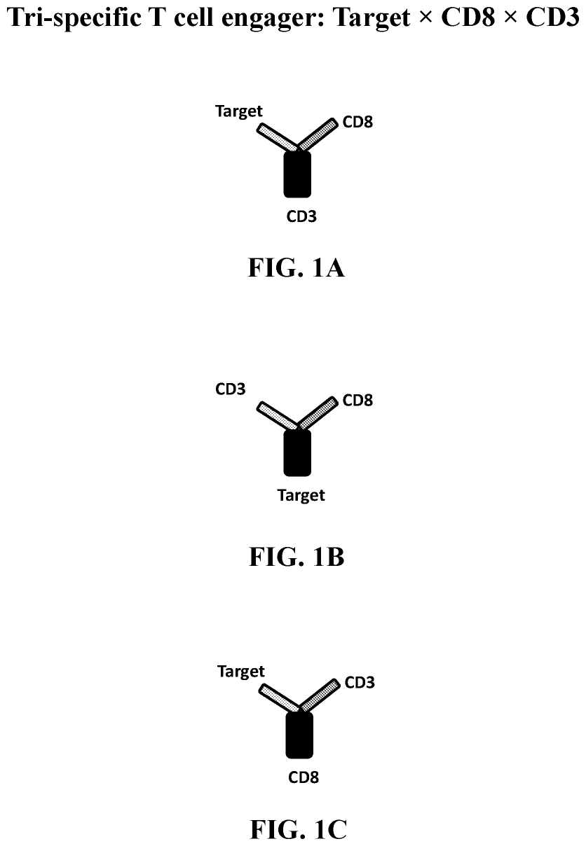

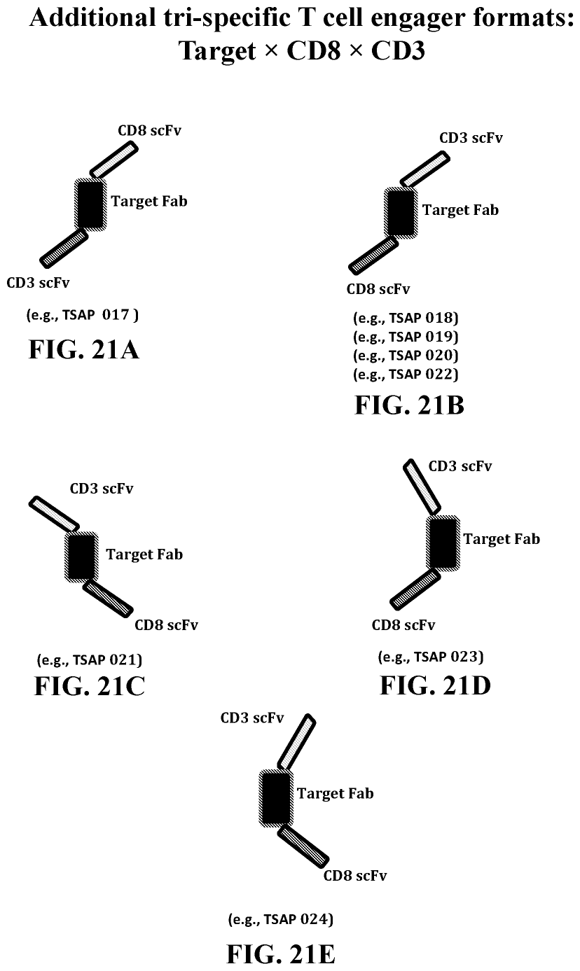

Accordingly, in one aspect, the present invention provides an MSAP (e.g., TSAP) comprising: i) an anti-CD3 moiety that specifically binds to CD3; ii) an anti-CD8 moiety that specifically binds to CD8; and iii) an anti-target epitope moiety that specifically binds to a target epitope (e.g., TSA or TAA, such as CD19, EpCAM, or WT1), wherein a first moiety of the three moieties is a Fab fragment, wherein the Fab fragment comprises a first polypeptide comprising a VH and a CH1, and a second polypeptide comprising a VL and a CL; wherein a second moiety of the three moieties is a first antigen binding fragment (e.g., scFv); wherein a third moiety of the three moieties is a second antigen binding fragment (e.g., scFv); and wherein: 1) the first antigen binding fragment is fused to the N-terminus of the VH of the Fab fragment, and the second antigen binding fragment is fused to the N-terminus of the VL of the Fab fragment; 2) the first antigen binding fragment is fused to the C-terminus of the CH1 of the Fab fragment, and the second antigen binding fragment is fused to the N-terminus of the VL of the Fab fragment; 3) the first antigen binding fragment is fused to the N-terminus of the VH of the Fab fragment, and the second antigen binding fragment is fused to the C-terminus of the CL of the Fab fragment; 4) the first antigen binding fragment is fused to the N-terminus of the VH of the Fab fragment, and the second antigen binding fragment is fused to the C-terminus of the CH1 of the Fab fragment; or 5) the first antigen binding fragment is fused to the N-terminus of the VL of the Fab fragment, and the second antigen binding fragment is fused to the C-terminus of the CL of the Fab fragment. In some embodiments, a) the anti-CD3 moiety is the Fab fragment (“anti-CD3 Fab fragment”), the anti-CD8 moiety is the first antigen binding fragment (“anti-CD8 first antigen binding fragment”), and the anti-target epitope moiety is the second antigen binding fragment (“anti-target epitope second antigen binding fragment”); b) the anti-CD3 moiety is the Fab fragment, the anti-target epitope moiety is the first antigen binding fragment (“anti-target epitope first antigen binding fragment”), and the anti-CD8 moiety is the second antigen binding fragment (“anti-CD8 second antigen binding fragment”); c) the anti-CD8 moiety is the Fab fragment (“anti-CD8 Fab fragment”), the anti-CD3 moiety is the first antigen binding fragment (“anti-CD3 first antigen binding fragment”), and the anti-target epitope moiety is the second antigen binding fragment; d) the anti-CD8 moiety is the Fab fragment, the anti-target epitope moiety is the first antigen binding fragment, and the anti-CD3 moiety is the second antigen binding fragment (“anti-CD3 second antigen binding fragment”); e) the anti-target epitope moiety is the Fab fragment (“anti-target epitope Fab fragment”), the anti-CD3 moiety is the first antigen binding fragment, and the anti-CD8 moiety is the second antigen binding fragment; or f) the anti-target epitope moiety is the Fab fragment, the anti-CD8 moiety is the first antigen binding fragment, and the anti-CD3 moiety is the second antigen binding fragment. In some embodiments, the anti-target epitope moiety specifically recognizes a target epitope of a TAA or a TSA. In some embodiments, the TAA is CD19 or EpCAM. In some embodiments, the TSA is WT1. In some embodiments, the first antigen binding fragment and the second antigen binding fragment are both scFvs. Exemplary MSAPs are shown in FIGS. 1A-1C and 21A-21E.

In another aspect, the present invention provides novel anti-CD8 antibodies or antigen-binding fragments thereof, and novel anti-CD3 antibodies or antigen-binding fragments thereof. MSAPs (e.g., BSAP, TSAP) comprising thereof are also provided.

Also provided are pharmaceutical compositions and kits comprising any of the target×CD8×CD3 MSAPs (such as TSAPs), anti-CD8 antibodies or antigen-binding fragments thereof (or MSAP thereof), or anti-CD3 antibodies or antigen-binding fragments thereof (or MSAP thereof) described herein. Further provided are methods of use of the MSAPs or pharmaceutical compositions thereof, such as for treating cancers, e.g., CD19-positive, EpCAM-positive, or WT1-positive cancers.

### I. Definitions

The practice of the present invention will employ, unless indicated specifically to the contrary, conventional methods of virology, immunology, microbiology, molecular biology, and recombinant DNA techniques within the skill of the art, many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3rd ed., John Wiley & Sons, 1995; Sambrook and Russell, Molecular Cloning: A Laboratory Manual (3rd Edition, 2001); Maniatis et al., Molecular Cloning: A Laboratory Manual (1982); DNA Cloning: A Practical Approach, vol. I&II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, an “effective amount” refers to an amount of an agent or drug effective to treat a disease or disorder in a subject. In the case of cancer, the effective amount of the agent may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, an “individual” or a “subject” refers to a mammal, including, but not limited to, human, bovine, horse, feline, canine, rodent, or primate. In some embodiments, the individual is a human.

The term “antibody” is used in the broadest sense and specifically covers monoclonal antibodies (including full length monoclonal antibodies), multispecific antibodies (e.g., bispecific antibodies), and antibody fragments so long as they exhibit the desired biological activity or function. As used herein, the terms “immunoglobulin” (Ig) and “antibody” are used interchangeably.

The terms “native antibody,” “full length antibody,” “intact antibody,” and “whole antibody” are used herein interchangeably to refer to an antibody in its substantially intact form, not antibody fragments as defined below. The terms particularly refer to an antibody with heavy chains that contain an Fc region. Native antibodies are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the C_{H}1, C_{H}2 and C_{H}3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs, also referred to as CDRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“κ”) and lambda (“λ”), based on the amino acid sequences of their constant domains.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called a, 8, ¿, Y, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

“Antibody fragments” comprise a portion of an intact antibody, preferably comprising the antigen binding region thereof. In some embodiments, the antibody fragment described herein is an antigen binding fragment. Examples of antibody fragments or antigen binding fragments include Fab, Fab′, F(ab′)₂, and Fv fragments (such as single-chain variable fragment, scFv); diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment has two polypeptide chains, containing the heavy- and light-chain variable domains (VH, VL), and also containing the constant domain of the light chain (CL) and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, The Pharmacology of Monoclonal Antibodies. Springer Berlin Heidelberg, 1994. 269-315.

The “Fc” fragment comprises the carboxy-terminal portions of both heavy chains held together by di-sulfides. The effector functions of antibodies are determined by sequences in the Fc region, which region is also the part recognized by Fc receptors (FcR) found on certain types of cells.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In some embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14(3): 253-260 (1995), Harlow et al., Antibodies: A Laboratory Manual, (Cold Spring Harbor Laboratory Press, 2nd ed. 1988); Hammerling et al., Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature 352:624-628 (1991); Marks et al., J. Mol. Biol. 222:581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995)).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol. 227:381 (1991); Marks et al., J. Mol. Biol. 222:581 (1991). Also available for the preparation of human monoclonal antibodies are methods described in Cole et al., Monoclonal Antibodies and Cancer Therapy, Alan R. Liss, 77 (1985); Boerner et al., J. Immunol. 147(1): 86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996). HVR is also referred to as “CDR” or “complementarity determining region”.

The structures and locations of immunoglobulin variable regions may be determined by reference to Kabat, E. A. et al., Sequences of Proteins of Immunological Interest. 4th Edition. US Department of Health and Human Services. 1987, and updates thereof, now available on the Internet (immuno.bme.nwu.edu).

“Framework” or “FR” residues are those variable domain residues other than the HVR residues as herein defined.

The term “covalently linked” as used herein, refers to a direct linkage through one or more chemical bonds or an indirect linkage through one or more linkers. Any suitable chemical bond can be used to create a direct linkage, including but not limited to, a covalent bond such as a peptide bond and a disulfide bond, or a non-covalent bond such as a hydrogen bond, a hydrophobic bond, an ionic bond, or a van der Waals bond.

“Covalent bond” as used herein refers to a stable bond between two atoms sharing one or more electrons. Examples of covalent bonds include, but are not limited to, peptide bonds and disulfide bonds. As used herein, “peptide bond” refers to a covalent bond formed between a carboxyl group of an amino acid and an amine group of an adjacent amino acid. A “disulfide bond” as used herein refers to a covalent bond formed between two sulfur atoms, such as a combination of a heavy chain fragment CH1 and a light chain fragment CL by one or more disulfide bonds. One or more disulfide bonds may be formed between the two fragments by linking the thiol groups in the two fragments. In some embodiments, one or more disulfide bonds can be formed between one or more cysteines of the heavy chain fragment and the light chain fragment, respectively. Disulfide bonds can be formed by oxidation of two thiol groups. In some embodiments, the covalent linkage is directly linked by a covalent bond. In some embodiments, the covalent linkage is directly linked by a peptide bond or a disulfide bond.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In some embodiments, an antibody that specifically binds to a target has a dissociation constant (K_{d}) of ≤1 μM, ≤100 nM, ≤10 nM, ≤1 nM, or ≤0.1 nM. In some embodiments, an antibody specifically binds to an epitope on a protein that is conserved among the protein from different species. In another embodiment, specific binding can include, but does not require exclusive binding.

As used herein, “Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but is not limited to the replacement of one amino acid in a polypeptide with another amino acid. Exemplary substitutions are shown in Table 1. Amino acid substitutions may be introduced into an antibody of interest and the products screened for a desired activity, e.g., retained/improved antigen binding, decreased immunogenicity, or improved ADCC or CDC.

TABLE 1
Exemplary amino acid substitutions.

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

The term “multispecific” as used in conjunction with an antibody or antigen binding protein (such as a multispecific antigen binding protein, MSAP) refers to an antibody or antigen binding protein having polyepitopic specificity (i.e., is capable of specifically binding to two, three, or more, different epitopes on one biological molecule or is capable of specifically binding to epitopes on two, three, or more, different biological molecules). Unless otherwise indicated, the order in which the antigens bound by a multispecific antibody or MSAP are listed in a multispecific antibody or MSAP name is arbitrary. That is, the terms may be used interchangeably to refer to multispecific antibodies (such as MSAPs) that specifically bind to CD3, CD8, and a target epitope (e.g., a specific TSA or TAA (e.g., CD19, EpCAM, or WT1)). In some embodiments, an MSAP is a TSAP.

The term “bispecific” as used in conjunction with an antibody or antigen binding protein (such as a bispecific antigen binding protein, BSAP) refers to an antibody or antigen binding protein capable of specifically binding to two different epitopes on one biological molecule, or capable of specifically binding to epitopes on two different biological molecules. Unless otherwise indicated, the order in which the antigens bound by a bispecific antibody or BSAP are listed in a bispecific antibody or BSAP name is arbitrary. In some embodiments, the BSAP is bivalent. In some embodiments, the BSAP is multivalent (e.g., trivalent).

The term “trispecific” as used in conjunction with an antibody or antigen binding protein (such as a trispecific antigen binding protein, TSAP) refers to an antibody or antigen binding protein capable of specifically binding to three different epitopes on one biological molecule, or capable of specifically binding to epitopes on three different biological molecules. Unless otherwise indicated, the order in which the antigens bound by a trispecific antibody or TSAP are listed in a trispecific antibody or TSAP name is arbitrary. That is, the terms may be used interchangeably to refer to trispecific antibodies (such as TSAP) that specifically bind to CD3, CD8, and a target epitope (e.g., a specific TSA or TAA (e.g., CD19, EpCAM, or WT1)), for example, any of the terms “TAA (TSA)×CD3×CD8,” “TAA (TSA)×CD8×CD3,” “CD3×CD8×TAA (TSA),” “CD8×CD3×TAA (TSA),” “CD8×TAA (TSA)×CD3”, and “CD3×TAA (TSA)×CD8”. In some embodiments, the TSAP is bivalent. In some embodiments, the TSAP is multivalent (e.g., trivalent).

As used herein, the “C terminus” of a polypeptide refers to the last amino acid residue of the polypeptide which donates its amine group to form a peptide bond with the carboxyl group of its adjacent amino acid residue. “N terminus” of a polypeptide as used herein refers to the first amino acid of the polypeptide which donates its carboxyl group to form a peptide bond with the amine group of its adjacent amino acid residue.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “cell” includes the primary subject cell and its progeny.

The term “cytokine storm,” also known as a “cytokine cascade” or “hypercytokinemia,” is a potentially fatal immune reaction typically consisting of a positive feedback loop between cytokines and immune cells, with highly elevated levels of various cytokines (e.g., INF-γ, IL-10, IL-6, CCL2, etc.).

It will be understood by one of ordinary skill in the art that uracil and thymine can both be represented by ‘t’, instead of ‘u’ for uracil and ‘t’ for thymine; in the context of a ribonucleic acid, it will be understood that ‘t’ is used to represent uracil unless otherwise indicated.

It is understood that embodiments of the invention described herein include “consisting of” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Multispecific Antigen Binding Proteins (MSAPs)

In one aspect, the present invention provides a multispecific antigen binding protein (MSAP) comprising: i) an anti-CD3 moiety that specifically binds to CD3; ii) an anti-CD8 moiety that specifically binds to CD8; and iii) an anti-target epitope moiety that specifically binds to a target epitope (e.g., TAA or TSA, such as CD19, WT1, or EpCAM); wherein a first moiety of the three moieties is a Fab fragment, wherein the Fab fragment comprises a first polypeptide comprising a VH and a CH1, and a second polypeptide comprising a VL and a CL; wherein a second moiety of the three moieties is a first antigen binding fragment (e.g., scFv); and wherein a third moiety of the three moieties is a second antigen binding fragment (e.g., scFv). In some embodiments, the first antigen binding fragment is fused to the N-terminus of the VH of the Fab fragment, and the second antigen binding fragment is fused to the N-terminus of the VL of the Fab fragment. In some embodiments, the first antigen binding fragment is fused to the N-terminus of the VL of the Fab fragment, and the second antigen binding fragment is fused to the N-terminus of the VH of the Fab fragment. In some embodiments, the first antigen binding fragment is fused to the C-terminus of the CH1 of the Fab fragment, and the second antigen binding fragment is fused to the C-terminus of the CL of the Fab fragment. In some embodiments, the first antigen binding fragment is fused to the C-terminus of the CL of the Fab fragment, and the second antigen binding fragment is fused to the C-terminus of the CH1 of the Fab fragment. In some embodiments, the first antigen binding fragment is fused to the N-terminus of the VH of the Fab fragment, and the second antigen binding fragment is fused to the C-terminus of the CL of the Fab fragment. In some embodiments, the first antigen binding fragment is fused to the C-terminus of the CL of the Fab fragment, and the second antigen binding fragment is fused to the N-terminus of the VH of the Fab fragment. In some embodiments, the first antigen binding fragment is fused to the C-terminus of the CH1 of the Fab fragment, and the second antigen binding fragment is fused to the N-terminus of the VL of the Fab fragment. In some embodiments, the first antigen binding fragment is fused to the N-terminus of the VL of the Fab fragment, and the second antigen binding fragment is fused to the C-terminus of the CH1 of the Fab fragment. In some embodiments, the first antigen binding fragment is fused to the N-terminus of the VH of the Fab fragment, and the second antigen binding fragment is fused to the C-terminus of the CH1 of the Fab fragment. In some embodiments, the first antigen binding fragment is fused to the C-terminus of the CH1 of the Fab fragment, and the second antigen binding fragment is fused to the N-terminus of the VH of the Fab fragment. In some embodiments, the first antigen binding fragment is fused to the N-terminus of the VL of the Fab fragment, and the second antigen binding fragment is fused to the C-terminus of the CL of the Fab fragment. In some embodiments, the first antigen binding fragment is fused to the C-terminus of the CL of the Fab fragment, and the second antigen binding fragment is fused to the N-terminus of the VL of the Fab fragment. In some embodiments, the MSAP comprises two or more first antigen binding fragments (e.g., scFv), and/or two or more second antigen binding fragments (e.g., scFv). In some embodiments, the two or more first antigen binding fragments (e.g., scFv) are fused with each other in tandem, then fused to the Fab fragment (e.g., N′ of VH, N′ of VL, C′ of CH1, and/or C′ of CL). In some embodiments, the two or more second antigen binding fragments (e.g., scFv) are fused with each other in tandem, then fused to the Fab fragment (e.g., N′ of VH, N′ of VL, C′ of CH1, and/or C′ of CL). In some embodiments, the two or more first antigen binding fragments (e.g., scFv) are fused to the N-terminus of the Fab fragment (e.g., one at N′ of VH, and/or another one at N′ of VL). In some embodiments, the two or more second antigen binding fragments (e.g., scFv) are fused to the C-terminus of the Fab fragment (e.g., one at C′ of CH1, and/or another one at C′ of CL). In some embodiments, the two or more second antigen binding fragments (e.g., scFv) are fused to the N-terminus of the Fab fragment (e.g., one at N′ of VH, and/or another one at N′ of VL). In some embodiments, the two or more first antigen binding fragments (e.g., scFv) are fused to the C-terminus of the Fab fragment (e.g., one at C′ of CH1, and/or another one at C′ of CL).

In some embodiments, there is provided an MSAP comprising: i) an anti-CD3 moiety that specifically binds to CD3; ii) an anti-CD8 moiety that specifically binds to CD8; and iii) an anti-target epitope moiety that specifically binds to a target epitope (e.g., TAA or TSA, such as CD19, WT1, or EpCAM), wherein a first moiety of the three moieties is a Fab fragment, wherein the Fab fragment comprises a first polypeptide comprising a VH and a CH1, and a second polypeptide comprising a VL and a CL; wherein a second moiety of the three moieties is a first antigen binding fragment (e.g., scFv); and wherein a third moiety of the three moieties is a second antigen binding fragment (e.g., scFv); and wherein: 1) the first antigen binding fragment is fused to the N-terminus of the VH of the Fab fragment, and the second antigen binding fragment is fused to the N-terminus of the VL of the Fab fragment; 2) the first antigen binding fragment is fused to the C-terminus of the CH1 of the Fab fragment, and the second antigen binding fragment is fused to the N-terminus of the VL of the Fab fragment; 3) the first antigen binding fragment is fused to the N-terminus of the VH of the Fab fragment, and the second antigen binding fragment is fused to the C-terminus of the CL of the Fab fragment; 4) the first antigen binding fragment is fused to the N-terminus of the VH of the Fab fragment, and the second antigen binding fragment is fused to the C-terminus of the CH1 of the Fab fragment; or 5) the first antigen binding fragment is fused to the N-terminus of the VL of the Fab fragment, and the second antigen binding fragment is fused to the C-terminus of the CL of the Fab fragment. In some embodiments, a) the anti-CD3 moiety is the Fab fragment (“anti-CD3 Fab fragment”), the anti-CD8 moiety is the first antigen binding fragment (“anti-CD8 first antigen binding fragment”), and the anti-target epitope moiety is the second antigen binding fragment (“anti-target epitope second antigen binding fragment”); b) the anti-CD3 moiety is the Fab fragment, the anti-target epitope moiety is the first antigen binding fragment (“anti-target epitope first antigen binding fragment”), and the anti-CD8 moiety is the second antigen binding fragment (“anti-CD8 second antigen binding fragment”); c) the anti-CD8 moiety is the Fab fragment (“anti-CD8 Fab fragment”), the anti-CD3 moiety is the first antigen binding fragment (“anti-CD3 first antigen binding fragment”), and the anti-target epitope moiety is the second antigen binding fragment; d) the anti-CD8 moiety is the Fab fragment, the anti-target epitope moiety is the first antigen binding fragment, and the anti-CD3 moiety is the second antigen binding fragment (“anti-CD3 second antigen binding fragment”); e) the anti-target epitope moiety is the Fab fragment (“anti-target epitope Fab fragment”), the anti-CD3 moiety is the first antigen binding fragment, and the anti-CD8 moiety is the second antigen binding fragment; or f) the anti-target epitope moiety is the Fab fragment, the anti-CD8 moiety is the first antigen binding fragment, and the anti-CD3 moiety is the second antigen binding fragment. In some embodiments, the first antigen binding fragment and the second antigen binding fragment are independently selected from the group consisting of Fab, Fab′, Fab′-SH, F(ab′)2, Fv, sdAb, scFv, and any combination thereof. In some embodiments, the first antigen binding fragment and the second antigen binding fragment are both scFv. In some embodiments, the CH1 comprises the amino acid sequence of SEQ ID NO: 93, and wherein the CL comprises the amino acid sequence of SEQ ID NO: 94 or 165. In some embodiments, the first antigen binding fragment is fused to the Fab fragment via an optional first linker, and/or the second antigen binding fragment is fused to the Fab fragment via an optional second linker. In some embodiments, the first linker and/or the second linker comprises an amino acid sequence independently selected from the group consisting of GG and SEQ ID NOs: 108-110. In some embodiments, the anti-CD8 moiety comprises: i) an HVR-H1 comprising the amino acid sequence of SEQ ID NO: 13, an HVR-H2 comprising the amino acid sequence of SEQ ID NO: 14, an HVR-H3 comprising the amino acid sequence of SEQ ID NO: 15, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; ii) an HVR-H1 comprising the amino acid sequence of SEQ ID NO: 1, an HVR-H2 comprising the amino acid sequence of SEQ ID NO: 16, an HVR-H3 comprising the amino acid sequence of SEQ ID NO: 3, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iii) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iv) an HVR-H1 comprising the amino acid sequence of SEQ ID NO: 1, an HVR-H2 comprising the amino acid sequence of SEQ ID NO: 18, an HVR-H3 comprising the amino acid sequence of SEQ ID NO: 19, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; v) an HVR-H1 comprising the amino acid sequence of SEQ ID NO: 7, an HVR-H2 comprising the amino acid sequence of SEQ ID NO: 8, an HVR-H3 comprising the amino acid sequence of SEQ ID NO: 9, an HVR-L1 comprising the amino acid sequence of SEQ ID NO: 10, an HVR-L2 comprising the amino acid sequence of SEQ ID NO: 11, and an HVR-L3 comprising the amino acid sequence of SEQ ID NO: 12; or vi) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23. In some embodiments, the anti-CD8 moiety comprises: i) a VH comprising the amino acid sequence of SEQ ID NO: 24, and a VL comprising the amino acid sequence of SEQ ID NO: 25; ii) a VH comprising the amino acid sequence of SEQ ID NO: 26, and a VL comprising the amino acid sequence of SEQ ID NO: 27; iii) a VH comprising the amino acid sequence of SEQ ID NO: 28, and a VL comprising the amino acid sequence of SEQ ID NO: 29; iv) a VH comprising the amino acid sequence of SEQ ID NO: 30, and a VL comprising the amino acid sequence of SEQ ID NO: 25; v) a VH comprising the amino acid sequence of SEQ ID NO: 32, and a VL comprising the amino acid sequence of SEQ ID NO: 33; or vi) a VH comprising the amino acid sequence of SEQ ID NO: 34, and a VL comprising the amino acid sequence of SEQ ID NO: 35. In some embodiments, the anti-CD8 moiety is an anti-CD8 scFv, wherein the anti-CD8 scFv comprises the amino acid sequence of any one of SEQ ID NOs: 21, 44-49, and 51-55, such as SEQ ID NO: 46, 49, 52, or 55. In some embodiments, the anti-CD8 moiety is an anti-CD8 Fab fragment, wherein the anti-CD8 Fab fragment comprises: i) a first polypeptide comprising the amino acid sequence of SEQ ID NO: 147, and a second polypeptide comprising the amino acid sequence of SEQ ID NO: 148; ii) a first polypeptide comprising the amino acid sequence of SEQ ID NO: 149, and a second polypeptide comprising the amino acid sequence of SEQ ID NO: 150; iii) a first polypeptide comprising the amino acid sequence of SEQ ID NO: 91, and a second polypeptide comprising the amino acid sequence of SEQ ID NO: 92; iv) a first polypeptide comprising the amino acid sequence of SEQ ID NO: 151, and a second polypeptide comprising the amino acid sequence of SEQ ID NO: 148; v) a first polypeptide comprising the amino acid sequence of SEQ ID NO: 153, and a second polypeptide comprising the amino acid sequence of SEQ ID NO: 154; or vi) a first polypeptide comprising the amino acid sequence of SEQ ID NO: 155, and a second polypeptide comprising the amino acid sequence of SEQ ID NO: 156. In some embodiments, the anti-CD3 moiety comprises: (a) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (b) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (c) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the anti-CD3 moiety comprises: (a) a VH comprising the amino acid sequence of SEQ ID NO: 42, and a VL comprising the amino acid sequence of SEQ ID NO: 43; (b) a VH comprising the amino acid sequence of SEQ ID NO: 159, and a VL comprising the amino acid sequence of SEQ ID NO: 43; or (c) a VH comprising the amino acid sequence of SEQ ID NO: 167, and a VL comprising the amino acid sequence of SEQ ID NO: 43. In some embodiments, the anti-CD3 moiety is an anti-CD3 scFv, wherein the anti-CD3 scFv comprises the amino acid sequence of any of SEQ ID NOs: 50, 56, 163, 164, 183, and 184. In some embodiments, the anti-CD3 moiety is an anti-CD3 Fab fragment, wherein the anti-CD3 Fab fragment comprises: (i) a first polypeptide comprising the amino acid sequence of SEQ ID NO: 89, and a second polypeptide comprising the amino acid sequence of SEQ ID NO: 90 or 179; (ii) a first polypeptide comprising the amino acid sequence of SEQ ID NO: 160, and a second polypeptide comprising the amino acid sequence of SEQ ID NO: 90 or 179; or (iii) a first polypeptide comprising the amino acid sequence of SEQ ID NO: 168, and a second polypeptide comprising the amino acid sequence of SEQ ID NO: 90 or 179. In some embodiments, the anti-target epitope moiety specifically recognizes CD19 (anti-CD19 moiety). In some embodiments, the anti-CD19 moiety comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD19 moiety comprises a VH comprising the amino acid sequence of SEQ ID NO: 63, and a VL comprising the amino acid sequence of SEQ ID NO: 64. In some embodiments, the anti-CD19 moiety is an anti-CD19 scFv, wherein the anti-CD19 scFv comprises the amino acid sequence of SEQ ID NO: 65 or 66. In some embodiments, the anti-CD19 moiety is an anti-CD19 Fab fragment, wherein the anti-CD19 Fab fragment comprises a first polypeptide comprising the amino acid sequence of SEQ ID NO: 137, and a second polypeptide comprising the amino acid sequence of SEQ ID NO: 139 or 169. In some embodiments, the anti-target epitope moiety specifically recognizes EpCAM (anti-EpCAM moiety). In some embodiments, the anti-EpCAM moiety comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, the anti-EpCAM moiety comprises a VH comprising the amino acid sequence of SEQ ID NO: 73, and a VL comprising the amino acid sequence of SEQ ID NO: 74. In some embodiments, the anti-EpCAM moiety is an anti-EpCAM scFv, wherein the anti-EpCAM scFv comprises the amino acid sequence of SEQ ID NO: 75 or 78. In some embodiments, the anti-EpCAM moiety is an anti-EpCAM Fab fragment, wherein the anti-EpCAM Fab fragment comprises a first polypeptide comprising the amino acid sequence of SEQ ID NO: 76, and a second polypeptide comprising the amino acid sequence of SEQ ID NO: 77. In some embodiments, the anti-target epitope moiety specifically recognizes WT1 (anti-WT1 moiety). In some embodiments, the anti-WT1 moiety comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the anti-WT1 moiety comprises a VH comprising the amino acid sequence of SEQ ID NO: 85, and a VL comprising the amino acid sequence of SEQ ID NO: 86. In some embodiments, the anti-WT1 moiety is an anti-WT1 scFv, wherein the anti-WT1 scFv comprises the amino acid sequence of SEQ ID NO: 87 or 88. In some embodiments, the anti-WT1 moiety is an anti-WT1 Fab fragment, wherein the anti-WT1 Fab fragment comprises a first polypeptide comprising the amino acid sequence of SEQ ID NO: 145, and a second polypeptide comprising the amino acid sequence of SEQ ID NO: 146. In some embodiments, the MSAP is trivalent and trispecific, hereinafter referred to as “TSAP” or, wherein the anti-target epitope moiety recognizes a target epitope of a TAA or TSA, then referred to as “TAA (TSA)×CD8×CD3 TSAP.” Exemplary TAA (TSA)×CD8×CD3 MSAPs (e.g., TSAPs) are shown in FIGS. 1A-1C and 21A-21E and Examples.

In some embodiments, there is provided an MSAP (e.g., TSAP) comprising: i) an anti-CD3 Fab fragment, ii) an anti-CD8 first antigen binding fragment (e.g., scFv), and iii) an anti-target epitope (e.g., TAA or TSA, such as CD19, WT1, or EpCAM) second antigen binding fragment (e.g., scFv); wherein the anti-CD3 Fab fragment comprises a first polypeptide comprising a VH and a CH1, and a second polypeptide comprising a VL and a CL; wherein the anti-CD8 first antigen binding fragment is fused to the N-terminus of the VH of the anti-CD3 Fab fragment via an optional first linker; and wherein the anti-target epitope second antigen binding fragment is fused to the N-terminus of the VL of the anti-CD3 Fab fragment via an optional second linker. In some embodiments, the anti-CD8 first antigen binding fragment and the anti-target epitope second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD3 Fab fragment, ii) an anti-target epitope (e.g., TAA or TSA, such as CD19, WT1, or EpCAM) first antigen binding fragment (e.g., scFv), and iii) an anti-CD8 second antigen binding fragment (e.g., scFv); wherein the anti-CD3 Fab fragment comprises a first polypeptide comprising a VH and a CH1, and a second polypeptide comprising a VL and a CL; wherein the anti-target epitope first antigen binding fragment is fused to the N-terminus of the VH of the anti-CD3 Fab fragment via an optional first linker; and wherein the anti-CD8 second antigen binding fragment is fused to the N-terminus of the VL of the anti-CD3 Fab fragment via an optional second linker. In some embodiments, the anti-target epitope first antigen binding fragment and the anti-CD8 second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD8 Fab fragment, ii) an anti-CD3 first antigen binding fragment (e.g., scFv), and iii) an anti-target epitope (e.g., TAA or TSA, such as CD19, WT1, or EpCAM) second antigen binding fragment (e.g., scFv); wherein the anti-CD8 Fab fragment comprises a first polypeptide comprising a VH and a CH1, and a second polypeptide comprising a VL and a CL; wherein the anti-CD3 first antigen binding fragment is fused to the N-terminus of the VH of the anti-CD8 Fab fragment via an optional first linker; and wherein the anti-target epitope second antigen binding fragment is fused to the N-terminus of the VL of the anti-CD8 Fab fragment via an optional second linker. In some embodiments, the anti-CD3 first antigen binding fragment and the anti-target epitope second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD8 Fab fragment, ii) an anti-target epitope (e.g., TAA or TSA, such as CD19, WT1, or EpCAM) first antigen binding fragment (e.g., scFv), and iii) an anti-CD3 second antigen binding fragment (e.g., scFv); wherein the anti-CD8 Fab fragment comprises a first polypeptide comprising a VH and a CH1, and a second polypeptide comprising a VL and a CL; wherein the anti-target epitope first antigen binding fragment is fused to the N-terminus of the VH of the anti-CD8 Fab fragment via an optional first linker; and wherein the anti-CD3 second antigen binding fragment is fused to the N-terminus of the VL of the anti-CD8 Fab fragment via an optional second linker. In some embodiments, the anti-target epitope first antigen binding fragment and the anti-CD3 second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-target epitope (e.g., TAA or TSA, such as CD19, WT1, or EpCAM) Fab fragment, ii) an anti-CD3 first antigen binding fragment (e.g., scFv), and iii) an anti-CD8 second antigen binding fragment (e.g., scFv); wherein the anti-target epitope Fab fragment comprises a first polypeptide comprising a VH and a CH1, and a second polypeptide comprising a VL and a CL; wherein the anti-CD3 first antigen binding fragment is fused to the N-terminus of the VH of the anti-target epitope Fab fragment via an optional first linker; and wherein the anti-CD8 second antigen binding fragment is fused to the N-terminus of the VL of the anti-target epitope Fab fragment via an optional second linker. In some embodiments, the anti-CD3 first antigen binding fragment and the anti-CD8 second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-target epitope Fab fragment, ii) an anti-CD8 first antigen binding fragment (e.g., scFv), and iii) an anti-CD3 second antigen binding fragment (e.g., scFv); wherein the anti-target epitope (e.g., TAA or TSA, such as CD19, WT1, or EpCAM) Fab fragment comprises a first polypeptide comprising a VH and a CH1, and a second polypeptide comprising a VL and a CL; wherein the anti-CD8 first antigen binding fragment is fused to the N-terminus of the VH of the anti-target epitope Fab fragment via an optional first linker; and wherein the anti-CD3 second antigen binding fragment is fused to the N-terminus of the VL of the anti-target epitope Fab fragment via an optional second linker. In some embodiments, the anti-CD8 first antigen binding fragment and the anti-CD3 second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-target epitope (e.g., TAA or TSA, such as CD19, WT1, or EpCAM) Fab fragment, ii) an anti-CD3 first antigen binding fragment (e.g., scFv), and iii) an anti-CD8 second antigen binding fragment (e.g., scFv); wherein the anti-target epitope Fab fragment comprises a first polypeptide comprising a VH and a CH1, and a second polypeptide comprising a VL and a CL; and wherein the anti-CD3 first antigen binding fragment is fused to the C-terminus of the CH1 of the anti-target epitope Fab fragment via an optional first linker, and the anti-CD8 second antigen binding fragment is fused to the N-terminus of the VL of the anti-target epitope Fab fragment via an optional second linker. In some embodiments, the anti-CD3 first antigen binding fragment and the anti-CD8 second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-target epitope (e.g., TAA or TSA, such as CD19, WT1, or EpCAM) Fab fragment, ii) an anti-CD8 first antigen binding fragment (e.g., scFv), and iii) an anti-CD3 second antigen binding fragment (e.g., scFv); wherein the anti-target epitope Fab fragment comprises a first polypeptide comprising a VH and a CH1, and a second polypeptide comprising a VL and a CL; and wherein the anti-CD8 first antigen binding fragment is fused to the C-terminus of the CH1 of the anti-target epitope Fab fragment via an optional first linker, and the anti-CD3 second antigen binding fragment is fused to the N-terminus of the VL of the anti-target epitope Fab fragment via an optional second linker. In some embodiments, the anti-CD8 first antigen binding fragment and the anti-CD3 second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD3 Fab fragment; ii) an anti-CD8 first antigen binding fragment (e.g., scFv); and iii) an anti-target epitope (e.g., TAA or TSA, such as CD19, WT1, or EpCAM) second antigen binding fragment (e.g., scFv); wherein the anti-CD3 Fab fragment comprises a first polypeptide comprising a VH and a CH1, and a second polypeptide comprising a VL and a CL; and wherein the anti-CD8 first antigen binding fragment is fused to the C-terminus of the CH1 of the anti-CD3 Fab fragment via an optional first linker, and the anti-target epitope second antigen binding fragment is fused to the N-terminus of the VL of the anti-CD3 Fab fragment via an optional second linker. In some embodiments, the anti-CD8 first antigen binding fragment and the anti-target epitope second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD3 Fab fragment; ii) an anti-target epitope (e.g., TAA or TSA, such as CD19, WT1, or EpCAM) first antigen binding fragment (e.g., scFv); and iii) an anti-CD8 second antigen binding fragment (e.g., scFv); wherein the anti-CD3 Fab fragment comprises a first polypeptide comprising a VH and a CH1, and a second polypeptide comprising a VL and a CL; and wherein the anti-target epitope first antigen binding fragment is fused to the C-terminus of the CH1 of the anti-CD3 Fab fragment via an optional first linker, and the anti-CD8 second antigen binding fragment is fused to the N-terminus of the VL of the anti-CD3 Fab fragment via an optional second linker. In some embodiments, the anti-target epitope first antigen binding fragment and the anti-CD8 second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD8 Fab fragment, ii) an anti-CD3 first antigen binding fragment (e.g., scFv), and iii) an anti-target epitope (e.g., TAA or TSA, such as CD19, WT1, or EpCAM) second antigen binding fragment (e.g., scFv); wherein the anti-CD8 Fab fragment comprises a first polypeptide comprising a VH and a CH1, and a second polypeptide comprising a VL and a CL; and wherein the anti-CD3 first antigen binding fragment is fused to the C-terminus of the CH1 of the anti-CD8 Fab fragment via an optional first linker, and the anti-target epitope second antigen binding fragment is fused to the N-terminus of the VL of the anti-CD8 Fab fragment via an optional second linker. In some embodiments, the anti-CD3 first antigen binding fragment and the anti-target epitope second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD8 Fab fragment, ii) an anti-target epitope (e.g., TAA or TSA, such as CD19, WT1, or EpCAM) first antigen binding fragment (e.g., scFv), and iii) an anti-CD3 second antigen binding fragment (e.g., scFv); wherein the anti-CD8 Fab fragment comprises a first polypeptide comprising a VH and a CH1, and a second polypeptide comprising a VL and a CL; and wherein the anti-target epitope first antigen binding fragment is fused to the C-terminus of the CH1 of the anti-CD8 Fab fragment via an optional first linker, and the anti-CD3 second antigen binding fragment is fused to the N-terminus of the VL of the anti-CD8 Fab fragment via an optional second linker. In some embodiments, the anti-CD3 second antigen binding fragment and the anti-target epitope first antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-target epitope (e.g., TAA or TSA, such as CD19, WT1, or EpCAM) Fab fragment, ii) an anti-CD3 first antigen binding fragment (e.g., scFv), and iii) an anti-CD8 second antigen binding fragment (e.g., scFv); wherein the anti-target epitope Fab fragment comprises a first polypeptide comprising a VH and a CH1, and a second polypeptide comprising a VL and a CL; and wherein the anti-CD3 first antigen binding fragment is fused to the N-terminus of the VH of the anti-target epitope Fab fragment via an optional first linker, and the anti-CD8 second antigen binding fragment is fused to the C-terminus of the CL of the anti-target epitope Fab fragment via an optional second linker. In some embodiments, the anti-CD3 first antigen binding fragment and the anti-CD8 second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-target epitope (e.g., TAA or TSA, such as CD19, WT1, or EpCAM) Fab fragment, ii) an anti-CD8 first antigen binding fragment (e.g., scFv), and iii) an anti-CD3 second antigen binding fragment (e.g., scFv); wherein the anti-target epitope Fab fragment comprises a first polypeptide comprising a VH and a CH1, and a second polypeptide comprising a VL and a CL; and wherein the anti-CD8 first antigen binding fragment is fused to the N-terminus of the VH of the anti-target epitope Fab fragment via an optional first linker, and the anti-CD3 second antigen binding fragment is fused to the C-terminus of the CL of the anti-target epitope Fab fragment via an optional second linker. In some embodiments, the anti-CD8 first antigen binding fragment and the anti-CD3 second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD3 Fab fragment; ii) an anti-CD8 first antigen binding fragment (e.g., scFv); and iii) an anti-target epitope (e.g., TAA or TSA, such as CD19, WT1, or EpCAM) second antigen binding fragment (e.g., scFv); wherein the anti-CD3 Fab fragment comprises a first polypeptide comprising a VH and a CH1, and a second polypeptide comprising a VL and a CL; and wherein the anti-CD8 first antigen binding fragment is fused to the N-terminus of the VH of the anti-CD3 Fab fragment via an optional first linker, and the anti-target epitope second antigen binding fragment is fused to the C-terminus of the CL of the anti-CD3 Fab fragment via an optional second linker. In some embodiments, the anti-CD8 first antigen binding fragment and the anti-target epitope second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD3 Fab fragment; ii) an anti-target epitope (e.g., TAA or TSA, such as CD19, WT1, or EpCAM) first antigen binding fragment (e.g., scFv); and iii) an anti-CD8 second antigen binding fragment (e.g., scFv); wherein the anti-CD3 Fab fragment comprises a first polypeptide comprising a VH and a CH1, and a second polypeptide comprising a VL and a CL; and wherein the anti-target epitope first antigen binding fragment is fused to the N-terminus of the VH of the anti-CD3 Fab fragment via an optional first linker, and the anti-CD8 second antigen binding fragment is fused to the C-terminus of the CL of the anti-CD3 Fab fragment via an optional second linker. In some embodiments, the anti-CD8 second antigen binding fragment and the anti-target epitope first antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD8 Fab fragment, ii) an anti-CD3 first antigen binding fragment (e.g., scFv), and iii) an anti-target epitope (e.g., TAA or TSA, such as CD19, WT1, or EpCAM) second antigen binding fragment (e.g., scFv); wherein the anti-CD8 Fab fragment comprises a first polypeptide comprising a VH and a CH1, and a second polypeptide comprising a VL and a CL; and wherein the anti-CD3 first antigen binding fragment is fused to the N-terminus of the VH of the anti-CD8 Fab fragment via an optional first linker, and the anti-target epitope second antigen binding fragment is fused to the C-terminus of the CL of the anti-CD8 Fab fragment via an optional second linker. In some embodiments, the anti-CD3 first antigen binding fragment and the anti-target epitope second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD8 Fab fragment, ii) an anti-target epitope (e.g., TAA or TSA, such as CD19, WT1, or EpCAM) first antigen binding fragment (e.g., scFv), and iii) an anti-CD3 second antigen binding fragment (e.g., scFv); wherein the anti-CD8 Fab fragment comprises a first polypeptide comprising a VH and a CH1, and a second polypeptide comprising a VL and a CL; and wherein the anti-target epitope first antigen binding fragment is fused to the N-terminus of the VH of the anti-CD8 Fab fragment via an optional first linker, and the anti-CD3 second antigen binding fragment is fused to the C-terminus of the CL of the anti-CD8 Fab fragment via an optional second linker. In some embodiments, the anti-CD3 second antigen binding fragment and the anti-target epitope first antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-target epitope (e.g., TAA or TSA, such as CD19, WT1, or EpCAM) Fab fragment, ii) an anti-CD3 first antigen binding fragment (e.g., scFv), and iii) an anti-CD8 second antigen binding fragment (e.g., scFv); wherein the anti-target epitope Fab fragment comprises a first polypeptide comprising a VH and a CH1, and a second polypeptide comprising a VL and a CL; and wherein the anti-CD3 first antigen binding fragment is fused to the N-terminus of the VH of the anti-target epitope Fab fragment via an optional first linker, and the anti-CD8 second antigen binding fragment is fused to the C-terminus of the CH1 of the anti-target epitope Fab fragment via an optional second linker. In some embodiments, the anti-CD3 first antigen binding fragment and the anti-CD8 second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-target epitope (e.g., TAA or TSA, such as CD19, WT1, or EpCAM) Fab fragment, ii) an anti-CD8 first antigen binding fragment (e.g., scFv), and iii) an anti-CD3 second antigen binding fragment (e.g., scFv); wherein the anti-target epitope Fab fragment comprises a first polypeptide comprising a VH and a CH1, and a second polypeptide comprising a VL and a CL; and wherein the anti-CD8 first antigen binding fragment is fused to the N-terminus of the VH of the anti-target epitope Fab fragment via an optional first linker, and the anti-CD3 second antigen binding fragment is fused to the C-terminus of the CH1 of the anti-target epitope Fab fragment via an optional second linker. In some embodiments, the anti-CD8 first antigen binding fragment and the anti-CD3 second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD3 Fab fragment, ii) an anti-CD8 first antigen binding fragment (e.g., scFv), and iii) an anti-target epitope (e.g., TAA or TSA, such as CD19, WT1, or EpCAM) second antigen binding fragment (e.g., scFv); wherein the anti-CD3 Fab fragment comprises a first polypeptide comprising a VH and a CH1, and a second polypeptide comprising a VL and a CL; and wherein the anti-CD8 first antigen binding fragment is fused to the N-terminus of the VH of the anti-CD3 Fab fragment via an optional first linker, and the anti-target epitope second antigen binding fragment is fused to the C-terminus of the CH1 of the anti-CD3 Fab fragment via an optional second linker. In some embodiments, the anti-CD8 first antigen binding fragment and the anti-target epitope second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD3 Fab fragment; ii) an anti-target epitope (e.g., TAA or TSA, such as CD19, WT1, or EpCAM) first antigen binding fragment (e.g., scFv); and iii) an anti-CD8 second antigen binding fragment (e.g., scFv); wherein the anti-CD3 Fab fragment comprises a first polypeptide comprising a VH and a CH1, and a second polypeptide comprising a VL and a CL; and wherein the anti-target epitope first antigen binding fragment is fused to the N-terminus of the VH of the anti-CD3 Fab fragment via an optional first linker, and the anti-CD8 second antigen binding fragment is fused to the C-terminus of the CH1 of the anti-CD3 Fab fragment via an optional second linker. In some embodiments, the anti-CD8 second antigen binding fragment and the anti-target epitope first antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD8 Fab fragment, ii) an anti-CD3 first antigen binding fragment (e.g., scFv), and iii) an anti-target epitope (e.g., TAA or TSA, such as CD19, WT1, or EpCAM) second antigen binding fragment (e.g., scFv); wherein the anti-CD8 Fab fragment comprises a first polypeptide comprising a VH and a CH1, and a second polypeptide comprising a VL and a CL; and wherein the anti-CD3 first antigen binding fragment is fused to the N-terminus of the VH of the anti-CD8 Fab fragment via an optional first linker, and the anti-target epitope second antigen binding fragment is fused to the C-terminus of the CH1 of the anti-CD8 Fab fragment via an optional second linker. In some embodiments, the anti-CD3 first antigen binding fragment and the anti-target epitope second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD8 Fab fragment, ii) an anti-target epitope (e.g., TAA or TSA, such as CD19, WT1, or EpCAM) first antigen binding fragment (e.g., scFv), and iii) an anti-CD3 second antigen binding fragment (e.g., scFv); wherein the anti-CD8 Fab fragment comprises a first polypeptide comprising a VH and a CH1, and a second polypeptide comprising a VL and a CL; and wherein the an anti-target epitope first antigen binding fragment is fused to the N-terminus of the VH of the anti-CD8 Fab fragment via an optional first linker, and the anti-CD3 second antigen binding fragment is fused to the C-terminus of the CH1 of the anti-CD8 Fab fragment via an optional second linker. In some embodiments, the anti-CD3 second antigen binding fragment and the anti-target epitope first antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-target epitope (e.g., TAA or TSA, such as CD19, WT1, or EpCAM) Fab fragment, ii) an anti-CD3 first antigen binding fragment (e.g., scFv), and iii) an anti-CD8 second antigen binding fragment (e.g., scFv); wherein the anti-target epitope Fab fragment comprises a first polypeptide comprising a VH and a CH1, and a second polypeptide comprising a VL and a CL; and wherein the anti-CD3 first antigen binding fragment is fused to the N-terminus of the VL of the anti-target epitope Fab fragment via an optional first linker, and the anti-CD8 second antigen binding fragment is fused to the C-terminus of the CL of the anti-target epitope Fab fragment via an optional second linker. In some embodiments, the anti-CD3 first antigen binding fragment and the anti-CD8 second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-target epitope (e.g., TAA or TSA, such as CD19, WT1, or EpCAM) Fab fragment, ii) an anti-CD8 first antigen binding fragment (e.g., scFv), and iii) an anti-CD3 second antigen binding fragment (e.g., scFv); wherein the anti-target epitope Fab fragment comprises a first polypeptide comprising a VH and a CH1, and a second polypeptide comprising a VL and a CL; and wherein the anti-CD8 first antigen binding fragment is fused to the N-terminus of the VL of the anti-target epitope Fab fragment via an optional first linker, and the anti-CD3 second antigen binding fragment is fused to the C-terminus of the CL of the anti-target epitope Fab fragment via an optional second linker. In some embodiments, the anti-CD8 first antigen binding fragment and the anti-CD3 second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD3 Fab fragment; ii) an anti-CD8 first antigen binding fragment (e.g., scFv); and iii) an anti-target epitope (e.g., TAA or TSA, such as CD19, WT1, or EpCAM) second antigen binding fragment (e.g., scFv); wherein the anti-CD3 Fab fragment comprises a first polypeptide comprising a VH and a CH1, and a second polypeptide comprising a VL and a CL; and wherein the anti-CD8 first antigen binding fragment is fused to the N-terminus of the VL of the anti-CD3 Fab fragment via an optional first linker, and the anti-target epitope second antigen binding fragment is fused to the C-terminus of the CL of the anti-CD3 Fab fragment via an optional second linker. In some embodiments, the anti-CD8 first antigen binding fragment and the anti-target epitope second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD3 Fab fragment; ii) an anti-target epitope (e.g., TAA or TSA, such as CD19, WT1, or EpCAM) first antigen binding fragment (e.g., scFv); and iii) an anti-CD8 second antigen binding fragment (e.g., scFv); wherein the anti-CD3 Fab fragment comprises a first polypeptide comprising a VH and a CH1, and a second polypeptide comprising a VL and a CL; and wherein the anti-target epitope first antigen binding fragment is fused to the N-terminus of the VL of the anti-CD3 Fab fragment via an optional first linker, and the anti-CD8 second antigen binding fragment is fused to the C-terminus of the CL of the anti-CD3 Fab fragment via an optional second linker. In some embodiments, the anti-CD8 second antigen binding fragment and the anti-target epitope first antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD8 Fab fragment, ii) an anti-CD3 first antigen binding fragment (e.g., scFv), and iii) an anti-target epitope (e.g., TAA or TSA, such as CD19, WT1, or EpCAM) second antigen binding fragment (e.g., scFv); wherein the anti-CD8 Fab fragment comprises a first polypeptide comprising a VH and a CH1, and a second polypeptide comprising a VL and a CL; and wherein the anti-CD3 first antigen binding fragment is fused to the N-terminus of the VL of the anti-CD8 Fab fragment via an optional first linker, and the anti-target epitope second antigen binding fragment is fused to the C-terminus of the CL of the anti-CD8 Fab fragment via an optional second linker. In some embodiments, the anti-CD3 first antigen binding fragment and the anti-target epitope second antigen binding fragment are both scFv. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: i) an anti-CD8 Fab fragment, ii) an anti-target epitope (e.g., TAA or TSA, such as CD19, WT1, or EpCAM) first antigen binding fragment (e.g., scFv), and iii) an anti-CD3 second antigen binding fragment (e.g., scFv); wherein the anti-CD8 Fab fragment comprises a first polypeptide comprising a VH and a CH1, and a second polypeptide comprising a VL and a CL; and wherein the anti-target epitope first antigen binding fragment is fused to the N-terminus of the VL of the anti-CD8 Fab fragment via an optional first linker, and the anti-CD3 second antigen binding fragment is fused to the C-terminus of the CL of the anti-CD8 Fab fragment via an optional second linker. In some embodiments, the anti-CD3 second antigen binding fragment and the anti-target epitope first antigen binding fragment are both scFv. In some embodiments, the first linker and/or the second linker comprises an amino acid sequence independently selected from the group consisting of GG and SEQ ID NOs: 108-110.

In some embodiments, the MSAP (or TSAP) comprises a first fusion polypeptide comprising the structure: N′-anti-target epitope (e.g., TAA or TSA) VH-optional L1-anti-target epitope VL-optional L2-anti-CD3 VH-CH1-C′, and a second fusion polypeptide comprising the structure: N′-anti-CD8 VH-optional L3-anti-CD8 VL-optional L4-anti-CD 3 VL-CL-C′. In some embodiments, the MSAP comprises a first fusion polypeptide comprising the structure: N′-anti-target epitope (e.g., TAA or TSA) VH-optional L1-anti-target epitope VL-optional L2-anti-CD3 VH-CH1-C′, and a second fusion polypeptide comprising the structure: N′-anti-CD8 VL-optional L3-anti-CD8 VH-optional L4-anti-CD3 VL-CL-C′. In some embodiments, the MSAP comprises a first fusion polypeptide comprising the structure: N′-anti-target epitope (e.g., TAA or TSA) VL-optional L1-anti-target epitope VH-optional L2-anti-CD3 VH-CH1-C′, and a second fusion polypeptide comprising the structure: N′-anti-CD8 VH-optional L3-anti-CD8 VL-optional L4-anti-CD3 VL-CL-C′. In some embodiments, the MSAP comprises a first fusion polypeptide comprising the structure: N′-anti-target epitope (e.g., TAA or TSA) VL-optional L1-anti-target epitope VH-optional L2-anti-CD3 VH-CH1-C′, and a second fusion polypeptide comprising the structure: N′-anti-CD8 VL-optional L3-anti-CD8 VH-optional L4-anti-CD3 VL-CL-C′. In some embodiments, the MSAP (or TSAP) comprises a first fusion polypeptide comprising the structure: N′-anti-CD8 VH-optional L1-anti-CD8 VL-optional L2-anti-CD3 VH-CH1-C′, and a second fusion polypeptide comprising the structure: N′-anti-target epitope (e.g., TAA or TSA) VH-optional L3-anti-target epitope VL-optional L4-anti-CD3 VL-CL-C′. In some embodiments, the MSAP comprises a first fusion polypeptide comprising the structure: N′-anti-CD8 VH-optional L1-anti-CD8 VL-optional L2-anti-CD3 VH-CH1-C′, and a second fusion polypeptide comprising the structure: N′-anti-target epitope (e.g., TAA or TSA) VL-optional L3-anti-target epitope VH-optional L4-anti-CD3 VL-CL-C′. In some embodiments, the MSAP comprises a first fusion polypeptide comprising the structure: N′-anti-CD8 VL-optional L1-anti-CD8 VH-optional L2-anti-CD3 VH-CH1-C′, and a second fusion polypeptide comprising the structure: N′-anti-target epitope (e.g., TAA or TSA) VH-optional L3-anti-target epitope VL-optional L4-anti-CD3 VL-CL-C′. In some embodiments, the MSAP (such as TSAP) comprises a first fusion polypeptide comprising the structure: N′-anti-CD8 VL-optional L1-anti-CD8 VH-optional L2-anti-CD3 VH-CH1-C′, and a second fusion polypeptide comprising the structure: N′-anti-target epitope (e.g., TAA or TSA) VL-optional L3-anti-target epitope VH-optional L4-anti-CD3 VL-CL-C′. In some embodiments, the MSAP (or TSAP) comprises a first fusion polypeptide comprising the structure: N′-anti-CD3 VH-optional L1-anti-CD3 VL-optional L2-anti-CD8 VH-CH1-C′, and a second fusion polypeptide comprising the structure: N′-anti-target epitope (e.g., TAA or TSA) VH-optional L3-anti-target epitope VL-optional L4-anti-CD8 VL-CL-C′. In some embodiments, the MSAP comprises a first fusion polypeptide comprising the structure: N′-anti-CD3 VH-optional L1-anti-CD3 VL-optional L2-anti-CD8 VH-CH1-C′, and a second fusion polypeptide comprising the structure: N′-anti-target epitope (e.g., TAA or TSA) VL-optional L3-anti-target epitope VH-optional L4-anti-CD8 VL-CL-C′. In some embodiments, the MSAP comprises a first fusion polypeptide comprising the structure: N′-anti-CD3 VL-optional L1-anti-CD3 VH-optional L2-anti-CD8 VH-CH1-C′, and a second fusion polypeptide comprising the structure: N′-anti-target epitope (e.g., TAA or TSA) VH-optional L3-anti-target epitope VL-optional L4-anti-CD8 VL-CL-C′. In some embodiments, the MSAP comprises a first fusion polypeptide comprising the structure: N′-anti-CD3 VL-optional L1-anti-CD3 VH-optional L2-anti-CD8 VH-CH1-C′, and a second fusion polypeptide comprising the structure: N′-anti-target epitope (e.g., TAA or TSA) VL-optional L3-anti-target epitope VH-optional L4-anti-CD8 VL-CL-C′. In some embodiments, the MSAP (or TSAP) comprises a first fusion polypeptide comprising the structure: N′-anti-CD3 VH-optional L1-anti-CD3 VL-optional L2-anti-target epitope (e.g., TAA or TSA) VH-CH1-C′, and a second fusion polypeptide comprising the structure: N′-anti-CD8 VH-optional L3-anti-CD8 VL-optional L4-anti-target epitope VL-CL-C′. In some embodiments, the MSAP comprises a first fusion polypeptide comprising the structure: N′-anti-CD3 VH-optional L1-anti-CD3 VL-optional L2-anti-target epitope (e.g., TAA or TSA) VH-CH1-C′, and a second fusion polypeptide comprising the structure: N′-anti-CD8 VL-optional L3-anti-CD8 VH-optional L4-anti-target epitope VL-CL-C′. In some embodiments, the MSAP comprises a first fusion polypeptide comprising the structure: N′-anti-CD3 VL-optional L1-anti-CD3 VH-optional L2-anti-target epitope (e.g., TAA or TSA) VH-CH1-C′, and a second fusion polypeptide comprising the structure: N′-anti-CD8 VH-optional L3-anti-CD8 VL-optional L4-anti-target epitope VL-CL-C′. In some embodiments, the MSAP comprises a first fusion polypeptide comprising the structure: N′-anti-CD3 VL-optional L1-anti-CD3 VH-optional L2-anti-target epitope (e.g., TAA or TSA) VH-CH1-C′, and a second fusion polypeptide comprising the structure: N′-anti-CD8 VL-optional L3-anti-CD8 VH-optional L4-anti-target VL-CL-C′. In some embodiments, the MSAP (or TSAP) comprises a first fusion polypeptide comprising the structure: N′-anti-target epitope (e.g., TAA or TSA) VH-optional L1-anti-target epitope VL-optional L2-anti-CD8 VH-CH1-C′, and a second fusion polypeptide comprising the structure: N′-anti-CD3 VH-optional L3-anti-CD3 VL-optional L4-anti-CD8 VL-CL-C′. In some embodiments, the MSAP comprises a first fusion polypeptide comprising the structure: N′-anti-target epitope (e.g., TAA or TSA) VH-optional L1-anti-target epitope VL-optional L2-anti-CD8 VH-CH1-C′, and a second fusion polypeptide comprising the structure: N′-anti-CD3 VL-optional L3-anti-CD3 VH-optional L4-anti-CD8 VL-CL-C′. In some embodiments, the MSAP comprises a first fusion polypeptide comprising the structure: N′-anti-target epitope (e.g., TAA or TSA) VL-optional L1-anti-target epitope VH-optional L2-anti-CD8 VH-CH1-C′, and a second fusion polypeptide comprising the structure: N′-anti-CD3 VH-optional L3-anti-CD3 VL-optional L4-anti-CD8 VL-CL-C′. In some embodiments, the MSAP comprises a first fusion polypeptide comprising the structure: N′-anti-target epitope (e.g., TAA or TSA) VL-optional L1-anti-target epitope VH-optional L2-anti-CD8 VH-CH1-C′, and a second fusion polypeptide comprising the structure: N′-anti-CD3 VL-optional L3-anti-CD3 VH-optional L4-anti-CD8 VL-CL-C′. In some embodiments, the MSAP (or TSAP) comprises a first fusion polypeptide comprising the structure: N′-anti-CD8 VH-optional L1-anti-CD8 VL-optional L2-anti-target epitope (e.g., TAA or TSA) VH-CH1-C′, and a second fusion polypeptide comprising the structure: N′-anti-CD3 VH-optional L3-anti-CD3 VL-optional L4-anti-target epitope VL-CL-C′. In some embodiments, the MSAP comprises a first fusion polypeptide comprising the structure: N′-anti-CD8 VH-optional L1-anti-CD8 VL-optional L2-anti-target epitope (e.g., TAA or TSA) VH-CH1-C′, and a second fusion polypeptide comprising the structure: N′-anti-CD3 VL-optional L3-anti-CD3 VH-optional L4-anti-target epitope VL-CL-C′. In some embodiments, the MSAP comprises a first fusion polypeptide comprising the structure: N′-anti-CD8 VL-optional L1-anti-CD8 VH-optional L2-anti-target epitope (e.g., TAA or TSA) VH-CH1-C′, and a second fusion polypeptide comprising the structure: N′-anti-CD3 VH-optional L3-anti-CD3 VL-optional L4-anti-target epitope VL-CL-C′. In some embodiments, the MSAP comprises a first fusion polypeptide comprising the structure: N′-anti-CD8 VL-optional L1-anti-CD8 VH-optional L2-anti-target epitope (e.g., TAA or TSA) VH-CH1-C′, and a second fusion polypeptide comprising the structure: N′-anti-CD3 VL-optional L3-anti-CD3 VH-optional L4-anti-target epitope VL-CL-C′. In some embodiments, the MSAP comprises a first fusion polypeptide comprising the structure: N′-anti-CD8 scFv (VH-optional L1-VL or VL-optional L1-VH)-optional L2-anti-target epitope (e.g., TAA or TSA) VH-CH1-C′, and a second fusion polypeptide comprising the structure: N′-anti-target epitope VL-CL-optional L3-anti-CD3 scFv (VH-optional L4-VL or VL-optional L4-VH)-C′. In some embodiments, the MSAP comprises a first fusion polypeptide comprising the structure: N′-anti-CD3 scFv (VH-optional L1-VL or VL-optional L1-VH)-optional L2-anti-target epitope (e.g., TAA or TSA) VH-CH1-C′, and a second fusion polypeptide comprising the structure: N′-anti-target epitope VL-CL-optional L3-anti-CD8 scFv (VH-optional L4-VL or VL-optional L4-VH)-C′. In some embodiments, the MSAP comprises a first fusion polypeptide comprising the structure: N′-anti-CD8 scFv (VH-optional L1-VL or VL-optional L1-VH)-optional L2-anti-CD3 VH-CH1-C′, and a second fusion polypeptide comprising the structure: N′-anti-CD3 VL-CL-optional L3-anti-target epitope (e.g., TAA or TSA) scFv (VH-optional L4-VL or VL-optional L4-VH)-C′. In some embodiments, the MSAP comprises a first fusion polypeptide comprising the structure: N′-anti-target epitope (e.g., TAA or TSA) scFv (VH-optional L1-VL or VL-optional L1-VH)-optional L2-anti-CD3 VH-CH1-C′, and a second fusion polypeptide comprising the structure: N′-anti-CD3 VL-CL-optional L3-anti-CD8 scFv (VH-optional L4-VL or VL-optional L4-VH)-C′. In some embodiments, the MSAP comprises a first fusion polypeptide comprising the structure: N′-anti-CD3 scFv (VH-optional L1-VL or VL-optional L1-VH)-optional L2-anti-CD8 VH-CH1-C′, and a second fusion polypeptide comprising the structure: N′-anti-CD8 VL-CL-optional L3-anti-target epitope (e.g., TAA or TSA) scFv (VH-optional L4-VL or VL-optional L4-VH)-C′. In some embodiments, the MSAP comprises a first fusion polypeptide comprising the structure: N′-anti-target epitope (e.g., TAA or TSA) scFv (VH-optional L1-VL or VL-optional L1-VH)-optional L2-anti-CD8 VH-CH1-C′, and a second fusion polypeptide comprising the structure: N′-anti-CD8 VL-CL-optional L3-anti-CD3 scFv (VH-optional L4-VL or VL-optional L4-VH)-C′. In some embodiments, the MSAP comprises a first fusion polypeptide comprising the structure: N′-anti-target epitope (e.g., TAA or TSA) VH-CH1-optional L1-anti-CD3 scFv (VH-optional L2-VL or VL-optional L2-VH)-C′, and a second fusion polypeptide comprising the structure: N′-anti-CD8 scFv (VH-optional L3-VL or VL-optional L3-VH)-optional L4-anti-target epitope VL-CL-C′. In some embodiments, the MSAP comprises a first fusion polypeptide comprising the structure: N′-anti-target epitope (e.g., TAA or TSA) VH-CH1-optional L1-anti-CD8 scFv (VH-optional L2 -VL or VL-optional L2-VH)-C′, and a second fusion polypeptide comprising the structure: N′-anti-CD3 scFv (VH-optional L3-VL or VL-optional L3-VH)-optional L4-anti-target epitope VL-CL-C′. In some embodiments, the MSAP comprises a first fusion polypeptide comprising the structure: N′-anti-CD3 VH-CH1-optional L1-anti-CD8 scFv (VH-optional L2-VL or VL-optional L2-VH)-C′, and a second fusion polypeptide comprising the structure: N′-anti-target epitope (e.g., TAA or TSA) scFv (VH-optional L3-VL or VL-optional L3-VH)-optional L4-anti-CD3 VL-CL-C′. In some embodiments, the MSAP comprises a first fusion polypeptide comprising the structure: N′-anti-CD3 VH-CH1-optional L1-anti-target epitope (e.g., TAA or TSA) scFv (VH-optional L2-VL or VL-optional L2-VH)-C′, and a second fusion polypeptide comprising the structure: N′-anti-CD8 scFv (VH-optional L3-VL or VL-optional L3-VH)-optional L4-anti-CD3 VL-CL-C′. In some embodiments, the MSAP comprises a first fusion polypeptide comprising the structure: N′-anti-CD8 VH-CH1-optional L1-anti-CD3 scFv (VH-optional L2-VL or VL-optional L2-VH)-C′, and a second fusion polypeptide comprising the structure: N′-anti-target epitope (e.g., TAA or TSA) scFv (VH-optional L3-VL or VL-optional L3-VH)-optional L4-anti-CD8 VL-CL-C′. In some embodiments, the MSAP comprises a first fusion polypeptide comprising the structure: N′-anti-CD8 VH-CH1-optional L1-anti-target epitope (e.g., TAA or TSA) scFv (VH-optional L2-VL or VL-optional L2-VH)-C′, and a second fusion polypeptide comprising the structure: N′-anti-CD3 scFv (VH-optional L3-VL or VL-optional L3-VH)-optional L4-anti-CD8 VL-CL-C′. In some embodiments, the MSAP comprises a fusion polypeptide comprising the structure: N′-anti-CD8 scFv (VH-optional L1-VL or VL-optional L1-VH)-optional L2-anti-target epitope (e.g., TAA or TSA) VH-CH1-optional L3-anti-CD3 scFv (VH-optional L4-VL or VL-optional L4-VH)-C′, and a polypeptide comprising the structure: N′-anti-target epitope VL-CL-C′. In some embodiments, the MSAP comprises a fusion polypeptide comprising the structure: N′-anti-CD3 scFv (VH-optional L1-VL or VL-optional L1-VH)-optional L2-anti-target epitope (e.g., TAA or TSA) VH-CH1-optional L3-anti-CD8 scFv (VH-optional L4-VL or VL-optional L4-VH)-C′, and a polypeptide comprising the structure: N′-anti-target epitope VL-CL-C′. In some embodiments, the MSAP comprises a fusion polypeptide comprising the structure: N′-anti-CD8 scFv (VH-optional L1-VL or VL-optional L1-VH)-optional L2-anti-CD3 VH-CH1-optional L3-anti-target epitope (e.g., TAA or TSA) scFv (VH-optional L4-VL or VL-optional L4-VH)-C′, and a polypeptide comprising the structure: N′-anti-CD3 VL-CL-C′. In some embodiments, the MSAP comprises a fusion polypeptide comprising the structure: N′-anti-target epitope (e.g., TAA or TSA) scFv (VH-optional L1-VL or VL-optional L1-VH)-optional L2-anti-CD3 VH-CH1-optional L3-anti-CD8 scFv (VH-optional L4-VL or VL-optional L4-VH)-C′, and a polypeptide comprising the structure: N′-anti-CD3 VL-CL-C′. In some embodiments, the MSAP comprises a fusion polypeptide comprising the structure: N′-anti-CD3 scFv (VH-optional L1-VL or VL-optional L1-VH)-optional L2-anti-CD8 VH-CH1-optional L3-anti-target epitope (e.g., TAA or TSA) scFv (VH-optional L4-VL or VL-optional L4-VH)-C′, and a polypeptide comprising the structure: N′-anti-CD8 VL-CL-C′. In some embodiments, the MSAP comprises a fusion polypeptide comprising the structure: N′-anti-target epitope (e.g., TAA or TSA) scFv (VH-optional L1-VL or VL-optional L1-VH)-optional L2-anti-CD8 VH-CH1-optional L3-anti-CD3 scFv (VH-optional L4-VL or VL-optional L4-VH)-C′, and a polypeptide comprising the structure: N′-anti-CD8 VL-CL-C′. In some embodiments, the MSAP comprises a polypeptide comprising the structure: N′-anti-target epitope (e.g., TAA or TSA) VH-CH1-C′, and a fusion polypeptide comprising the structure: N′-anti-CD8 scFv (VH-optional L1-VL or VL-optional L1-VH)-optional L2-anti-target epitope VL-CL-optional L3-anti-CD3 scFv (VH-optional L4-VL or VL-optional L4-VH)-C′. In some embodiments, the MSAP comprises a polypeptide comprising the structure: N′-anti-target epitope (e.g., TAA or TSA) VH-CH1-C′, and a fusion polypeptide comprising the structure: N′-anti-CD3 scFv (VH-optional L1-VL or VL-optional L1-VH)-optional L2-anti-target epitope VL-CL-optional L3-anti-CD8 scFv (VH-optional L4-VL or VL-optional L4-VH)-C′. In some embodiments, the MSAP comprises a polypeptide comprising the structure: N′-anti-CD3 VH-CH1-C′, and a fusion polypeptide comprising the structure: N′-anti-CD8 scFv (VH-optional L1-VL or VL-optional L1-VH)-optional L2-anti-CD3 VL-CL-optional L3-anti-target epitope (e.g., TAA or TSA) scFv (VH-optional L4-VL or VL-optional L4-VH)-C′. In some embodiments, the MSAP comprises a polypeptide comprising the structure: N′-anti-CD3 VH-CH1-C′, and a fusion polypeptide comprising the structure: N′-anti-target epitope (e.g., TAA or TSA) scFv (VH-optional L1-VL or VL-optional L1-VH)-optional L2-anti-CD3 VL-CL-optional L3-anti-CD8 scFv (VH-optional L4-VL or VL-optional L4-VH)-C′. In some embodiments, the MSAP comprises a polypeptide comprising the structure: N′-anti-CD8 VH-CH1-C′, and a fusion polypeptide comprising the structure: N′-anti-CD3 scFv (VH-optional L1-VL or VL-optional L1-VH)-optional L2-anti-CD8 VL-CL-optional L3-anti-target epitope (e.g., TAA or TSA) scFv (VH-optional L4-VL or VL-optional L4-VH)-C′. In some embodiments, the MSAP comprises a polypeptide comprising the structure: N′-anti-CD8 VH-CH1-C′, and a fusion polypeptide comprising the structure: N′-anti-target epitope (e.g., TAA or TSA) scFv (VH-optional L1-VL or VL-optional L1-VH)-optional L2-anti-CD8 VL-CL-optional L3-anti-CD3 scFv (VH-optional L4-VL or VL-optional L4-VH)-C′. L1, L2, L3, and L4 are any possible linkers (such as peptide liners) described herein, e.g., any of GG and SEQ ID NOs: 108-110. L1-L4 can be the same or different. L1-L4 can be independently present or absent.

In some embodiments, there is provided an MSAP (such as TSAP) comprising: (i) an anti-CD3 moiety that specifically binds to CD3; (ii) an anti-CD8 moiety that specifically binds to CD8; and (iii) an anti-target epitope moiety that specifically binds to a target epitope (e.g., TAA or TSA, such as CD19, WT1, or EpCAM), wherein a first moiety of the three moieties is a Fab fragment, wherein the Fab fragment comprises a first polypeptide comprising a VH and a CH1, and a second polypeptide comprising a VL and a CL; wherein a second moiety of the three moieties is a first antigen binding fragment (e.g., scFv); wherein a third moiety of the three moieties is a second antigen binding fragment (e.g., scFv); wherein: (a) the first antigen binding fragment is fused to the N-terminus of the VH of the Fab fragment via an optional first linker, and the second antigen binding fragment is fused to the N-terminus of the VL of the Fab fragment via an optional second linker; (b) the first antigen binding fragment is fused to the C-terminus of the CH1 of the Fab fragment via an optional first linker, and the second antigen binding fragment is fused to the N-terminus of the VL of the Fab fragment via an optional second linker; (c) the first antigen binding fragment is fused to the N-terminus of the VH of the Fab fragment via an optional first linker, and the second antigen binding fragment is fused to the C-terminus of the CL of the Fab fragment via an optional second linker; (d) the first antigen binding fragment is fused to the N-terminus of the VH of the Fab fragment via an optional first linker, and the second antigen binding fragment is fused to the C-terminus of the CH1 of the Fab fragment via an optional second linker; or (e) the first antigen binding fragment is fused to the N-terminus of the VL of the Fab fragment via an optional first linker, and the second antigen binding fragment is fused to the C-terminus of the CL of the Fab fragment via an optional second linker; and wherein the anti-CD8 moiety comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23. In some embodiments, the first antigen binding fragment and the second antigen binding fragment are both scFv. In some embodiments, the anti-CD8 moiety comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD8 moiety is an anti-CD8 scFv, wherein the anti-CD8 scFv comprises the amino acid sequence of any one of SEQ ID NOs: 46, 49, 52, and 55. In some embodiments, the anti-CD8 moiety is an anti-CD8 Fab fragment, wherein the anti-CD8 Fab fragment comprises i) a first polypeptide comprising the amino acid sequence of SEQ ID NO: 91, and a second polypeptide comprising the amino acid sequence of SEQ ID NO: 92; or ii) a first polypeptide comprising the amino acid sequence of SEQ ID NO: 155, and a second polypeptide comprising the amino acid sequence of SEQ ID NO: 156. In some embodiments, the first linker and/or the second linker comprises an amino acid sequence independently selected from the group consisting of GG and SEQ ID NOs: 108-110.

In some embodiments, there is provided an MSAP (such as TSAP) comprising: i) an anti-CD3 moiety that specifically binds to CD3; ii) an anti-CD8 moiety that specifically binds to CD8; and iii) an anti-target epitope (e.g., TAA or TSA, such as CD19, WT1, or EpCAM) moiety that specifically binds to a target epitope, wherein a first moiety of the three moieties is a Fab fragment, wherein the Fab fragment comprises a first polypeptide comprising a VH and a CH1, and a second polypeptide comprising a VL and a CL; wherein a second moiety of the three moieties is a first antigen binding fragment (e.g., scFv); wherein a third moiety of the three moieties is a second antigen binding fragment (e.g., scFv); wherein: (a) the first antigen binding fragment is fused to the N-terminus of the VH of the Fab fragment via an optional first linker, and the second antigen binding fragment is fused to the N-terminus of the VL of the Fab fragment via an optional second linker; (b) the first antigen binding fragment is fused to the C-terminus of the CH1 of the Fab fragment via an optional first linker, and the second antigen binding fragment is fused to the N-terminus of the VL of the Fab fragment via an optional second linker; (c) the first antigen binding fragment is fused to the N-terminus of the VH of the Fab fragment via an optional first linker, and the second antigen binding fragment is fused to the C-terminus of the CL of the Fab fragment via an optional second linker; (d) the first antigen binding fragment is fused to the N-terminus of the VH of the Fab fragment via an optional first linker, and the second antigen binding fragment is fused to the C-terminus of the CH1 of the Fab fragment via an optional second linker; or (e) the first antigen binding fragment is fused to the N-terminus of the VL of the Fab fragment via an optional first linker, and the second antigen binding fragment is fused to the C-terminus of the CL of the Fab fragment via an optional second linker; and wherein the anti-CD3 moiety comprises: (1) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (2) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (3) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the first antigen binding fragment and the second antigen binding fragment are both scFv. In some embodiments, the anti-CD3 moiety comprises: a VH comprising the amino acid sequence of any of SEQ ID NOs: 42, 159, and 167, and a VL comprising the amino acid sequence of SEQ ID NO: 43. In some embodiments, the anti-CD3 moiety is an anti-CD3 scFv, wherein the anti-CD3 scFv comprises the amino acid sequence of any of SEQ ID NOs: 50, 56, 163, 164, 183, and 184. In some embodiments, the anti-CD3 moiety is an anti-CD3 Fab fragment, wherein the anti-CD3 Fab fragment comprises: a first polypeptide comprising the amino acid sequence of any of SEQ ID NOs: 89, 160, and 168 and a second polypeptide comprising the amino acid sequence of SEQ ID NO: 90 or 179. In some embodiments, the first linker and/or the second linker comprises an amino acid sequence independently selected from the group consisting of GG and SEQ ID NOs: 108-110.

In some embodiments, there is provided an MSAP (such as TSAP) comprising: (1) an anti-CD3 moiety that specifically binds to CD3; (2) an anti-CD8 moiety that specifically binds to CD8; and (3) an anti-target epitope (e.g., TAA or TSA, such as CD19, WT1, or EpCAM) moiety that specifically binds to a target epitope, wherein a first moiety of the three moieties is a Fab fragment, wherein the Fab fragment comprises a first polypeptide comprising a VH and a CH1, and a second polypeptide comprising a VL and a CL; wherein a second moiety of the three moieties is a first antigen binding fragment (e.g., scFv); wherein a third moiety of the three moieties is a second antigen binding fragment (e.g., scFv); wherein: (a) the first antigen binding fragment is fused to the N-terminus of the VH of the Fab fragment via an optional first linker, and the second antigen binding fragment is fused to the N-terminus of the VL of the Fab fragment via an optional second linker; (b) the first antigen binding fragment is fused to the C-terminus of the CH1 of the Fab fragment via an optional first linker, and the second antigen binding fragment is fused to the N-terminus of the VL of the Fab fragment via an optional second linker; (c) the first antigen binding fragment is fused to the N-terminus of the VH of the Fab fragment via an optional first linker, and the second antigen binding fragment is fused to the C-terminus of the CL of the Fab fragment via an optional second linker; (d) the first antigen binding fragment is fused to the N-terminus of the VH of the Fab fragment via an optional first linker, and the second antigen binding fragment is fused to the C-terminus of the CH1 of the Fab fragment via an optional second linker; or (e) the first antigen binding fragment is fused to the N-terminus of the VL of the Fab fragment via an optional first linker, and the second antigen binding fragment is fused to the C-terminus of the CL of the Fab fragment via an optional second linker; wherein the anti-CD8 moiety comprises: (i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; and wherein the anti-CD3 moiety comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the first antigen binding fragment and the second antigen binding fragment are both scFv. In some embodiments, the anti-CD8 moiety comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 moiety comprises: a VH comprising the amino acid sequence of any of SEQ ID NOs: 42, 159, and 167, and a VL comprising the amino acid sequence of SEQ ID NO: 43. In some embodiments, the anti-CD8 moiety is an anti-CD8 scFv, wherein the anti-CD8 scFv comprises the amino acid sequence of any one of SEQ ID NOs: 46, 49, 52, and 55. In some embodiments, the anti-CD3 moiety is an anti-CD3 scFv, wherein the anti-CD3 scFv comprises the amino acid sequence of any of SEQ ID NOs: 50, 56, 163, 164, 183, and 184. In some embodiments, the anti-CD8 moiety is an anti-CD8 Fab fragment, wherein the anti-CD8 Fab fragment comprises i) a first polypeptide comprising the amino acid sequence of SEQ ID NO: 91, and a second polypeptide comprising the amino acid sequence of SEQ ID NO: 92; or ii) a first polypeptide comprising the amino acid sequence of SEQ ID NO: 155, and a second polypeptide comprising the amino acid sequence of SEQ ID NO: 156. In some embodiments, the anti-CD3 moiety is an anti-CD3 Fab fragment, wherein the anti-CD3 Fab fragment comprises: a first polypeptide comprising the amino acid sequence of any of SEQ ID NOs: 89, 160, and 168, and a second polypeptide comprising the amino acid sequence of SEQ ID NO: 90 or 179. In some embodiments, the first linker and/or the second linker comprises an amino acid sequence independently selected from the group consisting of GG and SEQ ID NOs: 108-110. In some embodiments, the anti-target epitope moiety specifically recognizes CD19 (anti-CD19 moiety). In some embodiments, the anti-CD19 moiety comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD19 moiety comprises a VH comprising the amino acid sequence of SEQ ID NO: 63, and a VL comprising the amino acid sequence of SEQ ID NO: 64. In some embodiments, the anti-CD19 moiety is an anti-CD19 scFv, and wherein the anti-CD19 scFv comprises the amino acid sequence of SEQ ID NO: 65 or 66. In some embodiments, the anti-CD19 moiety is an anti-CD19 Fab fragment, and wherein the anti-CD19 Fab fragment comprises a first polypeptide comprising the amino acid sequence of SEQ ID NO: 137, and a second polypeptide comprising the amino acid sequence of SEQ ID NO: 139 or 169. In some embodiments, the anti-target epitope moiety specifically recognizing EpCAM (anti-EpCAM moiety). In some embodiments, the anti-EpCAM moiety comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, the anti-EpCAM moiety comprises a VH comprising the amino acid sequence of SEQ ID NO: 73, and a VL comprising the amino acid sequence of SEQ ID NO: 74. In some embodiments, the anti-EpCAM moiety is an anti-EpCAM scFv, and wherein the anti-EpCAM scFv comprises the amino acid sequence of SEQ ID NO: 75 or 78. In some embodiments, the anti-EpCAM moiety is an anti-EpCAM Fab fragment, and wherein the anti-EpCAM Fab fragment comprises a first polypeptide comprising the amino acid sequence of SEQ ID NO: 76, and a second polypeptide comprising the amino acid sequence of SEQ ID NO: 77. In some embodiments, the anti-target epitope moiety specifically recognizing WT1 (anti-WT1 moiety). In some embodiments, the anti-WT1 moiety comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the anti-WT1 moiety comprises a VH comprising the amino acid sequence of SEQ ID NO: 85, and a VL comprising the amino acid sequence of SEQ ID NO: 86. In some embodiments, the anti-WT1 moiety is an anti-WT1 scFv, and wherein the anti-WT1 scFv comprises the amino acid sequence of SEQ ID NO: 87 or 88. In some embodiments, the anti-WT1 moiety is an anti-WT1 Fab fragment, and wherein the anti-WT1 Fab fragment comprises a first polypeptide comprising the amino acid sequence of SEQ ID NO: 145, and a second polypeptide comprising the amino acid sequence of SEQ ID NO: 146.

In some embodiments, there is provided an MSAP (e.g., TSAP) comprising: a) an anti-CD3 Fab fragment, b) an anti-CD8 first antigen binding fragment (e.g., scFv), and c) an anti-CD19 second antigen binding fragment (e.g., scFv); wherein the anti-CD3 Fab fragment comprises a first polypeptide comprising a VH and a CH1, and a second polypeptide comprising a VL and a CL; wherein the anti-CD8 first antigen binding fragment is fused to the N-terminus of the VH of the anti-CD3 Fab fragment via an optional first linker; wherein the anti-CD19 second antigen binding fragment is fused to the N-terminus of the VL of the anti-CD3 Fab fragment via an optional second linker; wherein the anti-CD8 first antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 Fab fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-CD19 second antigen binding fragment comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD8 first antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 Fab fragment comprises: a VH comprising the amino acid sequence of any of SEQ ID NOs: 42, 159, and 167, and a VL comprising the amino acid sequence of SEQ ID NO: 43. In some embodiments, the anti-CD19 second antigen binding fragment comprises a VH comprising the amino acid sequence of SEQ ID NO: 63, and a VL comprising the amino acid sequence of SEQ ID NO: 64. In some embodiments, the anti-CD3 Fab fragment comprises: (i) a first polypeptide comprising the amino acid sequence of any of SEQ ID NOs: 89, 160, and 179, and a second polypeptide comprising the amino acid sequence of SEQ ID NO: 90 or 179. In some embodiments, the anti-CD8 first antigen binding fragment and the anti-CD19 second antigen binding fragment are both scFv. In some embodiments, the anti-CD8 scFv comprises the amino acid sequence of any one of SEQ ID NOs: 46, 49, 52, and 55. In some embodiments, the anti-CD19 scFv comprises the amino acid sequence of SEQ ID NO: 65 or 66. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: a) an anti-CD3 Fab fragment, b) an anti-CD19 first antigen binding fragment (e.g., scFv), and c) an anti-CD8 second antigen binding fragment (e.g., scFv); wherein the anti-CD3 Fab fragment comprises a first polypeptide comprising a VH and a CH1, and a second polypeptide comprising a VL and a CL; wherein the anti-CD19 first antigen binding fragment is fused to the N-terminus of the VH of the anti-CD3 Fab fragment via an optional first linker; wherein the anti-CD8 second antigen binding fragment is fused to the N-terminus of the VL of the anti-CD3 Fab fragment via an optional second linker; wherein the anti-CD8 second antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 Fab fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-CD19 first antigen binding fragment comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD8 second antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 Fab fragment comprises: a VH comprising the amino acid sequence of any of SEQ ID NOs: 42, 159, and 167, and a VL comprising the amino acid sequence of SEQ ID NO: 43. In some embodiments, the anti-CD19 first antigen binding fragment comprises a VH comprising the amino acid sequence of SEQ ID NO: 63, and a VL comprising the amino acid sequence of SEQ ID NO: 64. In some embodiments, the anti-CD3 Fab fragment comprises: (i) a first polypeptide comprising the amino acid sequence of any of SEQ ID NOs: 89, 160, and 168, and a second polypeptide comprising the amino acid sequence of SEQ ID NO: 90 or 179. In some embodiments, the anti-CD19 first antigen binding fragment and the anti-CD8 second antigen binding fragment are both scFv. In some embodiments, the anti-CD8 scFv comprises the amino acid sequence of any one of SEQ ID NOs: 46, 49, 52, and 55. In some embodiments, the anti-CD19 scFv comprises the amino acid sequence of SEQ ID NO: 65 or 66. In some embodiments, the first linker and/or the second linker comprises an amino acid sequence independently selected from the group consisting of GG and SEQ ID NOs: 108-110. In some embodiments, there is provided an MSAP (e.g., TSAP) comprising a first fusion polypeptide and a second fusion polypeptide, wherein the first fusion polypeptide comprises the amino acid sequence of SEQ ID NO: 103 or 161, and the second fusion polypeptide comprises the amino acid sequence of SEQ ID NO: 104 or 106. In some embodiments, there is provided an MSAP (e.g., TSAP) comprising a first fusion polypeptide and a second fusion polypeptide, wherein: i) the first fusion polypeptide comprises the amino acid sequence of SEQ ID NO: 103, and the second fusion polypeptide comprises the amino acid sequence of SEQ ID NO: 104; ii) the first fusion polypeptide comprises the amino acid sequence of SEQ ID NO: 103, and the second fusion polypeptide comprises the amino acid sequence of SEQ ID NO: 106; or iii) the first fusion polypeptide comprises the amino acid sequence of SEQ ID NO: 161, and the second fusion polypeptide comprises the amino acid sequence of SEQ ID NO: 106.

In some embodiments, there is provided an MSAP (e.g., TSAP), wherein the MSAP comprises a first fusion polypeptide comprising an anti-CD19 first scFv fused to the N-terminus of the VH of an anti-CD3 Fab fragment via a first linker, and a second fusion polypeptide comprising an anti-CD8 second scFv fused to the N-terminus of the VL of the anti-CD3 Fab fragment via a second linker; and wherein: i) the first fusion polypeptide comprises the amino acid sequence of SEQ ID NO: 103 (or an amino acid sequence having at least about 85% sequence identity to the sequence of SEQ ID NO: 103), and the second fusion polypeptide comprises the amino acid sequence of SEQ ID NO: 104 (or an amino acid sequence having at least about 85% sequence identity to the sequence of SEQ ID NO: 104); ii) the first fusion polypeptide comprises the amino acid sequence of SEQ ID NO: 103 (or an amino acid sequence having at least about 85% sequence identity to the sequence of SEQ ID NO: 103), and the second fusion polypeptide comprises the amino acid sequence of SEQ ID NO: 106 (or an amino acid sequence having at least about 85% sequence identity to the sequence of SEQ ID NO: 106); or iii) the first fusion polypeptide comprises the amino acid sequence of SEQ ID NO: 161 (or an amino acid sequence having at least about 85% sequence identity to the sequence of SEQ ID NO: 161), and the second fusion polypeptide comprises the amino acid sequence of SEQ ID NO: 106 (or an amino acid sequence having at least about 85% sequence identity to the sequence of SEQ ID NO: 106).

In some embodiments, there is provided an MSAP (e.g., TSAP) comprising: a) an anti-CD8 Fab fragment, b) an anti-CD3 first antigen binding fragment (e.g., scFv), and c) an anti-CD19 second antigen binding fragment (e.g., scFv); wherein the anti-CD8 Fab fragment comprises a first polypeptide comprising a VH and a CH1, and a second polypeptide comprising a VL and a CL; wherein the anti-CD3 first antigen binding fragment is fused to the N-terminus of the VH of the anti-CD8 Fab fragment via an optional first linker; wherein the anti-CD19 second antigen binding fragment is fused to the N-terminus of the VL of the anti-CD8 Fab fragment via an optional second linker; wherein the anti-CD8 Fab fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 first antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-CD19 second antigen binding fragment comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD8 Fab fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 first antigen binding fragment comprises: a VH comprising the amino acid sequence of any of SEQ ID NOs: 42, 159, and 167, and a VL comprising the amino acid sequence of SEQ ID NO: 43. In some embodiments, the anti-CD19 second antigen binding fragment comprises a VH comprising the amino acid sequence of SEQ ID NO: 63, and a VL comprising the amino acid sequence of SEQ ID NO: 64. In some embodiments, the anti-CD8 Fab fragment comprises i) a first polypeptide comprising the amino acid sequence of SEQ ID NO: 91, and a second polypeptide comprising the amino acid sequence of SEQ ID NO: 92; or ii) a first polypeptide comprising the amino acid sequence of SEQ ID NO: 155, and a second polypeptide comprising the amino acid sequence of SEQ ID NO: 156. In some embodiments, the anti-CD3 first antigen binding fragment and the anti-CD19 second antigen binding fragment are both scFv. In some embodiments, the anti-CD3 scFv comprises the amino acid sequence of any of SEQ ID NOs: 50, 56, 163, 164, 183, and 184. In some embodiments, the anti-CD19 scFv comprises the amino acid sequence of SEQ ID NO: 65 or 66. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: a) an anti-CD8 Fab fragment, b) an anti-CD19 first antigen binding fragment (e.g., scFv), and c) an anti-CD3 second antigen binding fragment (e.g., scFv); wherein the anti-CD8 Fab fragment comprises a first polypeptide comprising a VH and a CH1, and a second polypeptide comprising a VL and a CL; wherein the anti-CD19 first antigen binding fragment is fused to the N-terminus of the VH of the anti-CD8 Fab fragment via an optional first linker; wherein the anti-CD3 second antigen binding fragment is fused to the N-terminus of the VL of the anti-CD8 Fab fragment via an optional second linker; wherein the anti-CD8 Fab fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 second antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-CD19 first antigen binding fragment comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD8 Fab fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 second antigen binding fragment comprises: a VH comprising the amino acid sequence of any of SEQ ID NOs: 42, 159, and 167, and a VL comprising the amino acid sequence of SEQ ID NO: 43. In some embodiments, the anti-CD19 first antigen binding fragment comprises a VH comprising the amino acid sequence of SEQ ID NO: 63, and a VL comprising the amino acid sequence of SEQ ID NO: 64. In some embodiments, the anti-CD8 Fab fragment comprises i) a first polypeptide comprising the amino acid sequence of SEQ ID NO: 91, and a second polypeptide comprising the amino acid sequence of SEQ ID NO: 92; or ii) a first polypeptide comprising the amino acid sequence of SEQ ID NO: 155, and a second polypeptide comprising the amino acid sequence of SEQ ID NO: 156. In some embodiments, the anti-CD19 first antigen binding fragment and the anti-CD3 second antigen binding fragment are both scFv. In some embodiments, the anti-CD3 scFv comprises the amino acid sequence of any of SEQ ID NOs: 50, 56, 163, 164, 183, and 184. In some embodiments, the anti-CD19 scFv comprises the amino acid sequence of SEQ ID NO: 65 or 66. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: a) an anti-CD19 Fab fragment, b) an anti-CD3 first antigen binding fragment (e.g., scFv), and c) an anti-CD8 second antigen binding fragment (e.g., scFv); wherein the anti-CD19 Fab fragment comprises a first polypeptide comprising a VH and a CH1, and a second polypeptide comprising a VL and a CL; wherein the anti-CD3 first antigen binding fragment is fused to the N-terminus of the VH of the anti-CD19 Fab fragment via an optional first linker; wherein the anti-CD8 second antigen binding fragment is fused to the N-terminus of the VL of the anti-CD19 Fab fragment via an optional second linker; wherein the anti-CD8 second antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 first antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-CD19 Fab fragment comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD8 second antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 first antigen binding fragment comprises: a VH comprising the amino acid sequence of any of SEQ ID NOs: 42, 159, and 167, and a VL comprising the amino acid sequence of SEQ ID NO: 43. In some embodiments, the anti-CD19 Fab fragment comprises a VH comprising the amino acid sequence of SEQ ID NO: 63, and a VL comprising the amino acid sequence of SEQ ID NO: 64. In some embodiments, the anti-CD19 Fab fragment comprises a first polypeptide comprising the amino acid sequence of SEQ ID NO: 137, and a second polypeptide comprising the amino acid sequence of SEQ ID NO: 139 or 169. In some embodiments, the anti-CD3 first antigen binding fragment and the anti-CD8 second antigen binding fragment are both scFv. In some embodiments, the anti-CD8 scFv comprises the amino acid sequence of any one of SEQ ID NOs: 46, 49, 52, and 55. In some embodiments, the anti-CD3 scFv comprises the amino acid sequence of any of SEQ ID NOs: 50, 56, 163, 164, 183, and 184. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: a) an anti-CD19 Fab fragment, b) an anti-CD8 first antigen binding fragment (e.g., scFv), and c) an anti-CD3 second antigen binding fragment (e.g., scFv); wherein the anti-CD19 Fab fragment comprises a first polypeptide comprising a VH and a CH1, and a second polypeptide comprising a VL and a CL; wherein the anti-CD8 first antigen binding fragment is fused to the N-terminus of the VH of the anti-CD19 Fab fragment via an optional first linker; wherein the anti-CD3 second antigen binding fragment is fused to the N-terminus of the VL of the anti-CD19 Fab fragment via an optional second linker; wherein the anti-CD8 first antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 second antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-CD19 Fab fragment comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD8 first antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 second antigen binding fragment comprises: a VH comprising the amino acid sequence of any of SEQ ID NOs: 42, 159, and 167, and a VL comprising the amino acid sequence of SEQ ID NO: 43. In some embodiments, the anti-CD19 Fab fragment comprises a VH comprising the amino acid sequence of SEQ ID NO: 63, and a VL comprising the amino acid sequence of SEQ ID NO: 64. In some embodiments, the anti-CD19 Fab fragment comprises a first polypeptide comprising the amino acid sequence of SEQ ID NO: 137, and a second polypeptide comprising the amino acid sequence of SEQ ID NO: 139 or 169. In some embodiments, the anti-CD8 first antigen binding fragment and the anti-CD3 second antigen binding fragment are both scFv. In some embodiments, the anti-CD8 scFv comprises the amino acid sequence of any one of SEQ ID NOs: 46, 49, 52, and 55. In some embodiments, the anti-CD3 scFv comprises the amino acid sequence of any of SEQ ID NOs: 50, 56, 163, 164, 183, and 184. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: a) an anti-CD19 Fab fragment, b) an anti-CD3 first antigen binding fragment (e.g., scFv), and c) an anti-CD8 second antigen binding fragment (e.g., scFv); wherein the anti-CD19 Fab fragment comprises a first polypeptide comprising a VH and a CH1, and a second polypeptide comprising a VL and a CL; wherein the anti-CD3 first antigen binding fragment is fused to the C-terminus of the CH1 of the anti-CD19 Fab fragment via an optional first linker, and the anti-CD8 second antigen binding fragment is fused to the N-terminus of the VL of the anti-CD19 Fab fragment via an optional second linker; wherein the anti-CD8 second antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 first antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-CD19 Fab fragment comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD8 second antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 first antigen binding fragment comprises: a VH comprising the amino acid sequence of any of SEQ ID NOs: 42, 159, and 167, and a VL comprising the amino acid sequence of SEQ ID NO: 43. In some embodiments, the anti-CD19 Fab fragment comprises a VH comprising the amino acid sequence of SEQ ID NO: 63, and a VL comprising the amino acid sequence of SEQ ID NO: 64. In some embodiments, the anti-CD19 Fab fragment comprises a first polypeptide comprising the amino acid sequence of SEQ ID NO: 137, and a second polypeptide comprising the amino acid sequence of SEQ ID NO: 139 or 169. In some embodiments, the anti-CD3 first antigen binding fragment and the anti-CD8 second antigen binding fragment are both scFv. In some embodiments, the anti-CD8 scFv comprises the amino acid sequence of any one of SEQ ID NOs: 46, 49, 52, and 55. In some embodiments, the anti-CD3 scFv comprises the amino acid sequence of any of SEQ ID NOs: 50, 56, 163, 164, 183, and 184. In some embodiments, the first linker and/or the second linker comprises an amino acid sequence independently selected from the group consisting of GG and SEQ ID NOs: 108-110. In some embodiments, there is provided an MSAP (e.g., TSAP) comprising a first fusion polypeptide comprising the amino acid sequence of SEQ ID NO: 170, and a second fusion polypeptide comprising the amino acid sequence of SEQ ID NO: 171.

In some embodiments, there is provided an MSAP (e.g., TSAP), wherein the MSAP comprises a first fusion polypeptide comprising an anti-CD3 first scFv fused to the C-terminus of the CH1 of an anti-CD19 Fab fragment via a first linker, and a second fusion polypeptide comprising an anti-CD8 second scFv fused to the N-terminus of the VL of the anti-CD19 Fab fragment via a second linker; and wherein the first fusion polypeptide comprises the amino acid sequence of SEQ ID NO: 170 (or an amino acid sequence having at least about 85% sequence identity to the sequence of SEQ ID NO: 170), and the second fusion polypeptide comprises the amino acid sequence of SEQ ID NO: 171 (or an amino acid sequence having at least about 85% sequence identity to the sequence of SEQ ID NO: 171).

In some embodiments, there is provided an MSAP (e.g., TSAP) comprising: a) an anti-CD19 Fab fragment, b) an anti-CD8 first antigen binding fragment (e.g., scFv), and c) an anti-CD3 second antigen binding fragment (e.g., scFv); wherein the anti-CD19 Fab fragment comprises a first polypeptide comprising a VH and a CH1, and a second polypeptide comprising a VL and a CL; wherein the anti-CD8 first antigen binding fragment is fused to the C-terminus of the CH1 of the anti-CD19 Fab fragment via an optional first linker, and the anti-CD3 second antigen binding fragment is fused to the N-terminus of the VL of the anti-CD19 Fab fragment via an optional second linker; wherein the anti-CD8 first antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 second antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-CD19 Fab fragment comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD8 first antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 second antigen binding fragment comprises: a VH comprising the amino acid sequence of any of SEQ ID NOs: 42, 159, and 167, and a VL comprising the amino acid sequence of SEQ ID NO: 43. In some embodiments, the anti-CD19 Fab fragment comprises a VH comprising the amino acid sequence of SEQ ID NO: 63, and a VL comprising the amino acid sequence of SEQ ID NO: 64. In some embodiments, the anti-CD19 Fab fragment comprises a first polypeptide comprising the amino acid sequence of SEQ ID NO: 137, and a second polypeptide comprising the amino acid sequence of SEQ ID NO: 139 or 169. In some embodiments, the anti-CD3 second antigen binding fragment and the anti-CD8 first antigen binding fragment are both scFv. In some embodiments, the anti-CD8 scFv comprises the amino acid sequence of any one of SEQ ID NOs: 46, 49, 52, and 55. In some embodiments, the anti-CD3 scFv comprises the amino acid sequence of any of SEQ ID NOs: 50, 56, 163, 164, 183, and 184. In some embodiments, the first linker and/or the second linker comprises an amino acid sequence independently selected from the group consisting of GG and SEQ ID NOs: 108-110. In some embodiments, there is provided an MSAP (e.g., TSAP) comprising a first fusion polypeptide and a second fusion polypeptide, wherein: (a) the first fusion polypeptide comprises the amino acid sequence of SEQ ID NO: 172, and the second fusion polypeptide comprises the amino acid sequence of SEQ ID NO: 173; b) the first fusion polypeptide comprises the amino acid sequence of SEQ ID NO: 174, and the second fusion polypeptide comprises the amino acid sequence of SEQ ID NO: 175; c) the first fusion polypeptide comprises the amino acid sequence of SEQ ID NO: 172, and the second fusion polypeptide comprises the amino acid sequence of SEQ ID NO: 176; or d) the first fusion polypeptide comprises the amino acid sequence of SEQ ID NO: 172, and the second fusion polypeptide comprises the amino acid sequence of SEQ ID NO: 182.

In some embodiments, there is provided an MSAP (e.g., TSAP), wherein the MSAP comprises a first fusion polypeptide comprising an anti-CD8 first scFv fused to the C-terminus of the CH1 of an anti-CD19 Fab fragment via a first linker, and a second fusion polypeptide comprising an anti-CD3 second scFv fused to the N-terminus of the VL of the anti-CD19 Fab fragment via a second linker; and wherein: the first fusion polypeptide comprises the amino acid sequence of SEQ ID NO: 172 or 174 (or an amino acid sequence having at least about 85% sequence identity to the sequence of SEQ ID NO: 172 or 174), and the second fusion polypeptide comprises the amino acid sequence of any of SEQ ID NOs: 173, 175, 176, and 182 (or an amino acid sequence having at least about 85% sequence identity to the sequence of any of SEQ ID NOs: 173, 175, 176, and 182). In some embodiments, there is provided an MSAP (e.g., TSAP), wherein the MSAP comprises a first fusion polypeptide comprising an anti-CD8 first scFv fused to the C-terminus of the CH1 of an anti-CD19 Fab fragment via a first linker, and a second fusion polypeptide comprising an anti-CD3 second scFv fused to the N-terminus of the VL of the anti-CD19 Fab fragment via a second linker; and wherein: (a) the first fusion polypeptide comprises the amino acid sequence of SEQ ID NO: 172 (or an amino acid sequence having at least about 85% sequence identity to the sequence of SEQ ID NO: 172), and the second fusion polypeptide comprises the amino acid sequence of SEQ ID NO: 173 (or an amino acid sequence having at least about 85% sequence identity to the sequence of SEQ ID NO: 173); b) the first fusion polypeptide comprises the amino acid sequence of SEQ ID NO: 174 (or an amino acid sequence having at least about 85% sequence identity to the sequence of SEQ ID NO: 174), and the second fusion polypeptide comprises the amino acid sequence of SEQ ID NO: 175 (or an amino acid sequence having at least about 85% sequence identity to the sequence of SEQ ID NO: 175); c) the first fusion polypeptide comprises the amino acid sequence of SEQ ID NO: 172 (or an amino acid sequence having at least about 85% sequence identity to the sequence of SEQ ID NO: 172), and the second fusion polypeptide comprises the amino acid sequence of SEQ ID NO: 176 (or an amino acid sequence having at least about 85% sequence identity to the sequence of SEQ ID NO: 176); or d) the first fusion polypeptide comprises the amino acid sequence of SEQ ID NO: 172 (or an amino acid sequence having at least about 85% sequence identity to the sequence of SEQ ID NO: 172), and the second fusion polypeptide comprises the amino acid sequence of SEQ ID NO: 182 (or an amino acid sequence having at least about 85% sequence identity to the sequence of SEQ ID NO: 182).

In some embodiments, there is provided an MSAP (e.g., TSAP) comprising: a) an anti-CD19 Fab fragment, b) an anti-CD3 first antigen binding fragment (e.g., scFv), and c) an anti-CD8 second antigen binding fragment (e.g., scFv); wherein the anti-CD19 Fab fragment comprises a first polypeptide comprising a VH and a CH1, and a second polypeptide comprising a VL and a CL; wherein the anti-CD3 first antigen binding fragment is fused to the N-terminus of the VH of the anti-CD19 Fab fragment via an optional first linker, and the anti-CD8 second antigen binding fragment is fused to the C-terminus of the CL of the anti-CD19 Fab fragment via an optional second linker; wherein the anti-CD8 second antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 first antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-CD19 Fab fragment comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD8 second antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 first antigen binding fragment comprises: a VH comprising the amino acid sequence of any of SEQ ID NOs: 42, 159, and 167, and a VL comprising the amino acid sequence of SEQ ID NO: 43. In some embodiments, the anti-CD19 Fab fragment comprises a VH comprising the amino acid sequence of SEQ ID NO: 63, and a VL comprising the amino acid sequence of SEQ ID NO: 64. In some embodiments, the anti-CD19 Fab fragment comprises a first polypeptide comprising the amino acid sequence of SEQ ID NO: 137, and a second polypeptide comprising the amino acid sequence of SEQ ID NO: 139 or 169. In some embodiments, the anti-CD3 first antigen binding fragment and the anti-CD8 second antigen binding fragment are both scFv. In some embodiments, the anti-CD8 scFv comprises the amino acid sequence of any one of SEQ ID NOs: 46, 49, 52, and 55. In some embodiments, the anti-CD3 scFv comprises the amino acid sequence of any of SEQ ID NOs: 50, 56, 163, 164, 183, and 184. In some embodiments, the first linker and/or the second linker comprises an amino acid sequence independently selected from the group consisting of GG and SEQ ID NOs: 108-110. In some embodiments, there is provided an MSAP (e.g., TSAP) comprising a first fusion polypeptide comprising the amino acid sequence of SEQ ID NO: 177, and a second fusion polypeptide comprising the amino acid sequence of SEQ ID NO: 178.

In some embodiments, there is provided an MSAP (e.g., TSAP), wherein the MSAP comprises a first fusion polypeptide comprising an anti-CD3 first scFv fused to the N-terminus of the VH of an anti-CD19 Fab fragment via a first linker, and a second fusion polypeptide comprising an anti-CD8 second scFv fused to the C-terminus of the CL of the anti-CD 19 Fab fragment via a second linker; and wherein the first fusion polypeptide comprises the amino acid sequence of SEQ ID NO: 177 (or an amino acid sequence having at least about 85% sequence identity to the sequence of SEQ ID NO: 177), and the second fusion polypeptide comprises the amino acid sequence of SEQ ID NO: 178 (or an amino acid sequence having at least about 85% sequence identity to the sequence of SEQ ID NO: 178).

In some embodiments, there is provided an MSAP (e.g., TSAP) comprising: a) an anti-CD19 Fab fragment, b) an anti-CD3 first antigen binding fragment (e.g., scFv), and c) an anti-CD8 second antigen binding fragment (e.g., scFv); wherein the anti-CD19 Fab fragment comprises a first polypeptide comprising a VH and a CH1, and a second polypeptide comprising a VL and a CL; wherein the anti-CD3 first antigen binding fragment is fused to the N-terminus of VH of the anti-CD19 Fab fragment via an optional first linker, and the anti-CD8 second antigen binding fragment is fused to the C-terminus of the CH1 of the anti-CD19 Fab fragment via an optional second linker; wherein the anti-CD8 second antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 first antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-CD19 Fab fragment comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD8 second antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 first antigen binding fragment comprises: a VH comprising the amino acid sequence of any of SEQ ID NOs: 42, 159, and 167, and a VL comprising the amino acid sequence of SEQ ID NO: 43. In some embodiments, the anti-CD19 Fab fragment comprises a VH comprising the amino acid sequence of SEQ ID NO: 63, and a VL comprising the amino acid sequence of SEQ ID NO: 64. In some embodiments, the anti-CD19 Fab fragment comprises a first polypeptide comprising the amino acid sequence of SEQ ID NO: 137, and a second polypeptide comprising the amino acid sequence of SEQ ID NO: 139 or 169. In some embodiments, the anti-CD3 first antigen binding fragment and the anti-CD8 second antigen binding fragment are both scFv. In some embodiments, the anti-CD8 scFv comprises the amino acid sequence of any one of SEQ ID NOs: 46, 49, 52, and 55. In some embodiments, the anti-CD3 scFv comprises the amino acid sequence of any of SEQ ID NOs: 50, 56, 163, 164, 183, and 184. In some embodiments, the first linker and/or the second linker comprises an amino acid sequence independently selected from the group consisting of GG and SEQ ID NOs: 108-110. In some embodiments, there is provided an MSAP (e.g., TSAP) comprising a fusion polypeptide comprising the amino acid sequence of SEQ ID NO: 180, and a polypeptide comprising the amino acid sequence of SEQ ID NO: 169.

In some embodiments, there is provided an MSAP (e.g., TSAP), wherein the MSAP comprises: i) a fusion polypeptide comprising an anti-CD3 first scFv fused to the N-terminus of the VH of an anti-CD19 Fab fragment via a first linker, and an anti-CD8 second scFv fused to the C-terminus of the CH1 of the anti-CD19 Fab fragment via a second linker; and ii) the second polypeptide of the anti-CD19 Fab fragment; and wherein the fusion polypeptide comprises the amino acid sequence of SEQ ID NO: 180 (or an amino acid sequence having at least about 85% sequence identity to the sequence of SEQ ID NO: 180), and the second polypeptide of the anti-CD19 Fab fragment comprises the amino acid sequence of SEQ ID NO: 169 (or an amino acid sequence having at least about 85% sequence identity to the sequence of SEQ ID NO: 169).

In some embodiments, there is provided an MSAP (e.g., TSAP) comprising: a) an anti-CD19 Fab fragment, b) an anti-CD3 first antigen binding fragment (e.g., scFv), and c) an anti-CD8 second antigen binding fragment (e.g., scFv); wherein the anti-CD19 Fab fragment comprises a first polypeptide comprising a VH and a CH1, and a second polypeptide comprising a VL and a CL; wherein the anti-CD3 first antigen binding fragment is fused to the N-terminus of the VL of the anti-CD19 Fab fragment via an optional first linker, and the anti-CD8 second antigen binding fragment is fused to the C-terminus of the CL of the anti-CD19 Fab fragment via an optional second linker; wherein the anti-CD8 second antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 first antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-CD19 Fab fragment comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD8 second antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 first antigen binding fragment comprises: a VH comprising the amino acid sequence of any of SEQ ID NOs: 42, 159, and 167, and a VL comprising the amino acid sequence of SEQ ID NO: 43. In some embodiments, the anti-CD19 Fab fragment comprises a VH comprising the amino acid sequence of SEQ ID NO: 63, and a VL comprising the amino acid sequence of SEQ ID NO: 64. In some embodiments, the anti-CD19 Fab fragment comprises a first polypeptide comprising the amino acid sequence of SEQ ID NO: 137, and a second polypeptide comprising the amino acid sequence of SEQ ID NO: 139 or 169. In some embodiments, the anti-CD3 first antigen binding fragment and the anti-CD8 second antigen binding fragment are both scFv. In some embodiments, the anti-CD8 scFv comprises the amino acid sequence of any one of SEQ ID NOs: 46, 49, 52, and 55. In some embodiments, the anti-CD3 scFv comprises the amino acid sequence of any of SEQ ID NOs: 50, 56, 163, 164, 183, and 184. In some embodiments, the first linker and/or the second linker comprises an amino acid sequence independently selected from the group consisting of GG and SEQ ID NOs: 108-110. In some embodiments, there is provided an MSAP (e.g., TSAP) comprising a polypeptide comprising the amino acid sequence of SEQ ID NO: 137, and a fusion polypeptide comprising the amino acid sequence of SEQ ID NO: 181.

In some embodiments, there is provided an MSAP (e.g., TSAP), wherein the MSAP comprises: i) the first polypeptide of an anti-CD19 Fab fragment; and ii) a fusion polypeptide comprising an anti-CD3 first scFv fused to the N-terminus of the VL of the anti-CD19 Fab fragment via a first linker, and an anti-CD8 second scFv fused to the C-terminus of the CL of the anti-CD19 Fab fragment via a second linker; and wherein the first polypeptide of the anti-CD19 Fab fragment comprises the amino acid sequence of SEQ ID NO: 137 (or an amino acid sequence having at least about 85% sequence identity to the sequence of SEQ ID NO: 137), and the fusion polypeptide comprises the amino acid sequence of SEQ ID NO: 181 (or an amino acid sequence having at least about 85% sequence identity to the sequence of SEQ ID NO: 181).

In some embodiments, there is provided an MSAP (e.g., TSAP) comprising: a) an anti-CD3 Fab fragment, b) an anti-CD8 first antigen binding fragment (e.g., scFv), and c) an anti-EpCAM second antigen binding fragment (e.g., scFv); wherein the anti-CD3 Fab fragment comprises a first polypeptide comprising a VH and a CH1, and a second polypeptide comprising a VL and a CL; wherein the anti-CD8 first antigen binding fragment is fused to the N-terminus of the VH of the anti-CD3 Fab fragment via an optional first linker; wherein the anti-EpCAM second antigen binding fragment is fused to the N-terminus of the VL of the anti-CD3 Fab fragment via an optional second linker; wherein the anti-CD8 first antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 Fab fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-EpCAM second antigen binding fragment comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, the anti-CD8 first antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 Fab fragment comprises: a VH comprising the amino acid sequence of any of SEQ ID NOs: 42, 159, and 167, and a VL comprising the amino acid sequence of SEQ ID NO: 43. In some embodiments, the anti-EpCAM second antigen binding fragment comprises a VH comprising the amino acid sequence of SEQ ID NO: 73, and a VL comprising the amino acid sequence of SEQ ID NO: 74. In some embodiments, the anti-CD3 Fab fragment comprises: a first polypeptide comprising the amino acid sequence of any of SEQ ID NOs: 89, 160, and 168, and a second polypeptide comprising the amino acid sequence of SEQ ID NO: 90 or 179. In some embodiments, the anti-CD8 first antigen binding fragment and the anti-EpCAM second antigen binding fragment are both scFv. In some embodiments, the anti-CD8 scFv comprises the amino acid sequence of any one of SEQ ID NOs: 46, 49, 52, and 55. In some embodiments, the anti-EpCAM scFv comprises the amino acid sequence of SEQ ID NO: 75 or 78. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: a) an anti-CD3 Fab fragment, b) an anti-EpCAM first antigen binding fragment (e.g., scFv), and c) an anti-CD8 second antigen binding fragment (e.g., scFv); wherein the anti-CD3 Fab fragment comprises a first polypeptide comprising a VH and a CH1, and a second polypeptide comprising a VL and a CL; wherein the anti-EpCAM first antigen binding fragment is fused to the N-terminus of the VH of the anti-CD3 Fab fragment via an optional first linker; wherein the anti-CD8 second antigen binding fragment is fused to the N-terminus of the VL of the anti-CD3 Fab fragment via an optional second linker; wherein the anti-CD8 second antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 Fab fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-EpCAM first antigen binding fragment comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, the anti-CD8 second antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 Fab fragment comprises: a VH comprising the amino acid sequence of any of SEQ ID NOs: 42, 159, and 167, and a VL comprising the amino acid sequence of SEQ ID NO: 43. In some embodiments, the anti-EpCAM first antigen binding fragment comprises a VH comprising the amino acid sequence of SEQ ID NO: 73, and a VL comprising the amino acid sequence of SEQ ID NO: 74. In some embodiments, the anti-CD3 Fab fragment comprises: a first polypeptide comprising the amino acid sequence of any of SEQ ID NOs: 89, 160, and 168, and a second polypeptide comprising the amino acid sequence of SEQ ID NO: 90 or 179. In some embodiments, the anti-EpCAM first antigen binding fragment and the anti-CD8 second antigen binding fragment are both scFv. In some embodiments, the anti-CD8 scFv comprises the amino acid sequence of any one of SEQ ID NOs: 46, 49, 52, and 55. In some embodiments, the anti-EpCAM scFv comprises the amino acid sequence of SEQ ID NO: 75 or 78. In some embodiments, the first linker and/or the second linker comprises an amino acid sequence independently selected from the group consisting of GG and SEQ ID NOs: 108-110. In some embodiments, there is provided an MSAP (e.g., TSAP) comprising a first fusion polypeptide and a second fusion polypeptide, wherein the first fusion polypeptide comprises the amino acid sequence of SEQ ID NO: 101, and the second fusion polypeptide comprises the amino acid sequence of SEQ ID NO: 102.

In some embodiments, there is provided an MSAP (e.g., TSAP), wherein the MSAP comprises a first fusion polypeptide comprising an anti-EpCAM first scFv fused to the N-terminus of the VH of an anti-CD3 Fab fragment via a first linker, and a second fusion polypeptide comprising an anti-CD8 second scFv fused to the N-terminus of the VL of the anti-CD3 Fab fragment via a second linker; wherein the first fusion polypeptide comprises the amino acid sequence of SEQ ID NO: 101 (or an amino acid sequence having at least about 85% sequence identity to the sequence of SEQ ID NO: 101), and the second fusion polypeptide comprises the amino acid sequence of SEQ ID NO: 102 (or an amino acid sequence having at least about 85% sequence identity to the sequence of SEQ ID NO: 102).

In some embodiments, there is provided an MSAP (e.g., TSAP) comprising: a) an anti-CD8 Fab fragment, b) an anti-CD3 first antigen binding fragment (e.g., scFv), and c) an anti-EpCAM second antigen binding fragment (e.g., scFv); wherein the anti-CD8 Fab fragment comprises a first polypeptide comprising a VH and a CH1, and a second polypeptide comprising a VL and a CL; wherein the anti-CD3 first antigen binding fragment is fused to the N-terminus of the VH of the anti-CD8 Fab fragment via an optional first linker; wherein the anti-EpCAM second antigen binding fragment is fused to the N-terminus of the VL of the anti-CD8 Fab fragment via an optional second linker; wherein the anti-CD8 Fab fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 first antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-EpCAM second antigen binding fragment comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, the anti-CD8 Fab fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 first antigen binding fragment comprises: a VH comprising the amino acid sequence of any of SEQ ID NOs: 42, 159, and 167, and a VL comprising the amino acid sequence of SEQ ID NO: 43. In some embodiments, the anti-EpCAM second antigen binding fragment comprises a VH comprising the amino acid sequence of SEQ ID NO: 73, and a VL comprising the amino acid sequence of SEQ ID NO: 74. In some embodiments, the anti-CD8 Fab fragment comprises i) a first polypeptide comprising the amino acid sequence of SEQ ID NO: 91, and a second polypeptide comprising the amino acid sequence of SEQ ID NO: 92; or ii) a first polypeptide comprising the amino acid sequence of SEQ ID NO: 155, and a second polypeptide comprising the amino acid sequence of SEQ ID NO: 156. In some embodiments, the anti-CD3 first antigen binding fragment and the anti-EpCAM second antigen binding fragment are both scFv. In some embodiments, the anti-CD3 scFv comprises the amino acid sequence of any of SEQ ID NOs: 50, 56, 163, 164, 183, and 184. In some embodiments, the anti-EpCAM scFv comprises the amino acid sequence of SEQ ID NO: 75 or 78. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: a) an anti-CD8 Fab fragment, b) an anti-EpCAM first antigen binding fragment (e.g., scFv), and c) an anti-CD3 second antigen binding fragment (e.g., scFv); wherein the anti-CD8 Fab fragment comprises a first polypeptide comprising a VH and a CH1, and a second polypeptide comprising a VL and a CL; wherein the anti-EpCAM first antigen binding fragment is fused to the N-terminus of the VH of the anti-CD8 Fab fragment via an optional first linker; wherein the anti-CD3 second antigen binding fragment is fused to the N-terminus of the VL of the anti-CD8 Fab fragment via an optional second linker; wherein the anti-CD8 Fab fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 second antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-EpCAM first antigen binding fragment comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, the anti-CD8 Fab fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 second antigen binding fragment comprises: a VH comprising the amino acid sequence of any of SEQ ID NOs: 42, 159, and 167, and a VL comprising the amino acid sequence of SEQ ID NO: 43. In some embodiments, the anti-EpCAM first antigen binding fragment comprises a VH comprising the amino acid sequence of SEQ ID NO: 73, and a VL comprising the amino acid sequence of SEQ ID NO: 74. In some embodiments, the anti-CD8 Fab fragment comprises i) a first polypeptide comprising the amino acid sequence of SEQ ID NO: 91, and a second polypeptide comprising the amino acid sequence of SEQ ID NO: 92; or ii) a first polypeptide comprising the amino acid sequence of SEQ ID NO: 155, and a second polypeptide comprising the amino acid sequence of SEQ ID NO: 156. In some embodiments, the anti-EpCAM first antigen binding fragment and the anti-CD3 second antigen binding fragment are both scFv. In some embodiments, the anti-CD3 scFv comprises the amino acid sequence of any of SEQ ID NOs: 50, 56, 163, 164, 183, and 184. In some embodiments, the anti-EpCAM scFv comprises the amino acid sequence of SEQ ID NO: 75 or 78. In some embodiments, the first linker and/or the second linker comprises an amino acid sequence independently selected from the group consisting of GG and SEQ ID NOs: 108-110. In some embodiments, there is provided an MSAP (e.g., TSAP) comprising a first fusion polypeptide and a second fusion polypeptide, wherein the first fusion polypeptide comprises the amino acid sequence of SEQ ID NO: 99, and the second fusion polypeptide comprises the amino acid sequence of SEQ ID NO: 100.

In some embodiments, there is provided an MSAP (e.g., TSAP), wherein the MSAP comprises a first fusion polypeptide comprising an anti-EpCAM first scFv fused to the N-terminus of the VH of an anti-CD8 Fab fragment via a first linker, and a second fusion polypeptide comprising an anti-CD3 second scFv fused to the N-terminus of the VL of the anti-CD8 Fab fragment via a second linker; wherein the first fusion polypeptide comprises the amino acid sequence of SEQ ID NO: 99 (or an amino acid sequence having at least about 85% sequence identity to the sequence of SEQ ID NO: 99), and the second fusion polypeptide comprises the amino acid sequence of SEQ ID NO: 100 (or an amino acid sequence having at least about 85% sequence identity to the sequence of SEQ ID NO: 100).

In some embodiments, there is provided an MSAP (e.g., TSAP) comprising: a) an anti-EpCAM Fab fragment, b) an anti-CD3 first antigen binding fragment (e.g., scFv), and c) an anti-CD8 second antigen binding fragment (e.g., scFv); wherein the anti-EpCAM Fab fragment comprises a first polypeptide comprising a VH and a CH1, and a second polypeptide comprising a VL and a CL; wherein the anti-CD3 first antigen binding fragment is fused to the N-terminus of the VH of the anti-EpCAM Fab fragment via an optional first linker; wherein the anti-CD8 second antigen binding fragment is fused to the N-terminus of the VL of the anti-EpCAM Fab fragment via an optional second linker; wherein the anti-CD8 second antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 first antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-EpCAM Fab fragment comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, the anti-CD8 second antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 first antigen binding fragment comprises: a VH comprising the amino acid sequence of any of SEQ ID NOs: 42, 159, and 167, and a VL comprising the amino acid sequence of SEQ ID NO: 43. In some embodiments, the anti-EpCAM Fab fragment comprises a VH comprising the amino acid sequence of SEQ ID NO: 73, and a VL comprising the amino acid sequence of SEQ ID NO: 74. In some embodiments, the anti-EpCAM Fab fragment comprises a first polypeptide comprising the amino acid sequence of SEQ ID NO: 76, and a second polypeptide comprising the amino acid sequence of SEQ ID NO: 77. In some embodiments, the anti-CD3 first antigen binding fragment and the anti-CD8 second antigen binding fragment are both scFv. In some embodiments, the anti-CD8 scFv comprises the amino acid sequence of any one of SEQ ID NOs: 46, 49, 52, and 55. In some embodiments, the anti-CD3 scFv comprises the amino acid sequence of any of SEQ ID NOs: 50, 56, 163, 164, 183, and 184. In some embodiments, the first linker and/or the second linker comprises an amino acid sequence independently selected from the group consisting of GG and SEQ ID NOs: 108-110. In some embodiments, there is provided an MSAP (e.g., TSAP) comprising a first fusion polypeptide and a second fusion polypeptide, wherein the first fusion polypeptide comprises the amino acid sequence of SEQ ID NO: 97, and the second fusion polypeptide comprises the amino acid sequence of SEQ ID NO: 98.

In some embodiments, there is provided an MSAP (e.g., TSAP), wherein the MSAP comprises a first fusion polypeptide comprising an anti-CD3 first scFv fused to the N-terminus of the VH of an anti-EpCAM Fab fragment via a first linker, and a second fusion polypeptide comprising an anti-CD8 second scFv fused to the N-terminus of the VL of the anti-EpCAM Fab fragment via a second linker; wherein the first fusion polypeptide comprises the amino acid sequence of SEQ ID NO: 97 (or an amino acid sequence having at least about 85% sequence identity to the sequence of SEQ ID NO: 97), and the second fusion polypeptide comprises the amino acid sequence of SEQ ID NO: 98 (or an amino acid sequence having at least about 85% sequence identity to the sequence of SEQ ID NO: 98).

In some embodiments, there is provided an MSAP (e.g., TSAP) comprising: a) an anti-EpCAM Fab fragment, b) an anti-CD8 first antigen binding fragment (e.g., scFv), and c) an anti-CD3 second antigen binding fragment (e.g., scFv); wherein the anti-EpCAM Fab fragment comprises a first polypeptide comprising a VH and a CH1, and a second polypeptide comprising a VL and a CL; wherein the anti-CD8 first antigen binding fragment is fused to the N-terminus of the VH of the anti-EpCAM Fab fragment via an optional first linker; wherein the anti-CD3 second antigen binding fragment is fused to the N-terminus of the VL of the anti-EpCAM Fab fragment via an optional second linker; wherein the anti-CD8 first antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 second antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L 1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-EpCAM Fab fragment comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, the anti-CD8 first antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 second antigen binding fragment comprises: a VH comprising the amino acid sequence of any of SEQ ID NOs: 42, 159, and 167, and a VL comprising the amino acid sequence of SEQ ID NO: 43. In some embodiments, the anti-EpCAM Fab fragment comprises a VH comprising the amino acid sequence of SEQ ID NO: 73, and a VL comprising the amino acid sequence of SEQ ID NO: 74. In some embodiments, the anti-EpCAM Fab fragment comprises a first polypeptide comprising the amino acid sequence of SEQ ID NO: 76, and a second polypeptide comprising the amino acid sequence of SEQ ID NO: 77. In some embodiments, the anti-CD8 first antigen binding fragment and the anti-CD3 second antigen binding fragment are both scFv. In some embodiments, the anti-CD8 scFv comprises the amino acid sequence of any one of SEQ ID NOs: 46, 49, 52, and 55. In some embodiments, the anti-CD3 scFv comprises the amino acid sequence of any of SEQ ID NOs: 50, 56, 163, 164, 183, and 184. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: a) an anti-CD3 Fab fragment, b) an anti-CD8 first antigen binding fragment (e.g., scFv), and c) an anti-WT1 second antigen binding fragment (e.g., scFv); wherein the anti-CD3 Fab fragment comprises a first polypeptide comprising a VH and a CH1, and a second polypeptide comprising a VL and a CL; wherein the anti-CD8 first antigen binding fragment is fused to the N-terminus of the VH of the anti-CD3 Fab fragment via an optional first linker; wherein the anti-WT1 second antigen binding fragment is fused to the N-terminus of the VL of the anti-CD3 Fab fragment via an optional second linker; wherein the anti-CD8 first antigen binding fragment comprises: i an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 Fab fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (i) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-WT1 second antigen binding fragment comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the anti-CD8 first antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 Fab fragment comprises: a VH comprising the amino acid sequence of any of SEQ ID NOs: 42, 159, and 167, and a VL comprising the amino acid sequence of SEQ ID NO: 43. In some embodiments, the anti-WT1 second antigen binding fragment comprises a VH comprising the amino acid sequence of SEQ ID NO: 85, and a VL comprising the amino acid sequence of SEQ ID NO: 86. In some embodiments, the anti-CD3 Fab fragment comprises: a first polypeptide comprising the amino acid sequence of any of SEQ ID NOs: 89, 160, and 168, and a second polypeptide comprising the amino acid sequence of SEQ ID NO: 90 or 179. In some embodiments, the anti-CD8 first antigen binding fragment and the anti-WT1 second antigen binding fragment are both scFv. In some embodiments, the anti-CD8 scFv comprises the amino acid sequence of any one of SEQ ID NOs: 46, 49, 52, and 55. In some embodiments, the anti-WT1 scFv comprises the amino acid sequence of SEQ ID NO: 87 or 88. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: a) an anti-CD3 Fab fragment, b) an anti-WT1 first antigen binding fragment (e.g., scFv), and c) an anti-CD8 second antigen binding fragment (e.g., scFv); wherein the anti-CD3 Fab fragment comprises a first polypeptide comprising a VH and a CH1, and a second polypeptide comprising a VL and a CL; wherein the anti-WT1 first antigen binding fragment is fused to the N-terminus of the VH of the anti-CD3 Fab fragment via an optional first linker; wherein the anti-CD8 second antigen binding fragment is fused to the N-terminus of the VL of the anti-CD3 Fab fragment via an optional second linker; wherein the anti-CD8 second antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 Fab fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-WT1 first antigen binding fragment comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the anti-CD8 second antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 Fab fragment comprises: a VH comprising the amino acid sequence of any of SEQ ID NOs: 42, 159, and 167, and a VL comprising the amino acid sequence of SEQ ID NO: 43. In some embodiments, the anti-WT1 first antigen binding fragment comprises a VH comprising the amino acid sequence of SEQ ID NO: 85, and a VL comprising the amino acid sequence of SEQ ID NO: 86. In some embodiments, the anti-CD3 Fab fragment comprises: a first polypeptide comprising the amino acid sequence of any of SEQ ID NOs: 89, 160, and 168, and a second polypeptide comprising the amino acid sequence of SEQ ID NO: 90 or 179. In some embodiments, the anti-WT1 first antigen binding fragment and the anti-CD8 second antigen binding fragment are both scFv. In some embodiments, the anti-CD8 scFv comprises the amino acid sequence of any one of SEQ ID NOs: 46, 49, 52, and 55. In some embodiments, the anti-WT1 scFv comprises the amino acid sequence of SEQ ID NO: 87 or 88. In some embodiments, the first linker and/or the second linker comprises an amino acid sequence independently selected from the group consisting of GG and SEQ ID NOs: 108-110. In some embodiments, there is provided an MSAP (e.g., TSAP) comprising a first fusion polypeptide and a second fusion polypeptide, wherein the first fusion polypeptide comprises the amino acid sequence of SEQ ID NO: 95, and the second fusion polypeptide comprises the amino acid sequence of SEQ ID NO: 96.

In some embodiments, there is provided an MSAP (e.g., TSAP), wherein the MSAP comprises a first fusion polypeptide comprising an anti-WT1 first scFv fused to the N-terminus of the VH of an anti-CD3 Fab fragment via a first linker, and a second fusion polypeptide comprising an anti-CD8 second scFv fused to the N-terminus of the VL of the anti-CD3 Fab fragment via a second linker; wherein the first fusion polypeptide comprises the amino acid sequence of SEQ ID NO: 95 (or an amino acid sequence having at least about 85% sequence identity to the sequence of SEQ ID NO: 95), and the second fusion polypeptide comprises the amino acid sequence of SEQ ID NO: 96 (or an amino acid sequence having at least about 85% sequence identity to the sequence of SEQ ID NO: 96).

In some embodiments, there is provided an MSAP (e.g., TSAP) comprising: a) an anti-CD8 Fab fragment, b) an anti-CD3 first antigen binding fragment (e.g., scFv), and c) an anti-WT1 second antigen binding fragment (e.g., scFv); wherein the anti-CD8 Fab fragment comprises a first polypeptide comprising a VH and a CH1, and a second polypeptide comprising a VL and a CL; wherein the anti-CD3 first antigen binding fragment is fused to the N-terminus of the VH of the anti-CD8 Fab fragment via an optional first linker; wherein the anti-WT1 second antigen binding fragment is fused to the N-terminus of the VL of the anti-CD8 Fab fragment via an optional second linker; wherein the anti-CD8 Fab fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 first antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-WT1 second antigen binding fragment comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the anti-CD8 Fab fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 first antigen binding fragment comprises: a VH comprising the amino acid sequence of any of SEQ ID NOs: 42, 159, and 167, and a VL comprising the amino acid sequence of SEQ ID NO: 43. In some embodiments, the anti-WT1 second antigen binding fragment comprises a VH comprising the amino acid sequence of SEQ ID NO: 85, and a VL comprising the amino acid sequence of SEQ ID NO: 86. In some embodiments, the anti-CD8 Fab fragment comprises i) a first polypeptide comprising the amino acid sequence of SEQ ID NO: 91, and a second polypeptide comprising the amino acid sequence of SEQ ID NO: 92; or ii) a first polypeptide comprising the amino acid sequence of SEQ ID NO: 155, and a second polypeptide comprising the amino acid sequence of SEQ ID NO: 156. In some embodiments, the anti-CD3 first antigen binding fragment and the anti-WT1 second antigen binding fragment are both scFv. In some embodiments, the anti-CD3 scFv comprises the amino acid sequence of any of SEQ ID NOs: 50, 56, 163, 164, 183, and 184. In some embodiments, the anti-WT1 scFv comprises the amino acid sequence of SEQ ID NO: 87 or 88. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: a) an anti-CD8 Fab fragment, b) an anti-WT1 first antigen binding fragment (e.g., scFv), and c) an anti-CD3 second antigen binding fragment (e.g., scFv); wherein the anti-CD8 Fab fragment comprises a first polypeptide comprising a VH and a CH1, and a second polypeptide comprising a VL and a CL; wherein the anti-WT1 first antigen binding fragment is fused to the N-terminus of the VH of the anti-CD8 Fab fragment via an optional first linker; wherein the anti-CD3 second antigen binding fragment is fused to the N-terminus of the VL of the anti-CD8 Fab fragment via an optional second linker; wherein the anti-CD8 Fab fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 second antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-WT1 first antigen binding fragment comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the anti-CD8 Fab fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 second antigen binding fragment comprises: a VH comprising the amino acid sequence of any of SEQ ID NOs: 42, 159, and 167, and a VL comprising the amino acid sequence of SEQ ID NO: 43. In some embodiments, the anti-WT1 first antigen binding fragment comprises a VH comprising the amino acid sequence of SEQ ID NO: 85, and a VL comprising the amino acid sequence of SEQ ID NO: 86. In some embodiments, the anti-CD8 Fab fragment comprises i) a first polypeptide comprising the amino acid sequence of SEQ ID NO: 91, and a second polypeptide comprising the amino acid sequence of SEQ ID NO: 92; or ii) a first polypeptide comprising the amino acid sequence of SEQ ID NO: 155, and a second polypeptide comprising the amino acid sequence of SEQ ID NO: 156. In some embodiments, the anti-WT1 first antigen binding fragment and the anti-CD3 second antigen binding fragment are both scFv. In some embodiments, the anti-CD3 scFv comprises the amino acid sequence of any of SEQ ID NOs: 50, 56, 163, 164, 183, and 184. In some embodiments, the anti-WT1 scFv comprises the amino acid sequence of SEQ ID NO: 87 or 88. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: a) an anti-WT1 Fab fragment, b) an anti-CD3 first antigen binding fragment (e.g., scFv), and c) an anti-CD8 second antigen binding fragment (e.g., scFv); wherein the anti-WT1 Fab fragment comprises a first polypeptide comprising a VH and a CH1, and a second polypeptide comprising a VL and a CL; wherein the anti-CD3 first antigen binding fragment is fused to the N-terminus of the VH of the anti-WT1 Fab fragment via an optional first linker; wherein the anti-CD8 second antigen binding fragment is fused to the N-terminus of the VL of the anti-WT1 Fab fragment via an optional second linker; wherein the anti-CD8 second antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 first antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-WT1 Fab fragment comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the anti-CD8 second antigen binding fragment comprises: i) a VH comprising the amino acid sequence of SEQ ID NO: 28, and a VL comprising the amino acid sequence of SEQ ID NO: 29; or ii) a VH comprising the amino acid sequence of SEQ ID NO: 34, and a VL comprising the amino acid sequence of SEQ ID NO: 35. In some embodiments, the anti-CD3 first antigen binding fragment comprises: a VH comprising the amino acid sequence of any of SEQ ID NOs: 42, 159, and 167, and a VL comprising the amino acid sequence of SEQ ID NO: 43. In some embodiments, the anti-WT1 Fab fragment comprises a VH comprising the amino acid sequence of SEQ ID NO: 85, and a VL comprising the amino acid sequence of SEQ ID NO: 86. In some embodiments, the anti-WT1 Fab fragment comprises a first polypeptide comprising the amino acid sequence of SEQ ID NO: 145, and a second polypeptide comprising the amino acid sequence of SEQ ID NO: 146. In some embodiments, the anti-CD3 first antigen binding fragment and the anti-CD8 second antigen binding fragment are both scFv. In some embodiments, the anti-CD8 scFv comprises the amino acid sequence of any one of SEQ ID NOs: 46, 49, 52, and 55. In some embodiments, the anti-CD3 scFv comprises the amino acid sequence of any of SEQ ID NOs: 50, 56, 163, 164, 183, and 184. In some embodiments, the first linker and/or the second linker comprises an amino acid sequence independently selected from the group consisting of GG and SEQ ID NOs: 108-110.

In some embodiments, there is provided an MSAP (e.g., TSAP) comprising: a) an anti-WT1 Fab fragment, b) an anti-CD8 first antigen binding fragment (e.g., scFv), and c) an anti-CD3 second antigen binding fragment (e.g., scFv); wherein the anti-WT1 Fab fragment comprises a first polypeptide comprising a VH and a CH1, and a second polypeptide comprising a VL and a CL; wherein the anti-CD8 first antigen binding fragment is fused to the N-terminus of the VH of the anti-WT1 Fab fragment via an optional first linker; wherein the anti-CD3 second antigen binding fragment is fused to the N-terminus of the VL of the anti-WT1 Fab fragment via an optional second linker; wherein the anti-CD8 first antigen binding fragment comprises: i) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; or ii) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; wherein the anti-CD3 second antigen binding fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and wherein the anti-WT1 Fab fragment comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the anti-CD8 first antigen binding fragment comprises: i) a VH comprising the amino acid sequence of SEQ ID NO: 28, and a VL comprising the amino acid sequence of SEQ ID NO: 29 or ii) a VH comprising the amino acid sequence of SEQ ID NO: 34, and a VL comprising the amino acid sequence of SEQ ID NO: 35. In some embodiments, the anti-CD3 second antigen binding fragment comprises: a VH comprising the amino acid sequence of any of SEQ ID NOs: 42, 159, and 167, and a VL comprising the amino acid sequence of SEQ ID NO: 43. In some embodiments, the anti-WT1 Fab fragment comprises a VH comprising the amino acid sequence of SEQ ID NO: 85, and a VL comprising the amino acid sequence of SEQ ID NO: 86. In some embodiments, the anti-WT1 Fab fragment comprises a first polypeptide comprising the amino acid sequence of SEQ ID NO: 145, and a second polypeptide comprising the amino acid sequence of SEQ ID NO: 146. In some embodiments, the anti-CD8 first antigen binding fragment and the anti-CD3 second antigen binding fragment are both scFv. In some embodiments, the anti-CD8 scFv comprises the amino acid sequence of any one of SEQ ID NOs: 46, 49, 52, and 55. In some embodiments, the anti-CD3 scFv comprises the amino acid sequence of any of SEQ ID NOs: 50, 56, 163, 164, 183, and 184. In some embodiments, the first linker and/or the second linker comprises an amino acid sequence independently selected from the group consisting of GG and SEQ ID NOs: 108-110.

In some embodiments, there is provided an MSAP (e.g., TSAP or BSAP) comprising a first fusion polypeptide and a second fusion polypeptide, wherein the first fusion polypeptide comprises the amino acid sequence of any of SEQ ID NOs: 95, 101, 103, and 161, or an amino acid sequence having at least about 85% (such as at least about any of 90%, 95%, 96%, 97%, 98%, 99%, or more) sequence identity to the sequence of any of SEQ ID NOs: 95, 101, 103, and 161; and wherein the second fusion polypeptide comprises the amino acid sequence of SEQ ID NO: 96 or 106, or an amino acid sequence having at least about 85% (such as at least about any of 90%, 95%, 96%, 97%, 98%, 99%, or more) sequence identity to the sequence of SEQ ID NOs: 96 or 106. In some embodiments, there is provided an MSAP (e.g., TSAP or BSAP) comprising a first fusion polypeptide and a second fusion polypeptide, wherein the first fusion polypeptide comprises the amino acid sequence of SEQ ID NO: 97, or an amino acid sequence having at least about 85% (such as at least about any of 90%, 95%, 96%, 97%, 98%, 99%, or more) sequence identity to the sequence of SEQ ID NO: 97; and wherein the second fusion polypeptide comprises the amino acid sequence of SEQ ID NO: 98, or an amino acid sequence having at least about 85% (such as at least about any of 90%, 95%, 96%, 97%, 98%, 99%, or more) sequence identity to the sequence of SEQ ID NOs: 98. In some embodiments, there is provided an MSAP (e.g., TSAP or BSAP) comprising a first fusion polypeptide and a second fusion polypeptide, wherein the first fusion polypeptide comprises the amino acid sequence of SEQ ID NO: 99, or an amino acid sequence having at least about 85% (such as at least about any of 90%, 95%, 96%, 97%, 98%, 99%, or more) sequence identity to the sequence of SEQ ID NO: 99; and wherein the second fusion polypeptide comprises the amino acid sequence of SEQ ID NO: 100, or an amino acid sequence having at least about 85% (such as at least about any of 90%, 95%, 96%, 97%, 98%, 99%, or more) sequence identity to the sequence of SEQ ID NOs: 100.

In some embodiments, the C-terminus of the first and/or the second polypeptide (e.g., fusion polypeptide) of the MSAP (such as TSAP) comprises a covalent binding region CPPC (SEQ ID NO: 143) or CPPCS (SEQ ID NO: 107) capable of forming an intermolecular disulfide bond. In some embodiments, the CPPCS (SEQ ID NO: 107) at the C-terminus of the first and/or the second polypeptide (e.g., fusion polypeptide) of any of the MSAPs described herein can be replaced with CPPC (SEQ ID NO: 143), or any other covalent binding region capable of forming an intermolecular disulfide bond. In some embodiments, the covalent binding region capable of forming an intermolecular disulfide bond is located at the C-terminus of CH1 and CL of the Fab fragment. In some embodiments, the C-terminus of the Fab fragment of the MSAP comprises a covalent binding region CPPC (SEQ ID NO: 143) or CPPCS (SEQ ID NO: 107) capable of forming an intermolecular disulfide bond. In some embodiments, the C-terminus of the first and/or the second polypeptide (e.g., fusion polypeptide) of the MSAP (such as TSAP) does not comprise a covalent binding region CPPC (SEQ ID NO: 143) or CPPCS (SEQ ID NO: 107) capable of forming an intermolecular disulfide bond. In some embodiments, the C-terminus of the Fab fragment of the MSAP does not comprise a covalent binding region CPPC (SEQ ID NO: 143) or CPPCS (SEQ ID NO: 107) capable of forming an intermolecular disulfide bond.

In some embodiments, the N-terminus and/or C-terminus of the first and/or the second polypeptide (e.g., fusion polypeptide) of the MSAP (e.g., TSAP) further comprises a purification tag (e.g., a histidine tag, HIS-tag) for protein purification.

In some embodiments, the N-terminus of the first and/or the second polypeptide (e.g., fusion polypeptide) of the MSAP (such as TSAP) further comprises a signal peptide (e.g., for better expression). In some embodiments, the signal peptide comprises the amino acid sequence of SEQ ID NO: 141. In some embodiments, the signal peptide is encoded by a nucleic acid sequence of SEQ ID NO: 142. In some embodiments, the signal peptide is cleaved in the mature MSAPs (e.g., secreted MSAPs from host cells).

Also provided are isolated nucleic acid(s) encoding any of the MSAPs (e.g., TSAPs) described herein, vector(s) (e.g., viral vector, such as lentiviral vector or AAV) comprising such isolated nucleic acid(s), and host cells (prokaryotic or eukaryotic) comprising such isolated nucleic acid(s) or vector(s) or expressing any of the MSAPs described herein.

The invention further provides fusion proteins comprising any of the MSAPs (such as TSAPs) described herein, MSAP conjugates (e.g., small molecule drug conjugates, or detection tag conjugates), or isolated cells expressing any of the MSAPs described herein.

### Antigen-Binding Moieties

MSAPs of the present invention comprise antigen-binding moieties that specifically bind to CD3, CD8, and a target epitope (e.g., TAA or TSA, such as CD19, WT1, or EpCAM). Any of the anti-CD3 moieties, the anti-CD8 moieties, and/or the anti-target epitope moieties described herein can be of any format and derived from any suitable antibodies or antigen-binding fragments thereof. For example, anti-CD3 moieties, anti-CD8 moieties, and/or anti-target epitope moieties can be independently selected from a full-length antibody, an scFv, a VH, a VL, an scFv-scFv, an Fv, a Fab, a Fab′, a (Fab′)₂, a minibody, a diabody, a domain antibody variant (dAb), a single domain antibody (sdAb), a camelid antibody (VHH), a fibronectin 3 domain variant, an ankyrin repeat variant, and other antigen-specific binding domains derived from other protein scaffolds. In some embodiments, the anti-CD3 moiety, the anti-CD8 moiety, and/or the anti-target epitope moiety is a Fab fragment (also referred herein as “anti-CD3 Fab fragment”, “anti-CD8 Fab fragment”, and “anti-target epitope Fab fragment”, respectively, e.g., “anti-CD19 Fab fragment,” “anti-EpCAM Fab fragment,” or “anti-WT1 Fab fragment). In some embodiments, the anti-CD3 moiety, the anti-CD8 moiety, and/or the anti-target epitope moiety is an scFv (also referred herein as “anti-CD3 scFv”, “anti-CD8 scFv”, and “anti-target epitope scFv”, respectively, e.g., “anti-CD19 scFv,” “anti-EpCAM scFv,” or “anti-WT1 scFv). Any of the anti-CD3 moieties (e.g., full-length, sdAb, scFv, or Fab), anti-CD8 moieties, and/or anti-target epitope moieties can be generated by a variety of methods known in the art (see, e.g., U.S. Pat. Nos. 6,291,161; 6,291,158). Sources of anti-CD3 moieties, anti-CD8 moieties, and/or anti-target epitope moieties include monoclonal antibody or antigen-binding fragments thereof from various species, including human, camelid (from camels, dromedaries, or llamas; Hamers-Casterman et al. (1993) Nature, 363:446 and Nguyen et al. (1998) J. Mol. Biol., 275:413), shark (Roux et al. (1998) Proc. Nat'l. Acad. Sci. (USA) 95:11804), fish (Nguyen et al. (2002) Immunogenetics, 54:39), rodent, avian, or ovine. In some embodiments, the anti-CD3 moiety, the anti-CD8 moiety, and/or the anti-target epitope moiety is independently derived from a human or humanized antibody. In some embodiments, the anti-CD3 moiety, the anti-CD8 moiety, and/or the anti-target epitope moiety is independently chimeric. In some embodiments, the anti-CD3 moiety, the anti-CD8 moiety, and/or the anti-target epitope moiety is independently derived from a fully human antibody, for example, developed using phage-display, yeast-display, or transgenic mice bearing human Ig genes. In some embodiments, the anti-CD3 moiety, the anti-CD8 moiety, and/or the anti-target epitope moiety is independently murine. In some embodiments, the anti-CD3 antibody or antigen-binding fragment thereof (or anti-CD3 moiety), the anti-CD8 antibody or antigen-binding fragment thereof (or anti-CD8 moiety), and/or the anti-target epitope antibody or antigen-binding fragment thereof (or anti-target epitope moiety) is independently a monospecific antibody, a multispecific antibody, a monoclonal antibody, a multivalent (e.g., bivalent) antibody, or a chimeric antibody. Any anti-CD3 moieties, anti-CD8 moieties, and/or anti-target epitope moieties available in the field can be used herein.

In some embodiments, the anti-CD3 moiety, the anti-CD8 moiety, and/or the anti-target epitope moiety is independently an scFv (e.g., anti-CD3 scFv, anti-CD8 scFv, or anti-target epitope scFv, respectively). In some embodiments, the anti-CD3 scFv, the anti-CD8 scFv, and/or the anti-target epitope scFv independently comprises from N-terminus to C-terminus: VH-optional linker-VL, or VL-optional linker-VH. Any linker in the “Linkers” subsection below can be used for linking VH and VL of the anti-CD3 scFv, the anti-CD8 scFv, and/or the anti-target epitope scFv.

In some embodiments, the anti-CD3 moiety, the anti-CD8 moiety, and/or the anti-target epitope moiety is independently a Fab fragment (anti-CD3 Fab fragment, anti-CD8 Fab fragment, or anti-target epitope Fab fragment, respectively), comprising a first polypeptide comprising VH-CH1, and a second polypeptide comprising VL-CL. In some embodiments, the configuration of the variable and constant regions within the anti-CD3 Fab fragment, the anti-CD8 Fab fragment, and/or the anti-target epitope Fab fragment may be different from what is found in a native Fab fragment. In some embodiments, the anti-CD3 Fab fragment, anti-CD8 Fab fragment, and/or anti-target epitope Fab fragment independently comprises a first polypeptide comprising VH-CL, and a second polypeptide comprising VL-CH1 (see, for example, Shaefer et al. (2011), PNAS, 108:111870-92, the content of which is incorporated herein by reference in its entirety).

In some embodiments, the anti-CD3 moiety, the anti-CD8 moiety, and/or the anti-target epitope moiety is independently a monoclonal antibody or a full-length antibody. Suitable monoclonal antibodies may be of any type, including IgA, IgM, IgD, IgG, IgE and subtypes thereof, such as IgG1, IgG2, IgG3, and IgG4. The light chain domains may be derived from the kappa or lambda chain. In some embodiments, the anti-CD3 moiety, the anti-CD8 moiety, and/or the anti-target epitope moiety is independently designed recombinantly.

In some embodiments, the anti-CD3 moiety, the anti-CD8 moiety, and/or the anti-target epitope moiety is independently a Fab fragment (e.g., anti-CD3 Fab fragment, anti-CD8 Fab fragment, or anti-target epitope Fab fragment, respectively) comprising a VH, a CH1, a VL, and a CL. In some embodiments, the CH1 and VH heterodimerize with the VL and CL, and are covalently linked by a disulfide bond between the heavy and light chain constant regions. In some embodiments, the Fab fragment has the basic structure NH₂-VL-CL-S-S-CH1-VH-NH₂. In some embodiments, the CH1 and the CL of the Fab fragment are connected by one or more disulfide bonds. In some embodiments, the number of disulfide bonds between CH1 and CL of the Fab fragment is at least one, such as 2, 3, 4, 5, or more. In some embodiments, cysteine residues are engineered in the Fab fragment (such as in the CH1 and CL regions) to introduce disulfide bonds.

In some embodiments, the Fab fragment (e.g., anti-CD3 Fab fragment, anti-CD8 Fab fragment, and/or anti-target epitope Fab fragment) does not comprise a disulfide bond at the C-terminus. For example, the heavy and light chains of the Fab fragment may be engineered in such a way so as to stably interact without the need for disulfide bonds. In some embodiments, the heavy chain or light chain can be engineered to remove a cysteine residue at the C-terminus, and the heavy and light chains still stably interact and function as a Fab. In some embodiments, mutations are made to facilitate stable interactions between the heavy and light chains of the Fab fragment. For example, a “knobs into holes” engineering strategy can be used to facilitate dimerization between the heavy and light chains of a Fab (see e.g., 1996 Protein Engineering, 9:617-621). Also contemplated for use herein are variant Fab fragments designed for a particular purpose, for example, amino acid changes in the constant domains of CH1 and/or CL, and removal of a disulfide bond or addition of tags for purification, etc.

In some embodiments, the CH1 and the CL of the Fab fragment (e.g., anti-CD3 Fab fragment, anti-CD8 Fab fragment, and/or anti-target epitope Fab fragment) are connected by about 1 to about 5 (such as 1, 2, 3, 4, or 5) disulfide bonds, such as about 2 disulfide bonds. In some embodiments, the Fab fragment comprises a human immunoglobulin CH1, e.g., comprising the amino acid sequence of SEQ ID NO: 93. In some embodiments, the Fab fragment comprises a human lambda light chain constant region, e.g., comprising the amino acid sequence of SEQ ID NO: 94. In some embodiments, the Fab fragment comprises a human kappa light chain constant region, e.g., comprising the amino acid sequence of SEQ ID NO: 165. In some embodiments, the C-terminus of the CH1 and/or the CL of the Fab fragment comprises a covalent binding sequence of CPPC (SEQ ID NO: 143) or CPPCS (SEQ ID NO: 107) capable of forming an intermolecular disulfide bond. In some embodiments, the C-terminus of the CH1 and/or the CL of the Fab fragment does not comprise a covalent binding sequence of SEQ ID NO: 107 or 143, or any covalent binding sequence capable of forming an intermolecular disulfide bond.

In some embodiments, the anti-CD3 moiety, the anti-CD8 moiety, and/or the anti-target epitope moiety (e.g., scFv, sdAb, or Fab) comprises a particular sequence or certain variants of these sequences. In some embodiments, the amino acid variations (e.g., substitutions) in the variant sequences do not substantially reduce (e.g., reducing at most about any of 50%, 40%, 30%, 20%, 10%, 5%, 1%, or less) the ability of the anti-CD3 moiety, the anti-CD8 moiety, and/or the anti-target epitope moiety to bind to the corresponding antigen (e.g., CD3, CD8, target epitope, respectively). Also contemplated are modifications that substantially improve (e.g., improving at least about any of 1.2, 1.5, 2, 5, 10, 20, 50-fold, or more) the binding affinity of the anti-CD3 moiety, the anti-CD8 moiety, and/or the anti-target epitope moiety to the corresponding antigen (e.g., CD3, CD8, target epitope, respectively) or other properties, such as specificity, immunogenicity, and/or cross-reactivity with variants or corresponding antigen (e.g., CD3, CD8, target epitope, respectively).

In some embodiments, a specific VH and/or VL of an anti-CD3 moiety, anti-CD8 moiety, and/or anti-target epitope moiety may be used to screen a library of the complementary variable region to identify VH/VL with desirable properties, such as increased affinity for CD3, CD8, and/or a target epitope, respectively. Such methods are described, for example, in Portolano et al., J. Immunol. (1993) 150:880-887; Clarkson et al., Nature (1991) 352:624-628; and Klimka et al., British Journal of Cancer (2000) 83:252-260; Beiboer et al., J. Mol. Biol. (2000) 296:833-849; and Rader et al., PNAS (1998) 95:8910-8915; the contents of each of which are incorporated herein by reference in their entirety.

In some embodiments, the anti-CD3 antibody or antigen binding fragment thereof (or anti-CD3 moiety), anti-CD8 antibody or antigen binding fragment thereof (or anti-CD8 moiety), and/or anti-target epitope antibody or antigen binding fragment thereof (or anti-target epitope moiety) independently comprises one or more post-translational modifications.

Functional epitopes can be mapped by combinatorial alanine scanning. In this process, a combinatorial alanine-scanning strategy can be used to identify amino acids in the antigen protein (e.g., CD3, CD8, or target epitope protein) that are necessary for interaction with the antigen-binding moiety (e.g., anti-CD3 moiety, anti-CD8 moiety, or anti-target epitope moiety, respectively). In some embodiments, the epitope is conformational, and crystal structure of the antigen-binding moiety (e.g., anti-CD3 antibody or antigen-binding fragment thereof, anti-CD8 antibody or antigen-binding fragment thereof, or anti-target epitope antibody or antigen-binding fragment thereof) bound to its antigen (e.g., CD3, CD8, or target epitope, respectively) may be employed to identify the epitopes.

In some embodiments, there is also provided an anti-CD3 antibody or antigen binding fragment thereof (or anti-CD3 moiety) which competes with any one of the anti-CD3 antibodies or antigen-binding fragments thereof described herein for binding to CD3 (e.g., same CD3 epitope, overlapping CD3 epitope). In some embodiments, there is also provided an anti-CD8 antibody or antigen binding fragment thereof (or anti-CD8 moiety) which competes with any one of the anti-CD8 antibodies or antigen-binding fragments thereof described herein for binding to CD8 (e.g., same CD8 epitope, overlapping CD8 epitope). In some embodiments, there is also provided an anti-target epitope antibody or antigen binding fragment thereof (or anti-target epitope moiety) which competes with any one of the anti-target epitope antibodies or antigen-binding fragments thereof described herein for binding to target epitope (e.g., same target epitope, overlapping target epitope).

Competition assays may be used to identify an antibody or antigen binding fragment thereof that competes with any of the antibodies or antigen-binding fragments thereof described herein (e.g., anti-CD3 antibody or antigen-binding fragment thereof, anti-CD8 antibody or antigen-binding fragment thereof, or anti-target epitope antibody or antigen-binding fragment thereof) for binding to its target (e.g., CD3, CD8, or target epitope, respectively). Competition assays can be used to determine whether two antibodies or antigen binding fragments thereof bind the same epitope by recognizing identical or sterically overlapping epitopes, or one antibody (or antigen binding fragment thereof) competitively inhibits binding of another antibody (or antigen binding fragment thereof) to the antigen. In certain embodiments, such a competing antibody (or antigen binding fragment thereof) binds to the same epitope that is bound by any of the antibodies or antigen-binding fragments thereof described herein. Exemplary competition assays include, but are not limited to, routine assays such as those provided in Harlow and Lane (1988) Antibodies: A Laboratory Manual ch.14 (Cold Spring Harbor Laboratory, Cold Spring Harbor, N.Y.). Detailed exemplary methods for mapping an epitope to which an antibody (or antigen binding fragment thereof) binds are provided in Morris (1996) “Epitope Mapping Protocols,” in Methods in Molecular Biology vol. 66 (Humana Press, Totowa, N.J.). In some embodiments, two antibodies (or antigen binding fragments thereof) are said to bind to the same epitope if each blocks binding of the other by about 50% or more (e.g., at least about any of 60%, 70%, 80%, 90%, 95%, or more). In some embodiments, the antibody (or antigen binding fragments thereof) that competes with any of the antibodies or antigen-binding fragments thereof described herein is a humanized antibody, a human antibody, a multispecific antibody, a monoclonal antibody, a multivalent antibody, a conjugated antibody, or a chimeric antibody. In some embodiments, the competing antibody or antigen-binding fragment thereof is a full-length antibody, a Fab, a Fab′, a Fab′-SH, a F(ab′)₂, an Fv, an scFv, or any combinations thereof.

In some embodiments, the MSAPs (such as TSAPs) described herein have an increased (e.g., increasing at least about any of 1.5, 2, 3, 4, 5, 6, 7, 8, 9, 10, or more times) in vivo half-life compared to: the anti-CD3 moiety (e.g., Fab, scFv) alone, and/or the anti-CD8 moiety (e.g., Fab, scFv) alone, and/or the anti-target epitope moiety (e.g., Fab, scFv) alone.

### Anti-CD3 Moiety

The present invention also provides anti-CD3 antibodies or antigen binding fragments thereof that specifically bind to CD3. Any of the anti-CD3 antibodies or antigen binding fragments thereof described herein can be used as an anti-CD3 moiety in any of the MSAPs described herein, or in the anti-CD3 constructs described herein. The terms “anti-CD3 antibody or antigen binding fragment thereof” and “anti-CD3 moiety” are used interchangeably herein. Also see “IV. Anti-CD3 Constructs” section.

CD3 is a multi-protein complex of six chains (see, Abbas and Lichtman, 2003; Janeway et al., p172 and 178, 1999). In mammals, the T cell receptor (TCR) complex comprises a CD3γ chain, a CD3δ chain, two CD3ε chains, and a homodimer of CD35 chains. The CD3Y, CD3δ, and CD3& chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged TCR chains. The intracellular tails of the CD3Y, CD3δ, and CD3δ chains each contain a single conserved motif known as an immunoreceptor tyrosine-based activation motif or ITAM, whereas each CD35 chain has three. Without being bound by theory, it is believed the ITAMs are important for the signaling capacity of a TCR complex. CD3 as used herein may be from various animal species, including human, primate, mouse, rat, rabbit, or other mammals. In some embodiments, the anti-CD3 moiety specifically recognizes a human CD3. In some embodiments, the anti-CD3 moiety specifically recognizes a cynomolgus monkey CD3. In some embodiments, the anti-CD3 moiety specifically recognizes both human and cynomolgus monkey CD3. CD3 as used herein can be wild-type or mutant (e.g., has insertion, deletion, and/or amino acid substitution compared to wild-type). In some embodiments, the anti-CD3 moiety specifically binds to the extracellular domain of one or more of CD3ε, CD3γ, and CD3δ. In some embodiments, the anti-CD3 moiety specifically binds to the extracellular domain of CD3ε. In some embodiments, the anti-CD3 moiety specifically binds to the extracellular domain of CD3γ. In some embodiments, the anti-CD3 moiety specifically binds to the extracellular domain of CD38.

In some embodiments, the anti-CD3 moiety (e.g., full-length, sdAb, scFv, or Fab) binds to CD3 with an equilibrium binding constant (K_{d})≤1 μM, such as ≤100 nM, preferably ≤10 nM, more preferably ≤1 nM. For example, the K_{d} value of the anti-CD3 moiety in binding to CD3 is between about 1 nM and about 1 μM. In some embodiments, the anti-CD3 moiety binds to human CD3 and/or a monkey (e.g., cynomolgus) CD3 with a K_{d} of from about 1×10⁻¹² M to about 1×10⁻⁷ M (e.g., from about 1×10⁻¹² M to about 1×10⁻¹⁰ M, from about 1×10⁻¹⁰ M to about 1×10⁻⁹ M, from about 1×10⁻¹⁰ M to about 1×10⁻⁸ M, from about 1×10⁻¹⁰ M to about 1×10⁻⁷ M, from about 1×10⁻⁹ M to about 1×10⁻⁷ M, from about 1×10⁻⁹ M to about 1×10⁻⁸ M, from about 1×10⁻¹¹ M to about 1×10⁻⁷ M, or from about 1×10⁻⁸ M to about 1×10⁻⁷ M).

In some embodiments, the anti-CD3 moiety (e.g., full-length, sdAb, scFv, or Fab) specifically binds to an individual CD3 chain, such as CD3γ chain, CD3δ chain, or CD3δ chain. In some embodiments, the anti-CD3 moiety specifically binds to a complex formed from two or more individual CD3 chains (e.g., a complex of more than one CD3& chains, a complex of a CD3γ and CD3& chain, or a complex of a CD38 and CD3& chain).

In some embodiments, the anti-CD3 moiety (e.g., full-length, sdAb, scFv, or Fab) specifically binds to both human and non-human primates (such as cynomolgus monkey) CD3. Exemplary anti-human CD3 antibodies and antigen binding fragments thereof with cross reactivity to human and monkey CD3 include, but are not limited to, SP34 mouse monoclonal antibody (see, for example, Pressano, S. The EMBO J. 4:337-344, 1985; Alarcon, B. EMBO J. 10:903-912, 1991; Salmeron A. et al., J. Immunol. 147:3047-52, 1991; Yoshino N. et al., Exp. Anim 49:97-110, 2000; Conrad M L. et al., Cytometry 71A: 925-33, 2007; Yang et al., J. Immunol. 137:1097-1100:1986; U.S. Pat. Nos. 8,846,042; 11,013,800; and 10,870,701). MSAPs (such as TSAPs, BSAPs) employing such anti-CD3 moieties with cross-reactivity to monkey CD3 may facilitate toxicity studies in non-human primates, which can provide more relevant safety assessments for human clinical trial candidates, without having to perform toxicity studies in chimpanzees or using surrogate molecules.

In some embodiments, the anti-CD3 moiety (e.g., full-length, sdAb, scFv, or Fab) is derived from an anti-CD3 antibody or antigen binding fragment thereof that does not have cross-reactivity to non-human primates. Such exemplary anti-CD3 moieties include the Cris-7 monoclonal antibody (Reinherz, E. L. et al. (eds.), Leukocyte typing II, Springer Verlag, New York, (1986)), BC3 monoclonal antibody (Anasetti et al. (1990) J. Exp. Med. 172:1691), OKT3 (Ortho multicenter Transplant Study Group (1985) N. Engl. J. Med. 313:337) and derivatives thereof such as OKT3 ala-ala (Herold et al. (2003) J. Clin. Invest. 11:409), visilizumab (Carpenter et al. (2002) Blood 99:2712), and 145-2C11 monoclonal antibody (Hirsch et al. (1988) J. Immunol. 140:3766). Further CD3 binding molecules contemplated herein include UCHT-1 (Beverley, P C and Callard, R. E. (1981) Eur. J. Immunol. 11:329-334) and CD3 binding molecules described in WO2004/106380; WO2010/037838; WO2008/119567; WO2007/042261; WO2010/0150918; the contents of each of which are incorporated herein by reference in their entirety.

In some embodiments, there is provided an anti-CD3 antibody or antigen binding fragment thereof (or anti-CD3 moiety) comprising a means for specifically recognizing CD3.

In some embodiments, there is provided an anti-CD3 antibody or antigen binding fragment thereof (e.g., full-length antibody, Fab, scFv) that specifically binds to CD3 (or anti-CD3 moiety), wherein the anti-CD3 antibody or antigen binding fragment thereof comprises: (a) a VH comprising one, two, or three HVRs (or CDRs) from a reference VH comprising the sequence of SEQ ID NO: 42, and/or a VL comprising one, two, or three HVRs (or CDRs) from a reference VL comprising the sequence of SEQ ID NO: 43; (b) a VH comprising one, two, or three HVRs (or CDRs) from a reference VH comprising the sequence of SEQ ID NO: 159, and/or a VL comprising one, two, or three HVRs (or CDRs) from a reference VL comprising the sequence of SEQ ID NO: 43; or (c) a VH comprising one, two, or three HVRs (or CDRs) from a reference VH comprising the sequence of SEQ ID NO: 167, and/or a VL comprising one, two, or three HVRs (or CDRs) from a reference VL comprising the sequence of SEQ ID NO: 43. In some embodiments, there is provided an anti-CD3 antibody or antigen binding fragment thereof that specifically binds to CD3 (or anti-CD3 moiety), wherein the anti-CD3 antibody or antigen binding fragment thereof comprises: (a) a VH comprising three HVRs from a reference VH comprising the sequence of SEQ ID NO: 42, and/or a VL comprising three HVRs from a reference VL comprising the sequence of SEQ ID NO: 43; (b) a VH comprising three HVRs from a reference VH comprising the sequence of SEQ ID NO: 159, and/or a VL comprising three HVRs from a reference VL comprising the sequence of SEQ ID NO: 43; or (c) a VH comprising three HVRs from a reference VH comprising the sequence of SEQ ID NO: 167, and/or a VL comprising three HVRs from a reference VL comprising the sequence of SEQ ID NO: 43. In some embodiments, there is provided an anti-CD3 antibody or antigen binding fragment thereof that specifically binds to CD3 (or anti-CD3 moiety), wherein the anti-CD3 antibody or antigen binding fragment thereof comprises: (a) a VH comprising one, two, or three HVRs selected from any of SEQ ID NOs: 36-38, and/or a VL comprising one, two, or three HVRs selected from any of SEQ ID NOs: 39-41; (b) a VH comprising one, two, or three HVRs selected from any of SEQ ID NOs: 36, 157, and 158, and/or a VL comprising one, two, or three HVRs selected from any of SEQ ID NOs: 39-41; or (c) a VH comprising one, two, or three HVRs selected from any of SEQ ID NOs: 36, 38, and 166, and/or a VL comprising one, two, or three HVRs selected from any of SEQ ID NOs: 39-41.

In some embodiments, there is provided an anti-CD3 antibody or antigen binding fragment thereof (e.g., full-length antibody, Fab, scFv) that specifically binds to CD3 (or anti-CD3 moiety), wherein the anti-CD3 antibody or antigen binding fragment thereof comprises: an HVR-H1 comprising the amino acid sequence of SEQ ID NO: 36, or a variant thereof comprising up to about 5 (e.g., 1, 2, 3, 4, or 5) amino acid variations (e.g., insertion(s), deletion(s), and/or substitution(s), such as conservative substitution(s)); an HVR-H2 comprising the amino acid sequence of any of SEQ ID NOs: 37, 157, and 166, or a variant thereof comprising up to about 5 (e.g., 1, 2, 3, 4, or 5) amino acid variations (e.g., insertion(s), deletion(s), and/or substitution(s), such as conservative substitution(s)); an HVR-H3 comprising the amino acid sequence of SEQ ID NO: 38 or 158, or a variant thereof comprising up to about 5 (e.g., 1, 2, 3, 4, or 5) amino acid variations (e.g., insertion(s), deletion(s), and/or substitution(s), such as conservative substitution(s)); an HVR-L1 comprising the amino acid sequence of SEQ ID NO: 39, or a variant thereof comprising up to about 5 (e.g., 1, 2, 3, 4, or 5) amino acid variations (e.g., insertion(s), deletion(s), and/or substitution(s), such as conservative substitution(s)); an HVR-L2 comprising the amino acid sequence of SEQ ID NO: 40, or a variant thereof comprising up to about 5 (e.g., 1, 2, 3, 4, or 5) amino acid variations (e.g., insertion(s), deletion(s), and/or substitution(s), such as conservative substitution(s)); and an HVR-L3 comprising the amino acid sequence of SEQ ID NO: 41, or a variant thereof comprising up to about 5 (e.g., 1, 2, 3, 4, or 5) amino acid variations (e.g., insertion(s), deletion(s), and/or substitution(s), such as conservative substitution(s)).

In some embodiments, there is provided an anti-CD3 antibody or antigen binding fragment thereof (e.g., full-length antibody, Fab, scFv) that specifically binds to CD3 (or anti-CD3 moiety), wherein the anti-CD3 antibody or antigen binding fragment thereof comprises: (a) an HVR-H1 comprising the amino acid sequence of SEQ ID NO: 36, or a variant thereof comprising up to about 5 (e.g., 1, 2, 3, 4, or 5) amino acid variations (e.g., insertion(s), deletion(s), and/or substitution(s), such as conservative substitution(s)); an HVR-H2 comprising the amino acid sequence of SEQ ID NO: 37, or a variant thereof comprising up to about 5 (e.g., 1, 2, 3, 4, or 5) amino acid variations (e.g., insertion(s), deletion(s), and/or substitution(s), such as conservative substitution(s)); an HVR-H3 comprising the amino acid sequence of SEQ ID NO: 38, or a variant thereof comprising up to about 5 (e.g., 1, 2, 3, 4, or 5) amino acid variations (e.g., insertion(s), deletion(s), and/or substitution(s), such as conservative substitution(s)); an HVR-L1 comprising the amino acid sequence of SEQ ID NO: 39, or a variant thereof comprising up to about 5 (e.g., 1, 2, 3, 4, or 5) amino acid variations (e.g., insertion(s), deletion(s), and/or substitution(s), such as conservative substitution(s)); an HVR-L2 comprising the amino acid sequence of SEQ ID NO: 40, or a variant thereof comprising up to about 5 (e.g., 1, 2, 3, 4, or 5) amino acid variations (e.g., insertion(s), deletion(s), and/or substitution(s), such as conservative substitution(s)); and an HVR-L3 comprising the amino acid sequence of SEQ ID NO: 41, or a variant thereof comprising up to about 5 (e.g., 1, 2, 3, 4, or 5) amino acid variations (e.g., insertion(s), deletion(s), and/or substitution(s), such as conservative substitution(s)); (b) an HVR-H1 comprising the amino acid sequence of SEQ ID NO: 36, or a variant thereof comprising up to about 5 (e.g., 1, 2, 3, 4, or 5) amino acid variations (e.g., insertion(s), deletion(s), and/or substitution(s), such as conservative substitution(s)); an HVR-H2 comprising the amino acid sequence of SEQ ID NO: 157, or a variant thereof comprising up to about 5 (e.g., 1, 2, 3, 4, or 5) amino acid variations (e.g., insertion(s), deletion(s), and/or substitution(s), such as conservative substitution(s)); an HVR-H3 comprising the amino acid sequence of SEQ ID NO: 158, or a variant thereof comprising up to about 5 (e.g., 1, 2, 3, 4, or 5) amino acid variations (e.g., insertion(s), deletion(s), and/or substitution(s), such as conservative substitution(s)); an HVR-L1 comprising the amino acid sequence of SEQ ID NO: 39, or a variant thereof comprising up to about 5 (e.g., 1, 2, 3, 4, or 5) amino acid variations (e.g., insertion(s), deletion(s), and/or substitution(s), such as conservative substitution(s)); an HVR-L2 comprising the amino acid sequence of SEQ ID NO: 40, or a variant thereof comprising up to about 5 (e.g., 1, 2, 3, 4, or 5) amino acid variations (e.g., insertion(s), deletion(s), and/or substitution(s), such as conservative substitution(s)); and an HVR-L3 comprising the amino acid sequence of SEQ ID NO: 41, or a variant thereof comprising up to about 5 (e.g., 1, 2, 3, 4, or 5) amino acid variations (e.g., insertion(s), deletion(s), and/or substitution(s), such as conservative substitution(s)); or (c) an HVR-H1 comprising the amino acid sequence of SEQ ID NO: 36, or a variant thereof comprising up to about 5 (e.g., 1, 2, 3, 4, or 5) amino acid variations (e.g., insertion(s), deletion(s), and/or substitution(s), such as conservative substitution(s)); an HVR-H2 comprising the amino acid sequence of SEQ ID NO: 166, or a variant thereof comprising up to about 5 (e.g., 1, 2, 3, 4, or 5) amino acid variations (e.g., insertion(s), deletion(s), and/or substitution(s), such as conservative substitution(s)); an HVR-H3 comprising the amino acid sequence of SEQ ID NO: 38, or a variant thereof comprising up to about 5 (e.g., 1, 2, 3, 4, or 5) amino acid variations (e.g., insertion(s), deletion(s), and/or substitution(s), such as conservative substitution(s)); an HVR-L1 comprising the amino acid sequence of SEQ ID NO: 39, or a variant thereof comprising up to about 5 (e.g., 1, 2, 3, 4, or 5) amino acid variations (e.g., insertion(s), deletion(s), and/or substitution(s), such as conservative substitution(s)); an HVR-L2 comprising the amino acid sequence of SEQ ID NO: 40, or a variant thereof comprising up to about 5 (e.g., 1, 2, 3, 4, or 5) amino acid variations (e.g., insertion(s), deletion(s), and/or substitution(s), such as conservative substitution(s)); and an HVR-L3 comprising the amino acid sequence of SEQ ID NO: 41, or a variant thereof comprising up to about 5 (e.g., 1, 2, 3, 4, or 5) amino acid variations (e.g., insertion(s), deletion(s), and/or substitution(s), such as conservative substitution(s)).

In some embodiments, there is provided an anti-CD3 antibody or antigen binding fragment thereof (e.g., full-length antibody, Fab, scFv) that specifically binds to CD3 (or anti-CD3 moiety), wherein the anti-CD3 antibody or antigen binding fragment thereof comprises: (a) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (b) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (c) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the anti-CD3 antibody or antigen binding fragment thereof comprises: (a) a VH comprising the amino acid sequence of SEQ ID NO: 42, or a variant thereof (e.g., comprising insertion(s), deletion(s), and/or substitution(s), such as conservative substitution(s)) having at least about 80% (such as at least about any of 85%, 90%, 95%, 96%, 97%, 98%, 99%, or more) sequence identity to the amino acid sequence of SEQ ID NO: 42; and/or a VL comprising the amino acid sequence of SEQ ID NO: 43, or a variant thereof (e.g., comprising insertion(s), deletion(s), and/or substitution(s), such as conservative substitution(s)) having at least about 80% (such as at least about any of 85%, 90%, 95%, 96%, 97%, 98%, 99%, or more) sequence identity to the amino acid sequence of SEQ ID NO: 43; (b) a VH comprising the amino acid sequence of SEQ ID NO: 159, or a variant thereof (e.g., comprising insertion(s), deletion(s), and/or substitution(s), such as conservative substitution(s)) having at least about 80% (such as at least about any of 85%, 90%, 95%, 96%, 97%, 98%, 99%, or more) sequence identity to the amino acid sequence of SEQ ID NO: 159; and/or a VL comprising the amino acid sequence of SEQ ID NO: 43, or a variant thereof (e.g., comprising insertion(s), deletion(s), and/or substitution(s), such as conservative substitution(s)) having at least about 80% (such as at least about any of 85%, 90%, 95%, 96%, 97%, 98%, 99%, or more) sequence identity to the amino acid sequence of SEQ ID NO: 43; or (c) a VH comprising the amino acid sequence of SEQ ID NO: 167, or a variant thereof (e.g., comprising insertion(s), deletion(s), and/or substitution(s), such as conservative substitution(s)) having at least about 80% (such as at least about any of 85%, 90%, 95%, 96%, 97%, 98%, 99%, or more) sequence identity to the amino acid sequence of SEQ ID NO: 167; and/or a VL comprising the amino acid sequence of SEQ ID NO: 43, or a variant thereof (e.g., comprising insertion(s), deletion(s), and/or substitution(s), such as conservative substitution(s)) having at least about 80% (such as at least about any of 85%, 90%, 95%, 96%, 97%, 98%, 99%, or more) sequence identity to the amino acid sequence of SEQ ID NO: 43. In some embodiments, the one or more variations reside in one or more of the HVRs. In some embodiments, the one or more variations reside in regions outside the HVRs (i.e., in the framework regions, “FRs”). In some embodiments, the anti-CD3 antibody or antigen binding fragment thereof comprises: a VH comprising the amino acid sequence of any of SEQ ID NOs: 42, 159, and 167, and a VL comprising the amino acid sequence of SEQ ID NO: 43.

In some embodiments, the anti-CD3 antigen binding fragment (or anti-CD3 moiety) is an anti-CD3 scFv. In some embodiments, there is provided an anti-CD3 scFv comprising the amino acid sequence of any of SEQ ID NOs: 50, 56, 163, 164, 183, and 184, or a variant thereof (e.g., comprising insertion(s), deletion(s), and/or substitution(s), such as conservative substitution(s)) having at least about 80% (such as at least about any one of 85%, 90%, 95%, 96%, 97%, 98%, 99%, or more) sequence identity to the amino acid sequence of any of SEQ ID NOs: 50, 56, 163, 164, 183, and 184. In some embodiments, the anti-CD3 scFv comprises the amino acid sequence of SEQ ID NO: 50, 56, 163, 164, 183, or 184.

In some embodiments, the anti-CD3 antigen binding fragment (or anti-CD3 moiety) is an anti-CD3 Fab fragment. In some embodiments, there is provided an anti-CD3 Fab fragment, wherein the anti-CD3 Fab fragment comprises: (a) a first polypeptide comprising the amino acid sequence of any of SEQ ID NOs: 89, 160, and 168, or a variant thereof (e.g., comprising insertion(s), deletion(s), and/or substitution(s), such as conservative substitution(s)) having at least about 80% (such as at least about any one of 85%, 90%, 95%, 96%, 97%, 98%, 99%, or more) sequence identity to the amino acid sequence of any of SEQ ID NOs: 89, 160, and 168; and/or a second polypeptide comprising the amino acid sequence of SEQ ID NO: 90 or 179, or a variant thereof (e.g., comprising insertion(s), deletion(s), and/or substitution(s), such as conservative substitution(s)) having at least about 80% (such as at least about any one of 85%, 90%, 95%, 96%, 97%, 98%, 99%, or more) sequence identity to the amino acid sequence of SEQ ID NO: 90 or 179. In some embodiments, the anti-CD3 Fab fragment comprises: a first polypeptide comprising the amino acid sequence of any of SEQ ID NOs: 89, 160, and 168, and a second polypeptide comprising the amino acid sequence of SEQ ID NO: 90 or 179. In some embodiments, the first polypeptide and/or the second polypeptide of the anti-CD3 Fab fragment do not comprise a hinge or portion thereof (e.g., SEQ ID NO: 107) at the C-terminus of the first polypeptide and/or the second polypeptide.

### Anti-CD8 Moiety

The present invention also provides anti-CD8 antibodies or antigen binding fragments thereof that specifically bind to CD8 (e.g., CD8α). Any of the anti-CD8 antibodies or antigen binding fragments thereof described herein can be used as an anti-CD8 moiety in any of the MSAPs described herein, or in the anti-CD8 constructs described herein. The terms “anti-CD8 antibody or antigen binding fragment thereof” and “anti-CD8 moiety” are used interchangeably herein. Also see “III. Anti-CD8 Constructs” section.

CD8 is a disulfide-linked dimer of α and β chain, two α chains, or two β chains (see, Weber and Cantor, Encyclopedia of Immunology, p475-478, 1998; Li et al., Front Immunol. 4:206, 2013). The most common form of CD8 is composed of a CD8-α and CD8-β chain. In mammals, the CD8 receptor acts as a co-receptor with the TCR complex. The CD8α and CD8β chains display high homology to immunoglobulin variable domains. The CD8αβ and CD8aa dimers bind to MHC class I molecules and help stabilize ligand binding during antigen presentation and T cell receptor activation. CD8 and the TCR work cooperatively to enhance binding specificity and peptide discrimination at the TCR complex. The intracellular tail of the CD8 co-receptor recruits the tyrosine Lck to the CD8α chain to promote phosphorylation of ITAMs in the cytoplasmic tails of CD3 subunits associated with the TCR. In some embodiments, the anti-CD8 moiety specifically recognizes a human CD8. In some embodiments, the anti-CD8 moiety specifically recognizes a cynomolgus monkey CD8. In some embodiments, the anti-CD8 moiety specifically recognizes both human and cynomolgus monkey CD8. CD8 as used herein may be from various animal species, including human, primate, mouse, rat, rabbit, or other mammals. CD8 as used herein can be wild-type or mutant (e.g., has insertion, deletion, and/or amino acid substitution compared to wild-type). In some embodiments, the anti-CD8 moiety specifically binds to the extracellular domain of one or both of CD8α and CD8B. In some embodiments, the anti-CD8 moiety specifically binds to the extracellular domain of CD8α. In some embodiments, the anti-CD8 moiety specifically binds to the extracellular domain of CD8B.

In some embodiments, the anti-CD8 moiety (e.g., full-length, sdAb, scFv, or Fab) binds to CD8 with a K_{d}≤1 μM, such as ≤100 nM, preferably ≤10 nM, more preferably ≤1 nM. For example, the K_{d} value of the anti-CD8 moiety in binding to CD8 is between about 1 nM and about 1 μM. In some embodiments, the anti-CD8 moiety binds to human CD8 and/or a monkey (e.g., cynomolgus) CD8 with a K_{d} of from about 1×10⁻¹² M to about 1×10⁻⁷ M (e.g., from about 1×10⁻¹² M to about 1×10⁻¹⁰ M, from about 1×10⁻¹⁰ M to about 1×10⁻⁹ M, from about 1×10⁻¹⁰ M to about 1×10⁻⁸ M, from about 1×10⁻¹⁰ M to about 1×10⁻⁷ M, from about 1×10⁻⁹ M to about 1×10⁻⁷ M, from about 1×10⁻⁹ M to about 1×10⁻⁸ M, from about 1×10⁻¹¹ M to about 1×10⁻⁷ M, or from about 1×10⁻⁸ M to about 1×10⁻⁷ M).

In some embodiments, the anti-CD8 moiety (e.g., full-length, sdAb, scFv, or Fab) specifically binds to an individual CD8 chain, such as CD8α chain or CD8β chain. In some embodiments, the anti-CD8 fragment specifically binds to a complex formed from two or more individual CD8 chains (e.g., a complex of more than one CD8α chains, a complex of a CD8α and CD8B chain, a complex of more than one CD8β chains).

In some embodiments, the anti-CD8 moiety (e.g., full-length, sdAb, scFv, or Fab) specifically binds to both human and non-human primates (such as cynomolgus monkey) CD8. Exemplary anti-human CD8 antibodies and antigen binding fragments thereof with cross reactivity to human and monkey CD8 include, but are not limited to, cM-T807 mouse-human chimeric monoclonal antibody (see, for example, Schmitz, J. E. et al., Am J Pathol. 154(6):1923-1932, 1999), REGN5054 human monoclonal antibody (see, for example, Fredriksson, F. et al., J Nucl Med. 61(1): abstract 158), 3B5 mouse monoclonal antibody (see, for example, Woolridge, L. et al., J. Immunol. 171(12):6650-6660, 2003), LT8 mouse monoclonal antibody (see, for example, Sestak, K. et al., Vet Immunol Immunopathol. 119 (1-2): 21-26, 2007), FK18 mouse monoclonal antibody (see, for example, Stevens, H. P. J. D. et al., J Med Primatol. 20(8):386-393, 1991), or RPA-T8 monoclonal mouse antibody (see, for example, Bozkus, C. C. et al., STAR Protoc. 2 (3): 100758, 2021). MSAPs (such as TSAPs, BSAPs) employing such anti-CD8 moieties with cross-reactivity to monkey CD8 may facilitate toxicity studies in non-human primates, which can provide more relevant safety assessments for human clinical trial candidates, without having to perform toxicity studies in chimpanzees or using surrogate molecules.

In some embodiments, the anti-CD8 moiety (e.g., full-length, sdAb, scFv, or Fab) is derived from an anti-CD8 antibody or antigen binding fragment thereof that does not have cross-reactivity to non-human primates. Such exemplary anti-CD8 moieties include, but are not limited to, the QA18A37 mouse monoclonal antibody (see, for example, Paulikat, A. D. et al. J Innate Immun.14 (5): 569-580, 2022), MEM-31 mouse monoclonal antibody (see, for example, Horejsí, V. et al., Folia Biol. 34(1):23-24, 1988), UCHT-4 mouse monoclonal antibody (see, for example, Willemsen, R. A. et al. J Immunol. 177(2):991-998, 2006), and OKT8 mouse monoclonal antibody (see, for example, Shiratsuchi, H. and Tsuyuguchi, I., Clin Exp Immunol. 57(2):271-278, 1984). Further CD8 binding molecules contemplated herein include CD8 binding molecules described in U.S. Pat. No. 11,535,869; WO2021/046159; WO2019/023148; WO2019/033043; WO1993/008817; WO2014/164553; the contents of each of which are incorporated herein by reference in their entirety.

In some embodiments, there is provided an anti-CD8 antibody or antigen binding fragment thereof (or anti-CD8 moiety) comprising a means for specifically recognizing CD8.

In some embodiments, there is provided an anti-CD8 antibody or antigen binding fragment thereof (e.g., full-length antibody, Fab, scFv) that specifically binds to CD8 (or anti-CD8 moiety), wherein the anti-CD8 antibody or antigen binding fragment thereof comprises: a VH comprising one, two, or three HVRs (or CDRs) from a reference VH comprising the sequence of any of SEQ ID NOs: 24, 26, 28, 30, 32, and 34, and/or a VL comprising one, two, or three HVRs (or CDRs) from a reference VL comprising the sequence of any of SEQ ID NOs: 25, 27, 29, 33, and 35. In some embodiments, there is provided an anti-CD8 antibody or antigen binding fragment thereof that specifically binds to CD8 (or anti-CD8 moiety), wherein the anti-CD8 antibody or antigen binding fragment thereof comprises: i) a VH comprising three HVRs from a reference VH comprising the sequence of SEQ ID NO: 24, and/or a VL comprising three HVRs from a reference VL comprising the sequence of SEQ ID NO: 25; ii) a VH comprising three HVRs from a reference VH comprising the sequence of SEQ ID NO: 26, and/or a VL comprising three HVRs from a reference VL comprising the sequence of SEQ ID NO: 27; iii) a VH comprising three HVRs from a reference VH comprising the sequence of SEQ ID NO: 28, and/or a VL comprising three HVRs from a reference VL comprising the sequence of SEQ ID NO: 29; iv) a VH comprising three HVRs from a reference VH comprising the sequence of SEQ ID NO: 30, and/or a VL comprising three HVRs from a reference VL comprising the sequence of SEQ ID NO: 25; v) a VH comprising three HVRs from a reference VH comprising the sequence of SEQ ID NO: 32, and/or a VL comprising three HVRs from a reference VL comprising the sequence of SEQ ID NO: 33; or vi) a VH comprising three HVRs from a reference VH comprising the sequence of SEQ ID NO: 34, and/or a VL comprising three HVRs from a reference VL comprising the sequence of SEQ ID NO: 35. In some embodiments, there is provided an anti-CD8 antibody or antigen binding fragment thereof that specifically binds to CD8 (or anti-CD8 moiety), wherein the anti-CD8 antibody or antigen binding fragment thereof comprises: i) a VH comprising one, two, or three HVRs selected from any of SEQ ID NOs: 13-15, and a VL comprising one, two, or three HVRs selected from any of SEQ ID NOs: 4-6; ii) a VH comprising one, two, or three HVRs selected from any of SEQ ID NOs: 1, 16, and 3, and a VL comprising one, two, or three HVRs selected from any of SEQ ID NOs: 4-6; iii) a VH comprising one, two, or three HVRs selected from any of SEQ ID NOs: 1, 2, and 17, and a VL comprising one, two, or three HVRs selected from any of SEQ ID NOs: 4-6; iv) a VH comprising one, two, or three HVRs selected from any of SEQ ID NOs: 1, 18, and 19, and a VL comprising one, two, or three HVRs selected from any of SEQ ID NOs: 4-6; v) a VH comprising one, two, or three HVRs selected from any of SEQ ID NOs: 7-9, and a VL comprising one, two, or three HVRs selected from any of SEQ ID NOs: 10-12; or vi) a VH comprising one, two, or three HVRs selected from any of SEQ ID NOs: 20, 2, and 22, and a VL comprising one, two, or three HVRs selected from any of SEQ ID NOs: 4, 5, and 23.

In some embodiments, there is provided an anti-CD8 antibody or antigen binding fragment thereof (e.g., full-length antibody, Fab, scFv) that specifically binds to CD8 (or anti-CD8 moiety), wherein the anti-CD8 antibody or antigen binding fragment thereof comprises an HVR-H1 comprising the amino acid sequence of any of SEQ ID NOs: 1, 7, 13, and 20, or a variant thereof comprising up to about 5 (e.g., 1, 2, 3, 4, or 5) amino acid variations (e.g., insertion(s), deletion(s), and/or substitution(s), such as conservative substitution(s)); an HVR-H2 comprising the amino acid sequence of any of SEQ ID NOs: 2, 8, 14, 16, and 18, or a variant thereof comprising up to about 5 (e.g., 1, 2, 3, 4, or 5) amino acid variations (e.g., insertion(s), deletion(s), and/or substitution(s), such as conservative substitution(s)); an HVR-H3 comprising the amino acid sequence of any of SEQ ID NOs: 3, 9, 15, 17, 19, and 22, or a variant thereof comprising up to about 5 (e.g., 1, 2, 3, 4, or 5) amino acid variations (e.g., insertion(s), deletion(s), and/or substitution(s), such as conservative substitution(s)); an HVR-L1 comprising the amino acid sequence of SEQ ID NO: 4 or 10, or a variant thereof comprising up to about 5 (e.g., 1, 2, 3, 4, or 5) amino acid variations (e.g., insertion(s), deletion(s), and/or substitution(s), such as conservative substitution(s)); an HVR-L2 comprising the amino acid sequence of SEQ ID NO: 5 or 11, or a variant thereof comprising up to about 5 (e.g., 1, 2, 3, 4, or 5) amino acid variations (e.g., insertion(s), deletion(s), and/or substitution(s), such as conservative substitution(s)); and an HVR-L3 comprising the amino acid sequence of any of SEQ ID NOs: 6, 12, and 23, or a variant thereof comprising up to about 5 (e.g., 1, 2, 3, 4, or 5) amino acid variations (e.g., insertion(s), deletion(s), and/or substitution(s), such as conservative substitution(s)). In some embodiments, there is provided an anti-CD8 antibody or antigen binding fragment thereof that specifically binds to CD8 (or anti-CD8 moiety), wherein the anti-CD8 antibody or antigen binding fragment thereof comprises: an HVR-H1 comprising the amino acid sequence of any of SEQ ID NOs: 1, 7, 13, and 20; an HVR-H2 comprising the amino acid sequence of any of SEQ ID NOs: 2, 8, 14, 16, and 18; an HVR-H3 comprising the amino acid sequence of any of SEQ ID NOs: 3, 9, 15, 17, 19, and 22; an HVR-L1 comprising the amino acid sequence of SEQ ID NO: 4 or 10; an HVR-L2 comprising the amino acid sequence of SEQ ID NO: 5 or 11; and an HVR-L3 comprising the amino acid sequence of any of SEQ ID NOs: 6, 12, and 23.

In some embodiments, there is provided an anti-CD8 antibody or antigen binding fragment thereof (e.g., full-length antibody, Fab, scFv) that specifically binds to CD8 (or anti-CD8 moiety), wherein the anti-CD8 antibody or antigen binding fragment thereof comprises: i) an HVR-H1 comprising the amino acid sequence of SEQ ID NO: 13, or a variant thereof comprising up to about 5 (e.g., 1, 2, 3, 4, or 5) amino acid variations (e.g., insertion(s), deletion(s), and/or substitution(s), such as conservative substitution(s)), an HVR-H2 comprising the amino acid sequence of SEQ ID NO: 14, or a variant thereof comprising up to about 5 (e.g., 1, 2, 3, 4, or 5) amino acid variations (e.g., insertion(s), deletion(s), and/or substitution(s), such as conservative substitution(s)), an HVR-H3 comprising the amino acid sequence of SEQ ID NO: 15, or a variant thereof comprising up to about 5 (e.g., 1, 2, 3, 4, or 5) amino acid variations (e.g., insertion(s), deletion(s), and/or substitution(s), such as conservative substitution(s)), an HVR-L1 comprising the amino acid sequence of SEQ ID NO: 4, or a variant thereof comprising up to about 5 (e.g., 1, 2, 3, 4, or 5) amino acid variations (e.g., insertion(s), deletion(s), and/or substitution(s), such as conservative substitution(s)), an HVR-L2 comprising the amino acid sequence of SEQ ID NO: 5, or a variant thereof comprising up to about 5 (e.g., 1, 2, 3, 4, or 5) amino acid variations (e.g., insertion(s), deletion(s), and/or substitution(s), such as conservative substitution(s)), and an HVR-L3 comprising the amino acid sequence of SEQ ID NO: 6, or a variant thereof comprising up to about 5 (e.g., 1, 2, 3, 4, or 5) amino acid variations (e.g., insertion(s), deletion(s), and/or substitution(s), such as conservative substitution(s)); ii) an HVR-H1 comprising the amino acid sequence of SEQ ID NO: 1, or a variant thereof comprising up to about 5 (e.g., 1, 2, 3, 4, or 5) amino acid variations (e.g., insertion(s), deletion(s), and/or substitution(s), such as conservative substitution(s)), an HVR-H2 comprising the amino acid sequence of SEQ ID NO: 16, or a variant thereof comprising up to about 5 (e.g., 1, 2, 3, 4, or 5) amino acid variations (e.g., insertion(s), deletion(s), and/or substitution(s), such as conservative substitution(s)), an HVR-H3 comprising the amino acid sequence of SEQ ID NO: 3, or a variant thereof comprising up to about 5 (e.g., 1, 2, 3, 4, or 5) amino acid variations (e.g., insertion(s), deletion(s), and/or substitution(s), such as conservative substitution(s)), an HVR-L1 comprising the amino acid sequence of SEQ ID NO: 4, or a variant thereof comprising up to about 5 (e.g., 1, 2, 3, 4, or 5) amino acid variations (e.g., insertion(s), deletion(s), and/or substitution(s), such as conservative substitution(s)), an HVR-L2 comprising the amino acid sequence of SEQ ID NO: 5, or a variant thereof comprising up to about 5 (e.g., 1, 2, 3, 4, or 5) amino acid variations (e.g., insertion(s), deletion(s), and/or substitution(s), such as conservative substitution(s)), and an HVR-L3 comprising the amino acid sequence of SEQ ID NO: 6, or a variant thereof comprising up to about 5 (e.g., 1, 2, 3, 4, or 5) amino acid variations (e.g., insertion(s), deletion(s), and/or substitution(s), such as conservative substitution(s)); iii) an HVR-H1 comprising the amino acid sequence of SEQ ID NO: 1, or a variant thereof comprising up to about 5 (e.g., 1, 2, 3, 4, or 5) amino acid variations (e.g., insertion(s), deletion(s), and/or substitution(s), such as conservative substitution(s)), an HVR-H2 comprising the amino acid sequence of SEQ ID NO: 2, or a variant thereof comprising up to about 5 (e.g., 1, 2, 3, 4, or 5) amino acid variations (e.g., insertion(s), deletion(s), and/or substitution(s), such as conservative substitution(s)), an HVR-H3 comprising the amino acid sequence of SEQ ID NO: 17, or a variant thereof comprising up to about 5 (e.g., 1, 2, 3, 4, or 5) amino acid variations (e.g., insertion(s), deletion(s), and/or substitution(s), such as conservative substitution(s)), an HVR-L1 comprising the amino acid sequence of SEQ ID NO: 4, or a variant thereof comprising up to about 5 (e.g., 1, 2, 3, 4, or 5) amino acid variations (e.g., insertion(s), deletion(s), and/or substitution(s), such as conservative substitution(s)), an HVR-L2 comprising the amino acid sequence of SEQ ID NO: 5, or a variant thereof comprising up to about 5 (e.g., 1, 2, 3, 4, or 5) amino acid variations (e.g., insertion(s), deletion(s), and/or substitution(s), such as conservative substitution(s)), and an HVR-L3 comprising the amino acid sequence of SEQ ID NO: 6, or a variant thereof comprising up to about 5 (e.g., 1, 2, 3, 4, or 5) amino acid variations (e.g., insertion(s), deletion(s), and/or substitution(s), such as conservative substitution(s)); iv) an HVR-H1 comprising the amino acid sequence of SEQ ID NO: 1, or a variant thereof comprising up to about 5 (e.g., 1, 2, 3, 4, or 5) amino acid variations (e.g., insertion(s), deletion(s), and/or substitution(s), such as conservative substitution(s)), an HVR-H2 comprising the amino acid sequence of SEQ ID NO: 18, or a variant thereof comprising up to about 5 (e.g., 1, 2, 3, 4, or 5) amino acid variations (e.g., insertion(s), deletion(s), and/or substitution(s), such as conservative substitution(s)), an HVR-H3 comprising the amino acid sequence of SEQ ID NO: 19, or a variant thereof comprising up to about 5 (e.g., 1, 2, 3, 4, or 5) amino acid variations (e.g., insertion(s), deletion(s), and/or substitution(s), such as conservative substitution(s)), an HVR-L1 comprising the amino acid sequence of SEQ ID NO: 4, or a variant thereof comprising up to about 5 (e.g., 1, 2, 3, 4, or 5) amino acid variations (e.g., insertion(s), deletion(s), and/or substitution(s), such as conservative substitution(s)), an HVR-L2 comprising the amino acid sequence of SEQ ID NO: 5, or a variant thereof comprising up to about 5 (e.g., 1, 2, 3, 4, or 5) amino acid variations (e.g., insertion(s), deletion(s), and/or substitution(s), such as conservative substitution(s)), and an HVR-L3 comprising the amino acid sequence of SEQ ID NO: 6, or a variant thereof comprising up to about 5 (e.g., 1, 2, 3, 4, or 5) amino acid variations (e.g., insertion(s), deletion(s), and/or substitution(s), such as conservative substitution(s)); v) an HVR-H1 comprising the amino acid sequence of SEQ ID NO: 7, or a variant thereof comprising up to about 5 (e.g., 1, 2, 3, 4, or 5) amino acid variations (e.g., insertion(s), deletion(s), and/or substitution(s), such as conservative substitution(s)), an HVR-H2 comprising the amino acid sequence of SEQ ID NO: 8, or a variant thereof comprising up to about 5 (e.g., 1, 2, 3, 4, or 5) amino acid variations (e.g., insertion(s), deletion(s), and/or substitution(s), such as conservative substitution(s)), an HVR-H3 comprising the amino acid sequence of SEQ ID NO: 9, or a variant thereof comprising up to about 5 (e.g., 1, 2, 3, 4, or 5) amino acid variations (e.g., insertion(s), deletion(s), and/or substitution(s), such as conservative substitution(s)), an HVR-L1 comprising the amino acid sequence of SEQ ID NO: 10, or a variant thereof comprising up to about 5 (e.g., 1, 2, 3, 4, or 5) amino acid variations (e.g., insertion(s), deletion(s), and/or substitution(s), such as conservative substitution(s)), an HVR-L2 comprising the amino acid sequence of SEQ ID NO: 11, or a variant thereof comprising up to about 5 (e.g., 1, 2, 3, 4, or 5) amino acid variations (e.g., insertion(s), deletion(s), and/or substitution(s), such as conservative substitution(s)), and an HVR-L3 comprising the amino acid sequence of SEQ ID NO: 12, or a variant thereof comprising up to about 5 (e.g., 1, 2, 3, 4, or 5) amino acid variations (e.g., insertion(s), deletion(s), and/or substitution(s), such as conservative substitution(s)); or vi) an HVR-H1 comprising the amino acid sequence of SEQ ID NO: 20, or a variant thereof comprising up to about 5 (e.g., 1, 2, 3, 4, or 5) amino acid variations (e.g., insertion(s), deletion(s), and/or substitution(s), such as conservative substitution(s)), an HVR-H2 comprising the amino acid sequence of SEQ ID NO: 2, or a variant thereof comprising up to about 5 (e.g., 1, 2, 3, 4, or 5) amino acid variations (e.g., insertion(s), deletion(s), and/or substitution(s), such as conservative substitution(s)), an HVR-H3 comprising the amino acid sequence of SEQ ID NO: 22, or a variant thereof comprising up to about 5 (e.g., 1, 2, 3, 4, or 5) amino acid variations (e.g., insertion(s), deletion(s), and/or substitution(s), such as conservative substitution(s)), an HVR-L1 comprising the amino acid sequence of SEQ ID NO: 4, or a variant thereof comprising up to about 5 (e.g., 1, 2, 3, 4, or 5) amino acid variations (e.g., insertion(s), deletion(s), and/or substitution(s), such as conservative substitution(s)), an HVR-L2 comprising the amino acid sequence of SEQ ID NO: 5, or a variant thereof comprising up to about 5 (e.g., 1, 2, 3, 4, or 5) amino acid variations (e.g., insertion(s), deletion(s), and/or substitution(s), such as conservative substitution(s)), and an HVR-L3 comprising the amino acid sequence of SEQ ID NO: 23, or a variant thereof comprising up to about 5 (e.g., 1, 2, 3, 4, or 5) amino acid variations (e.g., insertion(s), deletion(s), and/or substitution(s), such as conservative substitution(s)).

In some embodiments, there is provided an anti-CD8 antibody or antigen binding fragment thereof (e.g., full-length antibody, Fab, scFv) that specifically binds to CD8 (or anti-CD8 moiety), wherein the anti-CD8 antibody or antigen binding fragment thereof comprises: i) an HVR-H1 comprising the amino acid sequence of SEQ ID NO: 13, an HVR-H2 comprising the amino acid sequence of SEQ ID NO: 14, an HVR-H3 comprising the amino acid sequence of SEQ ID NO: 15, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; ii) an HVR-H1 comprising the amino acid sequence of SEQ ID NO: 1, an HVR-H2 comprising the amino acid sequence of SEQ ID NO: 16, an HVR-H3 comprising the amino acid sequence of SEQ ID NO: 3, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iii) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iv) an HVR-H1 comprising the amino acid sequence of SEQ ID NO: 1, an HVR-H2 comprising the amino acid sequence of SEQ ID NO: 18, an HVR-H3 comprising the amino acid sequence of SEQ ID NO: 19, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; v) an HVR-H1 comprising the amino acid sequence of SEQ ID NO: 7, an HVR-H2 comprising the amino acid sequence of SEQ ID NO: 8, an HVR-H3 comprising the amino acid sequence of SEQ ID NO: 9, an HVR-L1 comprising the amino acid sequence of SEQ ID NO: 10, an HVR-L2 comprising the amino acid sequence of SEQ ID NO: 11, and an HVR-L3 comprising the amino acid sequence of SEQ ID NO: 12; or vi) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23.

In some embodiments, there is provided an anti-CD8 antibody or antigen binding fragment thereof (e.g., full-length antibody, Fab, scFv) that specifically binds to CD8 (or anti-CD8 moiety), wherein the anti-CD8 antibody or antigen binding fragment thereof comprises: a VH comprising the amino acid sequence of any of SEQ ID NOs: 24, 26, 28, 30, 32, and 34, or a variant thereof (e.g., comprising insertion(s), deletion(s), and/or substitution(s), such as conservative substitution(s)) having at least about 80% (such as at least about any of 85%, 90%, 95%, 96%, 97%, 98%, 99%, or more) sequence identity to the amino acid sequence of any of SEQ ID NOs: 24, 26, 28, 30, 32, and 34; and/or a VL comprising the amino acid sequence of any of SEQ ID NOs: 25, 27, 29, 33, and 35, or a variant thereof (e.g., comprising insertion(s), deletion(s), and/or substitution(s), such as conservative substitution(s)) having at least about 80% (such as at least about any of 85%, 90%, 95%, 96%, 97%, 98%, 99%, or more) sequence identity to the amino acid sequence of any of SEQ ID NOs: 25, 27, 29, 33, and 35. In some embodiments, there is provided an anti-CD8 antibody or antigen binding fragment thereof that specifically binds to CD8 (or anti-CD8 moiety), wherein the anti-CD8 antibody or antigen binding fragment thereof comprises: i) a VH comprising the amino acid sequence of SEQ ID NO: 24, or a variant thereof having at least about 80% sequence identity to the amino acid sequence of SEQ ID NO: 24, and a VL comprising the amino acid sequence of SEQ ID NO: 25, or a variant thereof having at least about 80% sequence identity to the amino acid sequence of SEQ ID NO: 25; ii) a VH comprising the amino acid sequence of SEQ ID NO: 26, or a variant thereof having at least about 80% sequence identity to the amino acid sequence of SEQ ID NO: 26, and a VL comprising the amino acid sequence of SEQ ID NO: 27, or a variant thereof having at least about 80% sequence identity to the amino acid sequence of SEQ ID NO: 27; iii) a VH comprising the amino acid sequence of SEQ ID NO: 28, or a variant thereof having at least about 80% sequence identity to the amino acid sequence of SEQ ID NO: 28, and a VL comprising the amino acid sequence of SEQ ID NO: 29, or a variant thereof having at least about 80% sequence identity to the amino acid sequence of SEQ ID NO: 29; iv) a VH comprising the amino acid sequence of SEQ ID NO: 30, or a variant thereof having at least about 80% sequence identity to the amino acid sequence of SEQ ID NO: 30, and a VL comprising the amino acid sequence of SEQ ID NO: 25, or a variant thereof having at least about 80% sequence identity to the amino acid sequence of SEQ ID NO: 25; v) a VH comprising the amino acid sequence of SEQ ID NO: 32, or a variant thereof having at least about 80% sequence identity to the amino acid sequence of SEQ ID NO: 32, and a VL comprising the amino acid sequence of SEQ ID NO: 33, or a variant thereof having at least about 80% sequence identity to the amino acid sequence of SEQ ID NO: 33; or vi) a VH comprising the amino acid sequence of SEQ ID NO: 34, or a variant thereof having at least about 80% sequence identity to the amino acid sequence of SEQ ID NO: 34, and a VL comprising the amino acid sequence of SEQ ID NO: 35, or a variant thereof having at least about 80% sequence identity to the amino acid sequence of SEQ ID NO: 35. In some embodiments, the one or more variations (e.g., insertion(s), deletion(s), and/or substitution(s), such as conservative substitution(s)) reside in one or more of the HVRs. In some embodiments, the one or more variations reside in regions outside the HVRs (i.e., in the framework regions, “FRs”). In some embodiments, the anti-CD8 antibody or antigen binding fragment thereof comprises a VH comprising the amino acid sequence of any of SEQ ID NOs: 24, 26, 28, 30, 32, and 34, and/or a VL comprising the amino acid sequence of any of SEQ ID NOs: 25, 27, 29, 33, and 35. In some embodiments, the anti-CD8 antibody or antigen binding fragment thereof comprises: i) a VH comprising the amino acid sequence of SEQ ID NO: 24, and a VL comprising the amino acid sequence of SEQ ID NO: 25; ii) a VH comprising the amino acid sequence of SEQ ID NO: 26, and a VL comprising the amino acid sequence of SEQ ID NO: 27; iii) a VH comprising the amino acid sequence of SEQ ID NO: 28, and a VL comprising the amino acid sequence of SEQ ID NO: 29; iv) a VH comprising the amino acid sequence of SEQ ID NO: 30, and a VL comprising the amino acid sequence of SEQ ID NO: 25; v) a VH comprising the amino acid sequence of SEQ ID NO: 32, and a VL comprising the amino acid sequence of SEQ ID NO: 33; or vi) a VH comprising the amino acid sequence of SEQ ID NO: 34, and a VL comprising the amino acid sequence of SEQ ID NO: 35.

In some embodiments, the anti-CD8 antigen binding fragment (or anti-CD8 moiety) is an anti-CD8 scFv. In some embodiments, there is provided an anti-CD8 scFv comprising the amino acid sequence of any of SEQ ID NOs: 21, 44-49, and 51-55, or a variant thereof (e.g., comprising insertion(s), deletion(s), and/or substitution(s), such as conservative substitution(s)) having at least about 80% (such as at least about any one of 85%, 90%, 95%, 96%, 97%, 98%, 99%, or more) sequence identity to the amino acid sequence of any of SEQ ID NOs: 21, 44-49, and 51-55. In some embodiments, the anti-CD8 scFv comprises the amino acid sequence of any of SEQ ID NOs: 21, 44-49, and 51-55, such as any of SEQ ID NOs: 44-49.

In some embodiments, the anti-CD8 antigen binding fragment (or anti-CD8 moiety) is an anti-CD8 Fab fragment. In some embodiments, there is provided an anti-CD8 Fab fragment, wherein the anti-CD8 Fab fragment comprises a first polypeptide comprising the amino acid sequence of any of SEQ ID NOs: 91, 147, 149, 151, 153, and 155, or a variant thereof (e.g., comprising insertion(s), deletion(s), and/or substitution(s), such as conservative substitution(s)) having at least about 80% (such as at least about any one of 85%, 90%, 95%, 96%, 97%, 98%, 99%, or more) sequence identity to the amino acid sequence of any of SEQ ID NOs: 91, 147, 149, 151, 153, and 155; and/or a second polypeptide comprising the amino acid sequence of any of SEQ ID NOs: 92, 148, 150, 154, and 156, or a variant thereof (e.g., comprising insertion(s), deletion(s), and/or substitution(s), such as conservative substitution(s)) having at least about 80% (such as at least about any one of 85%, 90%, 95%, 96%, 97%, 98%, 99%, or more) sequence identity to the amino acid sequence of any of SEQ ID NOs: 92, 148, 150, 154, and 156. In some embodiments, the anti-CD8 Fab fragment comprises: i) a first polypeptide comprising the amino acid sequence of SEQ ID NO: 147, or a variant thereof having at least about 80% sequence identity to the amino acid sequence of SEQ ID NO: 147, and a second polypeptide comprising the amino acid sequence of SEQ ID NO: 148, or a variant thereof having at least about 80% sequence identity to the amino acid sequence of SEQ ID NO: 148; ii) a first polypeptide comprising the amino acid sequence of SEQ ID NO: 149, or a variant thereof having at least about 80% sequence identity to the amino acid sequence of SEQ ID NO: 149, and a second polypeptide comprising the amino acid sequence of SEQ ID NO: 150, or a variant thereof having at least about 80% sequence identity to the amino acid sequence of SEQ ID NO: 150; iii) a first polypeptide comprising the amino acid sequence of SEQ ID NO: 91, or a variant thereof having at least about 80% sequence identity to the amino acid sequence of SEQ ID NO: 91, and a second polypeptide comprising the amino acid sequence of SEQ ID NO: 92, or a variant thereof having at least about 80% sequence identity to the amino acid sequence of SEQ ID NO: 92; iv) a first polypeptide comprising the amino acid sequence of SEQ ID NO: 151, or a variant thereof having at least about 80% sequence identity to the amino acid sequence of SEQ ID NO: 151, and a second polypeptide comprising the amino acid sequence of SEQ ID NO:148, or a variant thereof having at least about 80% sequence identity to the amino acid sequence of SEQ ID NO: 148; v) a first polypeptide comprising the amino acid sequence of SEQ ID NO: 153, or a variant thereof having at least about 80% sequence identity to the amino acid sequence of SEQ ID NO: 153, and a second polypeptide comprising the amino acid sequence of SEQ ID NO: 154, or a variant thereof having at least about 80% sequence identity to the amino acid sequence of SEQ ID NO: 154; or vi) a first polypeptide comprising the amino acid sequence of SEQ ID NO: 155, or a variant thereof having at least about 80% sequence identity to the amino acid sequence of SEQ ID NO: 155, and a second polypeptide comprising the amino acid sequence of SEQ ID NO: 156, or a variant thereof having at least about 80% sequence identity to the amino acid sequence of SEQ ID NO: 156. In some embodiments, there is provided an anti-CD8 Fab fragment, wherein the anti-CD8 Fab fragment comprises: i) a first polypeptide comprising the amino acid sequence of SEQ ID NO: 147, and a second polypeptide comprising the amino acid sequence of SEQ ID NO: 148; ii) a first polypeptide comprising the amino acid sequence of SEQ ID NO: 149, and a second polypeptide comprising the amino acid sequence of SEQ ID NO: 150; iii) a first polypeptide comprising the amino acid sequence of SEQ ID NO: 91, and a second polypeptide comprising the amino acid sequence of SEQ ID NO: 92; iv) a first polypeptide comprising the amino acid sequence of SEQ ID NO: 151, and a second polypeptide comprising the amino acid sequence of SEQ ID NO: 148; v) a first polypeptide comprising the amino acid sequence of SEQ ID NO: 153, and a second polypeptide comprising the amino acid sequence of SEQ ID NO: 154; or vi) a first polypeptide comprising the amino acid sequence of SEQ ID NO: 155, and a second polypeptide comprising the amino acid sequence of SEQ ID NO: 156. In some embodiments, the first polypeptide and/or the second polypeptide of the anti-CD8 Fab fragment do not comprise a hinge or portion thereof (e.g., SEQ ID NO: 107) at the C-terminus of the first polypeptide and/or the second polypeptide.

### Anti-Target Epitope Moiety

Any of the anti-target epitope antibodies or antigen binding fragments thereof described herein can be used as an anti-target epitope moiety in any of the MSAPs described herein, or in the anti-CD8 constructs or anti-CD3 constructs described herein. The terms “anti-target epitope antibody or antigen binding fragment thereof” and “anti-target epitope moiety” are used interchangeably herein.

Anti-target epitope moieties can be designed against any known target epitope in the art, for example designed against known cancer antigens (e.g., tumor-associated antigens (TAA) or tumor-specific antigens (TSA)), or antigens of bacteria, viruses, fungi, auto-antigens, alloantigens, allergens, superantigens, tolerogens, haptens, neoantigens, and toxins.

In some embodiments, the anti-target epitope moiety specifically binds a target epitope of a target antigen on a cell surface. In some embodiments, the cell is a cancer cell. In some embodiments, the cancer cell is in a solid cancer. In some embodiments, the cancer cell is in a liquid cancer, e.g., hematological cancer. In some embodiments, the cancer cell is a metastatic cancer cell, such as a metastatic solid tumor malignancy, and the like.

In some embodiments, the anti-target epitope moiety specifically recognizes a target epitope of a TAA. A TAA includes any molecule (e.g., peptide, carbohydrate, lipid, nucleic acid) that can be found in some healthy cells (e.g., at low levels or during a specific developmental stage or stage of cellular differentiation) and that is perturbed in cancer cells, for example the overexpression of a specific peptide (e.g., HER2, CD19, CD20, EpCAM). Examples of TAAs include, but are not limited to, CD19, EpCAM, HER2, CD20, AFP, GPC3, CEA, PSA, MUC1, TRP-2, EGFR, mesothelin, and CA-125. In some embodiments, the TAA is CD19 or EpCAM.

In some embodiments, the anti-target epitope moiety specifically recognizes a target epitope of a TSA. A TSA includes a molecule (e.g., peptide, carbohydrate, lipid, nucleic acid) that is only found in tumor cells and is not present in normal cells. Examples of TSAs include, but are not limited to, WT1, MAGEA1, CTAG1B, mutated p53, mutated Ras, and BCR/ABL fusion protein. In some embodiments, the TSA is Wilms' tumor 1 (WT1).

WT1 is overexpressed on Wilms' tumors and is associated with worse patient prognosis. The transmembrane protein B-lymphocyte antigen CD19 (CD19), also known as CD19 molecule, B-Lymphocyte Surface Antigen B4, T-Cell Surface Antigen Leu-12 and CVID3, is a B cell marker and is overexpressed on hematological cancers. The epithelial cell adhesion molecule (EpCAM or CD326) is overexpressed on rapidly proliferating tumors of epithelial origin and is associated with worse patient prognosis. These exemplary antigens can be used as biomarkers for tumor development, prognostic indicators, and immunotherapeutic targets for antigen-expressing cancers.

The MSAPs (such as TSAPs) described herein comprise one or more (e.g., 1, 2, 3, 4, 5, or more, such as 1 or 2) anti-target epitope moieties (e.g., scFv or Fab) that specifically bind to a target epitope of a target antigen (e.g., CD19, EpCAM, or WT1). In some embodiments, the MSAP comprises two or more anti-target epitope moieties (e.g., scFvs). In some embodiments, the two or more anti-target epitope moieties (e.g., scFvs) are fused with each other in tandem, then fused to the Fab fragment (e.g., N′ of VH, N′ of VL, C′ of CH1, and/or C′ of CL).

Target epitopes or target antigens (e.g., TAAs (e.g., CD19 or EpCAM) or TSAs (e.g., WT1)) may be from various animal species, including human, primate, mouse, rat, rabbit, or other mammals. Target epitopes or target antigens can be wild-type or mutant (e.g., has insertion, deletion, and/or amino acid substitution compared to wild-type).

In some embodiments, the anti-target epitope moiety (e.g., full-length, sdAb, scFv, or Fab) binds to a target epitope with a K_{d}≤1 μM, such as ≤100 nM, preferably ≤10 nM, more preferably ≤1 nM. For example, the K_{d} value of the anti-target epitope moiety is between about 1 nM and about 1 μM. In some embodiments, the anti-target epitope moiety binds to a human target epitope and/or a monkey (e.g., cynomolgus) target epitope with a K_{d} of from about 1×10⁻¹² M to about 1×10⁻⁷ M (e.g., from about 1×10⁻¹² M to about 1×10⁻¹⁰ M, from about 1×10⁻¹¹ M to about 1×10⁻¹⁰ M, from about 1×10⁻¹¹ M to about 1×10⁻⁹ M, from about 1×10⁻¹¹ M to about 1×10⁻⁸ M, from about 1×10⁻¹⁰ M to about 1×10⁻⁹ M, from about 1×10⁻¹⁰ M to about 1×10⁻⁸ M, from about 1×10⁻¹⁰ M to about 1×10⁻⁷ M, from about 1×10⁻⁹ M to about 1×10⁻⁷ M, from about 1×10⁻⁹ M to about 1×10⁻⁸ M, from about 1×10⁻¹¹ M to about 1×10⁻⁷ M, or from about 1×10⁻⁸ M to about 1×10⁻⁷ M).

In some embodiments, the anti-target epitope moiety (e.g., scFv, sdAb, or Fab) specifically binds to both human and non-human primates (such as cynomolgus monkey) target epitopes or target antigens (e.g., CD19, EpCAM, or WT1). MSAPs (such as TSAPs or BSAPs) employing such anti-target epitope moieties with cross-reactivity to monkey target may facilitate toxicity studies in non-human primates, which can provide more relevant safety assessments for human clinical trial candidates, without having to perform toxicity studies in chimpanzees or using surrogate molecules. In some embodiments, the anti-target epitope moiety only binds to human target epitope or target antigen. In some embodiments, the anti-target epitope moiety binds to human target epitope or target antigen stronger (such as at least about any of 1.5, 2, 5, 10, 20, 50, 100, 500, 1000-fold, or more) than non-human (e.g., cynomolgus monkey) target epitope or target antigen (e.g., CD19, EpCAM, or WT1).

The anti-target epitope moiety (e.g., scFv, sdAb, or Fab) may fully or partially modulate, block, inhibit, reduce, antagonize, neutralize, or interfere with the functional activity of the target antigen (e.g., CD19, EpCAM, or WT1). When the functional activity of the target antigen is reduced by at least about 95% (such as at least about any of 96%, 97%, 98%, 99%, or 100%) in the presence of an anti-target epitope moiety compared to not bound by an anti-target epitope moiety, the anti-target epitope moiety is considered capable of fully modulating, blocking, inhibiting, reducing, antagonizing, neutralizing, or interfering with the functional activity of the target antigen. When the functional activity of the target antigen is reduced by at least about 50% (such as at least about any of 55%, 60%, 70%, 75%, 80%, 85%, or 90%) in the presence of an anti-target epitope moiety compared to not bound by anti-target epitope moiety, the anti-target epitope moiety is considered capable of significantly modulating, blocking, inhibiting, reducing, antagonizing, neutralizing or interfering with the functional activity of the target antigen. When the functional activity of the target antigen is reduced by less than about 95% (such as reduced by less than about any of 10%, 20%, 25%, 30%, 40%, 50%, 60%, 75%, 80%, 85%, or 90%) in the presence of an anti-target epitope moiety compared to not bound by an anti-target epitope moiety, the anti-target epitope moiety is considered capable of partially modulating, blocking, inhibiting, reducing, antagonizing, neutralizing, or interfering with the functional activity of the target antigen.

In some embodiments, the anti-target epitope moiety (e.g., scFv or Fab) specifically recognizes CD19 (anti-CD19 moiety). In some embodiments, the anti-CD19 antibody or antigen binding fragment thereof (or anti-CD19 moiety) comprises a means for specifically recognizing CD19. In some embodiments, the anti-CD19 moiety comprises a VH comprising one, two or three HVRs (or CDRs) from a reference VH comprising the sequence of SEQ ID NO: 63, and/or a VL comprising one, two or three HVRs (or CDRs) from a reference VL comprising the sequence of SEQ ID NO: 64. In some embodiments, the anti-CD19 moiety comprises a VH comprising three HVRs from a reference VH comprising the sequence of SEQ ID NO: 63, and/or a VL comprising three HVRs from a reference VL comprising the sequence of SEQ ID NO: 64. In some embodiments, the anti-CD19 moiety comprises: a VH comprising one, two, or three HVRs selected from any of SEQ ID NOs: 57-59, and/or a VL comprising one, two, or three HVRs selected from any of SEQ ID NOs: 60-62. In some embodiments, the anti-CD19 moiety comprises: an HVR-H1 comprising the amino acid sequence of SEQ ID NO: 57, or a variant thereof comprising up to about 5 (e.g., 1, 2, 3, 4, or 5) amino acid variations (e.g., insertion(s), deletion(s), and/or substitution(s), such as conservative substitution(s)); an HVR-H2 comprising the amino acid sequence of SEQ ID NO: 58, or a variant thereof comprising up to about 5 (e.g., 1, 2, 3, 4, or 5) amino acid variations (e.g., insertion(s), deletion(s), and/or substitution(s), such as conservative substitution(s)); an HVR-H3 comprising the amino acid sequence of SEQ ID NO: 59, or a variant thereof comprising up to about 5 (e.g., 1, 2, 3, 4, or 5) amino acid variations (e.g., insertion(s), deletion(s), and/or substitution(s), such as conservative substitution(s)); an HVR-L1 comprising the amino acid sequence of SEQ ID NO: 60, or a variant thereof comprising up to about 5 (e.g., 1, 2, 3, 4, or 5) amino acid variations (e.g., insertion(s), deletion(s), and/or substitution(s), such as conservative substitution(s)); an HVR-L2 comprising the amino acid sequence of SEQ ID NO: 61, or a variant thereof comprising up to about 5 (e.g., 1, 2, 3, 4, or 5) amino acid variations (e.g., insertion(s), deletion(s), and/or substitution(s), such as conservative substitution(s)); and an HVR-L3 comprising the amino acid sequence of SEQ ID NO: 62, or a variant thereof comprising up to about 5 (e.g., 1, 2, 3, 4, or 5) amino acid variations (e.g., insertion(s), deletion(s), and/or substitution(s), such as conservative substitution(s)). In some embodiments, the anti-CD19 moiety comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD19 moiety comprises a VH comprising the amino acid sequence of SEQ ID NO: 63, or a variant thereof (e.g., comprising insertion(s), deletion(s), and/or substitution(s), such as conservative substitution(s)) having at least about 80% (such as at least about any of 85%, 90%, 95%, 96%, 97%, 98%, or 99%) sequence identity to the amino acid sequence of SEQ ID NO: 63; and/or a VL comprising the amino acid sequence of SEQ ID NO: 64, or a variant thereof (e.g., comprising insertion(s), deletion(s), and/or substitution(s), such as conservative substitution(s)) having at least about 80% (such as at least about any of 85%, 90%, 95%, 96%, 97%, 98%, or 99%) sequence identity to the amino acid sequence of SEQ ID NO: 64. In some embodiments, the anti-CD19 moiety comprises a VH comprising the amino acid sequence of SEQ ID NO: 63, and a VL comprising the amino acid sequence of SEQ ID NO: 64. In some embodiments, the anti-CD19 moiety is an scFv. In some embodiments, the anti-CD19 scFv comprises the amino acid sequence of SEQ ID NO: 65 or 66, or a variant thereof having at least about 80% (such as at least about any one of 85%, 90%, 95%, 99%, or more) sequence identity to the amino acid sequence of SEQ ID NO: 65 or 66. In some embodiments, the anti-CD19 scFv comprises the amino acid sequence of SEQ ID NO: 65 or 66. In some embodiments, the anti-CD19 moiety is a Fab fragment. In some embodiments, the anti-CD19 Fab fragment comprises a first polypeptide comprising the amino acid sequence of SEQ ID NO: 137, or a variant thereof having at least about 80% (such as at least about any one of 85%, 90%, 95%, 99%, or more) sequence identity to the amino acid sequence of SEQ ID NO: 137; and/or a second polypeptide comprising the amino acid sequence of SEQ ID NO: 139 or 169, or a variant thereof having at least about 80% (such as at least about any one of 85%, 90%, 95%, 99%, or more) sequence identity to the amino acid sequence of SEQ ID NO: 139 or 169. In some embodiments, the anti-CD19 Fab fragment comprises a first polypeptide comprising the amino acid sequence of SEQ ID NO: 137, and a second polypeptide comprising the amino acid sequence of SEQ ID NO: 139 or 169. In some embodiments, the first polypeptide and/or the second polypeptide of the anti-CD19 Fab fragment do not comprise a hinge or portion thereof (e.g., SEQ ID NO: 107) at the C-terminus of the first polypeptide and/or the second polypeptide.

In some embodiments, the anti-target epitope moiety (e.g., scFv or Fab) specifically recognizes EpCAM (anti-EpCAM moiety). In some embodiments, the anti-EpCAM antibody or antigen binding fragment thereof (or anti-EpCAM moiety) comprises a means for specifically recognizing EpCAM. In some embodiments, the anti-EpCAM moiety comprises a VH comprising one, two or three HVRs (or CDRs) from a reference VH comprising the sequence of SEQ ID NO: 73, and/or a VL comprising one, two or three HVRs (or CDRs) from a reference VL comprising the sequence of SEQ ID NO: 74. In some embodiments, the anti-EpCAM moiety comprises a VH comprising three HVRs from a reference VH comprising the sequence of SEQ ID NO: 73, and/or a VL comprising three HVRs from SEQ ID a reference VL comprising the sequence of NO: 74. In some embodiments, the anti-EpCAM moiety comprises: a VH comprising one, two, or three HVRs selected from any of SEQ ID NOs: 67-69, and/or a VL comprising one, two, or three HVRs selected from any of SEQ ID NOs: 70-72. In some embodiments, the anti-EpCAM moiety comprises: an HVR-H1 comprising the amino acid sequence of SEQ ID NO: 67, or a variant thereof comprising up to about 5 (e.g., 1, 2, 3, 4, or 5) amino acid variations (e.g., insertion(s), deletion(s), and/or substitution(s), such as conservative substitution(s)); an HVR-H2 comprising the amino acid sequence of SEQ ID NO: 68, or a variant thereof comprising up to about 5 (e.g., 1, 2, 3, 4, or 5) amino acid variations (e.g., insertion(s), deletion(s), and/or substitution(s), such as conservative substitution(s)); an HVR-H3 comprising the amino acid sequence of SEQ ID NO: 69, or a variant thereof comprising up to about 5 (e.g., 1, 2, 3, 4, or 5) amino acid variations (e.g., insertion(s), deletion(s), and/or substitution(s), such as conservative substitution(s)); an HVR-L1 comprising the amino acid sequence of SEQ ID NO: 70, or a variant thereof comprising up to about 5 (e.g., 1, 2, 3, 4, or 5) amino acid variations (e.g., insertion(s), deletion(s), and/or substitution(s), such as conservative substitution(s)); an HVR-L2 comprising the amino acid sequence of SEQ ID NO: 71, or a variant thereof comprising up to about 5 (e.g., 1, 2, 3, 4, or 5) amino acid variations (e.g., insertion(s), deletion(s), and/or substitution(s), such as conservative substitution(s)); and an HVR-L3 comprising the amino acid sequence of SEQ ID NO: 72, or a variant thereof comprising up to about 5 (e.g., 1, 2, 3, 4, or 5) amino acid variations (e.g., insertion(s), deletion(s), and/or substitution(s), such as conservative substitution(s)). In some embodiments, the anti-EpCAM moiety comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, the anti-EpCAM moiety comprises a VH comprising the amino acid sequence of SEQ ID NO: 73, or a variant thereof (e.g., comprising insertion(s), deletion(s), and/or substitution(s), such as conservative substitution(s)) having at least about 80% (such as at least about any of 85%, 90%, 95%, 96%, 97%, 98%, or 99%) sequence identity to the amino acid sequence of SEQ ID NO: 73; and/or a VL comprising the amino acid sequence of SEQ ID NO: 74, or a variant thereof (e.g., comprising insertion(s), deletion(s), and/or substitution(s), such as conservative substitution(s)) having at least about 80% (such as at least about any of 85%, 90%, 95%, 96%, 97%, 98%, or 99%) sequence identity to the amino acid sequence of SEQ ID NO: 74. In some embodiments, the anti-EpCAM moiety comprises a VH comprising the amino acid sequence of SEQ ID NO: 73, and a VL comprising the amino acid sequence of SEQ ID NO: 74. In some embodiments, the anti-EpCAM moiety is an scFv. In some embodiments, the anti-EpCAM scFv comprises the amino acid sequence of SEQ ID NO: 75 or 78, or a variant thereof having at least about 80% (such as at least about any one of 85%, 90%, 95%, 99%, or more) sequence identity to the amino acid sequence of SEQ ID NO: 75 or 78. In some embodiments, the anti-EpCAM scFv comprises the amino acid sequence of SEQ ID NO: 75 or 78. In some embodiments, the anti-EpCAM moiety is a Fab fragment. In some embodiments, the anti-EpCAM Fab fragment comprises a first polypeptide comprising the amino acid sequence of SEQ ID NO: 76, or a variant thereof having at least about 80% (such as at least about any one of 85%, 90%, 95%, 99%, or more) sequence identity to the amino acid sequence of SEQ ID NO: 76; and/or a second polypeptide comprising the amino acid sequence of SEQ ID NO: 77, or a variant thereof having at least about 80% (such as at least about any one of 85%, 90%, 95%, 99%, or more) sequence identity to the amino acid sequence of SEQ ID NO: 77. In some embodiments, the anti-EpCAM Fab fragment comprises a first polypeptide comprising the amino acid sequence of SEQ ID NO: 76, and a second polypeptide comprising the amino acid sequence of SEQ ID NO: 77. In some embodiments, the first polypeptide and/or the second polypeptide of the anti-EpCAM fragment do not comprise a hinge or portion thereof (e.g., SEQ ID NO: 107) at the C-terminus of the first polypeptide and/or the second polypeptide.

In some embodiments, the anti-target epitope moiety (e.g., scFv or Fab) specifically recognizes WT1 (anti-WT1 moiety). In some embodiments, the anti-WT1 antibody or antigen binding fragment thereof (or anti-WT1 moiety) comprises a means for specifically recognizing WT1. In some embodiments, the anti-WT1 moiety comprises a VH comprising one, two or three HVRs (or CDRs) from a reference VH comprising the sequence of SEQ ID NO: 85, and/or a VL comprising one, two or three HVRs (or CDRs) from a reference VL comprising the sequence of SEQ ID NO: 86. In some embodiments, the anti-WT1 moiety comprises a VH comprising three HVRs from a reference VH comprising the sequence of SEQ ID NO: 85, and/or a VL comprising three HVRs from a reference VL comprising the sequence of SEQ ID NO: 86. In some embodiments, the anti-WT1 moiety comprises: a VH comprising one, two, or three HVRs selected from any of SEQ ID NOs: 79-81, and/or a VL comprising one, two, or three HVRs selected from any of SEQ ID NOs: 82-84. In some embodiments, the anti-WT1 moiety comprises: an HVR-H1 comprising the amino acid sequence of SEQ ID NO: 79, or a variant thereof comprising up to about 5 (e.g., 1, 2, 3, 4, or 5) amino acid variations (e.g., insertion(s), deletion(s), and/or substitution(s), such as conservative substitution(s)); an HVR-H2 comprising the amino acid sequence of SEQ ID NO: 80, or a variant thereof comprising up to about 5 (e.g., 1, 2, 3, 4, or 5) amino acid variations (e.g., insertion(s), deletion(s), and/or substitution(s), such as conservative substitution(s)); an HVR-H3 comprising the amino acid sequence of SEQ ID NO: 81, or a variant thereof comprising up to about 5 (e.g., 1, 2, 3, 4, or 5) amino acid variations (e.g., insertion(s), deletion(s), and/or substitution(s), such as conservative substitution(s)); an HVR-L1 comprising the amino acid sequence of SEQ ID NO: 82, or a variant thereof comprising up to about 5 (e.g., 1, 2, 3, 4, or 5) amino acid variations (e.g., insertion(s), deletion(s), and/or substitution(s), such as conservative substitution(s)); an HVR-L2 comprising the amino acid sequence of SEQ ID NO: 83, or a variant thereof comprising up to about 5 (e.g., 1, 2, 3, 4, or 5) amino acid variations (e.g., insertion(s), deletion(s), and/or substitution(s), such as conservative substitution(s)); and an HVR-L3 comprising the amino acid sequence of SEQ ID NO: 84, or a variant thereof comprising up to about 5 (e.g., 1, 2, 3, 4, or 5) amino acid variations (e.g., insertion(s), deletion(s), and/or substitution(s), such as conservative substitution(s)). In some embodiments, the anti-WT1 moiety comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the anti-WT1 moiety comprises a VH comprising the amino acid sequence of SEQ ID NO: 85, or a variant thereof (e.g., comprising insertion(s), deletion(s), and/or substitution(s), such as conservative substitution(s)) having at least about 80% (such as at least about any of 85%, 90%, 95%, 96%, 97%, 98%, or 99%) sequence identity to the amino acid sequence of SEQ ID NO: 85; and/or a VL comprising the amino acid sequence of SEQ ID NO: 86, or a variant thereof (e.g., comprising insertion(s), deletion(s), and/or substitution(s), such as conservative substitution(s)) having at least about 80% (such as at least about any of 85%, 90%, 95%, 96%, 97%, 98%, or 99%) sequence identity to the amino acid sequence of SEQ ID NO: 86. In some embodiments, the anti-WT1 moiety comprises a VH comprising the amino acid sequence of SEQ ID NO: 85, and a VL comprising the amino acid sequence of SEQ ID NO: 86. In some embodiments, the anti-WT1 moiety is an scFv. In some embodiments, the anti-WT1 scFv comprises the amino acid sequence of SEQ ID NO: 87 or 88, or a variant thereof having at least about 80% (such as at least about any one of 85%, 90%, 95%, 99%, or more) sequence identity to the amino acid sequence of SEQ ID NO: 87 or 88. In some embodiments, the anti-WT1 scFv comprises the amino acid sequence of SEQ ID NO: 87 or 88. In some embodiments, the anti-WT1 moiety is a Fab fragment. In some embodiments, the anti-WT1 Fab fragment comprises a first polypeptide comprising the amino acid sequence of SEQ ID NO: 145, or a variant thereof having at least about 80% (such as at least about any one of 85%, 90%, 95%, 99%, or more) sequence identity to the amino acid sequence of SEQ ID NO: 145; and/or a second polypeptide comprising the amino acid sequence of SEQ ID NO: 146, or a variant thereof having at least about 80% (such as at least about any one of 85%, 90%, 95%, 99%, or more) sequence identity to the amino acid sequence of SEQ ID NO: 146. In some embodiments, the anti-WT1 Fab fragment comprises a first polypeptide comprising the amino acid sequence of SEQ ID NO: 145, and a second polypeptide comprising the amino acid sequence of SEQ ID NO: 146. In some embodiments, the first polypeptide and/or the second polypeptide of the anti-WT1 fragment do not comprise a hinge or portion thereof (e.g., SEQ ID NO: 107) at the C-terminus of the first polypeptide and/or the second polypeptide.

### Linkers

The MSAPs (such as TSAPs) described herein may comprise a linker (such as a peptide linker) connecting the Fab fragment (e.g., N′ of VH, N′ of VL, C′ of CH1, and/or C′ of CL) and the first antigen binding fragment (e.g., scFv), and/or connecting the Fab fragment (e.g., N′ of VH, N′ of VL, C′ of CH1, and/or C′ of CL) and the second antigen binding fragment (e.g., scFv). In some embodiments, the first antigen binding fragment and/or the second antigen binding fragment is an scFv. In some embodiments, the scFv comprises a linker (e.g., peptide linker) connecting VH and VL. In some embodiments, the first antigen binding fragment is directly fused to the Fab fragment, and/or the second antigen binding fragment is directly fused to the Fab fragment. In some embodiments, the first antigen binding fragment is fused to the Fab fragment via a first linker, and/or the second antigen binding fragment is fused to the Fab fragment via a second linker. In some embodiments, the first linker and the second linker are the same. In some embodiments, the first linker and the second linker are different. In some embodiments, the linker (such as peptide linker) connecting VH and VL of an antigen binding scFv may be the same or different from one or both of the linkers between the Fab fragment and the antigen binding scFv. Any linkers described herein can also be used in any of the anti-CD3 constructs or anti-CD8 constructs described herein, e.g., between a VH and a VL of an scFv, or connecting two or more moieties of the constructs, such as connecting a Fab fragment (e.g., N′ of VH, N′ of VL, C′ of CH1, and/or C′ of CL) and an antigen binding fragment (e.g., scFv).

In some embodiments, the linker is not a peptide linker. In some embodiments, the linker is a peptide linker.

The linker can be a peptide linker of any length. In some embodiments, the peptide linker is from about 1 to about 10 amino acids (aa) long, from about 2 to about 15 aa long, from about 3 to about 12 aa long, from about 4 to about 10 aa long, from about 5 to about 9 aa long, from about 1 to about 20 aa long, from about 21 to about 30 aa long, from about 1 to about 30 aa long, from about 10 to about 30 aa long, from about 2 to about 19 aa long, from about 2 to about 18 aa long, from about 2 to about 17 aa long, from about 2 to about 16 aa long, from about 2 to about 10 aa long, from about 2 to about 14 aa long, from about 2 to about 13 aa long, from about 2 to about 12 aa long, from about 2 to about 11 aa long, from about 2 to about 9 aa long, from about 2 to about 8 aa long, from about 2 to about 7 aa long, from about 2 to about 6 aa long, or from about 2 to about 5 aa long. In some embodiments, the peptide linker is any of 1, 2, 3, 4, 5, 6, 7, 8, 9, 10, 11, 12, 13, 14, 15, 16, 17, 18, 19, or 20 aa long. In some embodiments, the peptide linker is any of 21, 22, 23, 24, 25, 26, 27, 28, 29, or 30 aa long. In some embodiments, the peptide linker is about 2 to about 30 aa long, such as about 2 to about 15 amino acids long, about 15 amino acids long, about 6 amino acids long, about 2 aa long, or about 5 aa long.

A peptide linker can have a naturally occurring sequence or a non-naturally occurring sequence. For example, a sequence derived from the hinge region of a heavy chain only antibody can be used as a linker. See, for example, WO1996/34103. In some embodiments, the peptide linker is derived from a human IgG1 or IgG4 hinge. In some embodiments, the linker is a flexible linker. Exemplary flexible linkers include glycine polymers (G) n, glycine-serine polymers (including, for example, (GS)ₙ, (GSGGS)ₙ (SEQ ID NO: 116), (GGGS)ₙ (SEQ ID NO: 117), or (GGGGS)ₙ (SEQ ID NO: 113), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine and is much less restricted than residues with longer side chains (see Scheraga, Rev. Computational Chem. 11 173-142 (1992)). In some embodiments, the linker comprises amino acid residues selected form the group consisting of glycine, serine, arginine, and alanine. Exemplary flexible linkers include, but are not limited to GG, GGSG (SEQ ID NO: 118), GGSGG (SEQ ID NO: 119), GSGSG (SEQ ID NO: 120), GSGGG (SEQ ID NO: 121GGGSG (SEQ ID NO: 122), GSSSG (SEQ ID NO: 123), GGSGGS (SEQ ID NO: 112), SGGGGS (SEQ ID NO: 110), GRAGGGGAGGGG (SEQ ID NO: 114), GRAGGG (SEQ ID NO: 115), GGGGSGGGGSGGGGS (SEQ ID NO: 108), GGGGS (SEQ ID NO: 109), and the like. In some embodiments, the linker between the VH of the Fab fragment and the antigen binding fragment (e.g., scFv) is SEQ ID NO: 109 or 110, or GG. In some embodiments, the linker between the VL of the Fab fragment and the antigen binding fragment (e.g., scFv) is SEQ ID NO: 109 or 110, or GG. In some embodiments, the linker connecting the VH and VL of the first scFv or the second scFv is SEQ ID NO: 108. The ordinarily skilled artisan will recognize that design of a MSAP (such as TSAP) can include linkers that are all or partially flexible, such that the linker can include a flexible linker portion as well as one or more portions that confer less flexible structure to provide a desired MSAP (such as TSAP) structure.

In some embodiments, the linker between the Fab fragment and the first or second antigen binding fragment (e.g., scFv) is a stable linker (not cleavable by protease, especially MMPs).

In some embodiments, the linker is a cleavable linker. In some embodiments, the linker between the Fab fragment and the first or second antigen binding fragment (e.g., scFv) comprises a protease substrate cleavage sequence, for example, an MMP substrate cleavage sequence. Substrate sequences that can be cleaved by MMPs have been extensively studied. For example, the sequence of PLGLAG (SEQ ID NO: 140) can be cleaved by most MMPs. In some embodiments, the protease cleavage site is recognized by MMP-2, MMP-9, or a combination thereof.

### III. Anti-CD8 Constructs

In one aspect, the present application provides an anti-CD8 construct comprising (or consisting of, or consisting essentially of) any of the anti-CD8 antibodies or antigen-binding fragments thereof (or anti-CD8 moieties) described herein. In some embodiments, the anti-CD8 construct is a MSAP, such as any of the MSAPs comprising an anti-CD8 moiety described herein. Hence in some embodiments, there is provided an MSAP (e.g., TSAP, BSAP) comprising any of the anti-CD8 antibodies or antigen binding fragments thereof (e.g., scFv or Fab) described herein.

In some embodiments, the anti-CD8 construct comprises (or consists of, or consists essentially of) a full-length antibody, a Fab, a Fab′, a Fab′-SH, a F(ab′)₂, an Fv, an scFv, or a combination thereof. In some embodiments, the anti-CD8 construct is an anti-CD8 Fab fragment. In some embodiments, the anti-CD8 construct is an anti-CD8 scFv. In some embodiments, the anti-CD8 construct is an anti-CD8 full-length antibody comprising an Fc region of any antibody class or isotype, such as IgG1, IgG2, IgG3, or IgG4. In some embodiments, the Fc region has reduced or minimized effector function.

In some embodiments, the anti-CD8 construct is monospecific. In some embodiments, the anti-CD8 construct is multispecific (e.g., bispecific, or trispecific). In some embodiments, the anti-CD8 construct is monovalent. In some embodiments, the anti-CD8 construct is multivalent (e.g., bivalent, or trivalent). In some embodiments, the anti-CD8 construct is multivalent and monospecific. In some embodiments, the anti-CD8 construct is multivalent and multispecific.

In some embodiments, the anti-CD8 construct is selected from the group consisting of: a multispecific antibody construct (e.g., bispecific antibody construct, or trispecific antibody construct, such as MSAP), a bispecific T cell engager (BiTE), an antibody-drug conjugate (ADC), an antibody-siRNA conjugate (ARC), an antibody-oligonucleotide conjugate (AOC), an antibody-detection tag conjugate, an immunocytokine, a trispecific NK-cell engager (TriKE), a trispecific T-cell activation construct (TriTAC), and a tandem bivalent antibody.

In some embodiments, there is provided an anti-CD8 MSAP (e.g., BSAP, TSAP) comprising any of the anti-CD8 antibodies or antigen-binding fragments thereof (e.g., scFv or Fab) described herein. In some embodiments, there is provided an anti-CD8 construct (e.g., MSAP) comprising two or more anti-CD8 moieties described herein, such as connected in tandem (e.g., scFv-scFv). The two or more anti-CD8 moieties can recognize the same CD8 epitope or at least one different CD8 epitope. In some embodiments, there is provided an anti-CD8 construct (or an MSAP, e.g., BSAP) comprising: i) an anti-CD8 antibody or antigen binding fragment thereof (e.g., any of the anti-CD8 antibodies or antigen-binding fragments thereof described herein), and ii) a second binding moiety (e.g., scFv or Fab) that specifically binds to a non-CD8 target antigen (e.g., TSA or TAA, such as CD19, EpCAM, WT1). In some embodiments, there is provided an anti-CD8 construct (or an MSAP, e.g., BSAP) comprising: a) an anti-CD8 antibody or antigen binding fragment thereof comprising: i) an HVR-H1 comprising the amino acid sequence of SEQ ID NO: 13, an HVR-H2 comprising the amino acid sequence of SEQ ID NO: 14, an HVR-H3 comprising the amino acid sequence of SEQ ID NO: 15, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; ii) an HVR-H1 comprising the amino acid sequence of SEQ ID NO: 1, an HVR-H2 comprising the amino acid sequence of SEQ ID NO: 16, an HVR-H3 comprising the amino acid sequence of SEQ ID NO: 3, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iii) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iv) an HVR-H1 comprising the amino acid sequence of SEQ ID NO: 1, an HVR-H2 comprising the amino acid sequence of SEQ ID NO: 18, an HVR-H3 comprising the amino acid sequence of SEQ ID NO: 19, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; v) an HVR-H1 comprising the amino acid sequence of SEQ ID NO: 7, an HVR-H2 comprising the amino acid sequence of SEQ ID NO: 8, an HVR-H3 comprising the amino acid sequence of SEQ ID NO: 9, an HVR-L1 comprising the amino acid sequence of SEQ ID NO: 10, an HVR-L2 comprising the amino acid sequence of SEQ ID NO: 11, and an HVR-L 3 comprising the amino acid sequence of SEQ ID NO: 12; or vi) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; and b) a second binding moiety (e.g., scFv or Fab) that specifically binds to a non-CD8 target antigen (e.g., TSA or TAA, such as CD19, EpCAM, WT1). In some embodiments, the anti-CD8 antibody or antigen binding fragment thereof comprises: i) a VH comprising the amino acid sequence of SEQ ID NO: 24, and a VL comprising the amino acid sequence of SEQ ID NO: 25; ii) a VH comprising the amino acid sequence of SEQ ID NO: 26, and a VL comprising the amino acid sequence of SEQ ID NO: 27; iii) a VH comprising the amino acid sequence of SEQ ID NO: 28, and a VL comprising the amino acid sequence of SEQ ID NO: 29; iv) a VH comprising the amino acid sequence of SEQ ID NO: 30, and a VL comprising the amino acid sequence of SEQ ID NO: 25; v) a VH comprising the amino acid sequence of SEQ ID NO: 32, and a VL comprising the amino acid sequence of SEQ ID NO: 33; or vi) a VH comprising the amino acid sequence of SEQ ID NO: 34, and a VL comprising the amino acid sequence of SEQ ID NO: 35. In some embodiments, the anti-CD8 antibody or antigen binding fragment thereof and the second binding moiety are independently selected from the group consisting of a full-length antibody, a Fab, a Fab′, a Fab′-SH, a F(ab′)2, an Fv, an scFv, and any combination thereof. In some embodiments, the anti-CD8 antigen binding fragment is an anti-CD8 Fab fragment or an anti-CD8 scFv. The non-CD8 target antigen can be any target antigens (e.g., disease-associated target antigen) described in the “Anti-target epitope moiety” section above. In some embodiments, the anti-CD8 antibody or antigen binding fragment thereof is fused to the second binding moiety via an optional linker (e.g., peptide linker). In some embodiments, the anti-CD8 construct (or MSAP) further comprises a third binding moiety (e.g., scFv or Fab) that specifically binds to a second target antigen (e.g., immune cell surface molecule, such as CD3, CD4, or CD8; or TSA or TAA, such as CD19, EpCAM, WT1). The second binding moiety and/or the third binding moiety can be fused (e.g., via linker(s)) with the anti-CD8 antibody or antigen binding fragment thereof in any configurations, such as those described for MSAPs under section II. For example, when moiety A is a Fab fragment, moiety B and/or moiety C can be independently fused to N′ of VH, N′ of VL, C′ of CH1, and/or C′ of CL of moiety A Fab fragment.

In some embodiments, there is provided an anti-CD8 construct (or MSAP, e.g., BSAP), comprising i) an anti-CD8 Fab fragment (e.g., any of the anti-CD8 Fab fragments described herein), wherein the anti-CD8 Fab fragment comprises a first polypeptide comprising a VH and a CH1, and a second polypeptide comprising a VL and a CL, and ii) a second binding moiety (e.g., scFv or Fab) that specifically binds to a non-CD8 target antigen (e.g., TSA or TAA, such as CD19, EpCAM, or WT1). In some embodiments, there is provided an anti-CD8 construct (or MSAP, e.g., BSAP), comprising i) an anti-CD8 Fab fragment (e.g., any of the anti-CD8 Fab fragments described herein), wherein the anti-CD8 Fab fragment comprises a first polypeptide comprising a VH and a CH1, and a second polypeptide comprising a VL and a CL, and ii) a second binding moiety (e.g., scFv or Fab) that specifically binds to a non-CD8 target antigen (e.g., TSA or TAA, such as CD19, EpCAM, or WT1); wherein the second binding moiety is fused to the N-terminus of the VH or the VL of the anti-CD8 Fab fragment via an optional linker. In some embodiments, the second binding moiety is an scFv. In some embodiments, there is provided an anti-CD8 construct (or MSAP, e.g., BSAP or TSAP), comprising i) an anti-CD8 Fab fragment (e.g., any of the anti-CD8 Fab fragments described herein), wherein the anti-CD8 Fab fragment comprises a first polypeptide comprising a VH and a CH1, and a second polypeptide comprising a VL and a CL, ii) a second binding moiety (e.g., scFv or Fab) that specifically binds to a first non-CD8 target antigen (e.g., TSA or TAA, such as CD19, EpCAM, or WT1; or immune cell surface molecule, such as CD3), and iii) a third binding moiety (e.g., scFv or Fab) that specifically binds to a second non-CD8 target antigen (e.g., immune cell surface molecule, such as CD3; or TSA or TAA, such as CD19, EpCAM, or WT1); wherein the second binding moiety is fused to the N-terminus of the VH of the anti-CD8 Fab fragment via an optional first linker, and the third binding moiety is fused to the N-terminus of the VL of the anti-CD8 Fab fragment via an optional second linker. In some embodiments, the second binding moiety and the third binding moiety are both scFvs. In some embodiments, the first non-CD8 target antigen (or epitope) and the second non-CD8 target antigen (or epitope) are the same. In some embodiments, the first non-CD8 target antigen (or epitope) and the second non-CD8 target antigen (or epitope) are different. In some embodiments, the first non-CD8 target antigen and the second non-CD8 target antigen are both TSA or TAA, e.g., both EpCAM or both CD19 or both WT1 (either same epitope or different epitope). In some embodiments, the first non-CD8 target antigen is a TSA or TAA (e.g., EpCAM, CD19, or WT1), and the second non-CD8 target antigen is an immune cell surface molecule (e.g., CD3, CD4). In some embodiments, the second non-CD8 target antigen is a TSA or TAA (e.g., EpCAM, CD19, or WT1), and the first non-CD8 target antigen is an immune cell surface molecule (e.g., CD3, CD4). In some embodiments, the second binding moiety is an scFv, e.g., anti-EpCAM scFv, anti-CD19 scFv, anti-WT1 scFv, or anti-CD3 scFv. In some embodiments, the third binding moiety is an scFv, e.g., anti-CD3 scFv, anti-EpCAM scFv, anti-CD19 scFv, or anti-WT1 scFv. In some embodiments, the second binding moiety and the third binding moiety are both anti-EpCAM scFvs, both anti-CD19 scFvs, or both anti-WT1 scFvs. In some embodiments, the second binding moiety is an anti-CD3 scFv, and the third binding moiety is an anti-target epitope (e.g., TSA or TAA, such as EpCAM, CD19, or WT1) scFv. In some embodiments, the third binding moiety is an anti-CD3 scFv, and the second binding moiety is an anti-target epitope (e.g., TSA or TAA, such as EpCAM, CD19, or WT1) scFv. In some embodiments, the linker, the first linker, and/or the second linker comprises an amino acid sequence independently selected from the group consisting of GG and SEQ ID NOs: 108-110.

In some embodiments, there is provided an anti-CD8 construct (or MSAP, e.g., BSAP), comprising i) an anti-CD8 antibody or antigen binding fragment thereof (e.g., any of the anti-CD8 antibodies or antigen-binding fragments thereof described herein, such as scFv or Fab), and ii) a Fab fragment that specifically binds to a non-CD8 target antigen (e.g., TSA or TAA, such as CD19, EpCAM, or WT1). In some embodiments, there is provided an anti-CD8 construct (or MSAP, e.g., BSAP), comprising i) an anti-CD8 antibody or antigen binding fragment thereof (e.g., any of the anti-CD8 antibodies or antigen-binding fragments thereof described herein, such as scFv or Fab), and ii) a Fab fragment that specifically binds to a non-CD8 target antigen (e.g., TSA or TAA, such as CD19, EpCAM, or WT1); wherein the anti-CD8 antibody or antigen binding fragment thereof is fused to the N-terminus of the VH or the VL of the non-CD8 target antigen Fab fragment via an optional linker. In some embodiments, anti-CD8 antibody or antigen binding fragment thereof is an anti-CD8 scFv. In some embodiments, there is provided an anti-CD8 construct (or MSAP, e.g., BSAP or TSAP), comprising i) an anti-CD8 antibody or antigen binding fragment thereof (e.g., any of the anti-CD8 antibodies or antigen-binding fragments thereof described herein, such as scFv or Fab) ii) a Fab fragment that specifically binds to a first target antigen that is non-CD8 target antigen (e.g., TSA or TAA, such as CD19, EpCAM, or WT1; or immune cell surface molecule, such as CD3), and iii) a third binding moiety (e.g., scFv or Fab) that specifically binds to a second target antigen (e.g., immune cell surface molecule, such as CD3 or CD8; or TSA or TAA, such as CD19, EpCAM, or WT1); wherein: (1) the anti-CD8 antibody or antigen binding fragment thereof is fused to the N-terminus of the VH of the first target antigen Fab fragment via an optional first linker, and the third binding moiety is fused to the N-terminus of the VL of the first target antigen Fab fragment via an optional second linker; or (2) the anti-CD8 antibody or antigen binding fragment thereof is fused to the N-terminus of the VL of the first target antigen Fab fragment via an optional first linker, and the third binding moiety is fused to the N-terminus of the VH of the first target antigen Fab fragment via an optional second linker. In some embodiments, the anti-CD8 antibody or antigen binding fragment thereof and the third binding moiety are both scFvs. The first target antigen (or epitope) and the second target antigen (or epitope) can be the same or different. In some embodiments, the first target antigen and the second target antigen are both TSA or TAA, e.g., both EpCAM, both CD19, or both WT1 (either same epitope or different epitope). In some embodiments, the first target antigen is a TSA or TAA (e.g., EpCAM, CD19, or WT1), and the second target antigen is an immune cell surface molecule (e.g., CD3, CD4, or CD8). In some embodiments, the second target antigen is a TSA or TAA (e.g., EpCAM, CD19, or WT1), and the first target antigen is an immune cell surface molecule (e.g., CD3, CD4). In some embodiments, the anti-CD8 antigen binding fragment is an anti-CD8 scFv. In some embodiments, the third binding moiety is an scFv, e.g., anti-CD3 scFv, anti-CD8 scFv, anti-EpCAM scFv, anti-CD19 scFv, or anti-WT1 scFv. In some embodiments, the anti-CD8 antigen binding fragment and the third binding moiety are both anti-CD8 scFvs. The anti-CD8 antigen binding fragment (e.g., scFv) and the third binding moiety can be identical or different. In some embodiments, the anti-first target antigen Fab is an anti-CD3 Fab, and the third binding moiety is an anti-target epitope (e.g., TSA or TAA, such as EpCAM, CD19, or WT1) scFv. In some embodiments, the third binding moiety is an anti-CD3 or anti-CD8 scFv (e.g., any of the anti-CD3 or anti-CD8 scFvs described herein), and the anti-first target antigen Fab is an anti-target epitope (e.g., TSA or TAA, such as EpCAM, CD19, or WT1) Fab. In some embodiments, the linker, the first linker, and/or the second linker comprises an amino acid sequence independently selected from the group consisting of GG and SEQ ID NOs: 108-110.

In some embodiments, there is provided an anti-CD8 construct (or MSAP, e.g., BSAP), comprising a) an anti-CD8 Fab fragment (e.g., any of the anti-CD8 Fab fragments described herein), wherein the anti-CD8 Fab fragment comprises a first polypeptide comprising a VH and a CH1, and a second polypeptide comprising a VL and a CL, and b) a second binding moiety (e.g., scFv or Fab) that specifically binds to EpCAM (e.g., any of the anti-EpCAM moieties described herein); wherein the second binding moiety is fused to the N-terminus of the VH or VL of the anti-CD8 Fab fragment via an optional linker. In some embodiments, there is provided an anti-CD8 construct (or MSAP, e.g., BSAP or TSAP), comprising a) an anti-CD8 Fab fragment (e.g., any of the anti-CD8 Fab fragments described herein), wherein the anti-CD8 Fab fragment comprises a first polypeptide comprising a VH and a CH1, and a second polypeptide comprising a VL and a CL, b) a second binding moiety (e.g., scFv or Fab) that specifically binds to a first target epitope of EpCAM (e.g., any of the anti-EpCAM moieties described herein), and c) a third binding moiety (e.g., scFv or Fab) that specifically binds to a second target epitope of EpCAM (e.g., any of the anti-EpCAM moieties described herein); wherein: (1) the second binding moiety is fused to the N-terminus of the VH of the anti-CD8 Fab fragment via an optional first linker, and the third binding moiety is fused to the N-terminus of the VL of the anti-CD8 Fab fragment via an optional second linker; or (2) the second binding moiety is fused to the N-terminus of the VL of the anti-CD8 Fab fragment via an optional first linker, and the third binding moiety is fused to the N-terminus of the VH of the anti-CD8 Fab fragment via an optional second linker. In some embodiments, the anti-CD8 Fab fragment comprises: i) an HVR-H1 comprising the amino acid sequence of SEQ ID NO: 13, an HVR-H2 comprising the amino acid sequence of SEQ ID NO: 14, an HVR-H3 comprising the amino acid sequence of SEQ ID NO: 15, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; ii) an HVR-H1 comprising the amino acid sequence of SEQ ID NO: 1, an HVR-H2 comprising the amino acid sequence of SEQ ID NO: 16, an HVR-H3 comprising the amino acid sequence of SEQ ID NO: 3, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iii) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iv) an HVR-H1 comprising the amino acid sequence of SEQ ID NO: 1, an HVR-H2 comprising the amino acid sequence of SEQ ID NO: 18, an HVR-H3 comprising the amino acid sequence of SEQ ID NO: 19, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; v) an HVR-H1 comprising the amino acid sequence of SEQ ID NO: 7, an HVR-H2 comprising the amino acid sequence of SEQ ID NO: 8, an HVR-H3 comprising the amino acid sequence of SEQ ID NO: 9, an HVR-L1 comprising the amino acid sequence of SEQ ID NO: 10, an HVR-L2 comprising the amino acid sequence of SEQ ID NO: 11, and an HVR-L3 comprising the amino acid sequence of SEQ ID NO: 12; or vi) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23. In some embodiments, the anti-CD8 Fab fragment comprises: i) a VH comprising the amino acid sequence of SEQ ID NO: 24, and a VL comprising the amino acid sequence of SEQ ID NO: 25; ii) a VH comprising the amino acid sequence of SEQ ID NO: 26, and a VL comprising the amino acid sequence of SEQ ID NO: 27; iii) a VH comprising the amino acid sequence of SEQ ID NO: 28, and a VL comprising the amino acid sequence of SEQ ID NO: 29; iv) a VH comprising the amino acid sequence of SEQ ID NO: 30, and a VL comprising the amino acid sequence of SEQ ID NO: 25; v) a VH comprising the amino acid sequence of SEQ ID NO: 32, and a VL comprising the amino acid sequence of SEQ ID NO: 33; or vi) a VH comprising the amino acid sequence of SEQ ID NO: 34, and a VL comprising the amino acid sequence of SEQ ID NO: 35. In some embodiments, the anti-CD8 Fab fragment comprises: i) a first polypeptide comprising the amino acid sequence of SEQ ID NO: 147, and a second polypeptide comprising the amino acid sequence of SEQ ID NO: 148; ii) a first polypeptide comprising the amino acid sequence of SEQ ID NO: 149, and a second polypeptide comprising the amino acid sequence of SEQ ID NO: 150; iii) a first polypeptide comprising the amino acid sequence of SEQ ID NO: 91, and a second polypeptide comprising the amino acid sequence of SEQ ID NO: 92; iv) a first polypeptide comprising the amino acid sequence of SEQ ID NO: 151, and a second polypeptide comprising the amino acid sequence of SEQ ID NO: 148; v) a first polypeptide comprising the amino acid sequence of SEQ ID NO: 153, and a second polypeptide comprising the amino acid sequence of SEQ ID NO: 154; or vi) a first polypeptide comprising the amino acid sequence of SEQ ID NO: 155, and a second polypeptide comprising the amino acid sequence of SEQ ID NO: 156. In some embodiments, the first target epitope of EpCAM and the second target epitope of EpCAM are the same. In some embodiments, the first target epitope of EpCAM and the second target epitope of EpCAM are different. In some embodiments, the linker, the first linker, and/or the second linker comprises an amino acid sequence independently selected from the group consisting of GG and SEQ ID NOs: 108-110. In some embodiments, the second binding moiety and/or the third binding moiety comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, the second binding moiety and/or the third binding moiety comprises a VH comprising the amino acid sequence of SEQ ID NO: 73, and a VL comprising the amino acid sequence of SEQ ID NO: 74. In some embodiments, the second binding moiety and/or the third binding moiety is an anti-EpCAM scFv. In some embodiments, the anti-EpCAM scFv comprises the amino acid sequence of SEQ ID NO: 75 or 78. In some embodiments, the second binding moiety and the third binding moiety are identical. In some embodiments, the second binding moiety and the third binding moiety are different.

Hence in some embodiments, there is provided an anti-CD8 construct (or MSAP, e.g., BSAP), comprising a) an anti-CD8 Fab fragment, wherein the anti-CD8 Fab fragment comprises a first polypeptide comprising a VH and a CH1, and a second polypeptide comprising a VL and a CL, b) a second scFv that specifically binds to a first target epitope of EpCAM, and c) a third scFv that specifically binds to a second target epitope of EpCAM; wherein the second scFv is fused to the N-terminus of the VH of the anti-CD8 Fab fragment via a first linker to form a first fusion polypeptide, and the third scFv is fused to the N-terminus of the VL of the anti-CD8 Fab fragment via a second linker to form a second fusion polypeptide; and wherein i) the first fusion polypeptide comprises the amino acid sequence of SEQ ID NO: 124, and the second fusion polypeptide comprises the amino acid sequence of SEQ ID NO: 125; ii) the first fusion polypeptide comprises the amino acid sequence of SEQ ID NO: 126, and the second fusion polypeptide comprises the amino acid sequence of SEQ ID NO: 127; iii) the first fusion polypeptide comprises the amino acid sequence of SEQ ID NO: 128, and the second fusion polypeptide comprises the amino acid sequence of SEQ ID NO: 129; iv) the first fusion polypeptide comprises the amino acid sequence of SEQ ID NO: 130, and the second fusion polypeptide comprises the amino acid sequence of SEQ ID NO: 131; v) the first fusion polypeptide comprises the amino acid sequence of SEQ ID NO: 132, and the second fusion polypeptide comprises the amino acid sequence of SEQ ID NO: 133; or vi) the first fusion polypeptide comprises the amino acid sequence of SEQ ID NO: 134, and the second fusion polypeptide comprises the amino acid sequence of SEQ ID NO: 135.

In some embodiments, there is provided an anti-CD8 construct (or MSAP, e.g., BSAP) comprising: a) an anti-CD8 antibody or antigen binding fragment thereof (e.g., any of the anti-CD8 antibodies or antigen binding fragments thereof described herein, such as scFv or Fab), and b) a Fab fragment that specifically binds to EpCAM (e.g., any of the anti-EpCAM Fabs described herein); wherein the anti-CD8 antibody or antigen binding fragment thereof is fused to the N-terminus of the VH or VL of the anti-EpCAM Fab fragment via an optional linker. In some embodiments, there is provided an anti-CD8 construct (or MSAP, e.g., BSAP or TSAP), comprising a) an anti-CD8 antibody or antigen binding fragment thereof (e.g., any of the anti-CD8 antibodies or antigen binding fragments thereof described herein, such as scFv or Fab), b) a Fab fragment that specifically binds to a first target antigen that is EpCAM (e.g., any of the anti-EpCAM Fabs described herein), and c) a third binding moiety (e.g., scFv or Fab) that specifically binds to a second target antigen (e.g., immune cell surface molecule, such as CD3, CD4, or CD8); wherein: (1) the anti-CD8 antibody or antigen binding fragment thereof is fused to the N-terminus of the VH of the anti-EpCAM Fab fragment via an optional first linker, and the third binding moiety is fused to the N-terminus of the VL of the anti-EpCAM Fab fragment via an optional second linker; or (2) the anti-CD8 antibody or antigen binding fragment thereof is fused to the N-terminus of the VL of the anti-EpCAM Fab fragment via an optional first linker, and the third binding moiety is fused to the N-terminus of the VH of the anti-EpCAM Fab fragment via an optional second linker. In some embodiments, the anti-EpCAM Fab fragment comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, there is provided an anti-CD8 construct (or MSAP, e.g., BSAP) comprising: a) an anti-CD8 antibody or antigen binding fragment thereof (e.g., any of the anti-CD8 antibodies or antigen binding fragments thereof described herein, such as scFv or Fab), and b) a Fab fragment that specifically binds to WT1 (e.g., any of the anti-WT1 Fabs described herein); wherein the anti-CD8 antibody or antigen binding fragment thereof is fused to the N-terminus of the VH or VL of the anti-WT1 Fab fragment via an optional linker. In some embodiments, there is provided an anti-CD8 construct (or MSAP, e.g., BSAP or TSAP), comprising a) an anti-CD8 antibody or antigen binding fragment thereof (e.g., any of the anti-CD8 antibodies or antigen binding fragments thereof described herein, such as scFv or Fab), b) a Fab fragment that specifically binds to a first target antigen that is WT1 (e.g., any of the anti-WT1 Fabs described herein), and c) a third binding moiety (e.g., scFv or Fab) that specifically binds to a second target antigen (e.g., immune cell surface molecule, such as CD3, CD4, or CD8); wherein: (1) the anti-CD8 antibody or antigen binding fragment thereof is fused to the N-terminus of the VH of the anti-WT1 Fab fragment via an optional first linker, and the third binding moiety is fused to the N-terminus of the VL of the anti-WT1 Fab fragment via an optional second linker; or (2) the anti-CD8 antibody or antigen binding fragment thereof is fused to the N-terminus of the VL of the anti-WT1 Fab fragment via an optional first linker, and the third binding moiety is fused to the N-terminus of the VH of the anti-WT1 Fab fragment via an optional second linker. In some embodiments, the anti-WT1 Fab fragment comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the anti-CD8 antibody or antigen binding fragment thereof comprises: i) an HVR-H1 comprising the amino acid sequence of SEQ ID NO: 13, an HVR-H2 comprising the amino acid sequence of SEQ ID NO: 14, an HVR-H3 comprising the amino acid sequence of SEQ ID NO: 15, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; ii) an HVR-H1 comprising the amino acid sequence of SEQ ID NO: 1, an HVR-H2 comprising the amino acid sequence of SEQ ID NO: 16, an HVR-H3 comprising the amino acid sequence of SEQ ID NO: 3, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iii) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iv) an HVR-H1 comprising the amino acid sequence of SEQ ID NO: 1, an HVR-H2 comprising the amino acid sequence of SEQ ID NO: 18, an HVR-H3 comprising the amino acid sequence of SEQ ID NO: 19, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; v) an HVR-H1 comprising the amino acid sequence of SEQ ID NO: 7, an HVR-H2 comprising the amino acid sequence of SEQ ID NO: 8, an HVR-H3 comprising the amino acid sequence of SEQ ID NO: 9, an HVR-L1 comprising the amino acid sequence of SEQ ID NO: 10, an HVR-L2 comprising the amino acid sequence of SEQ ID NO: 11, and an HVR-L3 comprising the amino acid sequence of SEQ ID NO: 12; or vi) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23. In some embodiments, the anti-CD8 antigen binding fragment thereof and/or the third binding moiety is an anti-CD8 scFv (e.g., any of the anti-CD8 scFvs described herein). In some embodiments, the anti-CD8 antigen binding fragment thereof and the third binding moiety are identical. In some embodiments, the anti-CD8 antibody or antigen binding fragment thereof, and the third binding moiety are different. In some embodiments, the third binding moiety is an anti-CD3 scFv (e.g., any of the anti-CD3 scFvs described herein). In some embodiments, the linker, the first linker, and/or the second linker comprises an amino acid sequence independently selected from the group consisting of GG and SEQ ID NOs: 108-110.

In some embodiments, there is provided an anti-CD8 construct (or MSAP, e.g., BSAP), comprising a) an anti-CD8 Fab fragment (e.g., any of the anti-CD8 Fab fragments described herein), wherein the anti-CD8 Fab fragment comprises a first polypeptide comprising a VH and a CH1, and a second polypeptide comprising a VL and a CL, and b) a second binding moiety (e.g., scFv or Fab) that specifically binds to CD19 (e.g., any of the anti-CD19 moieties described herein); wherein the second binding moiety is fused to the N-terminus of the VH or VL of the anti-CD8 Fab fragment via an optional linker. In some embodiments, there is provided an anti-CD8 construct (or MSAP, e.g., BSAP or TSAP), comprising a) an anti-CD8 Fab fragment (e.g., any of the anti-CD8 Fab fragments described herein), wherein the anti-CD8 Fab fragment comprises a first polypeptide comprising a VH and a CH1, and a second polypeptide comprising a VL and a CL, b) a second binding moiety (e.g., scFv or Fab) that specifically binds to a first target epitope of CD19 (e.g., any of the anti-CD19 moieties described herein), and c) a third binding moiety (e.g., scFv or Fab) that specifically binds to a second target epitope of CD19 (e.g., any of the anti-CD19 moieties described herein); wherein: (1) the second binding moiety is fused to the N-terminus of the VH of the anti-CD8 Fab fragment via an optional first linker, and the third binding moiety is fused to the N-terminus of the VL of the anti-CD8 Fab fragment via an optional second linker; or (2) the second binding moiety is fused to the N-terminus of the VL of the anti-CD8 Fab fragment via an optional first linker, and the third binding moiety is fused to the N-terminus of the VH of the anti-CD8 Fab fragment via an optional second linker. In some embodiments, the anti-CD8 Fab fragment comprises: i) an HVR-H1 comprising the amino acid sequence of SEQ ID NO: 13, an HVR-H2 comprising the amino acid sequence of SEQ ID NO: 14, an HVR-H3 comprising the amino acid sequence of SEQ ID NO: 15, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; ii) an HVR-H1 comprising the amino acid sequence of SEQ ID NO: 1, an HVR-H2 comprising the amino acid sequence of SEQ ID NO: 16, an HVR-H3 comprising the amino acid sequence of SEQ ID NO: 3, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iii) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iv) an HVR-H1 comprising the amino acid sequence of SEQ ID NO: 1, an HVR-H2 comprising the amino acid sequence of SEQ ID NO: 18, an HVR-H3 comprising the amino acid sequence of SEQ ID NO: 19, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; v) an HVR-H1 comprising the amino acid sequence of SEQ ID NO: 7, an HVR-H2 comprising the amino acid sequence of SEQ ID NO: 8, an HVR-H3 comprising the amino acid sequence of SEQ ID NO: 9, an HVR-L1 comprising the amino acid sequence of SEQ ID NO: 10, an HVR-L2 comprising the amino acid sequence of SEQ ID NO: 11, and an HVR-L3 comprising the amino acid sequence of SEQ ID NO: 12; or vi) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23. In some embodiments, the first target epitope of CD19 and the second target epitope of CD19 are the same. In some embodiments, the first target epitope of CD19 and the second target epitope of CD19 are different. In some embodiments, the linker, the first linker, and/or the second linker comprises an amino acid sequence independently selected from the group consisting of GG and SEQ ID NOs: 108-110. In some embodiments, the second binding moiety and/or the third binding moiety comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the second binding moiety and/or the third binding moiety is an anti-CD19 scFv. In some embodiments, the second binding moiety and the third binding moiety are identical. In some embodiments, the second binding moiety and the third binding moiety are different.

In some embodiments, there is provided an anti-CD8 construct (or MSAP, e.g., BSAP) comprising: a) an anti-CD8 antibody or antigen binding fragment thereof (e.g., any of the anti-CD8 antibodies or antigen binding fragments thereof described herein, such as scFv or Fab), and b) a Fab fragment that specifically binds to CD19 (e.g., any of the anti-CD19 Fabs described herein); wherein the anti-CD8 antibody or antigen binding fragment thereof is fused to the N-terminus of the VH or VL of the anti-CD19 Fab fragment via an optional linker. In some embodiments, there is provided an anti-CD8 construct (or MSAP, e.g., BSAP or TSAP), comprising a) an anti-CD8 antibody or antigen binding fragment thereof (e.g., any of the anti-CD8 antibodies or antigen binding fragments thereof described herein, such as scFv or Fab), b) a Fab fragment that specifically binds to a first target antigen that is CD19 (e.g., any of the anti-CD19 Fabs described herein), and c) a third binding moiety (e.g., scFv or Fab) that specifically binds to a second target antigen (e.g., immune cell surface molecule, such as CD3, CD4, or CD8); wherein: (1) the anti-CD8 antibody or antigen binding fragment thereof is fused to the N-terminus of the VH of the anti-CD19 Fab fragment via an optional first linker, and the third binding moiety is fused to the N-terminus of the VL of the anti-CD19 Fab fragment via an optional second linker; or (2) the anti-CD8 antibody or antigen binding fragment thereof is fused to the N-terminus of the VL of the anti-CD19 Fab fragment via an optional first linker, and the third binding moiety is fused to the N-terminus of the VH of the anti-CD19 Fab fragment via an optional second linker. In some embodiments, the anti-CD8 antibody or antigen binding fragment thereof comprises: i) an HVR-H1 comprising the amino acid sequence of SEQ ID NO: 13, an HVR-H2 comprising the amino acid sequence of SEQ ID NO: 14, an HVR-H3 comprising the amino acid sequence of SEQ ID NO: 15, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; ii) an HVR-H1 comprising the amino acid sequence of SEQ ID NO: 1, an HVR-H2 comprising the amino acid sequence of SEQ ID NO: 16, an HVR-H3 comprising the amino acid sequence of SEQ ID NO: 3, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iii) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iv) an HVR-H1 comprising the amino acid sequence of SEQ ID NO: 1, an HVR-H2 comprising the amino acid sequence of SEQ ID NO: 18, an HVR-H3 comprising the amino acid sequence of SEQ ID NO: 19, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; v) an HVR-H1 comprising the amino acid sequence of SEQ ID NO: 7, an HVR-H2 comprising the amino acid sequence of SEQ ID NO: 8, an HVR-H3 comprising the amino acid sequence of SEQ ID NO: 9, an HVR-L1 comprising the amino acid sequence of SEQ ID NO: 10, an HVR-L2 comprising the amino acid sequence of SEQ ID NO: 11, and an HVR-L3 comprising the amino acid sequence of SEQ ID NO: 12; or vi) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23. In some embodiments, the anti-CD8 antibody or antigen binding fragment thereof comprises: i) a VH comprising the amino acid sequence of SEQ ID NO: 24, and a VL comprising the amino acid sequence of SEQ ID NO: 25; ii) a VH comprising the amino acid sequence of SEQ ID NO: 26, and a VL comprising the amino acid sequence of SEQ ID NO: 27; iii) a VH comprising the amino acid sequence of SEQ ID NO: 28, and a VL comprising the amino acid sequence of SEQ ID NO: 29; iv) a VH comprising the amino acid sequence of SEQ ID NO: 30, and a VL comprising the amino acid sequence of SEQ ID NO: 25; v) a VH comprising the amino acid sequence of SEQ ID NO: 32, and a VL comprising the amino acid sequence of SEQ ID NO: 33; or vi) a VH comprising the amino acid sequence of SEQ ID NO: 34, and a VL comprising the amino acid sequence of SEQ ID NO: 35. In some embodiments, the anti-CD8 antigen binding fragment thereof and/or the third binding moiety is an anti-CD8 scFv (e.g., any of the anti-CD8 scFvs described herein). In some embodiments, the anti-CD8 scFv comprises the amino acid sequence of any one of SEQ ID NOs: 21, 44-49, and 51-55, such as SEQ ID NO: 46, 49, 52, or 55. In some embodiments, the anti-CD8 antigen binding fragment thereof and the third binding moiety are identical. In some embodiments, the anti-CD8 antibody or antigen binding fragment thereof, and the third binding moiety are different. In some embodiments, the third binding moiety is an anti-CD3 scFv (e.g., any of the anti-CD3 scFvs described herein). In some embodiments, the linker, the first linker, and/or the second linker comprises an amino acid sequence independently selected from the group consisting of GG and SEQ ID NOs: 108-110. In some embodiments, the anti-CD19 Fab fragment comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD19 Fab fragment comprises a VH comprising the amino acid sequence of SEQ ID NO: 63, and a VL comprising the amino acid sequence of SEQ ID NO: 64. In some embodiments, the anti-CD19 Fab fragment comprises a first polypeptide comprising the amino acid sequence of SEQ ID NO: 137, and a second polypeptide comprising the amino acid sequence of SEQ ID NO: 139 or 169.

Hence in some embodiments, there is provided an anti-CD8 construct (or MSAP, e.g., BSAP) comprising: a) an anti-CD19 Fab fragment (e.g., any of the anti-CD19 Fabs described herein), wherein the anti-CD19 Fab fragment comprises a first polypeptide comprising a VH and a CH1, and a second polypeptide comprising a VL and a CL, and b) an anti-CD8 scFv (e.g., any of the anti-CD8 scFvs described herein); wherein the anti-CD8 scFv is fused to the N-terminus of the VH or VL of the anti-CD19 Fab fragment via an optional linker to form a fusion polypeptide. In some embodiments, there is provided an anti-CD8 construct (or MSAP, e.g., BSAP) comprising: a) an anti-CD19 Fab fragment, wherein the anti-CD19 Fab fragment comprises a first polypeptide comprising a VH and a CH1, and a second polypeptide comprising a VL and a CL, and b) an anti-CD8 scFv; wherein the anti-CD8 scFv is fused to the N-terminus of the VH of the anti-CD19 Fab fragment via a linker to form a fusion polypeptide; and wherein the fusion polypeptide comprises the amino acid sequence of SEQ ID NO: 162, and the second polypeptide comprises the amino acid sequence of SEQ ID NO: 139.

In some embodiments, there is provided an anti-CD8 construct (or MSAP, e.g., BSAP), comprising a) an anti-CD8 Fab fragment (any of the anti-CD8 Fab fragments described herein), wherein the anti-CD8 Fab fragment comprises a first polypeptide comprising a VH and a CH1, and a second polypeptide comprising a VL and a CL, and b) a second binding moiety (e.g., scFv or Fab) that specifically binds to WT1 (e.g., any of the anti-WT1 moieties described herein); wherein the second binding moiety is fused to the N-terminus of the VH or VL of the anti-CD8 Fab fragment via an optional linker. In some embodiments, there is provided an anti-CD8 construct (or MSAP, e.g., BSAP or TSAP), comprising a) an anti-CD8 Fab fragment (any of the anti-CD8 Fab fragments described herein), wherein the anti-CD8 Fab fragment comprises a first polypeptide comprising a VH and a CH1, and a second polypeptide comprising a VL and a CL, b) a second binding moiety (e.g., scFv or Fab) that specifically binds to a first target epitope of WT1 (e.g., any of the anti-WT1 moieties described herein), and c) a third binding moiety (e.g., scFv or Fab) that specifically binds to a second target epitope of WT1 (e.g., any of the anti-WT1 moieties described herein); wherein: (1) the second binding moiety is fused to the N-terminus of the VH of the anti-CD8 Fab fragment via an optional first linker, and the third binding moiety is fused to the N-terminus of the VL of the anti-CD8 Fab fragment via an optional second linker; or (2) the second binding moiety is fused to the N-terminus of the VL of the anti-CD8 Fab fragment via an optional first linker, and the third binding moiety is fused to the N-terminus of the VH of the anti-CD8 Fab fragment via an optional second linker. In some embodiments, the anti-CD8 Fab fragment comprises: i) an HVR-H1 comprising the amino acid sequence of SEQ ID NO: 13, an HVR-H2 comprising the amino acid sequence of SEQ ID NO: 14, an HVR-H3 comprising the amino acid sequence of SEQ ID NO: 15, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; ii) an HVR-H1 comprising the amino acid sequence of SEQ ID NO: 1, an HVR-H2 comprising the amino acid sequence of SEQ ID NO: 16, an HVR-H3 comprising the amino acid sequence of SEQ ID NO: 3, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iii) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; iv) an HVR-H1 comprising the amino acid sequence of SEQ ID NO: 1, an HVR-H2 comprising the amino acid sequence of SEQ ID NO: 18, an HVR-H3 comprising the amino acid sequence of SEQ ID NO: 19, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6; v) an HVR-H1 comprising the amino acid sequence of SEQ ID NO: 7, an HVR-H2 comprising the amino acid sequence of SEQ ID NO: 8, an HVR-H3 comprising the amino acid sequence of SEQ ID NO: 9, an HVR-L1 comprising the amino acid sequence of SEQ ID NO: 10, an HVR-L2 comprising the amino acid sequence of SEQ ID NO: 11, and an HVR-L3 comprising the amino acid sequence of SEQ ID NO: 12; or vi) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23. In some embodiments, the first target epitope of WT1 and the second target epitope of WT1 are the same. In some embodiments, the first target epitope of WT1 and the second target epitope of WT1 are different. In some embodiments, the linker, the first linker, and/or the second linker comprises an amino acid sequence independently selected from the group consisting of GG and SEQ ID NOs: 108-110. In some embodiments, the second binding moiety and/or the third binding moiety comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the second binding moiety and/or the third binding moiety is an anti-WT1 scFv. In some embodiments, the second binding moiety and the third binding moiety are identical. In some embodiments, the second binding moiety and the third binding moiety are different.

Also provided are isolated nucleic acid(s) encoding any of the anti-CD8 antibodies or antigen-binding fragments thereof (e.g., full-length antibody, scFv, or Fab) described herein, or any of the anti-CD8 constructs (e.g., anti-CD8 MSAPs, such as BSAP or TSAP) described herein; vector(s) (e.g., viral vector, such as lentiviral vector or AAV) comprising such isolated nucleic acid(s); and host cells (prokaryotic or eukaryotic) comprising such isolated nucleic acid(s) or vector(s), or expressing any of the anti-CD8 antibodies or antigen-binding fragments thereof described herein, or any of the anti-CD8 constructs (e.g., anti-CD8 MSAPs) described herein.

The invention further provides fusion proteins comprising any of the anti-CD8 antibodies or antigen-binding fragments thereof (e.g., full-length antibody, scFv, or Fab) described herein or anti-CD8 constructs described herein, and conjugates (e.g., small molecule drug conjugates, or detection tag conjugates) of any of the anti-CD8 antibodies or antigen-binding fragments thereof or any of the anti-CD8 constructs (e.g., MSAPs) described herein.

### IV. Anti-CD3 Constructs

In one aspect, the present application provides an anti-CD3 construct comprising (or consisting of, or consisting essentially of) any of the anti-CD3 antibodies or antigen-binding fragments thereof (or anti-CD3 moieties) described herein. In some embodiments, the anti-CD3 construct is a MSAP, such as any of the MSAPs comprising an anti-CD3 moiety described herein. Hence in some embodiments, there is provided an MSAP (e.g., TSAP, BSAP) comprising any of the anti-CD3 antibodies or antigen binding fragments thereof (e.g., scFv or Fab) described herein.

In some embodiments, the anti-CD3 construct comprises (or consists of, or consists essentially of) a full-length antibody, a Fab, a Fab′, a Fab′-SH, a F(ab′)₂, an Fv, an scFv, or a combination thereof. In some embodiments, the anti-CD3 construct is an anti-CD3 Fab fragment. In some embodiments, the anti-CD3 construct is an anti-CD3 scFv. In some embodiments, the anti-CD3 construct is an anti-CD3 full-length antibody comprising an Fc region of any antibody class or isotype, such as IgG1, IgG2, IgG3, or IgG4. In some embodiments, the Fc region has reduced or minimized effector function.

In some embodiments, the anti-CD3 construct is monospecific. In some embodiments, the anti-CD3 construct is multispecific (e.g., bispecific, or trispecific). In some embodiments, the anti-CD3 construct is monovalent. In some embodiments, the anti-CD3 construct is multivalent (e.g., bivalent, or trivalent). In some embodiments, the anti-CD3 construct is multivalent and monospecific. In some embodiments, the anti-CD3 construct is multivalent and multispecific.

In some embodiments, the anti-CD3 construct is selected from the group consisting of: a multispecific antibody construct (e.g., bispecific antibody construct, or trispecific antibody construct, such as MSAP), a bispecific T cell engager (BiTE), an antibody-drug conjugate (ADC), an antibody-siRNA conjugate (ARC), an antibody-oligonucleotide conjugate (AOC), an antibody-detection tag conjugate, an immunocytokine, a trispecific NK-cell engager (TriKE), a trispecific T-cell activation construct (TriTAC), and a tandem bivalent antibody.

In some embodiments, there is provided an anti-CD3 MSAP (e.g., BSAP, TSAP) comprising any of the anti-CD3 antibodies or antigen-binding fragments thereof (e.g., scFv or Fab) described herein. In some embodiments, there is provided an anti-CD3 construct (e.g., MSAP) comprising two or more anti-CD3 moieties described herein, such as connected in tandem (e.g., scFv-scFv). The two or more anti-CD3 moieties can recognize the same CD3 epitope or at least one different CD3 epitope. In some embodiments, there is provided an anti-CD3 construct (or an MSAP, e.g., BSAP) comprising: i) an anti-CD3 antibody or antigen binding fragment thereof (e.g., any of the anti-CD3 antibodies or antigen-binding fragments thereof described herein), and ii) a second binding moiety (e.g., scFv or Fab) that specifically binds to a non-CD3 target antigen (e.g., TSA or TAA, such as CD19, EpCAM, WT1). In some embodiments, there is provided an anti-CD3 construct (or an MSAP, e.g., BSAP) comprising: a) an anti-CD3 antibody or antigen binding fragment thereof comprising: i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and b) a second binding moiety (e.g., scFv or Fab) that specifically binds to a non-CD3 target antigen (e.g., TSA or TAA, such as CD19, EpCAM, WT1). In some embodiments, the anti-CD3 antibody or antigen binding fragment thereof comprises a VH comprising the amino acid sequence of any of SEQ ID NOs: 42, 159, and 167, and a VL comprising the amino acid sequence of SEQ ID NO: 43. In some embodiments, the anti-CD3 antibody or antigen binding fragment thereof and the second binding moiety are independently selected from the group consisting of a full-length antibody, a Fab, a Fab′, a Fab′-SH, a F(ab′)2, an Fv, an scFv, and any combination thereof. In some embodiments, the anti-CD3 antigen binding fragment is an anti-CD3 Fab fragment or an anti-CD3 scFv. The non-CD3 target antigen can be any target antigens (e.g., disease-associated target antigen) described in the “Anti-target epitope moiety” section above. In some embodiments, the anti-CD3 antibody or antigen binding fragment thereof is fused to the second binding moiety via a linker (e.g., peptide linker). In some embodiments, the anti-CD3 construct (or MSAP) further comprises a third binding moiety (e.g., scFv or Fab) that specifically binds to a second target antigen (e.g., immune cell surface molecule, such as CD3, CD4, CD8; or TSA or TAA, such as CD19, EpCAM, WT1). The second binding moiety and/or the third binding moiety can be fused (e.g., via linker(s)) with the anti-CD3 antibody or antigen binding fragment thereof in any configurations, such as those described for MSAPs under section II. For example, when moiety A is a Fab fragment, moiety B and/or moiety C can be independently fused to N′ of VH, N′ of VL, C′ of CH1, and/or C′ of CL of moiety A Fab fragment.

In some embodiments, there is provided an anti-CD3 construct (or MSAP, e.g., BSAP), comprising i) an anti-CD3 Fab fragment (e.g., any of the anti-CD3 Fab fragments described herein), wherein the anti-CD3 Fab fragment comprises a first polypeptide comprising a VH and a CH1, and a second polypeptide comprising a VL and a CL, and ii) a second binding moiety (e.g., scFv or Fab) that specifically binds to a non-CD3 target antigen (e.g., TSA or TAA, such as CD19, EpCAM, or WT1). In some embodiments, there is provided an anti-CD3 construct (or MSAP, e.g., BSAP), comprising i) an anti-CD3 Fab fragment (e.g., any of the anti-CD3 Fab fragments described herein), wherein the anti-CD3 Fab fragment comprises a first polypeptide comprising a VH and a CH1, and a second polypeptide comprising a VL and a CL, and ii) a second binding moiety (e.g., scFv or Fab) that specifically binds to a non-CD3 target antigen (e.g., TSA or TAA, such as CD19, EpCAM, or WT1); wherein the second binding moiety is fused to the N-terminus of the VH or the VL of the anti-CD3 Fab fragment via an optional linker. In some embodiments, the second binding moiety is an scFv. In some embodiments, there is provided an anti-CD3 construct (or MSAP, e.g., BSAP or TSAP), comprising i) an anti-CD3 Fab fragment (e.g., any of the anti-CD3 Fab fragments described herein), wherein the anti-CD3 Fab fragment comprises a first polypeptide comprising a VH and a CH1, and a second polypeptide comprising a VL and a CL, ii) a second binding moiety (e.g., scFv or Fab) that specifically binds to a first non-CD3 target antigen (e.g., TSA or TAA, such as CD19, EpCAM, or WT1; or immune cell surface molecule, such as CD8), and iii) a third binding moiety (e.g., scFv or Fab) that specifically binds to a second non-CD3 target antigen (e.g., immune cell surface molecule, such as CD8; or TSA or TAA, such as CD19, EpCAM, or WT1); wherein the first binding moiety is fused to the N-terminus of the VH of the anti-CD3 Fab fragment via an optional first linker, and the second binding moiety is fused to the N-terminus of the VL of the anti-CD3 Fab fragment via an optional second linker. In some embodiments, the second binding moiety and the third binding moiety are both scFvs. In some embodiments, the anti-CD3 Fab fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the anti-CD3 Fab fragment comprises a VH comprising the amino acid sequence of any one of SEQ ID NOs: 42, 159, and 167, and a VL comprising the amino acid sequence of SEQ ID NO: 43. In some embodiments, the anti-CD3 Fab fragment comprises a first polypeptide comprising the amino acid sequence of any one of SEQ ID NOs: 89, 160, and 168, and a second polypeptide comprising the amino acid sequence of SEQ ID NO: 90 or 179. In some embodiments, the first non-CD3 target antigen (or epitope) and the second non-CD3 target antigen (or epitope) are the same. In some embodiments, the first non-CD3 target antigen (or epitope) and the second non-CD3 target antigen (or epitope) are different. In some embodiments, the first non-CD3 target antigen and the second non-CD3 target antigen are both TSA or TAA, e.g., both CD19 or both EpCAM or both WT1 (either same epitope or different epitope). In some embodiments, the first non-CD3 target antigen is a TSA or TAA (e.g., CD19, EpCAM, or WT1), and the second non-CD3 target antigen is an immune cell surface molecule (e.g., CD4, CD8). In some embodiments, the second non-CD3 target antigen is a TSA or TAA (e.g., CD19, EpCAM, or WT1), and the first non-CD3 target antigen is an immune cell surface molecule (e.g., CD4, CD8). In some embodiments, the second binding moiety is an scFv, e.g., anti-EpCAM scFv, anti-CD19 scFv, anti-WT1 scFv, or anti-CD8 scFv. In some embodiments, the third binding moiety is an scFv, e.g., anti-CD8 scFv, anti-EpCAM scFv, anti-CD19 scFv, or anti-WT1 scFv. In some embodiments, the second binding moiety and the third binding moiety are both anti-EpCAM scFvs, both anti-CD19 scFvs, or both anti-WT1 scFvs. In some embodiments, the second binding moiety is an anti-CD8 scFv, and the third binding moiety is an anti-target epitope (e.g., TSA or TAA, such as EpCAM, CD19, or WT1) scFv. In some embodiments, the third binding moiety is an anti-CD8 scFv, and the second binding moiety is an anti-target epitope (e.g., TSA or TAA, such as EpCAM, CD19, or WT1) scFv. In some embodiments, the linker, the first linker, and/or the second linker comprises an amino acid sequence independently selected from the group consisting of GG and SEQ ID NOs: 108-110.

In some embodiments, there is provided an anti-CD3 construct (or MSAP, e.g., BSAP), comprising i) an anti-CD3 antibody or antigen binding fragment thereof (e.g., scFv or Fab), and ii) a Fab fragment that specifically binds to a non-CD3 target antigen (e.g., TSA or TAA, such as CD19, EpCAM, or WT1). In some embodiments, there is provided an anti-CD3 construct (or MSAP, e.g., BSAP), comprising i) an anti-CD3 antibody or antigen binding fragment thereof (e.g., scFv or Fab), and ii) a Fab fragment that specifically binds to a non-CD3 target antigen (e.g., TSA or TAA, such as CD19, EpCAM, or WT1); wherein the anti-CD3 antibody or antigen binding fragment thereof is fused to the N-terminus of the VH or the VL of the non-CD3 target antigen Fab fragment via an optional linker. In some embodiments, anti-CD3 antibody or antigen binding fragment thereof is an anti-CD3 scFv. In some embodiments, there is provided an anti-CD3 construct (or MSAP, e.g., BSAP or TSAP), comprising i) an anti-CD3 antibody or antigen binding fragment thereof (e.g., scFv or Fab), ii) a Fab fragment that specifically binds to a first target antigen that is non-CD3 target antigen (e.g., TSA or TAA, such as CD19, EpCAM, or WT1; or immune cell surface molecule, such as CD8), and iii) a third binding moiety (e.g., scFv or Fab) that specifically binds to a second target antigen (e.g., immune cell surface molecule, such as CD3 or CD8; or TSA or TAA, such as CD19, EpCAM, or WT1); wherein: (1) the anti-CD3 antibody or antigen binding fragment thereof is fused to the N-terminus of the VH of the first target antigen Fab fragment via an optional first linker, and the third binding moiety is fused to the N-terminus of the VL of the first target antigen Fab fragment via an optional second linker; or (2) the anti-CD3 antibody or antigen binding fragment thereof is fused to the N-terminus of the VL of the first target antigen Fab fragment via an optional first linker, and the third binding moiety is fused to the N-terminus of the VH of the first target antigen Fab fragment via an optional second linker. In some embodiments, the anti-CD3 antibody or antigen binding fragment thereof and the third binding moiety are both scFvs. In some embodiments, the anti-CD3 antibody or antigen binding fragment thereof comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the anti-CD3 antibody or antigen binding fragment thereof comprises a VH comprising the amino acid sequence of any of SEQ ID NOs: 42, 159, and 167, and a VL comprising the amino acid sequence of SEQ ID NO: 43. In some embodiments, the anti-CD3 antigen binding fragment and/or the third binding moiety is an anti-CD3 scFv (e.g., any of the anti-CD3 scFvs described herein). In some embodiments, the anti-CD3 scFv comprises the amino acid sequence of any of SEQ ID NOs: 50, 56, 163, 164, 183, and 184. The first target antigen (or epitope) and the second target antigen (or epitope) can be the or different. In some embodiments, the first target antigen and second target antigen are both TSA or TAA, e.g., both CD19, both EpCAM, or both WT1 (either same epitope or different epitope). In some embodiments, the first target antigen is a TSA or TAA (e.g., CD19, EpCAM, or WT1), and the second target antigen is an immune cell surface molecule (e.g., CD3, CD4, CD8). In some embodiments, the second target antigen is a TSA or TAA (e.g., CD19, EpCAM, or WT1), and the first target antigen is an immune cell surface molecule (e.g., CD4, CD8). In some embodiments, the anti-CD3 antigen binding fragment is an anti-CD3 scFv. In some embodiments, the third binding moiety is an scFv, e.g., anti-CD3 scFv, anti-CD8 scFv, anti-EpCAM scFv, anti-CD19 scFv, or anti-WT1 scFv. In some embodiments, the anti-CD3 antigen binding fragment and the third binding moiety are both anti-CD3 scFvs. The anti-CD3 antigen binding fragment (e.g., scFv) and the third binding moiety can be identical or different. In some embodiments, the anti-first target antigen Fab is an anti-CD8 Fab, and the third binding moiety is an anti-target epitope (e.g., TSA or TAA, such as EpCAM, CD19, or WT1) scFv. In some embodiments, the third binding moiety is an anti-CD8 or anti-CD3 scFv (e.g., any of the anti-CD8 or anti-CD3 scFvs described herein), and the anti-first target antigen Fab is an anti-target epitope (e.g., TSA or TAA, such as EpCAM, CD19, or WT1) Fab. In some embodiments, the linker, the first linker, and/or the second linker comprises an amino acid sequence independently selected from the group consisting of GG and SEQ ID NOs: 108-110.

In some embodiments, there is provided an anti-CD3 construct (or MSAP, e.g., BSAP), comprising a) an anti-CD3 Fab fragment (e.g., any of the anti-CD3 Fab fragments described herein), wherein the anti-CD3 Fab fragment comprises a first polypeptide comprising a VH and a CH1, and a second polypeptide comprising a VL and a CL, and b) a second binding moiety (e.g., scFv or Fab) that specifically binds to EpCAM (e.g., any of the anti-EpCAM moieties described herein); wherein the second binding moiety is fused to the N-terminus of the VH or VL of the anti-CD3 Fab fragment via an optional linker. In some embodiments, there is provided an anti-CD3 construct (or MSAP, e.g., BSAP or TSAP), comprising a) an anti-CD3 Fab fragment (e.g., any of the anti-CD3 Fab fragments described herein), wherein the anti-CD3 Fab fragment comprises a first polypeptide comprising a VH and a CH1, and a second polypeptide comprising a VL and a CL, b) a second binding moiety (e.g., scFv or Fab) that specifically binds to a first target epitope of EpCAM (e.g., any of the anti-EpCAM moieties described herein), and c) a third binding moiety (e.g., scFv or Fab) that specifically binds to a second target epitope of EpCAM (e.g., any of the anti-EpCAM moieties described herein); wherein: (1) the second binding moiety is fused to the N-terminus of the VH of the anti-CD3 Fab fragment via an optional first linker, and the third binding moiety is fused to the N-terminus of the VL of the anti-CD3 Fab fragment via an optional second linker; or (2) the second binding moiety is fused to the N-terminus of the VL of the anti-CD3 Fab fragment via an optional first linker, and the third binding moiety is fused to the N-terminus of the VH of the anti-CD3 Fab fragment via an optional second linker. In some embodiments, the anti-CD3 Fab fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the anti-CD3 Fab fragment comprises a VH comprising the amino acid sequence of any of SEQ ID NOs: 42, 159, and 167, and a VL comprising the amino acid sequence of SEQ ID NO: 43. In some embodiments, the anti-CD3 Fab fragment comprises: a first polypeptide comprising the amino acid sequence of any of SEQ ID NOs: 89, 160, and 168, and a second polypeptide comprising the amino acid sequence of SEQ ID NO: 90 or 179. In some embodiments, the first target epitope of EpCAM and the second target epitope of EpCAM are the same. In some embodiments, the first target epitope of EpCAM and the second target epitope of EpCAM are different. In some embodiments, the linker, the first linker, and/or the second linker comprises an amino acid sequence independently selected from the group consisting of GG and SEQ ID NOs: 108-110. In some embodiments, the second binding moiety and/or the third binding moiety comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, the second binding moiety and/or the third binding moiety comprises a VH comprising the amino acid sequence of SEQ ID NO: 73, and a VL comprising the amino acid sequence of SEQ ID NO: 74. In some embodiments, the second binding moiety and/or the third binding moiety is an anti-EpCAM scFv. In some embodiments, the anti-EpCAM scFv comprises the amino acid sequence of SEQ ID NO: 75 or 78. In some embodiments, the second binding moiety and the third binding moiety are identical. In some embodiments, the second binding moiety and the third binding moiety are different.

In some embodiments, there is provided an anti-CD3 construct (or MSAP, e.g., BSAP) comprising: a) an anti-CD3 antibody or antigen binding fragment thereof (e.g., any of the anti-CD3 antibodies or antigen binding fragments thereof described herein, such as scFv or Fab), and b) a Fab fragment that specifically binds to EpCAM (e.g., any of the anti-EpCAM Fabs described herein); wherein the anti-CD3 antibody or antigen binding fragment thereof is fused to the N-terminus of the VH or VL of the anti-EpCAM Fab fragment via an optional linker. In some embodiments, there is provided an anti-CD3 construct (or MSAP, e.g., BSAP or TSAP), comprising a) an anti-CD3 antibody or antigen binding fragment thereof (e.g., any of the anti-CD3 antibodies or antigen binding fragments thereof described herein, such as scFv or Fab), b) a Fab fragment that specifically binds to a first target antigen that is EpCAM (e.g., any of the anti-EpCAM Fabs described herein), and c) a third binding moiety (e.g., scFv or Fab) that specifically binds to a second target antigen (e.g., immune cell surface molecule, such as CD3, CD4, or CD8); wherein: (1) the anti-CD3 antibody or antigen binding fragment thereof is fused to the N-terminus of the VH of the anti-EpCAM Fab fragment via an optional first linker, and the third binding moiety is fused to the N-terminus of the VL of the anti-EpCAM Fab fragment via an optional second linker; or (2) the anti-CD3 antibody or antigen binding fragment thereof is fused to the N-terminus of the VL of the anti-EpCAM Fab fragment via an optional first linker, and the third binding moiety is fused to the N-terminus of the VH of the anti-EpCAM Fab fragment via an optional second linker. In some embodiments, the anti-CD3 antibody or antigen binding fragment thereof comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the anti-CD3 antibody or antigen binding fragment thereof comprises a VH comprising the amino acid sequence of any of SEQ ID NOs: 42, 159, and 167, and a VL comprising the amino acid sequence of SEQ ID NO: 43. In some embodiments, the anti-CD3 antigen binding fragment thereof and/or the third binding moiety is an anti-CD3 scFv (e.g., any of the anti-CD3 scFvs described herein). In some embodiments, the anti-CD3 scFv comprises the amino acid sequence of any one of SEQ ID NOs: 50, 56, 163, 164, 183, and 184. In some embodiments, the anti-CD3 antigen binding fragment thereof and the third binding moiety are identical. In some embodiments, the anti-CD3 antibody or antigen binding fragment thereof, and the third binding moiety are different. In some embodiments, the third binding moiety is an anti-CD8 scFv (e.g., any of the anti-CD8 scFvs described herein). In some embodiments, the linker, the first linker, and/or the second linker comprises an amino acid sequence independently selected from the group consisting of GG and SEQ ID NOs: 108-110. In some embodiments, the anti-EpCAM Fab fragment comprises an HVR-H1 comprising the amino acid sequence of SEQ ID NO: 67, an HVR-H2 comprising the amino acid sequence of SEQ ID NO: 68, an HVR-H3 comprising the amino acid sequence of SEQ ID NO: 69, an HVR-L1 comprising the amino acid sequence of SEQ ID NO: 70, an HVR-L2 comprising the amino acid sequence of SEQ ID NO: 71, and an HVR-L3 comprising the amino acid sequence of SEQ ID NO: 72. In some embodiments, the anti-EpCAM Fab fragment comprises a VH comprising the amino acid sequence of SEQ ID NO: 73, and a VL comprising the amino acid sequence of SEQ ID NO: 74. In some embodiments, the anti-EpCAM Fab fragment comprises a first polypeptide comprising the amino acid sequence of SEQ ID NO: 76, and a second polypeptide comprising the amino acid sequence of SEQ ID NO: 77.

In some embodiments, there is provided an anti-CD3 construct (or MSAP, e.g., BSAP), comprising a) an anti-CD3 Fab fragment (e.g., any of the anti-CD3 Fab fragments described herein), wherein the anti-CD3 Fab fragment comprises a first polypeptide comprising a VH and a CH1, and a second polypeptide comprising a VL and a CL, and b) a second binding moiety (e.g., scFv or Fab) that specifically binds to CD19 (e.g., any of the anti-CD19 moieties described herein); wherein the second binding moiety is fused to the N-terminus of the VH or VL of the anti-CD3 Fab fragment via an optional linker. In some embodiments, there is provided an anti-CD3 construct (or MSAP, e.g., BSAP or TSAP), comprising a) an anti-CD3 Fab fragment (e.g., any of the anti-CD3 Fab fragments described herein), wherein the anti-CD3 Fab fragment comprises a first polypeptide comprising a VH and a CH1, and a second polypeptide comprising a VL and a CL, b) a second binding moiety (e.g., scFv or Fab) that specifically binds to a first target epitope of CD19 (e.g., any of the anti-CD19 moieties described herein), and c) a third binding moiety (e.g., scFv or Fab) that specifically binds to a second target epitope of CD19 (e.g., any of the anti-CD19 moieties described herein); wherein: (1) the second binding moiety is fused to the N-terminus of the VH of the anti-CD3 Fab fragment via an optional first linker, and the third binding moiety is fused to the N-terminus of the VL of the anti-CD3 Fab fragment via an optional second linker; or (2) the second binding moiety is fused to the N-terminus of the VL of the anti-CD3 Fab fragment via an optional first linker, and the third binding moiety is fused to the N-terminus of the VH of the anti-CD3 Fab fragment via an optional second linker. In some embodiments, the anti-CD3 Fab fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the anti-CD3 Fab fragment comprises a VH comprising the amino acid sequence of any of SEQ ID NOs: 42, 159, and 167, and a VL comprising the amino acid sequence of SEQ ID NO: 43. In some embodiments, the anti-CD3 Fab fragment comprises a first polypeptide comprising the amino acid sequence of any of SEQ ID NOs: 89, 160, and 168, and a second polypeptide comprising the amino acid sequence of SEQ ID NO: 90 or 179. In some embodiments, the first target epitope of CD19 and the second target epitope of CD19 are the same. In some embodiments, the first target epitope of CD19 and the second target epitope of CD19 are different. In some embodiments, the linker, the first linker, and/or the second linker comprises an amino acid sequence independently selected from the group consisting of GG and SEQ ID NOs: 108-110. In some embodiments, the second binding moiety and/or the third binding moiety comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the second binding moiety and/or the third binding moiety comprises a VH comprising the amino acid sequence of SEQ ID NO: 63, and a VL comprising the amino acid sequence of SEQ ID NO: 64. In some embodiments, the second binding moiety and/or the third binding moiety is an anti-CD19 scFv. In some embodiments, the anti-CD19 scFv comprises the amino acid sequence of SEQ ID NO: 65 or 66. In some embodiments, the second binding moiety and the third binding moiety are identical. In some embodiments, the second binding moiety and the third binding moiety are different.

In some embodiments, there is provided an anti-CD3 construct (or MSAP, e.g., BSAP) comprising: a) an anti-CD3 antibody or antigen binding fragment thereof (e.g., any of the anti-CD3 antibodies or antigen binding fragments thereof described herein, such as scFv or Fab), and b) a Fab fragment that specifically binds to CD19 (e.g., any of the anti-CD19 Fabs described herein); wherein the anti-CD3 antibody or antigen binding fragment thereof is fused to the N-terminus of the VH or VL of the anti-CD19 Fab fragment via an optional linker. In some embodiments, there is provided an anti-CD3 construct (or MSAP, e.g., BSAP or TSAP), comprising a) an anti-CD3 antibody or antigen binding fragment thereof (e.g., any of the anti-CD3 antibodies or antigen binding fragments thereof described herein, such as scFv or Fab), b) a Fab fragment that specifically binds to a first target antigen that is CD19, and c) a third binding moiety (e.g., scFv or Fab) that specifically binds to a second target antigen (e.g., immune cell surface molecule, such as CD3, CD4, or CD8); wherein: (1) the anti-CD3 antibody or antigen binding fragment thereof is fused to the N-terminus of the VH of the anti-CD19 Fab fragment via an optional first linker, and the third binding moiety is fused to the N-terminus of the VL of the anti-CD19 Fab fragment via an optional second linker; or (2) the anti-CD3 antibody or antigen binding fragment thereof is fused to the N-terminus of the VL of the anti-CD19 Fab fragment via an optional first linker, and the third binding moiety is fused to the N-terminus of the VH of the anti-CD19 Fab fragment via an optional second linker. In some embodiments, the anti-CD3 antibody or antigen binding fragment thereof comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the anti-CD3 antibody or antigen binding fragment thereof comprises a VH comprising the amino acid sequence of any of SEQ ID NOs: 42, 159, and 167, and a VL comprising the amino acid sequence of SEQ ID NO: 43. In some embodiments, the anti-CD3 antigen binding fragment and/or the third binding moiety is an anti-CD3 scFv (e.g., any of the anti-CD3 scFvs described herein). In some embodiments, the anti-CD3 scFv comprises the amino acid sequence of any of SEQ ID NOs: 50, 56, 163, 164, 183, and 184. In some embodiments, the anti-CD3 antigen binding fragment thereof and the third binding moiety are identical. In some embodiments, the anti-CD3 antibody or antigen binding fragment thereof, and the third binding moiety are different. In some embodiments, the third binding moiety is an anti-CD8 scFv (e.g., any of the anti-CD8 scFvs described herein). In some embodiments, the linker, the first linker, and/or the second linker comprises an amino acid sequence independently selected from the group consisting of GG and SEQ ID NOs: 108-110. In some embodiments, the anti-CD19 Fab fragment comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62. In some embodiments, the anti-CD19 Fab fragment comprises a VH comprising the amino acid sequence of SEQ ID NO: 63, and a VL comprising the amino acid sequence of SEQ ID NO: 64. In some embodiments, the anti-CD19 Fab fragment comprises a first polypeptide comprising the amino acid sequence of SEQ ID NO: 137, and a second polypeptide comprising the amino acid sequence of SEQ ID NO: 139 or 169.

Hence in some embodiments, there is provided an anti-CD3 construct (or MSAP, e.g., BSAP) comprising: a) an anti-CD19 Fab fragment (e.g., any of the anti-CD19 Fabs described herein), wherein the anti-CD19 Fab fragment comprises a first polypeptide comprising a VH and a CH1, and a second polypeptide comprising a VL and a CL, and b) an anti-CD3 scFv (e.g., any of the anti-CD3 scFvs described herein); wherein the anti-CD3 scFv is fused to the N-terminus of the VH or VL of the anti-CD19 Fab fragment via an optional linker to form a fusion polypeptide. In some embodiments, there is provided an anti-CD3 construct (or MSAP, e.g., BSAP) comprising: a) an anti-CD19 Fab fragment, wherein the anti-CD19 Fab fragment comprises a first polypeptide comprising a VH and a CH1, and a second polypeptide comprising a VL and a CL, and b) an anti-CD3 scFv; wherein the anti-CD3 scFv is fused to the N-terminus of the VH of the anti-CD19 Fab fragment via a linker to form a fusion polypeptide; and wherein the fusion polypeptide comprises the amino acid sequence of SEQ ID NO: 138, and the second polypeptide comprises the amino acid sequence of SEQ ID NO: 139.

In some embodiments, there is provided an anti-CD3 construct (or MSAP, e.g., BSAP), comprising a) an anti-CD3 Fab fragment (any of the anti-CD3 Fab fragments described herein), wherein the anti-CD3 Fab fragment comprises a first polypeptide comprising a VH and a CH1, and a second polypeptide comprising a VL and a CL, and b) a second binding moiety (e.g., scFv or Fab) that specifically binds to WT1 (e.g., any of the anti-WT1 moieties described herein); wherein the second binding moiety is fused to the N-terminus of the VH or VL of the anti-CD3 Fab fragment via an optional linker. In some embodiments, there is provided an anti-CD3 construct (or MSAP, e.g., BSAP or TSAP), comprising a) an anti-CD3 Fab fragment (any of the anti-CD3 Fab fragments described herein), wherein the anti-CD3 Fab fragment comprises a first polypeptide comprising a VH and a CH1, and a second polypeptide comprising a VL and a CL, b) a second binding moiety (e.g., scFv or Fab) that specifically binds to a first target epitope of WT1 (e.g., any of the anti-WT1 moieties described herein), and c) a third binding moiety (e.g., scFv or Fab) that specifically binds to a second target epitope of WT1 (e.g., any of the anti-WT1 moieties described herein); wherein (1) the second binding moiety is fused to the N-terminus of the VH of the anti-CD3 Fab fragment via an optional first linker, and the third binding moiety is fused to the N-terminus of the VL of the anti-CD3 Fab fragment via an optional second linker; or (2) the second binding moiety is fused to the N-terminus of the VL of the anti-CD3 Fab fragment via an optional first linker, and the third binding moiety is fused to the N-terminus of the VH of the anti-CD3 Fab fragment via an optional second linker. In some embodiments, the anti-CD3 Fab fragment comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the anti-CD3 Fab fragment comprises a VH comprising the amino acid sequence of any of SEQ ID NOs: 42, 159, and 167, and a VL comprising the amino acid sequence of SEQ ID NO: 43. In some embodiments, the anti-CD3 Fab fragment comprises a first polypeptide comprising the amino acid sequence of any of SEQ ID NOs: 89, 160, and 168, and a second polypeptide comprising the amino acid sequence of SEQ ID NO: 90 or 179. In some embodiments, the first target epitope of WT1 and the second target epitope of WT1 are the same. In some embodiments, the first target epitope of WT1 and the second target epitope of WT1 are different. In some embodiments, the linker, the first linker, and/or the second linker comprises an amino acid sequence independently selected from the group consisting of GG and SEQ ID NOs: 108-110. In some embodiments, the second binding moiety and/or the third binding moiety comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the second binding moiety and/or the third binding moiety comprises a VH comprising the amino acid sequence of SEQ ID NO: 85, and a VL comprising the amino acid sequence of SEQ ID NO: 86. In some embodiments, the second binding moiety and/or the third binding moiety is an anti-WT1 scFv. In some embodiments, the anti-WT1 scFv comprises the amino acid sequence of SEQ ID NO: 87 or 88. In some embodiments, the second binding moiety and the third binding moiety are identical. In some embodiments, the second binding moiety and the third binding moiety are different.

Hence in some embodiments, there is provided an anti-CD3 construct (or MSAP, e.g., BSAP), comprising a) an anti-CD3 Fab fragment, wherein the anti-CD3 Fab fragment comprises a first polypeptide comprising a VH and a CH1, and a second polypeptide comprising a VL and a CL, and b) an anti-WT1 scFv (e.g., any of the anti-WT1 scFvs described herein); wherein the anti-WT1 scFv is fused to the N-terminus of the VH or VL of the anti-CD3 Fab fragment via an optional linker. In some embodiments, there is provided an anti-CD3 construct (or MSAP, e.g., BSAP), comprising a) an anti-CD3 Fab fragment, wherein the anti-CD3 Fab fragment comprises a first polypeptide comprising a VH and a CH1, and a second polypeptide comprising a VL and a CL, and b) an anti-WT1 scFv; wherein the anti-WT1 scFv is fused to the N-terminus of the VH of the anti-CD3 Fab fragment via a linker to form a fusion polypeptide; and wherein the fusion polypeptide comprises the amino acid sequence of SEQ ID NO: 95, and the second polypeptide comprises the amino acid sequence of SEQ ID NO: 90.

In some embodiments, there is provided an anti-CD3 construct (or MSAP, e.g., BSAP) comprising: a) an anti-CD3 antibody or antigen binding fragment thereof (e.g., any of the anti-CD3 antibodies or antigen binding fragments thereof described herein, such as scFv or Fab), and b) a Fab fragment that specifically binds to WT1 (e.g., any of the anti-WT1 Fabs described herein); wherein the anti-CD3 antibody or antigen binding fragment thereof is fused to the N-terminus of the VH or VL of the anti-WT1 Fab fragment via an optional linker. In some embodiments, there is provided an anti-CD3 construct (or MSAP, e.g., BSAP or TSAP), comprising a) an anti-CD3 antibody or antigen binding fragment thereof (e.g., any of the anti-CD3 antibodies or antigen binding fragments thereof described herein, such as scFv or Fab), b) a Fab fragment that specifically binds to a first target antigen that is WT1, and c) a third binding moiety (e.g., scFv or Fab) that specifically binds to a second target antigen (e.g., immune cell surface molecule, such as CD3, CD4, or CD8); wherein: (1) the anti-CD3 antibody or antigen binding fragment thereof is fused to the N-terminus of the VH of the anti-WT1 Fab fragment via an optional first linker, and the third binding moiety is fused to the N-terminus of the VL of the anti-WT1 Fab fragment via an optional second linker; or (2) the anti-CD3 antibody or antigen binding fragment thereof is fused to the N-terminus of the VL of the anti-WT1 Fab fragment via an optional first linker, and the third binding moiety is fused to the N-terminus of the VH of the anti-WT1 Fab fragment via an optional second linker. In some embodiments, the anti-CD3 antibody or antigen binding fragment thereof comprises: (i) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; (ii) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or (iii) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41. In some embodiments, the anti-WT1 Fab fragment comprises an HVR-H1 comprising the amino acid sequence of SEQ ID NO: 79, an HVR-H2 comprising the amino acid sequence of SEQ ID NO: 80, an HVR-H3 comprising the amino acid sequence of SEQ ID NO: 81, an HVR-L1 comprising the amino acid sequence of SEQ ID NO: 82, an HVR-L2 comprising the amino acid sequence of SEQ ID NO: 83, and an HVR-L3 comprising the amino acid sequence of SEQ ID NO: 84. In some embodiments, the anti-CD3 antigen binding fragment thereof and/or the third binding moiety is an anti-CD3 scFv (e.g., any of the anti-CD3 scFvs described herein). In some embodiments, the anti-CD3 scFv comprises the amino acid sequence of any of SEQ ID NOs: 50, 56, 163, 164, 183, and 184. In some embodiments, the anti-CD3 antigen binding fragment thereof and the third binding moiety are identical. In some embodiments, the anti-CD3 antibody or antigen binding fragment thereof, and the third binding moiety are different. In some embodiments, the third binding moiety is an anti-CD8 scFv (e.g., any of the anti-CD8 scFvs described herein). In some embodiments, the anti-WT1 Fab fragment comprises a VH comprising the amino acid sequence of SEQ ID NO: 85, and a VL comprising the amino acid sequence of SEQ ID NO: 86. In some embodiments, the anti-WT1 Fab fragment comprises a first polypeptide comprising the amino acid sequence of SEQ ID NO: 145, and a second polypeptide comprising the amino acid sequence of SEQ ID NO: 146. In some embodiments, the linker, the first linker, and/or the second linker comprises an amino acid sequence independently selected from the group consisting of GG and SEQ ID NOs: 108-110.

Also provided are isolated nucleic acid(s) encoding any of the anti-CD3 antibodies or antigen-binding fragments thereof (e.g., full-length antibody, scFv, or Fab) described herein, or any of the anti-CD3 constructs (e.g., anti-CD3 MSAPs, such as BSAP or TSAP) described herein; vector(s) (e.g., viral vector, such as lentiviral vector or AAV) comprising such isolated nucleic acid(s); and host cells (prokaryotic or eukaryotic) comprising such isolated nucleic acid(s) or vector(s), or expressing any of the anti-CD3 antibodies or antigen-binding fragments thereof described herein, or any of the anti-CD3 constructs (e.g., anti-CD3 MSAPs) described herein.

The invention further provides fusion proteins comprising any of the anti-CD3 antibodies or antigen-binding fragments thereof (e.g., full-length antibody, scFv, or Fab) described herein or anti-CD3 constructs described herein, and conjugates (e.g., small molecule drug conjugates, or detection tag conjugates) of any of the anti-CD3 antibodies or antigen-binding fragments thereof or any of the anti-CD3 constructs (e.g., MSAPs) described herein.

### V. Methods of Preparation

Also provided are methods of making any of the MSAPs (e.g., BSAPs, TSAPs) described herein, any of the anti-CD8 antibodies or antigen-binding fragments thereof described herein, polypeptide portion of any of the anti-CD8 constructs (e.g., MSAP, ADC) described herein, any of the anti-CD3 antibodies or antigen-binding fragments thereof described herein, and polypeptide portion of any of the anti-CD3 constructs (e.g., MSAP, ADC) described herein. See Example 1 for exemplary methods.

The MSAPs, anti-CD8 or anti-CD3 antibodies or antigen-binding fragments thereof, polypeptide portion of anti-CD8 or anti-CD3 constructs described herein may be prepared by any of the known protein expression and purification methods in the art. For example, see Example 1. DNA sequence encoding the MSAPs, polypeptide portion of anti-CD8 or anti-CD3 constructs, anti-CD8 or anti-CD3 antibodies or antigen-binding fragments thereof can be fully synthesized. After obtaining such sequence, it is cloned into a suitable expression vector, then transfected into a suitable host cell. The transfected host cells are cultured, and the supernatant is harvested and purified to obtain the MSAPs, anti-CD8 or anti-CD3 antibodies or antigen-binding fragments thereof, or anti-CD8 or anti-CD3 constructs described herein.

In some embodiments, the present application provides isolated nucleic acids encoding one or more of the polypeptides of any one of the MSAPs (such as TSAPs or BSAPs), anti-CD8 or anti-CD3 antibodies or antigen-binding fragments thereof, or anti-CD8 or anti-CD3 constructs (e.g., MSAP) described herein. In some embodiments, the isolated nucleic acid further comprises at its 3′ end a sequence encoding a hinge or portion thereof, such as nucleic acid sequence of SEQ ID NO: 144. In some embodiments, the isolated nucleic acid at its 5′ end further comprises a nucleic acid encoding a signal peptide sequence, such as nucleic acid sequence of SEQ ID NO: 142. The isolated nucleic acids may be DNA or RNA.

In some embodiments, the isolated nucleic acid is inserted into a vector, such as an expression vector, a viral vector, or a cloning vector. Hence also provided are vectors comprising any of the isolated nucleic acids described herein. For expression of the nucleic acids, the vector may be introduced into a host cell to allow expression of the nucleic acids within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art. For example, the promoter sequences may be selected to promote the transcription of the polynucleotide in the vector. Suitable promoter sequences include, without limitation, T7 promoter, T3 promoter, SP6 promoter, beta-actin promoter. EF1a promoter, CMV promoter, and SV40 promoter. Enhancer sequences may be selected to enhance the transcription of the nucleic acids. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell. In some embodiments, the signal peptide comprises the amino acid sequence of SEQ ID NO: 141. In some embodiments, the nucleic acid sequence encoding the signal peptide comprises the nucleic acid sequence of SEQ ID NO: 142.

Suitable vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the transcription or expression of coding nucleotide sequences to which they are operatively linked. Such vectors are referred to herein as “expression vectors”.

In some embodiments, the expression vector is selected from the group consisting of a viral vector, a bacterial vector, and a mammalian cell vector.

Viral vector technology is well known in the art and is described, for example, in Sambrook et al. (2012), and in Ausubel et al. (1999), and in other virology and molecular biology manuals. Viruses, which are useful as vectors include, but are not limited to, retroviruses, adenoviruses, adeno-associated viruses (AAV), herpes viruses, and lentiviruses. In some embodiments, a murine stem cell virus (MSCV) vector is used to express a desired nucleic acid. MSCV vectors have been demonstrated to efficiently express desired nucleic acids in cells. Other examples of viral vectors are those based upon Moloney Murine Leukemia Virus (MoMuLV) and human immunodeficiency virus (HIV). In some embodiments, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193). In some embodiments, the vector is a recombinant adeno-associated virus (rAAV) vector. In some embodiments, the rAAV vector is a vector derived from an AAV serotype, including without limitation, AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV capsid serotype or the like. In some embodiments, the vector is encapsidated in a rAAV particle. In some embodiments, the vector is a lentiviral vector.

The nucleic acids described herein or the vectors (e.g., viral vectors) can be delivered directly into a host cell or an individual (e.g., human) for the purpose of gene therapy. Methods of non-viral delivery of nucleic acids include lipofection, nucleofection, microinjection, biolistics, virosomes, liposomes, immunoliposomes, polycation or lipid: nucleic acid conjugates, electroporation, nanoparticles, exosomes, microvesicles, or gene-gun, naked DNA and artificial virions. The use of RNA or DNA viral based systems for the delivery of nucleic acids has high efficiency in targeting a virus to specific cells and trafficking the viral payload to the cellular nuclei.

In some embodiments, there is provided an isolated host cell comprising any of the isolated nucleic acids or vectors encoding any of the MSAPs, anti-CD8 or anti-CD3 antibodies or antigen-binding fragments thereof, or polypeptide portion of anti-CD8 or anti-CD3 constructs (e.g., MSAP) described herein. In some embodiments, there is provided an isolated host cell expressing any of the MSAPs, anti-CD8 or anti-CD3 antibodies or antigen-binding fragments thereof, or polypeptide portion of anti-CD8 or anti-CD3 constructs (e.g., MSAP) described herein. In some embodiments, the two or more polypeptides of any of the MSAPs, anti-CD8 or anti-CD3 antibodies or antigen-binding fragments thereof, or anti-CD8 or anti-CD3 constructs (e.g., MSAP) described herein are encoded by a single vector. In some embodiments, the two or more polypeptides of any of the MSAPs, anti-CD8 or anti-CD3 antibodies or antigen-binding fragments thereof, or anti-CD8 or anti-CD3 constructs (e.g., MSAP) described herein are encoded by two or more vectors. The host cells containing the vector may be useful in expression or cloning of the isolated nucleic acids. Suitable host cells can include, without limitation, prokaryotic cells, fungal cells, yeast cells, or higher eukaryotic cells such as mammalian cells. The expression of antibodies and antigen-binding fragments in prokaryotic cells such as E. coli is well established in the art. For a review, see for example Pluckthun, A. BioTechnology 9:545-551 (1991). Expression in eukaryotic cells in culture is also available to those skilled in the art as an option for production of antibodies or antigen-binding fragments thereof, see recent reviews, for example Ref, M. E. (1993) Curr. Opinion Biotech. 4:573-576; Trill J. J. et al. (1995) Curr. Opinion Biotech 6:553-560. Higher eukaryotic cells, in particular, those derived from multicellular organisms can be used for expression of glycosylated polypeptides. Suitable higher eukaryotic cells include, without limitation, invertebrate cells and insect cells, and vertebrate cells. In some embodiments, the host cell is E. coli. In some embodiments, the host cell is a Chinese hamster ovary (CHO) cell or an HEK293 cell.

The vector can be introduced to the host cell using any suitable methods known in the art, including, but not limited to, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art. In some embodiments, the host cells comprise two or more vectors each encoding a polypeptide of any of the MSAPs described herein, anti-CD8 or anti-CD3 antibodies or antigen-binding fragments thereof described herein, or anti-CD8 or anti-CD3 constructs (e.g., MSAP) described herein. In some embodiments, the host cells comprise a single vector comprising isolated nucleic acids encoding two or more polypeptides of any of the MSAPs described herein, anti-CD8 or anti-CD3 antibodies or antigen-binding fragments thereof described herein, or anti-CD8 or anti-CD3 constructs (e.g., MSAP) described herein. In some embodiments, the two or more polypeptides of any of the MSAPs described herein, anti-CD8 or anti-CD3 antibodies or antigen-binding fragments thereof described herein, or anti-CD8 or anti-CD3 constructs (e.g., MSAP) described herein are expressed by different host cells.

In some embodiments, the present application provides methods of making any of the MSAPs (such as TSAPs), anti-CD8 antibodies or antigen-binding fragments thereof, polypeptide portion of anti-CD8 constructs (e.g., MSAP), anti-CD3 antibodies or antigen-binding fragments thereof, or polypeptide portion of anti-CD3 constructs (e.g., MSAP) described herein, comprising i) culturing an isolated host cell comprising any of the isolated nucleic acids described herein or any of the vectors described herein, or any of the isolated host cells described herein, under a condition suitable for the expression of any of the MSAPs described herein, anti-CD8 antibodies or antigen-binding fragments thereof described herein, polypeptide portion of anti-CD8 constructs (e.g., MSAP), anti-CD3 antibodies or antigen-binding fragments thereof described herein, or polypeptide portion of anti-CD3 constructs (e.g., MSAP) described herein, and ii) obtaining the expressed MSAPs, anti-CD8 antibodies or antigen-binding fragments thereof, polypeptide portion of anti-CD8 constructs (e.g., MSAP), anti-CD3 antibodies or antigen-binding fragments thereof, or polypeptide portion of anti-CD3 constructs (e.g., MSAP) from said host cell (e.g., from the cell culture). The isolated host cells are cultured under conditions that allow expression of the isolated nucleic acids inserted in the vectors. Suitable conditions for expression of polynucleotides may include, without limitation, suitable medium, suitable density of host cells in the culture medium, presence of necessary nutrients, presence of supplemental factors, suitable temperatures and humidity, and absence of microorganism contaminants. A person with ordinary skill in the art can select the suitable conditions as appropriate for the purpose of the expression. In some embodiments, the method of making further comprises purifying any of the obtained MSAPs, anti-CD8 antibodies or antigen-binding fragments thereof, polypeptide portion of anti-CD8 constructs (e.g., MSAP), anti-CD3 antibodies or antigen-binding fragments thereof, or polypeptide portion of anti-CD3 constructs (e.g., MSAP).

In some embodiments, the polypeptides expressed by the host cell can assemble together (e.g., form a polypeptide complex such as a dimer) and produce any of the MSAPs, anti-CD8 antibodies or antigen-binding fragments thereof, polypeptide portion of anti-CD8 constructs (e.g., MSAP), anti-CD3 antibodies or antigen-binding fragments thereof, or polypeptide portion of anti-CD3 constructs (e.g., MSAP) described herein. In some embodiments, the polypeptide complex may be formed inside the host cell. For example, the polypeptide complex may be formed inside the host cell with the aid of relevant enzymes and/or cofactors. In some embodiments, the polypeptide complex may be secreted out of the cell. In some embodiments, individual polypeptides may be secreted out of the host cell then form a polypeptide complex (such as any of the MSAPs, anti-CD8 antibodies or antigen-binding fragments thereof, polypeptide portion of anti-CD8 constructs (e.g., MSAP), anti-CD3 antibodies or antigen-binding fragments thereof, or polypeptide portion of anti-CD3 constructs (e.g., MSAP) described herein) outside of the host cell.

In some embodiments, the two or more polypeptides of any of the MSAPs, anti-CD8 antibodies or antigen-binding fragments thereof, polypeptide portion of anti-CD8 constructs (e.g., MSAP), anti-CD3 antibodies or antigen-binding fragments thereof, or polypeptide portion of anti-CD3 constructs (e.g., MSAP) described herein may be separately expressed and allowed to form (e.g., dimerize) the MSAPs, anti-CD8 antibodies or antigen-binding fragments thereof, polypeptide portion of anti-CD8 constructs (e.g., MSAP), anti-CD3 antibodies or antigen-binding fragments thereof, or polypeptide portion of anti-CD3 constructs (e.g., MSAP) under suitable conditions. For example, the first polypeptide and the second polypeptide may be combined in a suitable buffer and allow the first protein monomer and the second protein monomer to dimerize through appropriate interactions such as hydrophobic interactions. In some embodiments, the first polypeptide and the second polypeptide may be combined in a suitable buffer containing an enzyme and/or a cofactor which can promote the dimerization of the first polypeptide and the second polypeptide. In some embodiments, the first polypeptide and the second polypeptide may be combined in a suitable vehicle and allow them to react with each other in the presence of a suitable reagent and/or catalyst.

The expressed polypeptide(s) and/or the polypeptide complex can be collected using any suitable methods. The polypeptide(s) and/or the polypeptide complex can be expressed intracellularly, in the periplasmic space or be secreted outside of the cell into the medium. If the polypeptide and/or the polypeptide complex are expressed intracellularly, the host cells containing the polypeptide and/or the polypeptide complex may be lysed and polypeptide and/or the polypeptide complex may be isolated from the lysate by removing the unwanted debris by centrifugation or ultrafiltration. If the polypeptide and/or the polypeptide complex is secreted into periplasmic space of E. coli, the cell paste may be thawed in the presence of agents such as sodium acetate (pH 3.5), EDTA, and phenylmethylsulfonylfluoride (PMSF) for about 30 min, and cell debris can be removed by centrifugation (Carter et al., BioTechnology 10:163-167 (1992)). If the polypeptide and/or the polypeptide complex is secreted into the medium, the supernatant of the cell culture may be collected and concentrated using a commercially available protein concentration filter, for example, an Amincon or Millipore Pellicon ultrafiltration unit. A protease inhibitor and/or an antibiotic may be included in the collection and concentration steps to inhibit protein degradation and/or growth of contaminated microorganisms.

The expressed polypeptide(s) and/or the polypeptide complex can be further purified by a suitable method, such as without limitation, affinity chromatography, hydroxylapatite chromatography, size exclusion chromatography, gel electrophoresis, dialysis, ion exchange fractionation on an ion-exchange column, ethanol precipitation, reverse phase HPLC, chromatography on silica, chromatography on heparin sepharose, chromatography on an anion or cation exchange resin (such as a polyaspartic acid column), chromatofocusing, SDS-PAGE, and ammonium sulfate precipitation (see, for review, Bonner, P. L., Protein purification, published by Taylor & Francis. 2007; Janson, J. C., et al., Protein purification: principles, high resolution methods and applications, published by Wiley-VCH, 1998).

In some embodiments, the polypeptides and/or polypeptide complexes can be purified by affinity chromatography. In some embodiments, protein A chromatography or protein A/G (fusion protein of protein A and protein G) chromatography can be useful for purification of polypeptides and/or polypeptide complexes comprising a component derived from antibody CH2 domain and/or CH3 domain (Lindmark et al., J. Immunol. Meth. 62:1-13 (1983)); Zettlit, K. A., Antibody Engineering, Part V, 531-535, 2010). In some embodiments, protein G chromatography can be useful for purification of polypeptides and/or polypeptide complexes comprising IgG γ3 heavy chain (Guss et al., EMBO J. 5:1567 1575 (1986)). In some embodiments, protein L chromatography can be useful for purification of polypeptides and/or polypeptide complexes comprising k light chain (Sudhir, P., Antigen engineering protocols, Chapter 26, published by Humana Press, 1995; Nilson, B. H. K. et al., J. Biol. Chem., 267, 2234-2239 (1992)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where any of the MSAPs (such as TSAPs), anti-CD8 or anti-CD3 antibodies or fragments thereof, or anti-CD8 or anti-CD3 constructs comprises an additional CH3 domain, the Bakerbond ABX resin (J. T. Baker, Phillipsburg, N.J.) is useful for purification. In some embodiments, any of the MSAPs, anti-CD8 or anti-CD3 antibodies or antigen-binding fragments thereof, polypeptide portion of anti-CD8 or anti-CD3 constructs (e.g., MSAP) described herein is purified with IgG-CH1 Affinity Matrix, e.g., CaptureSelect™ IgG-CH1 Affinity Matrix (Thermo Scientific™).

### VI. Pharmaceutical Compositions, Unit Dosages, Articles of Manufacture, and Kits

Further provided by the present application are pharmaceutical compositions comprising i) any one of the MSAPs (such as TSAPs), anti-CD8 antibodies or antigen-binding fragments thereof, anti-CD8 constructs (e.g., MSAP, such as TSAP or BSAP), anti-CD3 antibodies or antigen-binding fragments thereof as described herein, or anti-CD3 constructs (e.g., MSAP, such as TSAP or BSAP), and ii) optionally a pharmaceutically acceptable carrier. Also provided are pharmaceutical compositions comprising i) isolated nucleic acids or vectors (e.g., viral vector, such as lentiviral vector or AAV) encoding any one of the MSAPs (such as TSAPs), anti-CD8 antibodies or antigen-binding fragments thereof, anti-CD8 constructs (e.g., MSAP, such as TSAP or BSAP), anti-CD3 antibodies or antigen-binding fragments thereof as described herein, or anti-CD3 constructs (e.g., MSAP, such as TSAP or BSAP), and ii) optionally a pharmaceutically acceptable carrier.

The MSAPs (such as TSAPs or BSAPs), anti-CD8 or anti-CD3 antibodies or antigen-binding fragments thereof, anti-CD8 or anti-CD3 constructs (e.g., MSAP), isolated nucleic acids or vectors (e.g., viral vector, such as lentiviral vector or AAV) encoding thereof, or pharmaceutical compositions thereof may be suitable for a variety of modes of administration described herein, including for example systemic or localized administration. In some embodiments, the pharmaceutical composition is formulated for intravenous administration. In some embodiments, the pharmaceutical composition is formulated for subcutaneous administration. In some embodiments, the pharmaceutical composition is formulated for local administration to a tumor site. In some embodiments, the pharmaceutical composition is formulated for intratumoral injection.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical composition is formulated to have a pH in the range of about 4.5 to about 9.0, including for example pH ranges of about any one of 5.0 to about 8.0, about 6.5 to about 7.5, or about 6.5 to about 7.0. In some embodiments, the pharmaceutical composition can also be made to be isotonic with blood by the addition of a suitable tonicity modifier, such as glycerol.

The pharmaceutical compositions to be used for in vivo administration are generally formulated as sterile, substantially isotonic, and in full compliance with all Good Manufacturing Practice (GMP) regulations of the U.S. Food and Drug Administration. Sterility is readily accomplished by filtration through sterile filtration membranes. In some embodiments, the composition is free of pathogen. For injection, the pharmaceutical composition can be in the form of liquid solutions, for example in physiologically compatible buffers such as Hank's solution or Ringer's solution. In addition, the pharmaceutical composition can be in a solid form and re-dissolved or suspended immediately prior to use. Lyophilized compositions are also included.

In some embodiment, the pharmaceutical composition is formulated in accordance with routine procedures as a pharmaceutical composition adapted for injection subcutaneously, intravenously, intraperitoneally, or intravitreally. Typically, compositions for injection are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In some embodiments, the pharmaceutical composition is suitable for administration to a mammal, such as a human. In some embodiments, the pharmaceutical composition is suitable for administration to a rodent (e.g., mice, rats) or non-human primates (e.g., Cynomolgus monkey). In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

Also provided are unit dosage forms of any of the MSAPs (such as TSAPs), anti-CD8 or anti-CD3 antibodies or antigen-binding fragments thereof, or anti-CD8 or anti-CD3 constructs (e.g., MSAP) described herein, isolated nucleic acids or vectors (e.g., viral vector, such as lentiviral vector or AAV) encoding thereof, or compositions (such as pharmaceutical compositions) comprising thereof. The term “unit dosage form” refers to a physically discrete unit suitable as unitary dosages for an individual (e.g., human), each unit containing a predetermined quantity of active material calculated to produce the desired therapeutic effect, in association with a suitable pharmaceutical carrier, diluent, or excipient. These unit dosage forms can be stored in a suitable packaging in single or multiple unit dosages and may also be further sterilized and sealed.

The present application further provides articles of manufacture comprising the compositions (such as pharmaceutical compositions) described herein in suitable packaging. Suitable packaging for compositions (such as pharmaceutical compositions) described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The present application also provides kits comprising compositions (such as pharmaceutical compositions) described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein.

### VII. Methods of Treating a Disease Associated with a Target Epitope

Also provided are methods of treating a disease associated with a target epitope or target antigen (e.g., TAA or TSA, such as CD19, WT1, or EpCAM) in an individual (e.g., human), comprising administering to the individual an effective amount of any of the MSAPs (e.g., TSAP or BSAP) described herein, anti-CD8 constructs (e.g., TAA (TSA)×CD8 MSAPs) described herein, anti-CD3 constructs (e.g., TAA (TSA)×CD3 MSAPs) described herein, isolated nucleic acids or vectors (e.g., viral vector, such as lentiviral vector or AAV) encoding thereof, or pharmaceutical compositions comprising thereof. In some embodiments, the MSAP (e.g., TSAP or BSAP), anti-CD8 construct, or anti-CD3 construct specifically binds to the target epitope or target antigen. In some embodiments, the method preferentially activates CD8+ T cells over CD4+ T cells. In some embodiments, the method does not induce high systemic levels of pro-inflammatory cytokines (e.g., IL-2) produced by CD4+ T cells, as measured by the cytokine levels circulating in peripheral blood. In some embodiments, the method does not induce high systemic levels of the pro-inflammatory TNFα or IL-6 cytokines, as measured by the cytokine levels circulating in peripheral blood. In some embodiments, the method does not induce cytokine release syndrome or cytokine storm. In some embodiments, the method comprising administering an MSAP comprising an anti-CD8 moiety (e.g., TAA (TSA)×CD8×CD3 TSAP) induces less cytokine release syndrome or cytokine storm (e.g., at least about any of 10%, 20%, 30%, 40%, 50%, 60%, 70%, 80%, 90% or less cytokine release syndrome or cytokine storm) compared to an MSAP not comprising an anti-CD8 moiety (e.g., TAA (TSA)×CD3 BSAP or TSAP).

A disease associated with the target epitope can be a disease caused by, or associate with, the expression of the target epitope or target antigen, e.g., over-expression compared to a healthy state, or mis-expression at a location different from a healthy state (e.g., on a different cell or tissue).

In some embodiments, the disease associated with a target epitope or target antigen is an infection, such as a microbial infection, for example a bacterial, fungal, or viral infection. In some embodiments, the disease associated with a target epitope or target antigen is an autoimmune disease, such as lupus, Sjogren's syndrome, multiple sclerosis, psoriasis, psoriatic arthritis, rheumatoid arthritis, sarcoidosis, ulcerative colitis, etc. In some embodiments, the disease associated with a target epitope or target antigen is another disease wherein immune function is implicated (e.g., endometriosis). In some embodiments, the target epitope or target antigen is an allergen such that the presence of the target epitope or target antigen is capable of inducing or otherwise causing an allergic response.

In some embodiments, the disease associated with a target epitope or target antigen is a cancer expressing the target epitope. In some embodiments, the target epitope is a target epitope of a TSA or TAA, e.g., CD19+, WT1+, or EpCAM+ cancer. In some embodiments, the cancer is a solid cancer, such as colorectal cancer or Wilms' tumor. In some embodiments, the cancer is a liquid cancer, such as Burkitt lymphoma. In some embodiments, the cancer is a B cell cancer (e.g., B cell lymphoma).

Hence in some embodiments, there is provided a method of treating a cancer expressing a target epitope or target antigen (e.g., a specific TSA/TAA+ cancer, such as CD19+, WT1+, or EpCAM+ cancer) in an individual (such as a human), comprising administering to the individual an effective amount of any of the MSAPs (such as TSAPs, for example TAA (TSA)×CD8×CD3 TSAPs, TAA (TSA)×CD8 MSAPs, or TAA (TSA)×CD3 MSAPs) described herein, isolated nucleic acids or vectors (e.g., viral vector, such as lentiviral vector or AAV) encoding thereof, or a composition (such as pharmaceutical composition) comprising thereof. In some embodiments, the MSAPs (such as TSAPs or BSAPs), the isolated nucleic acids or vectors (e.g., viral vector, such as lentiviral vector or AAV) encoding thereof, or the composition (such as pharmaceutical composition) comprising thereof, is administered intravenously, subcutaneously, or intratumorally. In some embodiments, the method preferentially activates CD8+ T cells over CD4+ T cells. In some embodiments, the method does not induce high systemic levels of pro-inflammatory cytokines (e.g., IL-2) produced by CD4+ T cells, as measured by the cytokine levels circulating in peripheral blood. In some embodiments, the method does not induce high systemic levels of the pro-inflammatory TNFα or IL-6 cytokines, as measured by the cytokine levels circulating in peripheral blood. In some embodiments, the method does not induce cytokine release syndrome or cytokine storm. In some embodiments, the method comprising administering an MSAP comprising an anti-CD8 moiety (e.g., TAA (TSA)×CD8×CD3 TSAP) induces less cytokine release syndrome or cytokine storm (e.g., at least about any of 10%, 20%, 30%, 40%, 50%, 60%, 70%, 80%, 90% or less cytokine release syndrome or cytokine storm) compared to an MSAP not comprising an anti-CD8 moiety (e.g., TAA (TSA)×CD3 BSAP or TSAP). In some embodiments, the cancer is a solid cancer, such as colorectal cancer or Wilms' tumor. In some embodiments, the cancer is a liquid cancer, such as Burkitt lymphoma.

In some embodiments, there is provided a method of treating a disease associated with a target epitope or target antigen (e.g., a specific TSA/TAA+ cancer, such as CD19+, WT1+, or EpCAM+ cancer) in an individual (such as a human), comprising administering to the individual an effective amount of an MSAP (e.g., any of the MSAPs described herein, such as TSAP; or an isolated nucleic acid or vector (e.g., viral vector, such as lentiviral vector or AAV) encoding thereof, or a pharmaceutical composition comprising thereof) comprising: i) an anti-CD3 moiety that specifically binds to CD3; ii) an anti-CD8 moiety that specifically binds to CD8; and iii) an anti-target epitope (e.g., TSA or TAA, such as CD19, WT1, or EpCAM) moiety that specifically binds to the target epitope or target antigen; wherein a first moiety of the three moieties is a Fab fragment, wherein the Fab fragment comprises a first polypeptide comprising a VH and a CH1, and a second polypeptide comprising a VL and a CL; wherein a second moiety of the three moieties is a first antigen binding fragment (e.g., scFv); wherein a third moiety of the three moieties is a second antigen binding fragment (e.g., scFv); and wherein (a) the first antigen binding fragment is fused to the N-terminus of the VH of the Fab fragment via an optional first linker, and the second antigen binding fragment is fused to the N-terminus of the VL of the Fab fragment via an optional second linker; (b) the first antigen binding fragment is fused to the C-terminus of the CH1 of the Fab fragment via an optional first linker, and the second antigen binding fragment is fused to the N-terminus of the VL of the Fab fragment via an optional second linker; (c) the first antigen binding fragment is fused to the N-terminus of the VH of the Fab fragment via an optional first linker, and the second antigen binding fragment is fused to the C-terminus of the CL of the Fab fragment via an optional second linker; (d) the first antigen binding fragment is fused to the N-terminus of the VH of the Fab fragment via an optional first linker, and the second antigen binding fragment is fused to the C-terminus of the CH1 of the Fab fragment via an optional second linker; or (e) the first antigen binding fragment is fused to the N-terminus of the VL of the Fab fragment via an optional first linker, and the second antigen binding fragment is fused to the C-terminus of the CL of the Fab fragment via an optional second linker. In some embodiments, the anti-CD3 moiety is the Fab fragment, and (i) the anti-CD8 moiety is the first antigen binding fragment, and the anti-target epitope moiety is the second antigen binding fragment; or (ii) the anti-target epitope moiety is the first antigen binding fragment, and the anti-CD8 moiety is the second antigen binding fragment. In some embodiments, the anti-CD8 moiety is the Fab fragment, and (i) the anti-CD3 moiety is the first antigen binding fragment, and the anti-target epitope moiety is the second antigen binding fragment; or (ii) the anti-target epitope moiety is the first antigen binding fragment, and the anti-CD3 moiety is the second antigen binding fragment. In some embodiments, the anti-target epitope moiety is the Fab fragment, and (i) the anti-CD3 moiety is the first antigen binding fragment, and the anti-CD8 moiety is the second antigen binding fragment; or (ii) the anti-target epitope moiety is the Fab fragment, the anti-CD8 moiety is the first antigen binding fragment, and the anti-CD3 moiety is the second antigen binding fragment. In some embodiments, the first antigen binding fragment and the second antigen binding fragment are both scFv. In some embodiments, the first linker and/or the second linker comprises an amino acid sequence independently selected from the group consisting of GG and SEQ ID NOs: 108-110. In some embodiments, the MSAPs (such as TSAPs or BSAPs) or the composition (such as pharmaceutical composition) thereof is administered intravenously, subcutaneously, or intratumorally. In some embodiments, the method preferentially activates CD8+ T cells over CD4+ T cells. In some embodiments, the method does not induce high systemic levels of pro-inflammatory cytokines (e.g., IL-2) produced by CD4+ T cells, as measured by the cytokine levels circulating in peripheral blood. In some embodiments, the method does not induce high systemic levels of the pro-inflammatory TNFα or IL-6 cytokines, as measured by the cytokine levels circulating in peripheral blood. In some embodiments, the method does not induce cytokine release syndrome or cytokine storm. In some embodiments, the method comprising administering an MSAP comprising an anti-CD8 moiety (e.g., TAA (TSA)×CD8×CD3 TSAP) does not induce cytotoxicity (or induces at most about 20% cytotoxicity) against CD8+ T cells compared to an MSAP not comprising an anti-CD8 moiety (e.g., TAA (TSA)×CD3 BSAP or TSAP). In some embodiments, the method comprising administering an MSAP comprising an anti-CD8 moiety (e.g., TAA (TSA)×CD8×CD3 TSAP) induces less cytokine release syndrome or cytokine storm (e.g., at least about any of 10%, 20%, 30%, 40%, 50%, 60%, 70%, 80%, 90% or less cytokine release syndrome or cytokine storm) compared to an MSAP not comprising an anti-CD8 moiety (e.g., TAA (TSA)×CD3 BSAP or TSAP).

In some embodiments, there is provided a method of treating a disease associated with a target epitope (or target antigen; e.g., a specific TSA/TAA+ cancer, such as CD19+, WT1+, or EpCAM+ cancer) in an individual (such as a human), comprising administering to the individual an effective amount of an MSAP (e.g., any of the anti-CD3 moiety containing MSAPs described herein, such as BSAP; or an isolated nucleic acid or vector (e.g., viral vector, such as lentiviral vector or AAV) encoding thereof, or a pharmaceutical composition comprising thereof) comprising: i) an anti-CD3 moiety that specifically binds to CD3 (e.g., any of the anti-CD3 antibodies or antigen binding fragments thereof described herein, such as Fab or scFv); and ii) an anti-target epitope moiety (e.g., scFv or Fab) that specifically binds to the target epitope (e.g., TSA or TAA, such as CD19, WT1, EpCAM). In some embodiments, the MSAPs (such as TSAPs or BSAPs), or the isolated nucleic acid or vector (e.g., viral vector, such as lentiviral vector or AAV) encoding thereof, or the composition (such as pharmaceutical composition) comprising thereof is administered intravenously, subcutaneously, or intratumorally. In some embodiments, the method does not induce cytokine release syndrome or cytokine storm.

In some embodiments, there is provided a method of treating a disease associated with a target epitope (or target antigen; e.g., a specific TSA/TAA+ cancer, such as CD19+, WT1+, or EpCAM+ cancer) in an individual (such as a human), comprising administering to the individual an effective amount of an MSAP (e.g., any of the anti-CD8 moiety containing MSAPs described herein, such as BSAP; or an isolated nucleic acid or vector (e.g., viral vector, such as lentiviral vector or AAV) encoding thereof, or a pharmaceutical composition comprising thereof) comprising: i) an anti-CD8 moiety that specifically binds to CD8 (e.g., any of the anti-CD8 antibodies or antigen binding fragments thereof described herein, such as Fab or scFv); and ii) an anti-target epitope moiety (e.g., scFv or Fab) that specifically binds to a target epitope (e.g., TSA or TAA, such as CD19, WT1, EpCAM). In some embodiments, the MSAP, or the isolated nucleic acid or vector (e.g., viral vector, such as lentiviral vector or AAV) encoding thereof, or the composition (such as pharmaceutical composition) comprising thereof, is administered intravenously, subcutaneously, or intratumorally. In some embodiments, the method does not induce cytokine release syndrome or cytokine storm.

In some embodiments, there is provided a method of: (1) depleting a target cell (e.g., B cells, such as CD19+ B cells), (2) reducing CD4+ T cell induced cytokine release, and/or (3) selectively activating CD8+ T cells in an individual (such as a human), comprising administering to the individual an effective amount of an MSAP (e.g., any of the MSAPs described herein, such as TSAP; or a pharmaceutical composition thereof) comprising: i) an anti-CD3 moiety that specifically binds to CD3; ii) an anti-CD8 moiety that specifically binds to CD8; and iii) an anti-target epitope moiety that specifically binds to the target epitope or target antigen (e.g., TSA or TAA, such as CD19, EpCAM, or WT1); wherein a first moiety of the three moieties is a Fab fragment, wherein the Fab fragment comprises a first polypeptide comprising a VH and a CH1, and a second polypeptide comprising a VL and a CL; wherein a second moiety of the three moieties is a first antigen binding fragment (e.g., scFv); wherein a third moiety of the three moieties is a second antigen binding fragment (e.g., scFv); and wherein (a) the first antigen binding fragment is fused to the N-terminus of the VH of the Fab fragment via an optional first linker, and the second antigen binding fragment is fused to the N-terminus of the VL of the Fab fragment via an optional second linker; (b) the first antigen binding fragment is fused to the C-terminus of the CH1 of the Fab fragment via an optional first linker, and the second antigen binding fragment is fused to the N-terminus of the VL of the Fab fragment via an optional second linker; (c) the first antigen binding fragment is fused to the N-terminus of the VH of the Fab fragment via an optional first linker, and the second antigen binding fragment is fused to the C-terminus of the CL of the Fab fragment via an optional second linker; (d) the first antigen binding fragment is fused to the N-terminus of the VH of the Fab fragment via an optional first linker, and the second antigen binding fragment is fused to the C-terminus of the CH1 of the Fab fragment via an optional second linker; or (e) the first antigen binding fragment is fused to the N-terminus of the VL of the Fab fragment via an optional first linker, and the second antigen binding fragment is fused to the C-terminus of the CL of the Fab fragment via an optional second linker. In some embodiments, the MSAP, or the isolated nucleic acid or vector (e.g., viral vector, such as lentiviral vector or AAV) encoding thereof, or the composition (such as pharmaceutical composition) comprising thereof is administered intravenously, subcutaneously, or intratumorally. In some embodiments, the method preferentially activates CD8+ T cells over CD4+ T cells. In some embodiments, the method preferentially activates CD8+ T cells over CD4+ T cells by at least about 2-fold (e.g., at least about any of 3-fold, 4-fold, 5-fold, 6-fold, 7-fold, 8-fold, 9-fold 10-fold, or higher). In some embodiments, the method preferentially induces the local production (i.e., at the site of the target cell) of CD8+ T cell cytokines over CD4+ T cell cytokines by at least about 2-fold (e.g., at least about any of 3-fold, 4-fold, 5-fold, 6-fold, 7-fold, 8-fold, 9-fold 10-fold, or higher). In some embodiments, the method does not induce high systemic levels of pro-inflammatory cytokines (e.g., IL-2, TNF-α, IFN-γ) produced by CD4+ T cells, as measured by the cytokine levels circulating in peripheral blood. In some embodiments, the method does not induce high systemic levels of the pro-inflammatory TNFα or IL-6 cytokines, as measured by the cytokine levels circulating in peripheral blood. In some embodiments, the method does not induce cytokine release syndrome or cytokine storm.

In some embodiments, there is provided a method of treating a cancer expressing WT1 in an individual (e.g., human), comprising administering to the individual an effective amount of an MSAP (or an isolated nucleic acid or vector (e.g., viral vector, such as lentiviral vector or AAV) encoding thereof, or a pharmaceutical composition comprising thereof), wherein the MSAP comprises a first polypeptide comprising the amino acid sequence of SEQ ID NO: 95, and a second polypeptide comprising the amino acid sequence of SEQ ID NO: 96. In some embodiments, the MSAP, or the isolated nucleic acid or vector (e.g., viral vector) encoding thereof, or the composition (such as pharmaceutical composition) comprising thereof is administered intravenously, subcutaneously, or intratumorally. In some embodiments, the method does not induce cytokine release syndrome or cytokine storm. In some embodiments, the MSAP or composition thereof induces less cytokine release syndrome or cytokine storm (e.g., at least about any of 10%, 20%, 30%, 40%, 50%, 60%, 70%, 80%, 90% or less cytokine release syndrome or cytokine storm) compared to a reference MSAP not comprising an anti-CD8 moiety (e.g., TAA (TSA)×CD3 BSAP or TSAP). In some embodiments, the cancer expressing WT1 is Wilms' tumor.

In some embodiments, there is provided a method of treating a cancer expressing WT1 in an individual (e.g., human), comprising administering to the individual an effective amount of an BSAP (or an isolated nucleic acid or vector (e.g., viral vector, such as lentiviral vector or AAV) encoding thereof, or a pharmaceutical composition comprising thereof), wherein the BSAP comprises a first polypeptide comprising the amino acid sequence of SEQ ID NO: 95, and a second polypeptide comprising the amino acid sequence of SEQ ID NO: 90. In some embodiments, the BSAP, or the isolated nucleic acid or vector (e.g., viral vector) encoding thereof, or the composition (such as pharmaceutical composition) comprising thereof is administered intravenously, subcutaneously, or intratumorally. In some embodiments, the method does not induce cytokine release syndrome or cytokine storm. In some embodiments, the cancer expressing WT1 is Wilms' tumor.

In some embodiments, there is provided a method of treating a cancer expressing EpCAM in an individual (e.g., human), comprising administering to the individual an effective amount of an MSAP (or an isolated nucleic acid or vector (e.g., viral vector, such as lentiviral vector or AAV) encoding thereof, or a pharmaceutical composition comprising thereof), wherein the MSAP comprises i) a first polypeptide comprising the amino acid sequence of SEQ ID NO: 97, and a second polypeptide comprising the amino acid sequence of SEQ ID NO: 98; ii) a first polypeptide comprising the amino acid sequence of SEQ ID NO: 99, and a second polypeptide comprising the amino acid sequence of SEQ ID NO: 100; or iii) a first polypeptide comprising the amino acid sequence of SEQ ID NO: 101, and a second polypeptide comprising the amino acid sequence of SEQ ID NO: 102. In some embodiments, the MSAP, or the isolated nucleic acid or vector (e.g., viral vector) encoding thereof, or the composition (such as pharmaceutical composition) comprising thereof, is administered intravenously, subcutaneously, or intratumorally. In some embodiments, the method preferentially activates CD8+ T cells over CD4+ T cells. In some embodiments, the method does not induce high systemic levels of pro-inflammatory cytokines (e.g., IL-2) produced by CD4+ T cells, as measured by the cytokine levels circulating in peripheral blood. In some embodiments, the method does not induce high systemic levels of the pro-inflammatory TNFα or IL-6 cytokines, as measured by the cytokine levels circulating in peripheral blood. In some embodiments, the method does not induce cytokine release syndrome or cytokine storm. In some embodiments, the MSAP or composition thereof induces less cytokine release syndrome or cytokine storm (e.g., at least about any of 10%, 20%, 30%, 40%, 50%, 60%, 70%, 80%, 90% or less cytokine release syndrome or cytokine storm) compared to a reference MSAP not comprising an anti-CD8 moiety (e.g., TAA (TSA)×CD3 BSAP or TSAP). In some embodiments, the cancer expressing EpCAM is colorectal cancer, breast cancer, gastric cancer, prostate cancer, ovarian cancer, gallbladder cancer, pancreatic cancer, prostate adenocarcinoma, ampullary cancer, esophageal cancer, renal cancer, thyroid cancer, squamous cell carcinoma, or lung cancer.

In some embodiments, there is provided a method of treating a cancer expressing EpCAM in an individual (e.g., human), comprising administering to the individual an effective amount of an BSAP (or an isolated nucleic acid or vector (e.g., viral vector, such as lentiviral vector or AAV) encoding thereof, or a pharmaceutical composition comprising thereof), wherein the BSAP comprises i) a first polypeptide comprising the amino acid sequence of SEQ ID NO: 124, and a second polypeptide comprising the amino acid sequence of SEQ ID NO: 125; ii) a first polypeptide comprising the amino acid sequence of SEQ ID NO: 126, and a second polypeptide comprising the amino acid sequence of SEQ ID NO: 127; iii) a first polypeptide comprising the amino acid sequence of SEQ ID NO: 128, and a second polypeptide comprising the amino acid sequence of SEQ ID NO: 129; iv) a first polypeptide comprising the amino acid sequence of SEQ ID NO: 130, and a second polypeptide comprising the amino acid sequence of SEQ ID NO: 131; v) a first polypeptide comprising the amino acid sequence of SEQ ID NO: 132, and a second polypeptide comprising the amino acid sequence of SEQ ID NO: 133; or vi) a first polypeptide comprising the amino acid sequence of SEQ ID NO: 134, and a second polypeptide comprising the amino acid sequence of SEQ ID NO: 135. In some embodiments, the BSAP, or the isolated nucleic acid or vector (e.g., viral vector) encoding thereof, or the composition (such as pharmaceutical composition) comprising thereof is administered intravenously, subcutaneously, or intratumorally. In some embodiments, the method preferentially activates CD8+ T cells over CD4+ T cells. In some embodiments, the method does not induce high systemic levels of pro-inflammatory cytokines (e.g., IL-2) produced by CD4+ T cells, as measured by the cytokine levels circulating in peripheral blood. In some embodiments, the method does not induce high systemic levels of the pro-inflammatory TNFα or IL-6 cytokines, as measured by the cytokine levels circulating in peripheral blood. In some embodiments, the method does not induce cytokine release syndrome or cytokine storm. In some embodiments, the cancer expressing EpCAM is colorectal cancer, breast cancer, gastric cancer, prostate cancer, ovarian cancer, gallbladder cancer, pancreatic cancer, prostate adenocarcinoma, ampullary cancer, esophageal cancer, renal cancer, thyroid cancer, squamous cell carcinoma, or lung cancer.

In some embodiments, there is provided a method of treating a cancer expressing CD19 in an individual (e.g., human), comprising administering to the individual an effective amount of an MSAP (or an isolated nucleic acid or vector (e.g., viral vector, such as lentiviral vector or AAV) encoding thereof, or a pharmaceutical composition comprising thereof), wherein the MSAP comprises i) a first fusion polypeptide comprising the amino acid sequence of SEQ ID NO: 103, and a second fusion polypeptide comprising the amino acid sequence of SEQ ID NO: 104; ii) a first fusion polypeptide comprising the amino acid sequence of SEQ ID NO: 103, and a second fusion polypeptide comprising the amino acid sequence of SEQ ID NO: 106; iii) a first fusion polypeptide comprising the amino acid sequence of SEQ ID NO: 161, and a second fusion polypeptide comprising the amino acid sequence of SEQ ID NO: 106; (iv) a first fusion polypeptide comprising the amino acid sequence of SEQ ID NO: 170, and a second fusion polypeptide comprising the amino acid sequence of SEQ ID NO: 171; (v) a first fusion polypeptide comprising the amino acid sequence of SEQ ID NO: 172, and a second fusion polypeptide comprising the amino acid sequence of SEQ ID NO: 173; (vi) a first fusion polypeptide comprising the amino acid sequence of SEQ ID NO: 174, and a second fusion polypeptide comprising the amino acid sequence of SEQ ID NO: 175; (vii) a first fusion polypeptide comprising the amino acid sequence of SEQ ID NO: 172, and a second fusion polypeptide comprising the amino acid sequence of SEQ ID NO: 176; (viii) a first fusion polypeptide comprising the amino acid sequence of SEQ ID NO: 172, and a second fusion polypeptide comprising the amino acid sequence of SEQ ID NO: 182; (ix) a first fusion polypeptide comprising the amino acid sequence of SEQ ID NO: 177, and a second fusion polypeptide comprising the amino acid sequence of SEQ ID NO: 178; (x) a first fusion polypeptide comprising the amino acid sequence of SEQ ID NO: 180, and a second polypeptide comprising the amino acid sequence of SEQ ID NO: 169; or (xi) a first polypeptide comprising the amino acid sequence of SEQ ID NO:137, and a second fusion polypeptide comprising the amino acid sequence of SEQ ID NO: 181. In some embodiments, the MSAP, or the isolated nucleic acid or vector (e.g., viral vector) encoding thereof, or the composition (such as pharmaceutical composition) comprising thereof, is administered intravenously, subcutaneously, or intratumorally. In some embodiments, the method preferentially activates CD8+ T cells over CD4+ T cells. In some embodiments, the method does not induce high systemic levels of pro-inflammatory cytokines (e.g., IL-2) produced by CD4+ T cells, as measured by the cytokine levels circulating in peripheral blood. In some embodiments, the method does not induce high systemic levels of the pro-inflammatory TNFα or IL-6 cytokines, as measured by the cytokine levels circulating in peripheral blood. In some embodiments, the method does not induce cytokine release syndrome or cytokine storm. In some embodiments, the MSAP or composition thereof induces less cytokine release syndrome or cytokine storm (e.g., at least about any of 10%, 20%, 30%, 40%, 50%, 60%, 70%, 80%, 90% or less cytokine release syndrome or cytokine storm) compared to a reference MSAP not comprising an anti-CD8 moiety (e.g., TAA (TSA)×CD3 BSAP or TSAP). In some embodiments, the cancer expressing CD19 is B cell lymphomas, acute lymphoblastic leukemia (ALL), chronic lymphocytic leukemia (CLL), Non-Hodgkin's Lymphoma, or Burkitt lymphoma.

In some embodiments, there is provided a method of treating a cancer expressing CD19 in an individual (e.g., human), comprising administering to the individual an effective amount of an BSAP (or an isolated nucleic acid or vector (e.g., viral vector, such as lentiviral vector or AAV) encoding thereof, or a pharmaceutical composition comprising thereof), wherein the BSAP comprises a first polypeptide comprising the amino acid sequence of SEQ ID NO: 138 or 162, and a second polypeptide comprising the amino acid sequence of SEQ ID NO: 139. In some embodiments, the BSAP, or the isolated nucleic acid or vector (e.g., viral vector) encoding thereof, or the composition (such as pharmaceutical composition) comprising thereof, is administered intravenously, subcutaneously, or intratumorally. In some embodiments, the method preferentially activates CD8+ T cells over CD4+ T cells. In some embodiments, the method does not induce high systemic levels of pro-inflammatory cytokines (e.g., IL-2) produced by CD4+ T cells, as measured by the cytokine levels circulating in peripheral blood. In some embodiments, the method does not induce high systemic levels of the pro-inflammatory TNFα or IL-6 cytokines, as measured by the cytokine levels circulating in peripheral blood. In some embodiments, the method does not induce cytokine release syndrome or cytokine storm. In some embodiments, the cancer expressing CD19 is B cell lymphomas, acute lymphoblastic leukemia (ALL), chronic lymphocytic leukemia (CLL), Non-Hodgkin's Lymphoma, or Burkitt lymphoma.

In some embodiments, the method of treating a cancer expressing a target epitope or target antigen (e.g., TSA/TAA-positive cancer) described herein can achieve one or more of the following biological activities: (1) killing (e.g., at least about any of 10%, 20%, 30%, 40%, 50%, 60%, 70%, 80%, 90%, or 100%) cancer cells; (2) inhibiting (e.g., at least about any of 10%, 20%, 30%, 40%, 50%, 60%, 70%, 80%, 90%, or 100%) proliferation of cancer cells; (3) inducing (e.g., at least about any of 10%, 20%, 30%, 40%, 50%, 60%, 70%, 80%, 90%, 100%, 1.5, 2, 3, 4, 5, 10, 20, or more folds) peripheral T cell redistribution (e.g., recruiting T cells to tissues or tumors that express the TSA or TAA); (4) reducing (e.g., at least about any of 10%, 20%, 30%, 40%, 50%, 60%, 70%, 80%, 90%, or 100%) tumor size; (5) alleviating (e.g., at least about any of 10%, 20%, 30%, 40%, 50%, 60%, 70%, 80%, 90%, or 100%) one or more symptoms in an individual having cancer; (6) inhibiting (e.g., at least about any of 10%, 20%, 30%, 40%, 50%, 60%, 70%, 80%, 90%, or 100%) tumor metastasis (e.g., metastasis to lymph nodes); (7) reducing (e.g., at least about any of 10%, 20%, 30%, 40%, 50%, 60%, 70%, 80%, 90%, or 100%) the presentation, incidence, or burden of pre-existing tumor metastasis (e.g., metastasis to lymph nodes); (8) prolonging survival, such as prolonging the survival of the individual by at least any of 1, 2, 3, 4, 5, 6, 7, 8, 9, 10, 11, 12, 18, or 24 months, or more; (9) prolonging time to cancer progression, such as prolonging the time to cancer progression by at least any of 1, 2, 3, 4, 5, 6, 7, 8, 9, 10, 11, or 12 weeks, or more; (10) preventing, inhibiting, or reducing (e.g., at least about any of 10%, 20%, 30%, 40%, 50%, 60%, 70%, 80%, 90%, or 100%) the likelihood of the recurrence of a cancer; (11) increasing, enhancing, or stimulating (e.g., at least about any of 10%, 20%, 30%, 40%, 50%, 60%, 70%, 80%, 90%, 100%, 1.5, 2, 3, 4, 5, 10, 20, or more folds) an immune response or function in a subject by activating effector cells (e.g., T cells, e.g., CD8+ T cells); (12) preferentially increasing (e.g., at least about any of 10%, 20%, 30%, 40%, 50%, 60%, 70%, 80%, 90%, 100%, 1.5, 2, 3, 4, 5, 10, 20, or more folds) CD8+ T cell activation and function over CD4+ T cells; (13) pharmacokinetic stability upon in vivo administration, such as by either subcutaneous (S.C.) or intravenous (I.V.) administration; (14) reducing (e.g., at least about any of 10%, 20%, 30%, 40%, 50%, 60%, 70%, 80%, 90%, 100%, 1.5, 2, 3, 4, 5, 10, 20, or more folds) systemic production of pro-inflammatory cytokines that can induce cytokine release syndrome or cytokine storm; and/or (15) not altering or only minimally increasing cytotoxicity against CD8+ T cells (e.g., by about 1% to about 15%, such as about any of 1%, 2%, 3%, 4%, 5%, 6%, 7%, 8%, 9%, 10%, 11%, 12%, 13%, 14%, or 15%). In some embodiments, the CD8+ T cells in the individual have increased or enhanced (e.g., increasing at least about any of 10%, 20%, 30%, 40%, 50%, 60%, 70%, 80%, 90%, 100%, 1.5, 2, 3, 4, 5, 10, 20, or more folds) priming, activation, proliferation, IFN-γ cytokine production, and/or cytolytic activity relative to prior to the administration of the MSAPs (such as TSAPs) described herein or pharmaceutical composition thereof, or relative to administration of a reference MSAP not comprising an anti-CD8 moiety (e.g., TAA (TSA)×CD3 BSAP or TSAP). In some embodiments, the method preferentially activates CD8+ T cells over CD4+ T cells. In some embodiments, the method does not induce high systemic levels of pro-inflammatory cytokines (e.g., IL-2) produced by CD4+ T cells, as measured by the cytokine levels circulating in peripheral blood. In some embodiments, the method does not induce high systemic levels of the pro-inflammatory TNFα or IL-6 cytokines, as measured by the cytokine levels circulating in peripheral blood. In some embodiments, the method does not induce cytokine release syndrome or cytokine storm. In some embodiments, the MSAP or composition thereof induces less cytokine release syndrome or cytokine storm (e.g., at least about any of 10%, 20%, 30%, 40%, 50%, 60%, 70%, 80%, 90% or less cytokine release syndrome or cytokine storm) compared to a reference MSAP not comprising an anti-CD8 moiety (e.g., TAA (TSA)×CD3 BSAP or TSAP). In some embodiments, the individual is a human.

In some embodiments, the method of treating a disease expressing a target epitope or target antigen (e.g., a disease associated or disease specific antigen) described herein can achieve (i.e., in vivo) one or more of the following biological activities: (1) killing (e.g., at least about any of 10%, 20%, 30%, 40%, 50%, 60%, 70%, 80%, 90%, or 100%) target cells; (2) inhibiting (e.g., at least about any of 10%, 20%, 30%, 40%, 50%, 60%, 70%, 80%, 90%, or 100%) proliferation of target cells (e.g., diseased cells or microbes such as bacteria or fungi); (3) inducing (e.g., at least about any of 10%, 20%, 30%, 40%, 50%, 60%, 70%, 80%, 90%, 100%, 1.5, 2, 3, 4, 5, 10, 20, or more folds) peripheral T cell (e.g., CD8+ T cell) redistribution (e.g., recruiting T cells to tissues or cells that express the disease-associated or disease-specific antigen); (4) reducing (e.g., at least about any of 10%, 20%, 30%, 40%, 50%, 60%, 70%, 80%, 90%, or 100%) disease severity; (5) alleviating (e.g., at least about any of 10%, 20%, 30%, 40%, 50%, 60%, 70%, 80%, 90%, or 100%) one or more symptoms in an individual having a disease (e.g., infection, autoimmune disorder, or allergy); (6) inhibiting (e.g., at least about any of 10%, 20%, 30%, 40%, 50%, 60%, 70%, 80%, 90%, or 100%) disease progression (e.g., progression from local to systemic infection); (7) reducing (e.g., at least about any of 10%, 20%, 30%, 40%, 50%, 60%, 70%, 80%, 90%, or 100%) the presentation, incidence, or burden of a disease (e.g., infection, autoimmune disorder, or allergy); (8) prolonging survival, such as prolonging the survival of the individual by at least any of 1, 2, 3, 4, 5, 6, 7, 8, 9, 10, 11, 12, 18, or 24 months, or more; (9) prolonging time to disease progression, such as prolonging the time to autoimmune disease or infection progression by at least any of 1, 2, 3, 4, 5, 6, 7, 8, 9, 10, 11, or 12 weeks, or more; (10) preventing, inhibiting, or reducing (e.g., at least about any of 10%, 20%, 30%, 40%, 50%, 60%, 70%, 80%, 90%, or 100%) the likelihood of the recurrence of a disease state; (11) increasing, enhancing, or stimulating (e.g., at least about any of 10%, 20%, 30%, 40%, 50%, 60%, 70%, 80%, 90%, 100%, 1.5, 2, 3, 4, 5, 10, 20, or more folds) an immune response or function in a subject by activating effector cells (e.g., T cells, e.g., CD8+ T cells); (12) preferentially increasing (e.g., at least about any of 10%, 20%, 30%, 40%, 50%, 60%, 70%, 80%, 90%, 100%, 1.5, 2, 3, 4, 5, 10, 20, or more folds) CD8+ T cell activation and function over CD4+ T cells; (13) pharmacokinetic stability upon in vivo administration, such as by either subcutaneous (S.C.) or intravenous (I.V.) administration; (14) reducing (e.g., at least about any of 10%, 20%, 30%, 40%, 50%, 60%, 70%, 80%, 90%, 100%, 1.5, 2, 3, 4, 5, 10, 20, or more folds) systemic production of pro-inflammatory cytokines that can induce cytokine release syndrome or cytokine storm; and/or (15) not altering or only minimally increasing cytotoxicity against CD8+ T cells (e.g., by about 1% to about 15%, such as about any of 1%, 2%, 3%, 4%, 5%, 6%, 7%, 8%, 9%, 10%, 11%, 12%, 13%, 14%, or 15%). In some embodiments, the CD8+ T cells in the individual have increased or enhanced (e.g., increasing at least about any of 10%, 20%, 30%, 40%, 50%, 60%, 70%, 80%, 90%, 100%, 1.5, 2, 3, 4, 5, 10, 20, or more folds) priming, activation, proliferation, IFN-γ cytokine production, and/or cytolytic activity at the site of target cell encounter relative to prior to the administration of the MSAPs (such as TSAPs) described herein, or isolated nucleic acid or vector (e.g., viral vector) encoding thereof, or pharmaceutical composition comprising thereof, or relative to administration of a reference MSAP not comprising an anti-CD8 moiety (e.g., target×CD3 BSAP or TSAP). In some embodiments, the method preferentially activates CD8+ T cells over CD4+ T cells. In some embodiments, the method does not induce high systemic levels of pro-inflammatory cytokines (e.g., IL-2) produced by CD4+ T cells, as measured by the cytokine levels circulating in peripheral blood. In some embodiments, the method does not induce high systemic levels of the pro-inflammatory TNFα or IL-6 cytokines, as measured by the cytokine levels circulating in peripheral blood. In some embodiments, the method does not induce cytokine release syndrome or cytokine storm. In some embodiments, the MSAP or composition thereof induces less cytokine release syndrome or cytokine storm (e.g., at least about any of 10%, 20%, 30%, 40%, 50%, 60%, 70%, 80%, 90% or less cytokine release syndrome or cytokine storm) compared to a reference MSAP not comprising an anti-CD8 moiety (e.g., TAA (TSA)×CD3 BSAP or TSAP). In some embodiments, the individual is a human.

In some embodiments, the MSAPs (such as TSAPs) described herein or pharmaceutical composition thereof are present in the individual for at least about 1 hr, 2 hrs, 3 hrs, 4 hrs, 5 hrs, 6 hrs, 7 hrs, 8 hrs (such as at least about any of 10 hrs, 11 hrs, 12 hrs, 13 hrs, 14 hrs, 15 hrs, 16 hrs, 17 hrs, 18 hrs, 19 hrs, 20 hrs, 24 hrs, 30 hrs, 36 hrs, 42 hrs, 48 hrs, 54 hrs, 60 hrs, 72 hrs, 84 hrs, 96 hrs, or longer) after administration. In some embodiments, the MSAPs (such as TSAPs) described herein or pharmaceutical composition thereof are present in the individual for at least about 8 hrs (such as at least about any of 10 hrs, 11 hrs, 12 hrs, 13 hrs, 14 hrs, 15 hrs, 16 hrs, 17 hrs, 18 hrs, 19 hrs, 20 hrs, 24 hrs, or longer) after subcutaneous administration. In some embodiments, the MSAPs (such as TSAPs) described herein or pharmaceutical composition thereof are present at a stable concentration in the individual for at least about 10 hrs (such as at least about any of 12 hrs, 14 hrs, 15 hrs, 20 hrs, 24 hrs, or longer) after subcutaneous administration. In some embodiments, the MSAPs (such as TSAPs) described herein or pharmaceutical composition thereof are present in the individual for at least about 24 hrs (such as at least about any of 30 hrs, 36 hrs, 42 hrs, 48 hrs, 54 hrs, 60 hrs, 72 hrs, 84 hrs, 96 hrs, or longer) after subcutaneous or intravenous administration. In some embodiments, the individual is a human.

The methods provided herein may be practiced in an adjuvant setting. In some embodiments, the method is practiced in a neoadjuvant setting, i.e., the method may be carried out before the primary/definitive therapy. In some embodiments, the method is used to treat an individual (such as human) who has previously been treated. Any of the methods of treatment provided herein may be used to treat an individual (such as human) who has not previously been treated. In some embodiments, the method is used as a first line therapy. In some embodiments, the method is used as a second line therapy.

The methods described herein are suitable for treating a variety of cancers, including both solid cancer and liquid cancer. The methods are applicable to cancers of all stages, including early-stage cancer, non-metastatic cancer, primary cancer, advanced cancer, locally advanced cancer, metastatic cancer, or cancer in remission. The methods described herein may be used as a first therapy, second therapy, third therapy, or combination therapy with other types of cancer therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting. In some embodiments, the cancer has been refractory to prior therapy.

Examples of cancers that may be treated by any of the treatment methods described herein include, but are not limited to, any TSA/TAA+ cancers (e.g., CD19+, WT1+, or EpCAM+ cancers), which may include colorectal cancer, breast cancer, gastric cancer, prostate cancer, ovarian cancer, gallbladder cancer, pancreatic cancer, prostate adenocarcinoma, ampullary cancer, esophageal cancer, renal cancer, thyroid cancer, squamous cell carcinoma, lung cancer, B cell lymphomas, acute lymphoblastic leukemia (ALL), chronic lymphocytic leukemia (CLL), Non-Hodgkin's Lymphoma (NHL), Burkitt lymphoma, Wilms' tumor, etc. In some embodiments, the cancer is a solid cancer, such as colorectal cancer, breast cancer, gastric cancer, prostate cancer, ovarian cancer, gallbladder cancer, pancreatic cancer, prostate adenocarcinoma, ampullary cancer, esophageal cancer, renal cancer, thyroid cancer, squamous cell carcinoma, lung cancer, and Wilms' tumor. In some embodiments, the cancer is a liquid cancer, such as B cell lymphomas, acute lymphoblastic leukemia (ALL), chronic lymphocytic leukemia (CLL), Non-Hodgkin's Lymphoma (NHL), Burkitt lymphoma.

In some embodiments, the method is suitable for treating cancers that express (e.g., overexpress) a specific TSA or TAA (e.g., CD19, WT1, or EpCAM) on the surface of the cancer cells. In some embodiments, the cancer cells in the individual express at least about any of more than 2, 5, 10, 20, 50, 100, 200, 500, 1000, or more fold of a specific TSA or TAA (e.g., CD19, WT1, or EpCAM) compared to normal cells.

Exemplary routes of administration of any of the MSAPs (such as TSAPs), anti-CD8 constructs (e.g., TAA (TSA)×CD8 MSAP), or anti-CD3 constructs (e.g., TAA (TSA)×CD3 MSAP) described herein (or an isolated nucleic acid or vector (e.g., viral vector, such as lentiviral vector or AAV) encoding thereof, or pharmaceutical composition comprising thereof) include, but are not limited to, oral, intravenous, intracavitary, intratumoral, intraarterial, intramuscular, subcutaneous, parenteral, transmucosal, transdermal, ocular, topical, intraperitoneal, intracranial, intrapleural, and epidermal routes, or be delivered into lymph glands, body spaces, organs or tissues known to contain cancer cells. In some embodiments, the MSAP, anti-CD8 construct (e.g., TAA (TSA)×CD8 MSAP), anti-CD3 construct (e.g., TAA (TSA)×CD3 MSAP), an isolated nucleic acid or vector (e.g., viral vector) encoding thereof, or pharmaceutical composition comprising thereof is administered intravenously, such as by infusion. In some embodiments, the MSAP, anti-CD8 construct (e.g., TAA (TSA)×CD8 MSAP), anti-CD3 construct (e.g., TAA (TSA)×CD3 MSAP), isolated nucleic acid or vector (e.g., viral vector) encoding thereof, or pharmaceutical composition comprising thereof is administered subcutaneously. In some embodiments, the MSAP, isolated nucleic acid or vector (e.g., viral vector) encoding thereof, or pharmaceutical composition comprising thereof is administered by injection. In some embodiments, the MSAP, isolated nucleic acid or vector (e.g., viral vector) encoding thereof, or pharmaceutical composition comprising thereof is administered to the target site, such as intratumorally.

In some embodiments, the MSAP (such as TSAP), anti-CD8 construct (e.g., TAA (TSA)×CD8 MSAP), or anti-CD3 construct (e.g., TAA (TSA)×CD3 MSAP) described herein (or pharmaceutical composition thereof) is administered by intravenous infusion. In some embodiments, the MSAP, anti-CD8 construct (e.g., TAA (TSA)×CD8 MSAP), anti-CD3 construct (e.g., TAA (TSA)×CD3 MSAP), or pharmaceutical composition thereof is infused to the individual over a period of time no more than about any of 24 hours, 20 hours, 18 hours, 10 hours, 8 hours, 6 hours, 4 hours, 2 hours, 1 hours, 30 minutes, or less. In some embodiments, the MSAP, anti-CD8 construct (e.g., TAA (TSA)×CD8 MSAP), anti-CD3 construct (e.g., TAA (TSA)×CD3 MSAP), or pharmaceutical composition thereof is infused to the individual over a period of time of any one of about 30 minutes to about 1 hour, about 1 to about 2 hours, about 2 to about 4 hours, about 4 to about 6 hours, about 6 to about 8 hours, about 8 to about 10 hours, about 10 to about 12 hours, about 12 to about 18 hours, about 18 to about 24 hours, about 30 minutes to about 2 hours, about 2 to about 5 hours, about 5 to about 10 hours, about 10 to about 20 hours, about 30 minutes to about 10 hours, or about 30 minutes to about 20 hours. The MSAP, anti-CD8 construct (e.g., TAA (TSA)×CD8 MSAP), anti-CD3 construct (e.g., TAA (TSA)×CD3 MSAP), or pharmaceutical composition thereof may be infused to the individual at any suitable rate. In some embodiments, the MSAP, anti-CD8 construct (e.g., TAA (TSA)×CD8 MSAP), anti-CD3 construct (e.g., TAA (TSA)×CD3 MSAP), or pharmaceutical composition thereof may be infused at a rate more than about any of 0.01 μg/kg/hr, 0.02 μg/kg/hr, 0.05 μg/kg/hr, 0.1 μg/kg/hr, 0.2 μg/kg/hr, 0.5 μg/kg/hr, 0.6 μg/kg/hr, 0.7 μg/kg/hr, 0.8 μg/kg/hr, 0.9 μg/kg/hr, 1 μg/kg/hr, 1.5 μg/kg/hr, 2 μg/kg/hr, 3 μg/kg/hr, 4 μg/kg/hr, 5 μg/kg/hr, 10 μg/kg/hr, 15 μg/kg/hr, 20 μg/kg/hr, 25 μg/kg/hr, 30 μg/kg/hr, 35 μg/kg/hr, 40 μg/kg/hr, 45 μg/kg/hr, 50 μg/kg/hr, 75 μg/kg/hr, 100 μg/kg/hr, 150 μg/kg/hr, 200 μg/kg/hr, 250 μg/kg/hr, or more.

The dosing regimen of the MSAPs (such as TSAPs), anti-CD8 constructs (e.g., TAA (TSA)×CD8 MSAP), or anti-CD3 constructs (e.g., TAA (TSA)×CD3 MSAP) described herein, isolated nucleic acid or vector (e.g., viral vector, such as lentiviral vector or AAV) encoding thereof, or pharmaceutical compositions comprising thereof administered to the individual (such as human) may vary with the particular composition, the method of administration, and the particular type and stage of cancer being treated. In some embodiments, that effective amount of the MSAP, anti-CD8 construct (e.g., TAA (TSA)×CD8 MSAP), or anti-CD3 construct (e.g., TAA (TSA)×CD3 MSAP) is below the level that induces a toxicological effect (i.e., an effect above a clinically acceptable level of toxicity) or is at a level where a potential side effect can be controlled or tolerated when the composition is administered to the individual.

In some embodiments, the effective amount of the MSAPs, anti-CD8 constructs (e.g., TAA (TSA)×CD8 MSAP), or anti-CD3 constructs (e.g., TAA (TSA)×CD3 MSAP) described herein (or isolated nucleic acid or vector (e.g., viral vector, such as lentiviral vector or AAV) encoding thereof, or pharmaceutical composition comprising thereof) is below the level that induces an adverse effect in the central nervous system. For example, an adverse effect observed in antibody therapy is the occurrence of infusion-related side effects, such as cytokine release syndrome (“CRS”), the severe cases of which are known as “cytokine storms.” When a “cytokine storm” is induced, the healthy individual's immune system is activated and releases large amounts of the pro-inflammatory cytokines, such as INF-γ, CCL2, IL-10, IL-6, etc. It is a potentially fatal immune reaction typically consisting of a positive feedback loop between cytokines and immune cells, with highly elevated levels of various cytokines. Other adverse side effects described to be associated with CRS are fatigue, vomiting, tachycardia, hypertension, back pain, but also central nervous system reactions (CNS reactions), such as seizures, encephalopathy, cerebral edema, aseptic meningitis, and headache. In some embodiments, the method preferentially activates CD8+ T cells over CD4+ T cells. In some embodiments, the method does not induce high systemic levels of pro-inflammatory cytokines (e.g., IL-2) produced by CD4+ T cells, as measured by the cytokine levels circulating in peripheral blood. In some embodiments, the method does not induce high systemic levels of the pro-inflammatory TNF-α or IL-6 cytokines, as measured by the cytokine levels circulating in peripheral blood. In some embodiments, the TSAPs, isolated nucleic acid or vector (e.g., viral vector) encoding thereof, or pharmaceutical composition comprising thereof induce less cytokine release syndrome in an individual than a BSAP antibody that targets the same target moiety. In some embodiments, the TSAPs, isolated nucleic acid or vector (e.g., viral vector) encoding thereof, or pharmaceutical composition comprising thereof induce minimal or no cytokine release syndrome in an individual. In some embodiments, the MSAPs (such as TSAPs or BSAPs; or isolated nucleic acid or vector (e.g., viral vector) encoding thereof, or pharmaceutical composition comprising thereof) is administered at a dose that does not induce cytokine release syndrome, such as cytokine storm. In some embodiments, the MSAPs (such as TSAPs or BSAPs; or isolated nucleic acid or vector (e.g., viral vector) encoding thereof, or pharmaceutical composition comprising thereof) is administered at a dose that does not induce significant release of one or more cytokines selected from the group consisting of IL-2, IL-4, IL-5, IL-6, TNF-α, and INF-γ. In some embodiments, a significant release of a cytokine is sustained release of a cytokine over the course of at least about any of 1 hour, 2 hours, 4 hours, 6 hours, 8 hours, 10 hours, 12 hours, 18 hours, 24 hours, or more. In some embodiments, a significant release of a cytokine is a serum or blood level of a cytokine at a concentration of at least about any of 1, 5, 10, 20, 50, 100, 200, 500, 1000, or more pg/mL. In some embodiments, the MSAPs (such as TSAPs or BSAPs) described herein (or pharmaceutical compositions thereof) require binding to a target epitope (e.g., a specific TSA or TAA) on the target (e.g., tumor) cell in order to recruit and activate T cells. Such requirement can greatly reduce unwanted cytokine storms, and unwanted activation of T cells in the absence of the desired target tumor cell.

In some embodiments, there is provided a method of treating a disease (e.g., a cancer) expressing a target antigen in an individual (e.g., human), comprising administering to the individual an effective amount of an MSAP (e.g., TSAP, or an isolated nucleic acid or vector (e.g., viral vector, such as lentiviral vector or AAV) encoding thereof, or a pharmaceutical composition comprising thereof), wherein the MSAP binds to CD8+ T cells at levels at least about 5-fold (e.g., at least about any of 6, 7, 8, 9, 10, 11, 12, 13, 14, 15, or more folds) higher than the MSAP binds to CD4+ T cells. In some embodiments, the MSAP (e.g., TSAP, or a pharmaceutical composition thereof) does not alter cytotoxicity against CD8+ T cells, or minimally increases cytotoxicity against CD8+ T cells by about 1% to about 15%, such as about any of 1%, 2%, 3%, 4%, 5%, 6%, 7%, 8%, 9%, 10%, 11%, 12%, 13%, 14%, or 15%. In some embodiments, the MSAP (e.g., TSAP, or a pharmaceutical composition thereof) minimally increases cytotoxicity against CD8+ T cells by about 1% to about 5%. In some embodiments, the MSAP (e.g., TSAP, or isolated nucleic acid or vector (e.g., viral vector) encoding thereof, or a pharmaceutical composition comprising thereof) increases the levels of TNF-α produced specifically in CD8+ T cells but not in CD4+ T cells by at least about 20% (e.g., at least about any of 25%, 30%, 35%, 40%, 50%, 60%, 70%, 80%, 90%, 95%, 96%, 97%, 98%, 99%, or more) compared to a reference MSAP not comprising an anti-CD8 moiety. In some embodiments, the MSAP (e.g., TSAP, or isolated nucleic acid or vector (e.g., viral vector) encoding thereof, or a pharmaceutical composition comprising thereof) increases the levels of IFN-γ specifically produced in CD8+ T cells but not in CD4+ T cells by at least about 20% (e.g., at least about any of 25%, 30%, 35%, 40%, 50%, 60%, 70%, 80%, 90%, 95%, 96%, 97%, 98%, 99%, or more) compared to a reference MSAP not comprising an anti-CD8 moiety. In some embodiments, the MSAP (e.g., TSAP, or isolated nucleic acid or vector (e.g., viral vector) encoding thereof, or a pharmaceutical composition comprising thereof) increases the levels of IL-2 specifically produced in CD8+ T cells but not in CD4+ T cells by at least about 20% (e.g., at least about any of 25%, 30%, 35%, 40%, 50%, 60%, 70%, 80%, 90%, 95%, 96%, 97%, 98%, 99%, or more) compared to a reference MSAP not comprising an anti-CD8 moiety. In some embodiments, the MSAP (e.g., TSAP, or isolated nucleic acid or vector (e.g., viral vector) encoding thereof, or a pharmaceutical composition comprising thereof) decreases the levels of TNF-α specifically produced in CD4+ T cells at least about 20% (e.g., at least about any of 25%, 30%, 35%, 40%, 50%, 60%, 70%, 80%, 90%, 95%, 96%, 97%, 98%, 99%, or more) compared to a reference MSAP not comprising an anti-CD8 moiety. In some embodiments, the MSAP (e.g., TSAP, or isolated nucleic acid or vector (e.g., viral vector) encoding thereof, or a pharmaceutical composition comprising thereof) decreases the levels of IFN-γ specifically produced in CD4+ T cells at least about 20% (e.g., at least about any of 25%, 30%, 35%, 40%, 50%, 60%, 70%, 80%, 90%, 95%, 96%, 97%, 98%, 99%, or more) compared to a reference MSAP not comprising an anti-CD8 moiety. In some embodiments, the MSAP (e.g., TSAP, or isolated nucleic acid or vector (e.g., viral vector) encoding thereof, or a pharmaceutical composition comprising thereof) decreases the levels of IL-2 specifically produced in CD4+ T cells at least about 20% (e.g., at least about any of 25%, 30%, 35%, 40%, 50%, 60%, 70%, 80%, 90%, 95%, 96%, 97%, 98%, 99%, or more) compared to a reference MSAP not comprising an anti-CD8 moiety. In some embodiments, the systemic levels (e.g., circulating in peripheral blood in vivo) of total IL-6 is decreased by at least about 20% (e.g., at least about any of 25%, 30%, 35%, 40%, 50%, 60%, 70%, 80%, 90%, 95%, 96%, 97%, 98%, 99%, or more) after administration of the MSAP (e.g., TSAP, or isolated nucleic acid or vector (e.g., viral vector) encoding thereof, or a pharmaceutical composition comprising thereof) compared to a reference MSAP not comprising an anti-CD8 moiety. In some embodiments, the systemic levels (e.g., circulating in peripheral blood in vivo) of total IL-2 is decreased by at least about 20% (e.g., at least about any of 25%, 30%, 35%, 40%, 50%, 60%, 70%, 80%, 90%, 95%, 96%, 97%, 98%, 99%, or more) after administration of the MSAP (e.g., TSAP, or isolated nucleic acid or vector (e.g., viral vector) encoding thereof, or a pharmaceutical composition comprising thereof) compared to a reference MSAP not comprising an anti-CD8 moiety. In some embodiments, the systemic levels (e.g., circulating in peripheral blood in vivo) of total IFN-γ is decreased by at least about 20% (e.g., at least about any of 25%, 30%, 35%, 40%, 50%, 60%, 70%, 80%, 90%, 95%, 96%, 97%, 98%, 99%, or more) after administration of the MSAP (e.g., TSAP, or isolated nucleic acid or vector (e.g., viral vector) encoding thereof, or a pharmaceutical composition comprising thereof) compared to a reference MSAP not comprising an anti-CD8 moiety.

In some embodiments, the MSAP (such as TSAPs), anti-CD8 construct (e.g., TAA (TSA)×CD8 MSAP), or anti-CD3 construct (e.g., TAA (TSA)×CD3 MSAP) described herein (or pharmaceutical composition thereof) is administered at a dose of about 1 μg/kg to about 1 mg/kg, for example a dose of about 10 μg/kg to about 500 μg/kg, or a human equivalent dose thereof. In some embodiments, the MSAP (such as TSAPs), anti-CD8 construct (e.g., TAA (TSA)×CD8 MSAP), or anti-CD3 construct (e.g., TAA (TSA)×CD3 MSAP) described herein (or pharmaceutical composition thereof) is administered at a dose of no more than about any one of 0.01 μg/kg, 0.05 μg/kg, 0.1 μg/kg, 0.5 μg/kg, 1 μg/kg, 2 μg/kg, 5 μg/kg, 10 μg/kg, 15 μg/kg, 20 μg/kg, 25 μg/kg, 30 μg/kg, 40 μg/kg, 50 μg/kg, 100 μg/kg, 150 μg/kg, 200 μg/kg, 250 μg/kg, 300 μg/kg, 400 μg/kg, 500 μg/kg, 600 μg/kg, 700 μg/kg, 800 μg/kg, 900 μg/kg, or 1 mg/kg. The doses described herein may refer to a suitable dose for cynomolgus monkeys, a human equivalent dose thereof, a human dose, or an equivalent dose for the specific species of the individual. In some embodiments, the MSAP (such as TSAPs), anti-CD8 construct (e.g., TAA (TSA)×CD8 MSAP), or anti-CD3 construct (e.g., TAA (TSA)×CD3 MSAP) described herein (or pharmaceutical composition thereof) is administered according to human equivalent dosing schedule of that shown in Examples 6 and 8.

The effective amount of the MSAPs (such as TSAPs), anti-CD8 construct (e.g., TAA (TSA)×CD8 MSAP), or anti-CD3 construct (e.g., TAA (TSA)×CD3 MSAP) described herein (or isolated nucleic acid or vector (e.g., viral vector, such as lentiviral vector or AAV) encoding thereof, or pharmaceutical composition comprising thereof) may be administered in a single dose or in multiple doses. For methods that comprise administration of the MSAP, or isolated nucleic acid or vector (e.g., viral vector) encoding thereof, or pharmaceutical composition comprising thereof in multiple doses, exemplary dosing frequencies include, but are not limited to, daily, daily without break, weekly, weekly without break, weekly for two out of three weeks, weekly for three out of four weeks, once every three weeks, once every two weeks, monthly, every six months, yearly, etc. In some embodiments, the MSAP or pharmaceutical composition thereof is administered about once every 2 weeks, once every 3 weeks, once every 4 weeks, once every 6 weeks, or once every 8 weeks. In some embodiments, the MSAP or pharmaceutical composition thereof is administered at least about any of 1×, 2×, 3×, 4×, 5×, 6×, or 7× (i.e., daily) a week. In some embodiments, the intervals between each administration are less than about any of 3 years, 2 years, 1 year, 12 months, 11 months, 10 months, 9 months, 8 months, 7 months, 6 months, 5 months, 4 months, 3 months, 2 months, 1 month, 4 weeks, 3 weeks, 2 weeks, 1 week, 7 days, 6 days, 5 days, 4 days, 3 days, 2 days, or 1 day. In some embodiments, the intervals between each administration are more than about any of 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 7 months, 8 months, 9 months, 10 months, 11 months, 12 months, 1 year, 2 years, or 3 years. In some embodiments, the intervals between each administration are about 1 to about 7 days, such as about any of 1 day, 2 days, 3 days, 4 days, 5 days, 6 days, or 7 days. In some embodiments, the intervals between each administration are about 2 weeks to about 6 months, such as about any of 3 weeks, 4 weeks, 1 month, 2 months, 3 months, 4 months, 5 months, or 6 months. In some embodiments, there is no break in the dosing schedule.

The administration of the MSAPs (such as TSAPs), anti-CD8 construct (e.g., TAA (TSA)×CD8 MSAP), or anti-CD3 construct (e.g., TAA (TSA)×CD3 MSAP) described herein (or pharmaceutical composition thereof) can occur over an extended period of time, such as from 1 day to about a week, from about a week to about a month, from about a month to about a year, from about a year to about several years. In some embodiments, the MSAP or pharmaceutical composition thereof is administered over a period of at least any of about 1 day, 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more.

### EXAMPLES

The examples below are intended to be purely exemplary of the invention and should therefore not be considered to limit the invention in any way. The following examples and detailed description are offered by way of illustration and not by way of limitation. For the embodiments in which details of the experimental methods are not described, such methods are carried out according to conventional conditions such as those described in Sambrook et al. Molecular Cloning: A Laboratory Manual (New York: Cold Spring Harbor Laboratory Press, 1989), or as suggested by the manufacturers.

### Example 1: Expression and Purification of Exemplary MSAPs

Exemplary trispecific antigen binding proteins (TSAPs; e.g., TAA (TSA)×CD8×CD3) comprising three antibody moieties that bind to different antigen targets were expressed using standard protocols. Amino acid sequences of polypeptide chains of the TAA (TSA)×CD8×CD3 TSAPs and nucleic acid sequences encoding thereof are shown in Table 2.

FIG. 1A depicts a first exemplary target×CD8×CD3 TSAP with a first anti-target scFv fused to the N-terminus of the VH of an anti-CD3 Fab fragment via a linker, and a second anti-CD8 scFv fused to the N-terminus of the VL of the anti-CD3 Fab fragment via a linker (e.g., constructs 001, 004, 005, and 006). FIG. 1B depicts a second exemplary CD3×CD8×target TSAP with a first anti-CD3 scFv fused to the N-terminus of the VH of an anti-target Fab fragment via a linker, and a second anti-CD8 scFv fused to the N-terminus of the VL of the anti-target Fab fragment via a linker (e.g., construct 002). FIG. 1C depicts a third exemplary target×CD3×CD8 TSAP with a first anti-target scFv fused to the N-terminus of the VH of an anti-CD8 Fab fragment via a linker, and a second anti-CD3 scFv fused to the N-terminus of the VL of the anti-CD8 Fab fragment via a linker (e.g., construct 003).

Additional exemplary trispecific antigen binding protein formats are provided in FIGS. 21A-21E. FIG. 21A depicts a fourth exemplary target×CD8×CD3 TSAP with a first anti-CD3 scFv fused to the C-terminus of the CH1 of an anti-target Fab fragment via a linker, and a second anti-CD8 scFv fused to the N-terminus of the VL of the anti-target Fab fragment via a linker (e.g., TSAP construct 017). FIG. 21B depicts a fifth exemplary target×CD8×CD3 TSAP with a first anti-CD8 scFv fused to the C-terminus of the CH1 of an anti-target Fab fragment via a linker, and a second anti-CD3 scFv fused to the N-terminus of the VL of the anti-target Fab fragment via a linker (e.g., TSAP constructs 018, 019, 020, and 022). FIG. 21C depicts a sixth exemplary target×CD8×CD3 TSAP with a first anti-CD3 scFv fused to the N-terminus of the VH of an anti-target Fab fragment via a linker, and a second anti-CD8 scFv fused to the C-terminus of the CL of the anti-target Fab fragment via a linker (e.g., TSAP construct 021). FIG. 21D depicts a seventh exemplary target×CD8×CD3 TSAP with a first anti-CD3 scFv fused to the N-terminus of the VH of an anti-target Fab fragment via a linker, and a second anti-CD8 scFv fused to the C-terminus of the CH1 of the anti-target Fab fragment via a linker (e.g., TSAP construct 023). FIG. 21E depicts an eighth exemplary target×CD8×CD3 TSAP with a first anti-CD3 scFv fused to the N-terminus of the VL of an anti-target Fab fragment via a linker, and a second anti-CD8 scFv fused to the C-terminus of the CL of the anti-target Fab fragment via a linker (e.g., TSAP construct 024).

TABLE 2
Exemplary TAA (TSA) × CD8 × CD3 TSAPs and BSAP controls

| | | |
|---|---|---|
| | Amino acid sequence of | Amino acid sequence of the |
| Construct | the first polypeptide chain | second polypeptide chain |

Exemplary TAA (TSA) × CD8 × CD3 TSAP constructs

| | | |
|---|---|---|
| 001 | SEQ ID NO: 95 | SEQ ID NO: 96 |
| (anti-WT1 scFv #1/anti-CD8 scFv #3/anti-CD3 Fab | | |
| #1) | | |
| 002 | SEQ ID NO: 97 | SEQ ID NO: 98 |
| (anti-CD3 scFv #1/anti-CD8 scFv #3/anti-EpCAM | | |
| Fab) | | |
| 003 | SEQ ID NO: 99 | SEQ ID NO: 100 |
| (anti-EpCAM scFv/anti-CD3 scFv #1/anti-CD8 Fab | | |
| #3) | | |
| 004 | SEQ ID NO: 101 | SEQ ID NO: 102 |
| (anti-EpCAM scFv/anti-CD8 scFv #3/anti-CD3 Fab | | |
| #1) | | |
| 005 | SEQ ID NO: 103 | SEQ ID NO: 104 |
| (anti-CD19 scFv/anti-CD8 scFv #3/anti-CD3 Fab #1) | | |
| 006 | SEQ ID NO: 103 | SEQ ID NO: 106 |
| (anti-CD19 scFv/anti-CD8 scFv #6/anti-CD3 Fab #1) | | |
| 015 | SEQ ID NO: 161 | SEQ ID NO: 106 |
| (anti-CD19 scFv/anti-CD8 scFv #6/anti-CD3 Fab #2) | | |
| 017 | SEQ ID NO: 170 | SEQ ID NO: 171 |
| (anti-CD8 scFv #6/anti-CD19 Fab/anti-CD3 scFv #3) | | |
| 018 | SEQ ID NO: 172 | SEQ ID NO: 173 |
| (anti-CD3 scFv #3/anti-CD19 Fab/anti-CD8 scFv #6) | | |
| 019 | SEQ ID NO: 174 | SEQ ID NO: 175 |
| (anti-CD3 scFv #1/anti-CD19 Fab/anti-CD8 scFv #3) | | |
| 020 | SEQ ID NO: 172 | SEQ ID NO: 176 |
| (anti-CD3 scFv #5/anti-CD19 Fab/anti-CD8 scFv #6) | | |
| 021 | SEQ ID NO: 177 | SEQ ID NO: 178 |
| (anti-CD3 scFv #5/anti-CD19 Fab/anti-CD8 scFv #6) | | |
| 022 | SEQ ID NO: 172 | SEQ ID NO: 182 |
| (anti-CD3 scFv #5/anti-CD19 Fab/anti-CD8 scFv #6) | | |
| 023 | SEQ ID NO: 180 | SEQ ID NO: 169 |
| (anti-CD3 scFv #5/anti-CD19 Fab/anti-CD8 scFv #6) | | |
| 024 | SEQ ID NO: 137 | SEQ ID NO: 181 |
| (anti-CD3 scFv #5/anti-CD19 Fab/anti-CD8 scFv #6) | | |

Exemplary BSAP control constructs

| | | |
|---|---|---|
| 007 (2 anti-EpCAM scFvs and anti-CD8 Fab #1) | SEQ ID NO: 124 | SEQ ID NO: 125 |
| 008 (2 anti-EpCAM scFvs and anti-CD8 Fab #2) | SEQ ID NO: 126 | SEQ ID NO: 127 |
| 009 (2 anti-EpCAM scFvs and anti-CD8 Fab #3) | SEQ ID NO: 128 | SEQ ID NO: 129 |
| 010 (2 anti-EpCAM scFvs and anti-CD8 Fab #4) | SEQ ID NO: 130 | SEQ ID NO: 131 |
| 011 (2 anti-EpCAM scFvs and anti-CD8 Fab #5) | SEQ ID NO: 132 | SEQ ID NO: 133 |
| 012 (2 anti-EpCAM scFvs and anti-CD8 Fab #6) | SEQ ID NO: 134 | SEQ ID NO: 135 |
| 013 (1 anti-WT1 scFv fused to anti-CD3 Fab #1 VH- | SEQ ID NO: 95 | SEQ ID NO: 90 |
| CH1) | | |
| 014 (1 anti-CD3 scFv #1 fused to anti-CD19 Fab | SEQ ID NO: 138 | SEQ ID NO: 139 |
| VH-CH1) | | |
| 016 (1 anti-CD8 scFv #3 fused to anti-CD19 Fab | SEQ ID NO: 162 | SEQ ID NO: 139 |
| VH-CH1) | | |

The anti-CD8 targeting antibody fragment was obtained from phage screening, and the anti-CD3 and anti-TSA or TAA targeting antibody fragments were synthesized. The first fusion polypeptide chain gene and the second fusion polypeptide chain gene were respectively amplified by overlapping extension PCR for the three different target antibody fragment sequences. Both peptide chains contained Kozak sequences and signal peptide sequences. The DNA fragments encoding the first and second fusion polypeptides of the trispecific antigen-binding protein were double-digested with restriction endonucleases (EcoR I and Not I) and cloned into a transient expression vector. The transient expression vector contained an ampicillin resistance gene and a strong promoter CMV gene, which promoted high-level expression of the target gene in eukaryotic cells. The colony was picked and sent for sequencing, and after sequence comparison, it was consistent with the theoretical sequence. The construction process of the expression vector of the control bispecific antibodies (TAA (TSA)×CD3, or TAA (TSA)×CD8) was similar to that of the trispecific antibody. The vector was then transformed into the competent cell DH5a, and 50 mL of E. coli culture medium was inoculated and cultured overnight; the bacteria were harvested the next day for plasmid extraction using the Plasmid Plus Midi Kit (Qiagen).

CHO-S cells were grown in Gibco™ ExpiCHO™ expression medium. 20 μg plasmid DNA was transfected into CHO-S cells (6×10⁶ cells/mL) using ExpiFectamine™ CHO Transfection Kit (Gibco™) according to the manufacturer's instruction. Transfected CHO-S cells were cultured in a CO₂ incubator (37° C., 5% CO₂) for 5 days.

On day 5, the cell culture was centrifuged. The supernatant was collected and filtered through a 0.22 μm sterile membrane. The filtered supernatant was then loaded onto CaptureSelect™ IgG-CH1 Affinity Matrix (Thermo Scientific™) equilibrated with phosphate buffered saline (PBS, pH 7.5), and eluted with 150 mM NaCl and 50 mM NaAc buffer (pH 3.5). The eluate was adjusted with 2M Tris to a pH of approximately 4.5 to 5.5. Purified protein was aliquoted and stored at 4° C.

### Example 2: Determination of Binding Affinities of Anti-CD8 Moieties of Exemplary MSAPs

Recombinant human CD8α protein (Beijing Sino Biological Technology Co., Ltd.) or cynomolgus monkey CD8α protein (Beijing Sino Biological Technology Co., Ltd.) was diluted to 1 μg/mL with PBS, and 100 μL was added to each well and incubated overnight at 4° C. After washing the plate twice with PBST, the CD8α proteins were then blocked with 1.5% BSA-PBST at room temperature for 1 h, prior to washing the plate three times with PBST. 10 μg/mL of the control bispecific antibody was added at 100 μL to the ELISA plate. The plate was read with an OD450 microplate reader. The obtained data were fitted with a four-parameter equation, graphed, and EC₅₀ values calculated (Table 3).

TABLE 3
EC₅₀ values for exemplary EpCAM scFv × CD8 Fab BSAPs.

| | EC₅₀(nM) | |
|---|---|---|
| Construct Name | Human CD8α | Cyno CD8α |
| 007 (2 EpCAM scFvs/CD8 Fab #1) | 28.1 | 7.41 |
| 008 (2 EpCAM scFvs/CD8 Fab #2) | 1.58 | 2.57 |
| 009 (2 EpCAM scFvs/CD8 Fab #3) | 1.52 | 1.3 |
| 010 (2 EpCAM scFvs/CD8 Fab #4) | 1.47 | 8.33 |
| 011 (2 EpCAM scFvs/CD8 Fab #5) | 11.3 | 22.5 |
| 012 (2 EpCAM scFvs/CD8 Fab #6) | 1.93 | 2.67 |

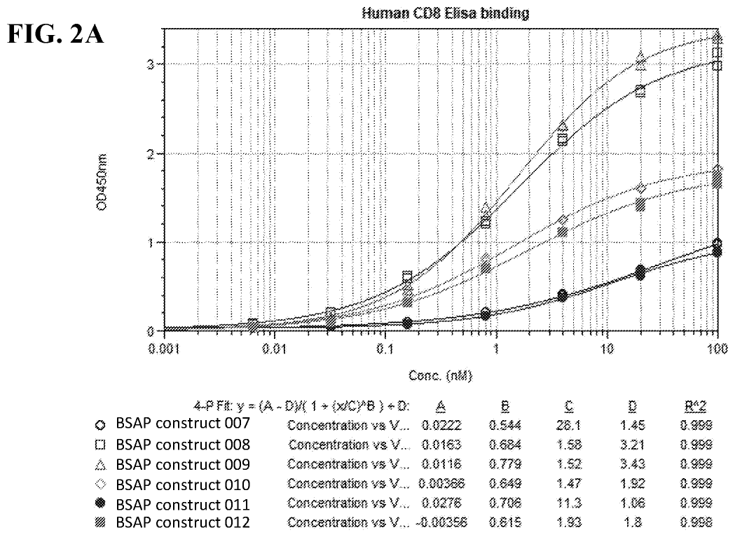

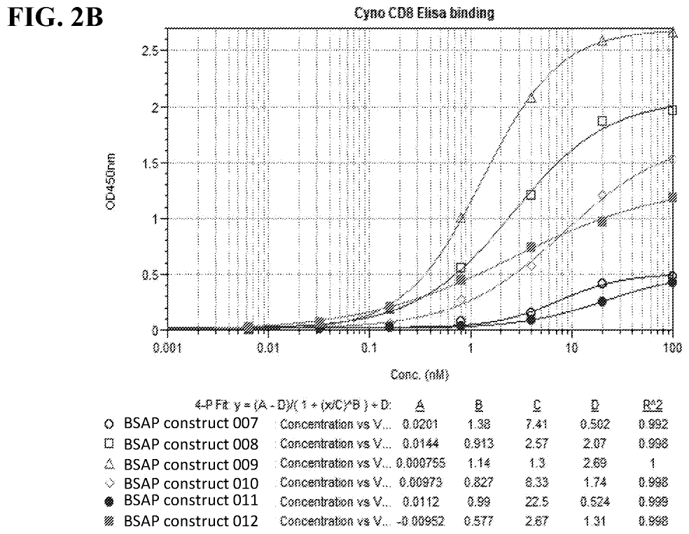

The exemplary EpCAM×CD8 BSAPs exhibited cross-reactivity towards both human and cynomolgus monkey CD8. FIGS. 2A-2B provide the OD450 results for each of the six exemplary BSAP constructs in Table 3 from the human CD8α (FIG. 2A) and cynomolgus monkey CD8α (FIG. 2B) binding assay. The cross-reactivity of anti-CD8 moieties can facilitate extrapolation of toxicity and efficacy study results from cynomolgus monkeys to human clinical studies.

### Example 3: TAA (TSA)×CD8×CD3 TSAPs Preferentially Bind to CD8+ T Cells

Human T Cell Preparation: Human peripheral blood mononuclear cells (hPBMCs) were purchased from OriBiotech and cultured in RPMI 1640 Medium (Gibco™) containing 10% FBS (hereinafter referred to as “complete medium”). hPBMCs were activated for 3 days with 5 μg/mL anti-CD3 antibody (eBioscience, 16-0037-85) and 1 μg/mL anti-CD28 antibody (eBioscience, 16-0289-85), then incubated with 10 ng/ml recombinant human IL-2 (PeproTech®, #200-02) for 3 days for T cell expansion. Cells were then collected and frozen for storage until used for experiments. Prior to experimental use, T cells were thawed in complete medium that was supplemented with 10 ng/mL recombinant human IL-2 and cultured for 3 days.

The CD8+ and CD4+ T cells to be used were diluted in PBS (flow buffer) containing 1% fetal bovine serum, and then 45 L was added per tube containing 1×10⁵ cells that were then inoculated in the EP tubes. Then 5 μL of gradient diluted TSAP (001, 005, 006) or control (013, 014) constructs were added, and the mixture was incubated at room temperature for 60 minutes. Then the cells were washed and resuspended in 50 μL flow buffer with FITC-anti-human CD4 antibody (Biolegend, 344604), APC-anti-human CD8 antibody (Biolegend, 344722), and PE-anti-human lambda light chain antibody (BD Pharmingen, 555797), and incubated for 60 min at room temperature in the dark. The cells were then analyzed by flow cytometry.

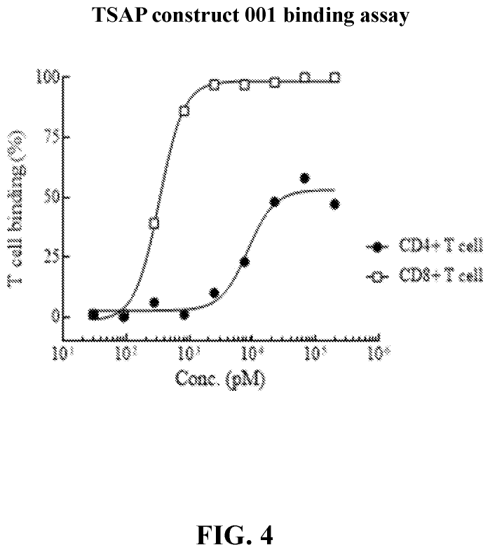

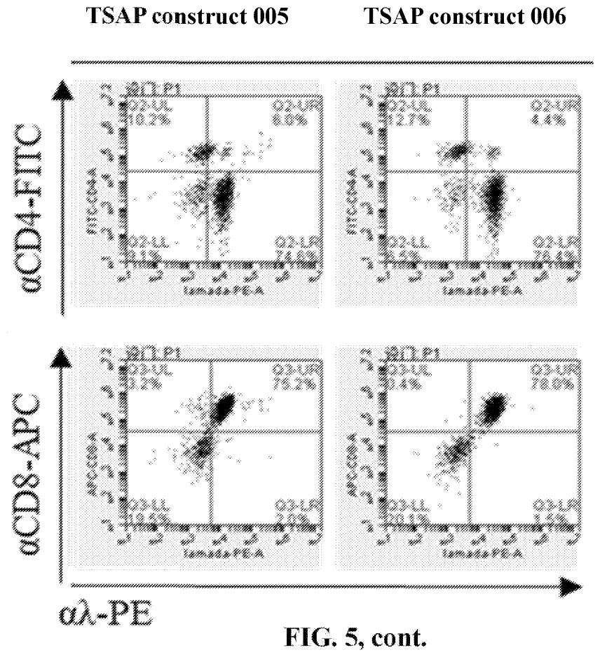

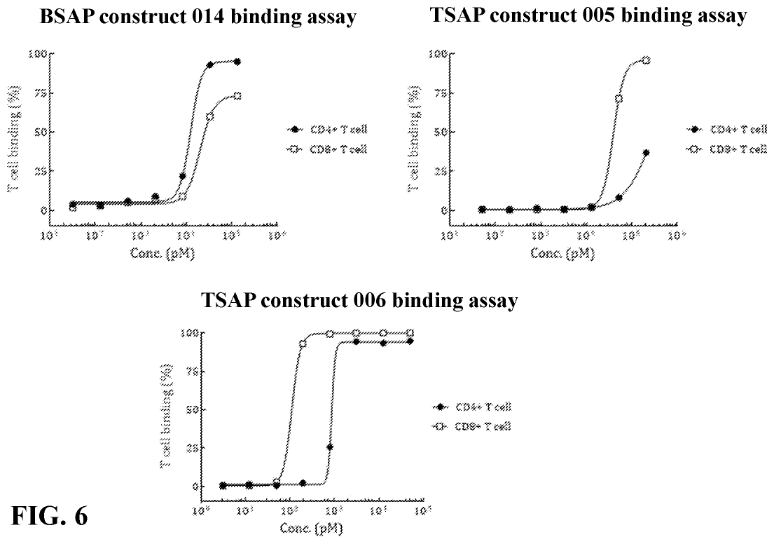

Since the TSAP constructs to be tested contain a human lambda light chain, the λ+ cells represent cells that bind to the added TSAP construct. The percentage of CD4+2+ cells in the CD4+ T cell population and the percentage of CD8+λ+ cells in the CD8+ T cell population were calculated. GraphPad Prism software was used to analyze the data, with the percent of T cell binding on the y-axis and the drug concentration on the x-axis in FIG. 4 and FIG. 6. The curves were fitted according to 4 parameters, and the graph curves and the EC₅₀ values was calculated (see Table 4).

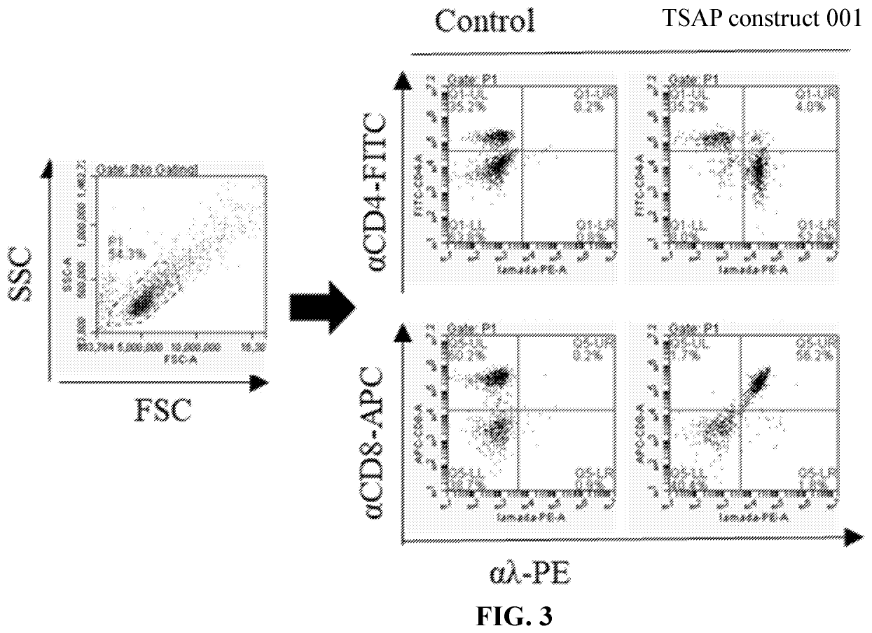

FIG. 3 shows the flow cytometry results, wherein exemplary TSAP construct 001 (anti-WT1 scFv/and anti-CD8 scFv #3/anti-CD3 Fab) preferentially bound to CD8+ T cells (compared to control BSAP construct 013-WT1 scFv×CD3 Fab), although TSAP construct 001 could still bind to some CD4+ T cells. FIG. 4 presents the T cell binding curve for CD8+ and CD4+ T cells based on TSAP construct 001 concentration, demonstrating stronger binding to CD8+ T cells.

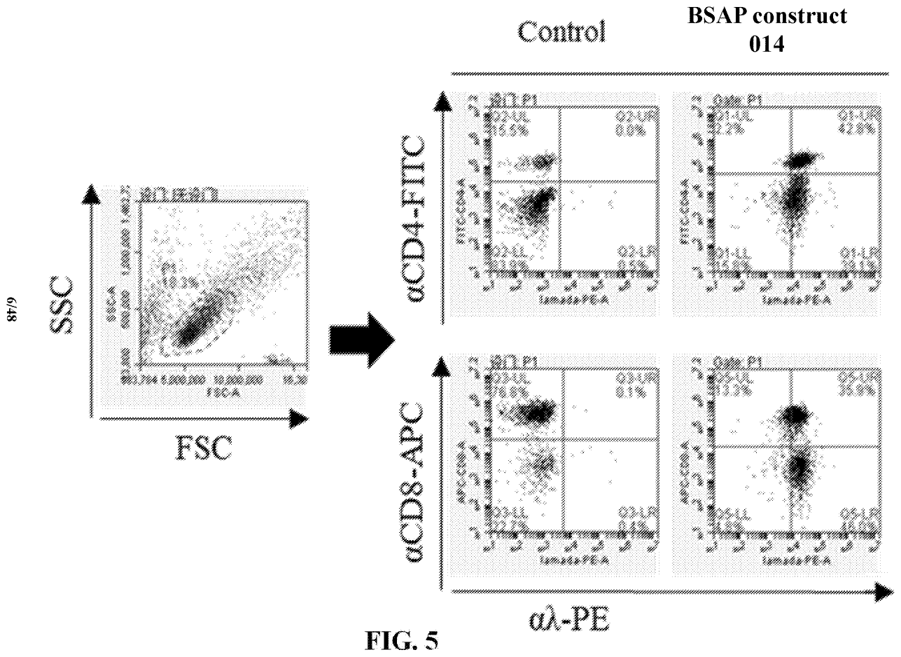

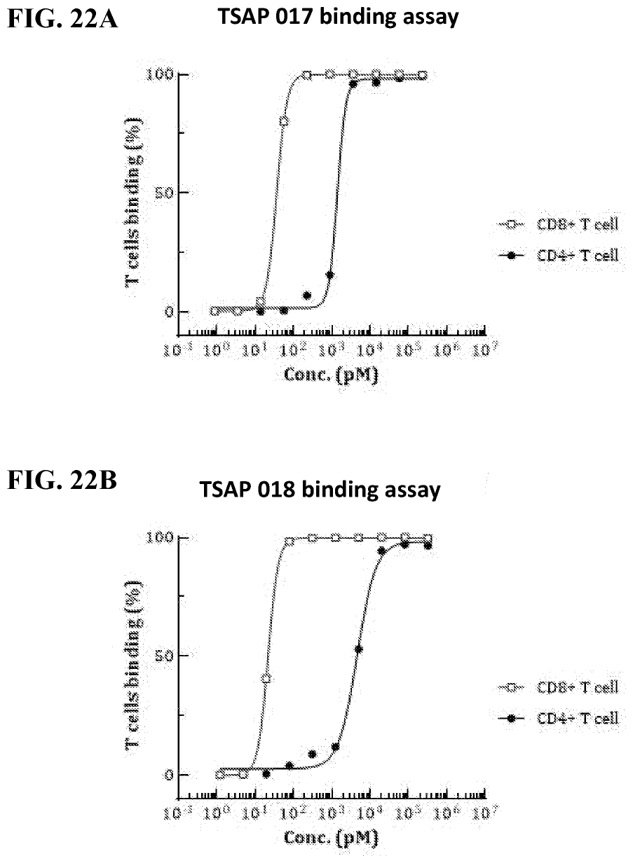

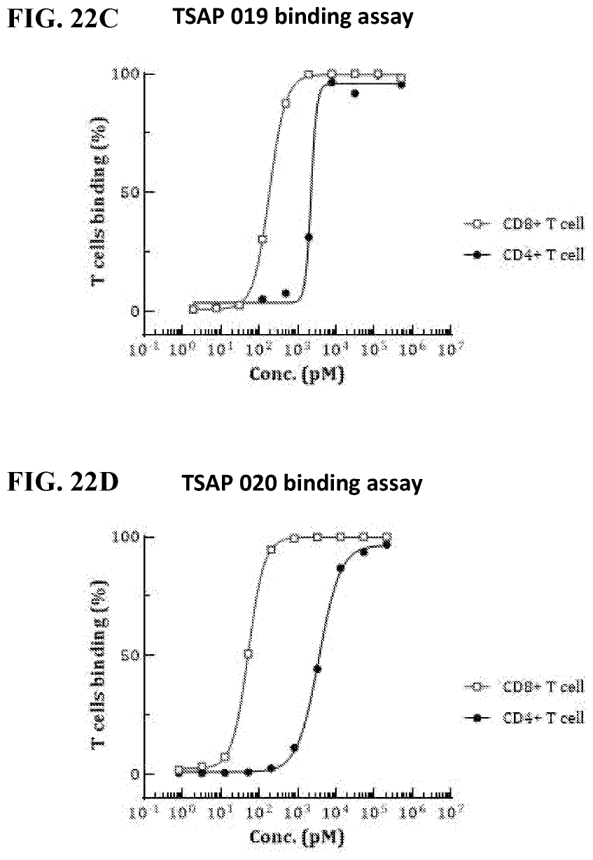

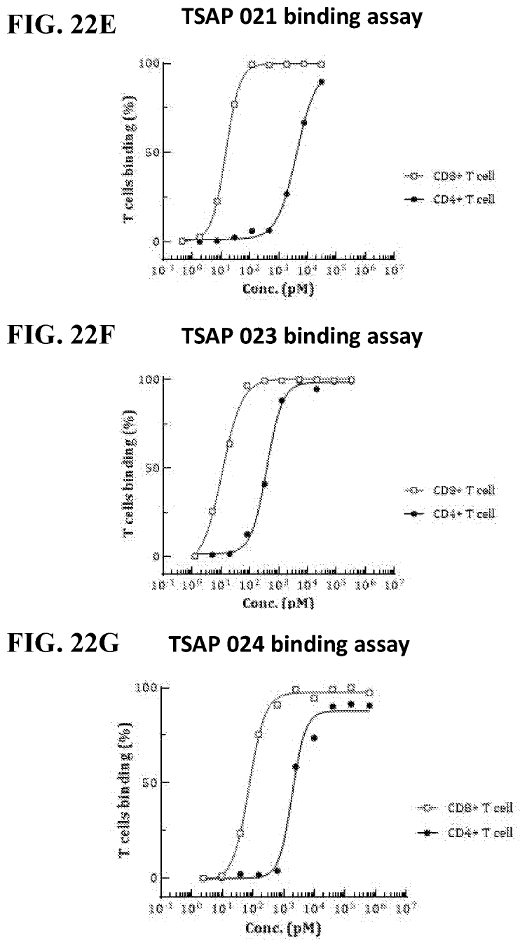

FIG. 5 shows the flow cytometry results, wherein TSAP constructs 005 and 006 preferentially bound to CD8+ T cells, although TSAP constructs 005 (anti-CD19 scFv/anti-CD8 scFv #3/anti-CD3 Fab) and 006 (anti-CD19 scFv/anti-CD8 scFv #6/anti-CD3 Fab) could still bind to some CD4+ T cells. Likewise, CD19 Fab×CD8 scFv BSAP (construct 016) control also showed high affinity binding to CD8+ T cells (data not shown). In contrast, the CD19 Fab×CD3 scFv BSAP control construct 014 bound both CD4+ and CD8+ T cells equally, although showing preferential CD4+ T cell binding. FIG. 6 and FIGS. 22A-22G present the T cell binding curves for CD8+ and CD4+ T cells based on construct concentration. As shown in FIG. 6, TSAP constructs demonstrated stronger binding to CD8+ T cells, compared to BSAP control. As shown in FIGS. 22A-22G, TSAP constructs of various formats all demonstrated selective binding to CD8+ T cells compared to CD4+ T cells.

As shown in Table 4, all tested TSAP constructs preferentially bound to CD8+ T cells compared to CD4+ T cells, while anti-CD3 BSAP controls did not show dramatic binding preference.

TABLE 4
EC₅₀ values for T cell binding of exemplary TAA
(TSA) × CD8 × CD3 TSAPs and BSAP controls.

| Construct | | Cell binding: EC₅₀ (M) | |
|---|---|---|---|
| Name | CD3 × TSA | CD4⁺ T | CD8⁺ T |
| 013 | CD3 Fab × WT1 scFv | 1.8 × 10⁻⁹ | 2.1 × 10⁻⁹ |
| 001 | CD3 Fab × CD8 scFv × WT1 scFv | 8.4 × 10⁻⁹ | 3.3 × 10⁻¹⁰ |

| | CD3 × TAA | CD4⁺ T | CD8⁺ T |
|---|---|---|---|
| 014 | CD3 scFv × CD19 Fab | 1.2 × 10⁻⁸ | 2.0 × 10⁻⁸ |
| 005 | CD3 Fab × CD8 scFv × CD19 scFv | >2.1 × 10⁻⁷ | 3.9 × 10⁻⁸ |
| 006 | | 8.7 × 10⁻¹⁰ | 1.1 × 10⁻¹⁰ |
| 017 | CD3 scFv × CD8 scFv × CD19 Fab | 1.4 × 10⁻⁹ | 3.7 × 10⁻¹¹ |
| 018 | CD3 scFv × CD8 scFv × CD19 Fab | 4.6 × 10⁻⁹ | 2.2 × 10⁻¹¹ |
| 019 | CD3 scFv × CD8 scFv × CD19 Fab | 2.3 × 10⁻⁹ | 1.9 × 10⁻¹⁰ |
| 020 | CD3 scFv × CD8 scFv × CD19 Fab | 3.6 × 10⁻⁹ | 5.2 × 10⁻¹¹ |
| 021 | CD3 scFv × CD8 scFv × CD19 Fab | 4.1 × 10⁻⁹ | 1.5 × 10⁻¹¹ |
| 023 | CD3 scFv × CD8 scFv × CD19 Fab | 0.4 × 10⁻⁹ | 1.1 × 10⁻¹¹ |
| 024 | CD3 scFv × CD8 scFv × CD19 Fab | 1.9 × 10⁻⁹ | 7.4 × 10⁻¹¹ |

### Example 4A: TAA (TSA)×CD8×CD3 TSAPs Mediate T Cell Cytotoxicity Towards Cancer Cells

Target Cell Preparation: The target cells used in this experiment were SW620 (expressing WT1) and Raji (expressing CD19) cancer cell lines, which are adherent and suspension cell lines, respectively.

### Cytolysis of Adherent Cancer Cells

Cultured SW620 target cells were digested with trypsin-EDTA (Gibco) and resuspended in complete medium. 90 μL of 1×10⁴ SW620 target cells per well were then added to a 96-well plate and incubated at 37° C., 5% CO₂ for 4 hrs. Then, 90 μL of 4×10⁴ activated T cells (prepared as above, resuspended in complete medium) per well were added to the 96-well plate containing the target cells (“experiment wells”). These resulted in T cell: target cell ratio of 4:1. Control wells were set up to contain: i) activated T cells only, ii) target cells only, or iii) cell culture medium, and supplemented with complete medium to arrive at the same volume as experiment wells.

20 μL of WT1×CD8×CD3 Fab TSAP (construct 001) diluted with complete medium to various concentrations was then added to the 96-well plate according to experimental design. WT1×CD3 Fab BSAP (construct 013) served as control. 20 μL complete medium was added to control wells to match the reaction volume. The 96-well plate was then incubated at 37° C., 5% CO₂ for 24 hours.

CytoTox 96® Non-Radioactive Cytotoxicity Assay (Promega, #G1781) was used as per manufacturer's protocol in order to compare the degree of lactate dehydrogenase (LDH) released from dead or dying target cells across experimental conditions. LDH is an enzyme that leaks out of cells when the cell membrane becomes compromised as a cell dies. Therefore, when the supernatant from a cell culture is incubated with LDH substrate in a coupled enzymatic assay, the conversion of the substrate into its downstream molecule can act as an indicator of the relative cytolysis of a sample, which is then used to calculate the specific lysis of target cells by effector T cells. Maximum LDH Release Control was set up using the control wells containing target cells only, and treated with lysis solution from the CytoTox 96® Non-Radioactive Cytotoxicity Assay kit following manual.

The 96-well plate was then centrifuged, and supernatants or cell lysates from each well were collected to analyze target cell killing rate (%). Briefly, supernatants or cell lysates were mixed with LDH substrate and incubated at room temperature for 30 minutes. After terminating the enzymatic reaction, a microplate reader (Multiskan SkyHigh®, Thermo Scientific) was used to measure the absorbance at 490 nm (OD490) for each sample.

Tumor cell killing rate (%) was calculated as below:

$$Cell Killing Rate ( \% ) = \frac{{[ OD 490 ]}_{sample} - {[ OD 490 ]}_{m i n}}{{[ OD 490 ]}_{ma x} - {[ OD 490 ]}_{background}} \times 100 \%$$

wherein, [OD490]ₛₐₘₚₗₑ is OD value measured from supernatant of experiment wells (target cells+ T cells+ TSAP); [OD490]ₘᵢₙ is OD value measured from supernatant of control wells (target cells+ T cells, no TSAP); [OD490]ₘₐₓ is OD value measured from cell lysates of target cell control wells (target cells only) treated with lysis solution, indicating the maximum LDH released from death of all target cells; and [OD490]_{background} is OD value measured from supernatant of control wells containing cell culture medium only.

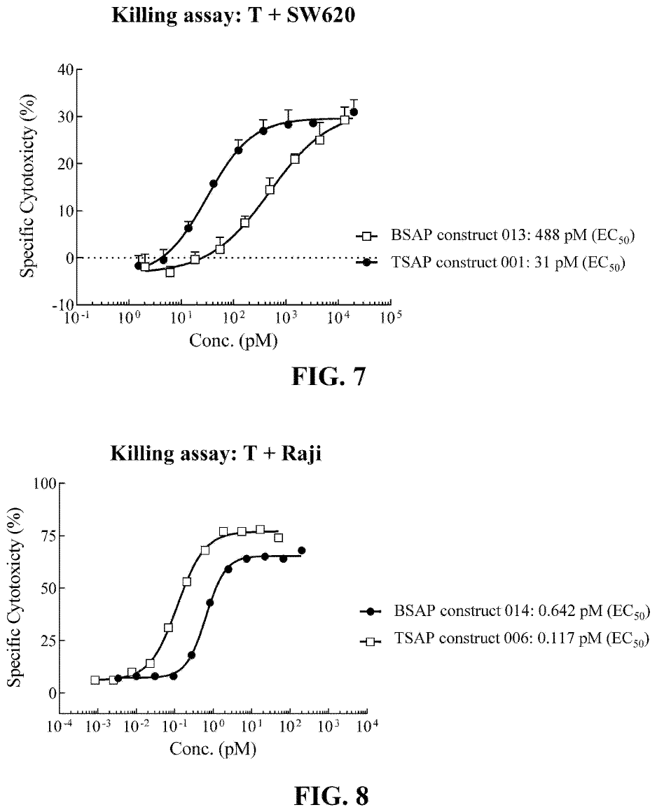

The level of target cell death (% specific cytotoxicity) was set as the y-axis, and TSAP concentration was set as the x-axis. The dose-response curves were fitted using a 4-parameter logistic model with GraphPad Prism software to determine EC₅₀, as shown in FIG. 7. As shown in FIG. 7, the WT1×CD8×CD3 Fab TSAP significantly outperformed the WT1×CD3 Fab BSAP control in SW620-targeted cytolysis.

### Cytolysis of Suspension Cancer Cells

Before co-culturing with T cells, Raji cells that expressed CD19 were stained with CFSE (carboxyfluorescein succinimidyl amino ester) (eBioscience, 65-0850-85) at room temperature for 10 mins; then the staining was terminated, and the cells were washed and resuspended in complete medium. 90 μL of 1×10⁴ Raji target cells per well were then added to a 96-well U-bottom plate and incubated at 37° C., 5% CO₂. Then, 90 μL of 4×10⁴ activated T cells (prepared as above, resuspended in complete medium) per well were added to the 96-well U-bottom plate containing the Raji target cells (“experiment wells”). These resulted in T cell: Raji target cell ratio of 4:1. Control wells were set up to contain: i) activated T cells only, ii) target cells only, or iii) cell culture medium, and supplemented with complete medium to arrive at the same volume as experiment wells.

20 μL of CD19×CD8 (#6)×CD3 Fab TSAP (construct 006) diluted with complete medium to various concentrations were then added to the 96-well plate according to experimental design. CD19 Fab×CD3 BSAP (construct 014) and CD19 Fab×CD8 (#3) BSAP (construct 016) served as control. 20 μL complete medium was added to control wells to match the reaction volume. The 96-well plate was then incubated at 37° C., 5% CO₂ for 24 hours. Then after 24 hr incubation, 7-AAD (BD, 51-68981E) was added to the corresponding wells of the 96-well U-bottom plate, and the co-cultured cells were stained for 15 mins and then analyzed by flow cytometry.

CFSE-positive (CFSE+) cells were target Raji cells, 7-AAD-negative (7-ADD-) cells were viable cells, and CFSE+7-AAD-cells were viable target cells. Cytotoxicity was then calculated according to the formula below.

$$Specific cytotoxicity ( \% ) = 100 \% - 100 \% \times \frac{{CFSE}^{+} 7 {AAD}^{-} / 7 {AAD}^{-} ( Sample )}{{CFSE}^{+} 7 {AAD}^{-} / 7 {AAD}^{-} ( Control )}$$

GraphPad Prism software was used to analyze the data. The cell killing rate (i.e., specific cytotoxicity) was plotted on the vertical axis, and the drug concentration was plotted on the horizontal axis of FIG. 8. The curve was fitted according to 4 parameters and the EC₅₀ values calculated. As shown in FIG. 8, the CD19×CD8×CD3 Fab TSAP significantly outperformed the CD19 Fab×CD3 BSAP control in Raji-targeted cytolysis.

The CD19 Fab×CD3 BSAP control showed preferential CD4+ T cell binding (see Example 3 above), CD4+ T cell activation (see Example 5 below), and CD19+target cell killing. Surprisingly, although the CD19 Fab×CD8 BSAP control showed high affinity binding to CD8+ T cells, no significant target cell killing was observed (data not shown). These demonstrate that only targeting CD8 could not induce effective T-cell mediated cytotoxicity.

### Example 4B: TAA (TSA)×CD8×CD3 TSAPs Mediate T Cell Cytotoxicity Towards TAA/TSA Expressing Cells

This experiment tests the cytotoxicity when both effector cells and target cells are human peripheral blood mononuclear cells (hPBMCs).

hPBMCs were purchased from OriBiotech and seeded onto a 96-well cell culture plate at 1×10⁵ cells/well. Both effector cells (i.e., CD8+ T cells) and target cells (i.e., CD19+/CD20+ B cells) are present in hPBMCs. 20 μL of (i) CD3 (clone #2)×CD8 (#6)×CD19 (construct 017), (ii) CD3 (clone #2)×CD8 (#6)×CD19 (construct 018), (iii) CD3 (clone #1)×CD8 (#3)×CD19 (construct 019), or (iv) CD3 (clone #3)×CD8 (#6)×CD19 (construct 020) diluted with complete medium to various concentrations were then added to the 96-well plate according to experimental design. 20 μL complete medium was added to control wells to match the reaction volume. The 96-well plate was then incubated at 37° C., 5% CO₂ for 24 hours. Then after 24 hr incubation, anti-CD20-APC antibody (BD Biosciences, 559776) and anti-CD8-PE antibody (Biolegend, 301008) were added to the corresponding wells of the 96-well U-bottom plate and stained for 45-60 minutes before adding 7-AAD (BD Biosciences, 51-68981E) and flow cytometry cell counting beads CytomStar YCT500-PE (Nanomicro, YCTFY-500), wherein the co-cultured cells were then stained for an additional 15 mins and analyzed by flow cytometry. 7-AAD stains non-viable cells.

CD20+ represented target B cells, CD8+ represented CD8+ T cells, 7-AAD-negative (7-AAD-) cells represented viable cells, and beads represented cell counting beads. CD20+/7-AAD- or CD8+/7-AAD-were viable target B cells or CD8+ T cells, respectively. Cytotoxicity was then calculated according to the formula below.

$$B cells : Specific cytotoxicity ( \% ) = 100 \% - 100 \% \times \frac{CD {20}^{+} 7 {AAD}^{-} / ( CD {20}^{+} 7 {AAD}^{-} + Bead ) ( Sample )}{CD {20}^{+} 7 {AAD}^{-} / ( CD {20}^{+} 7 {AAD}^{-} + Bead ) ( Control )}$$

$$T cells : Specific cytotoxicity ( \% ) = 100 \% - 100 \% \times \frac{CD {8}^{+} 7 {AAD}^{-} / ( CD {8}^{+} 7 {AAD}^{-} + Bead ) ( Sample )}{CD {8}^{+} 7 {AAD}^{-} / ( CD {8}^{+} 7 {AAD}^{-} + Bead ) ( Control )}$$

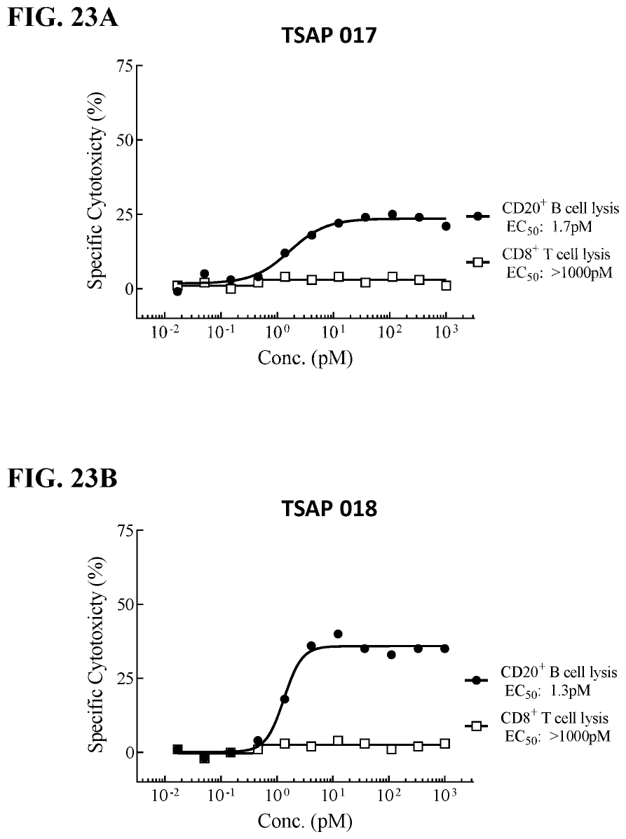

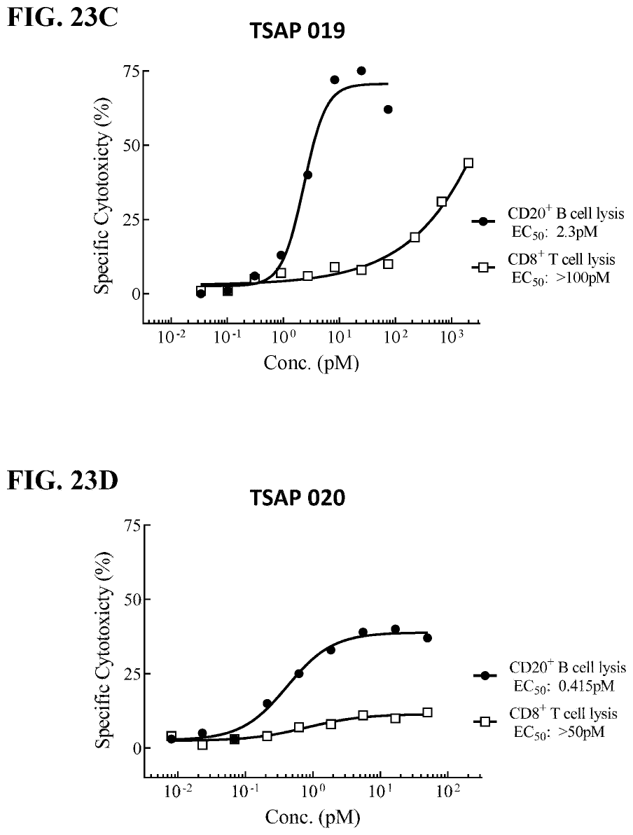

GraphPad Prism software was used to analyze the data. The cell killing rate (i.e., specific cytotoxicity) was plotted on the vertical axis, and the TSAP concentration was plotted on the horizontal axis of FIGS. 23A-23D (TSAP constructs 017 through 020, respectively). The curve was fitted according to 4 parameters and the EC₅₀ values calculated. As shown in FIGS. 23A-23D, the CD19×CD8×CD3 TSAPs of various structures all displayed B-cell targeted cytolysis. See Table 5 below for a summary of specific cytolysis.

TABLE 5
Summary results of specific cytotoxicity
against target B cells and CD8+ T cells.

Killing, EC₅₀(pM)

| TSAP Constructs | CD20+ B cell lysis | CD8+ T cell lysis |
|---|---|---|
| TSAP construct 017 | 1.7 | >1000 |
| TSAP construct 018 | 1.3 | >1000 |
| TSAP construct 019 | 2.3 | >100 |
| TSAP construct 020 | 0.415 | >50 |

Surprisingly, TSAP construct 019 showed different cytotoxicity profiles from the other TSAP constructs, as shown in FIG. 23C. The B cell specific cytolysis EC₅₀ was calculated by removing the highest three TSAP construct 019 concentration points. Further, the cytolysis of CD8+ T cells led to a decrease in B cell cytolysis rate due to the decrease in the number of effector CD8+ T cells present in the relevant experimental wells. Thus, TSAP construct 019 induced CD8+ T cells to kill both the target B cells and effector CD8+ T cells, demonstrating that construct 019 binds to both CD19 and CD8 as targets.

On the other hand, TSAP constructs 017, 018, and 020, shown in FIGS. 23A, 23B and 23D, induce lysis of target B cells but not effector CD8+ T cells, indicating that the sequences and structures of these TSAP constructs do not target CD8 in the same manner as CD19. TSAP constructs 017 and 018 comprise the same sequence but different structures. TSAP construct 018 displayed stronger lysis of B cells than TSAP construct 017, demonstrating superiority of the TSAP construct 018 structure over the TSAP construct 017 structure. TSAP constructs 018 and 020 comprise different sequences but the same structure. The EC₅₀ of TSAP construct 020 for cytolysis of B cells (0.415 μM) was found to be lower compared to that of TSAP construct 018 (1.3 μM), demonstrating the CD3 clone sequence superiority of TSAP construct 020 (CD3 clone #3) over 018 (CD3 clone #2).

### Example 5: CD19×CD8×CD3 TSAPs Mediate CD8+ T Cell Activation and Cytokine Production

Two T cell activation conditions were tested. In the first experiment, 90 μL of 1×10⁴ Raji target cells per well were added to a 96-well U-bottom plate and incubated for 24 hrs at 37° C., 5% CO₂, and 90 μL of 4×10⁴ T cells (resuspended in complete medium and not previously activated) per well were added to the 96-well U-bottom plate containing the Raji cells. In the second experiment, human PBMCs (hPBMCs) containing both effector cells and target cells were plated in a 96-well U-bottom plate and incubated for 24 hrs at 37° C., 5% CO₂. No CD3 antibody, CD28 antibody, or IL-2 cytokine were added to the cells in these experiments. Then 20 μL of CD19×CD8×CD3 Fab TSAP (construct 006; for both experiments) or 20 μL of CD19 Fab×CD8×CD3 TSAP (constructs 017 through 024; for the second experiment only) diluted with complete medium to various concentrations were added to the 96-well plate according to experimental design. CD19 Fab×CD3 BSAP (construct 014) and CD19 Fab×CD8 (#3) BSAP (construct 016) served as control for TSAP construct 006, and CD19 Fab×CD3 BSAP (construct 014) served as control for TSAP constructs 017 through 024. 20 μL complete medium was added to control wells to match the reaction volume. The 96-well plate was then incubated at 37° C., 5% CO₂ for 24 hours.

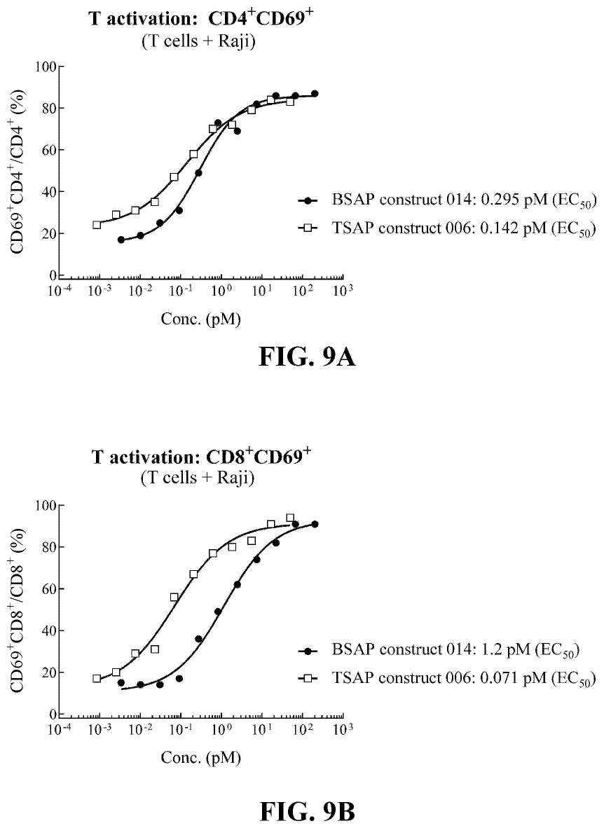

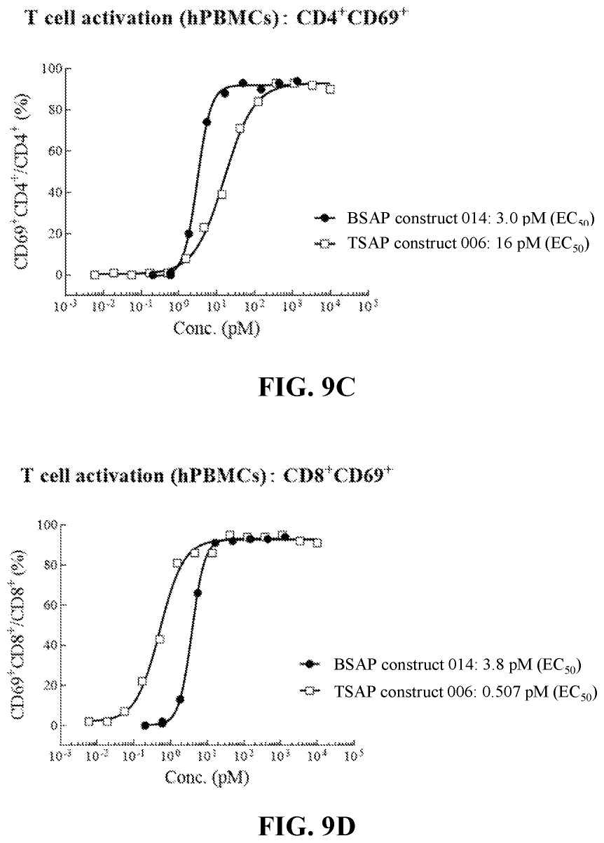

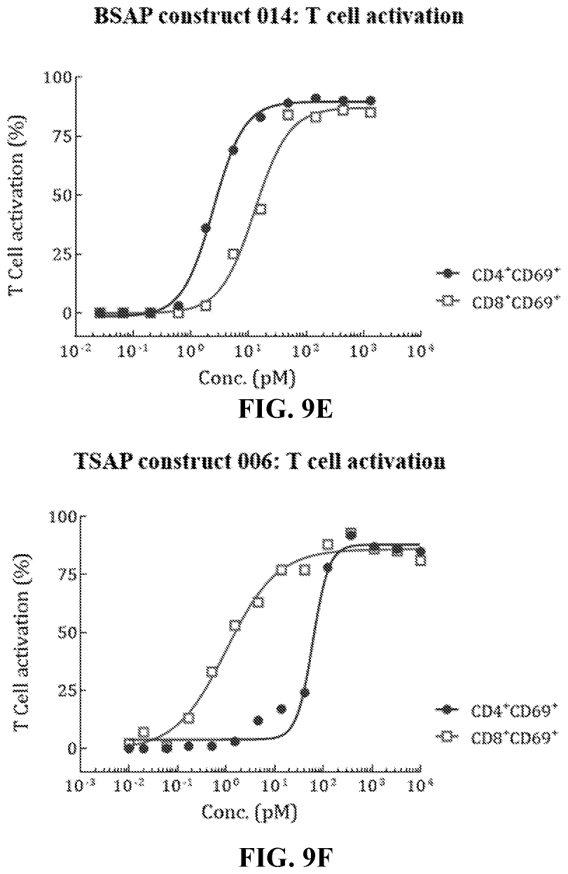

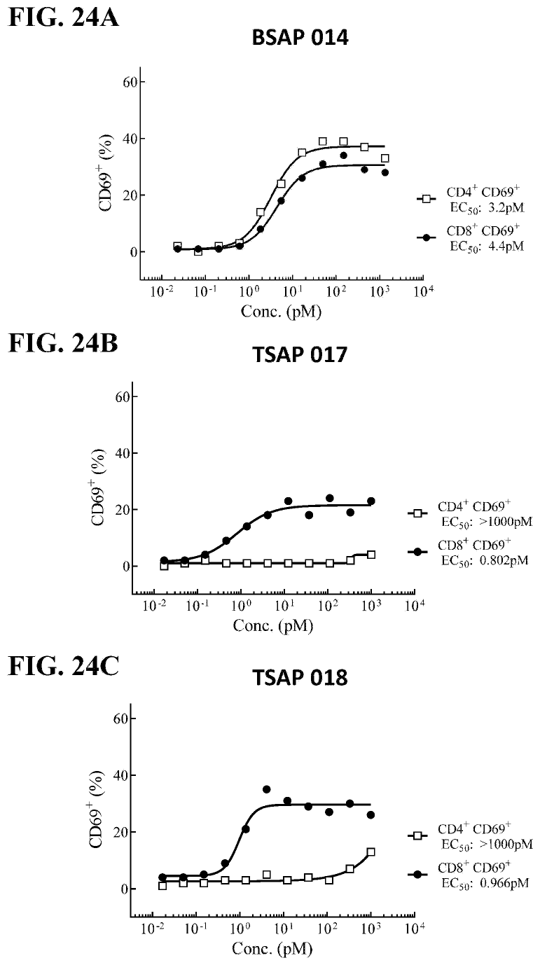

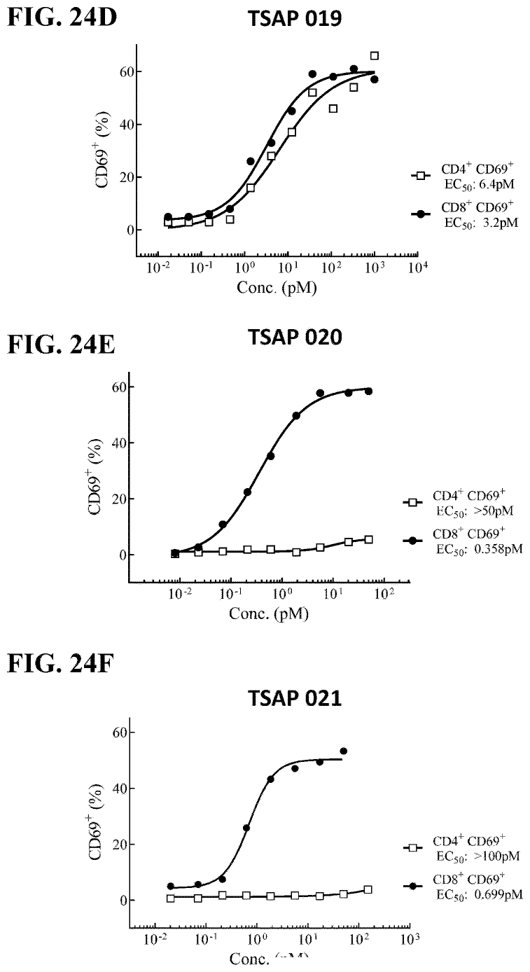

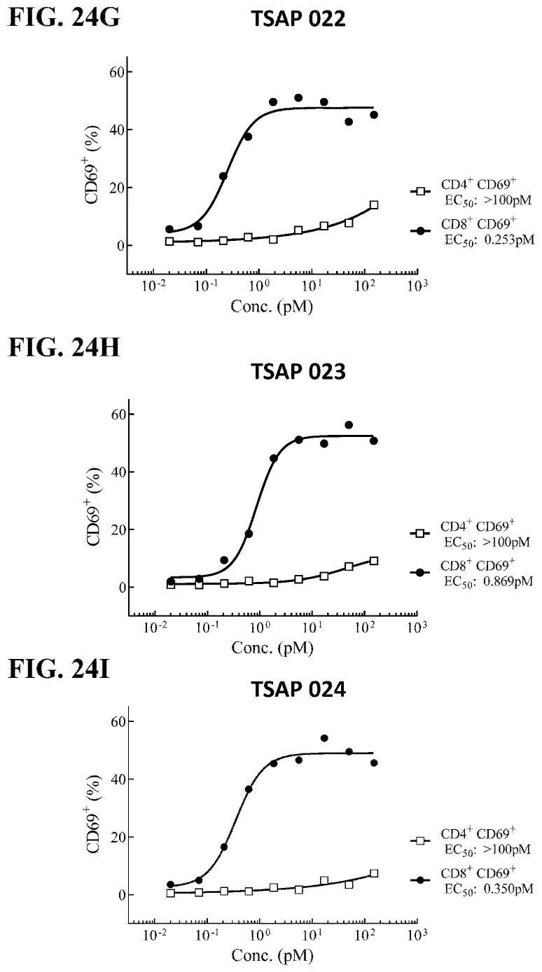

After 24 hours, the cells were stained with APC-anti-CD4 antibody, PE-anti-CD8 antibody, and FITC-anti-CD69 antibody, each purchased from BD Pharmingen. After staining for 30-45 min at room temperature, cells were analyzed by flow cytometry. The proportion of CD69+ cells in the CD4+ T or CD8+ T subsets was calculated to assess for the degree of T cell activation. Using GraphPad Prism software, the dose-response curves were fitted with a 4-parameter equation, and the EC₅₀ values were calculated, as shown in FIGS. 9A-9D and FIGS. 24A-24I.

FIGS. 9A and 9B show the results for T cell activation when co-cultured with CD19+ Raji cells in the presence of TSAP or BSAP. FIGS. 9C and 9D show the results for hPBMC activation in the presence of TSAP or BSAP. FIGS. 9E and 9F show the results T cell activation for the BSAP control construct 014 and the exemplary TSAP construct 006, respectively. When comparing the CD19 Fab×CD3 BSAP with the CD19×CD8×CD3 Fab TSAP, a 1-3 log increase of CD8 T cell activation was identified. The EC₅₀ of TSAP construct 006 for the induction of CD8+ T cell activation was much lower than BSAP control construct 014, but not for CD4+ T cell activation, demonstrating that the exemplary TSAP could preferentially enhance the activation of CD8+ T cells over CD4+ T cell activation. As shown in FIG. 9E, CD19 Fab×CD3 BSAP showed higher CD4+ T cell activation compared to CD8+ T cells.

FIG. 24A shows the results T cell activation for the CD19 Fab×CD3 BSAP control construct 014, indicating that both CD4+ T cells and CD8+ T cells were activated. FIGS. 24B-24I show the results for hPBMC activation in the presence of TSAP constructs 017 through 024 of various structures and sequences. These results can be viewed as belonging to one of two categories: (1) unable to specifically induce CD8+ T cell activation (i.e., BSAP construct 014 and TSAP construct 019; see FIGS. 24A and 24D); or (2) able to specifically induce (i.e., activate) CD8+ T cells (i.e., TSAP constructs 017, 018, and 20-24; see FIGS. 24B, 24C, and 24E-24I). Of the second group, TSAP constructs 017 and 018 comprise the same antibody sequence but different structures. TSAP constructs 018, 020, and 022 comprise the same structure but different sequences. TSAP constructs 020 and 022 showed slightly stronger CD8+ T cell activation compared to TSAP construct 018, likely due to stronger binding of the anti-CD3 clone #3 used in TSAP constructs 020 and 022 compared to the anti-CD3 clone #2 used in TSAP construct 018. TSAP constructs 017, 018, 020, 021, 022, 023, and 024 display similar abilities to induce CD8+ T cell activation in vitro (i.e., EC₅₀ of about 0.350-1 μM) without inducing of CD4 T cell activation in vitro. Table 6 below summarizes the T cell activation EC₅₀ results.

TABLE 6
T cell activation EC₅₀ results.

T cell activation, EC₅₀(pM)

| Construct | CD4+CD69+ | CD8+CD69+ |
|---|---|---|
| BSAP construct 014 | 3.2 | 4.4 |
| TSAP construct 017 | >1000 | 0.802 |
| TSAP construct 018 | >1000 | 0.966 |
| TSAP construct 019 | 6.4 | 3.2 |
| TSAP construct 020 | >50 | 0.358 |
| TSAP construct 021 | >100 | 0.699 |
| TSAP construct 022 | >100 | 0.253 |
| TSAP construct 023 | >100 | 0.869 |
| TSAP construct 024 | >100 | 0.350 |

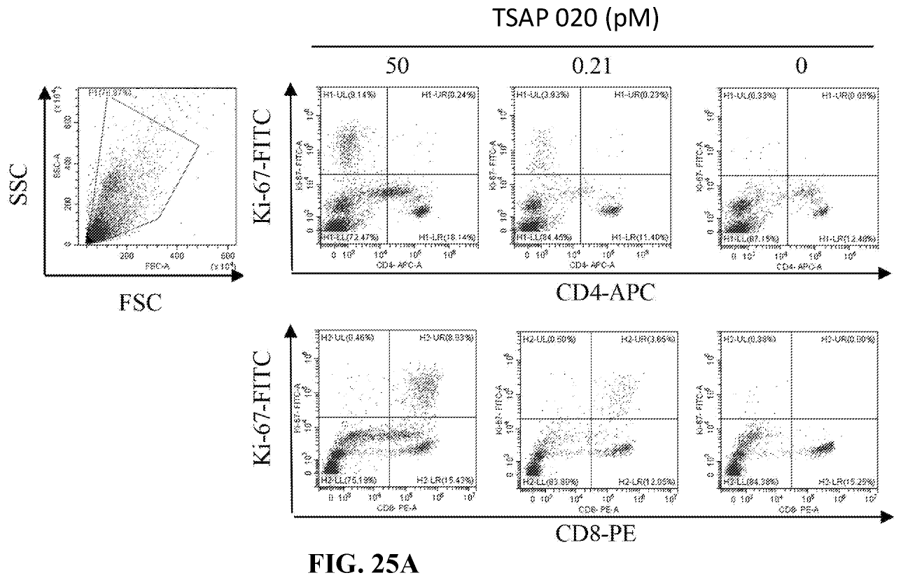

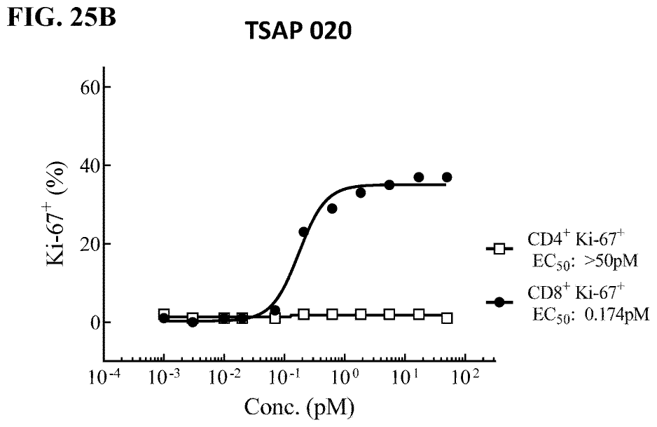

T cell proliferation was then assessed to confirm the T cell activation data above. In brief, hPBMCs were seeded into a 96-well cell culture plate, and 20 μL of CD19 Fab×CD8×CD3 TSAP construct 020 was diluted with complete medium to various concentrations and then added to the 96-well plate according to experimental design. Cells were then incubated at 37° C. and 5% CO₂. After 72 hours, the hPBMCs were collected, fixed and permeabilized using Foxp3/Transcription Factor Flow Cytometry Fixation and Permeabilization Buffer (eBioscience™) according to the manufacturer's instructions, and then stained for CD4, CD8, and Ki-67 using anti-CD4-APC antibody (Biolegend, 344614), anti-CD8-PE antibody (Biolegend, 301008), and anti-Ki-67-FITC antibody (BD Pharmingen, 556026). Cells were stained for 30-60 minutes and then analyzed by flow cytometry. FIG. 25A provides representative FACS plots of Ki-67, CD8, and CD4 staining of hPBMCs incubated with 50 μM, 0.21 μM, or OpM (control) TSAP construct 020. The proportion of Ki-67-positive (Ki-67+) cells in the CD4+ T cell or CD8+ T cell subsets were calculated. Using GraphPad Prism software, the dose-response curves were fitted with a 4-parameter equation, and the EC₅₀ values were calculated, as shown in FIG. 25B. These results showed that TSAP construct 020 could specifically induce CD8+ T cell proliferation but barely induce CD4+ T cell proliferation. These data support the finding that TSAP construct 020 specifically induces CD8+ T cell activation.

Further, the level of cytokine release was assessed using intracellular cytokine flow staining, wherein the cells were incubated with Golgi inhibitor to prevent the secretion of cytokines out of the cells during cell incubation, thereby allowing the level of cytokines in the cells to be detected by flow cytometry. In short, PBMCs and Raji cells were plated at a ratio of 10:1. The BSAP control construct 014 or exemplary TSAP constructs 005 or 006 were serially diluted and added to the wells of the 96-well U-bottom plate. The Golgi inhibitor was added simultaneously, and the cell co-cultures were incubated at 37° C. and 5% CO₂ for 4 hours. After the 4 hr incubation was completed, the cells were stained for CD4 and CD8 as described above, then fixed with cell fixative solution, and then stained for intracellular cytokines TNFα, IL-2, and IFNγ. The cells were then analyzed by flow cytometry to determine the percentage of T cells that were producing each of the three cytokines.

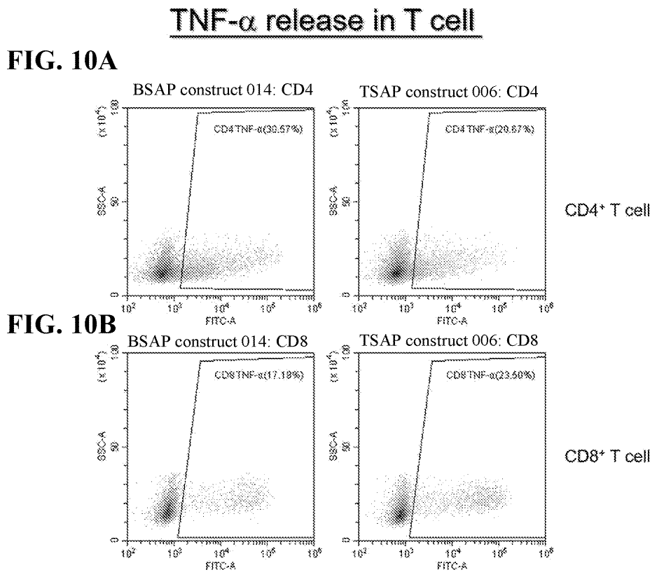

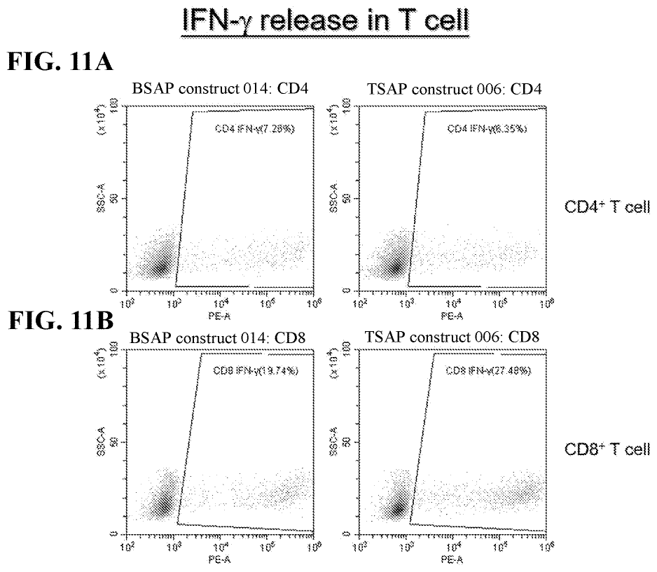

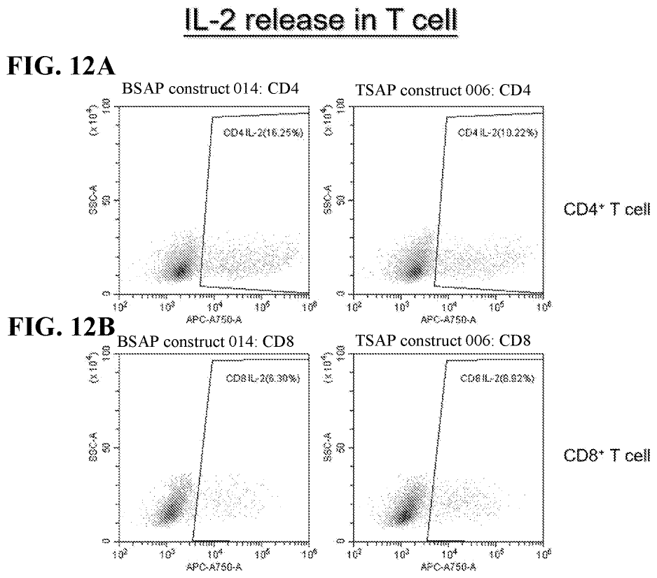

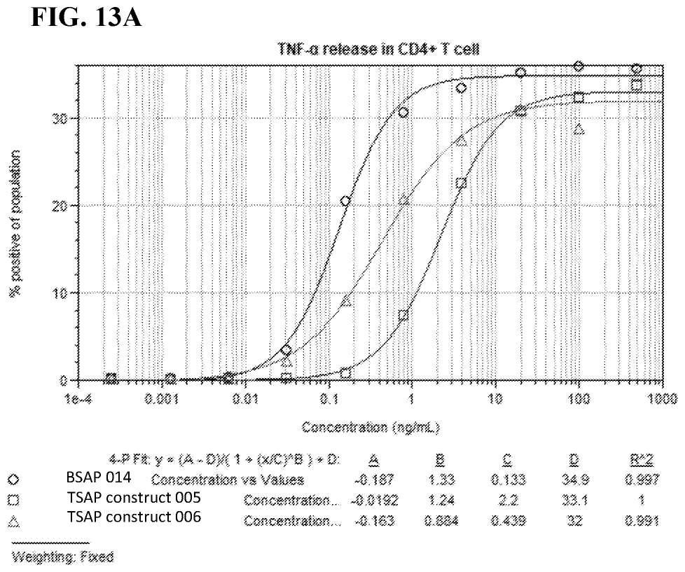

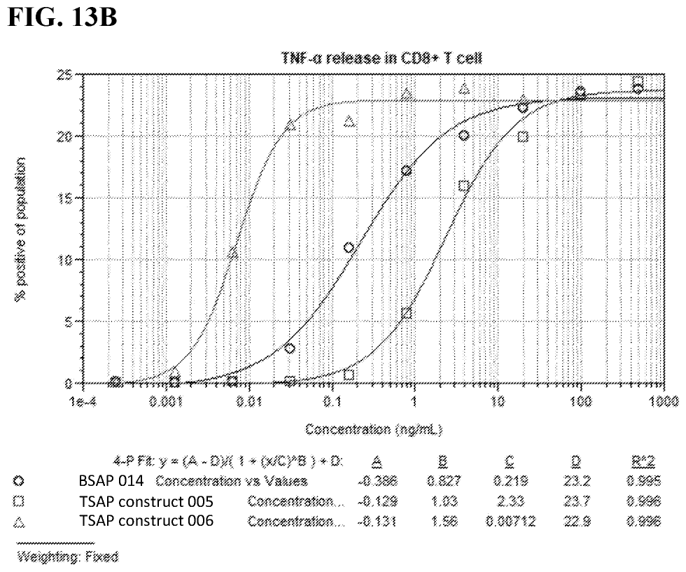

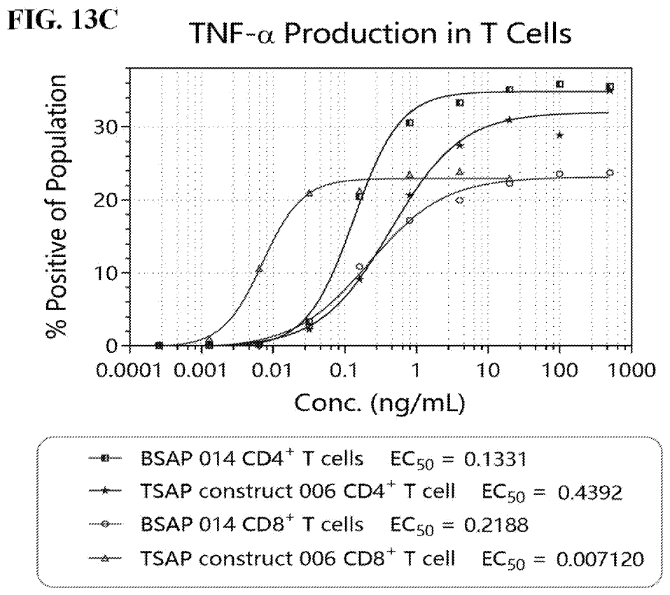

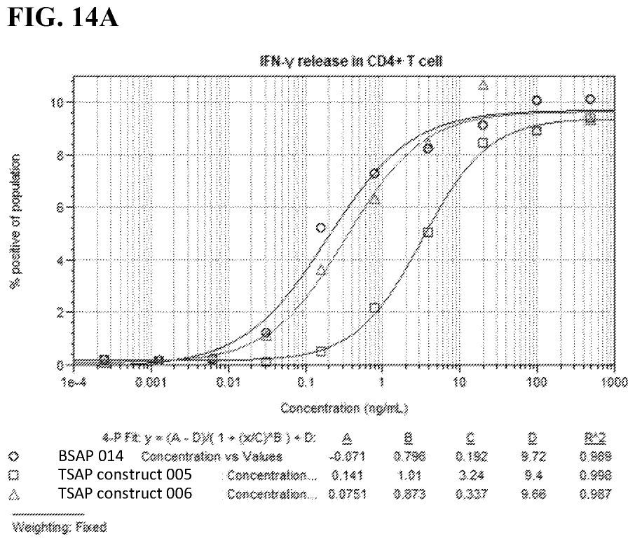

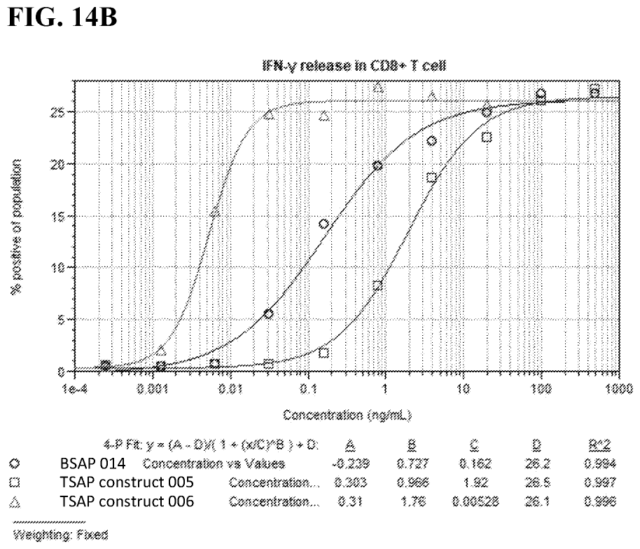

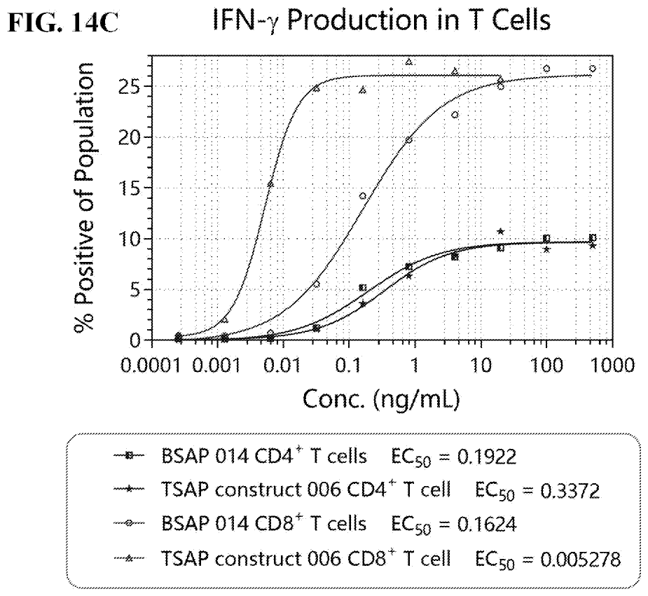

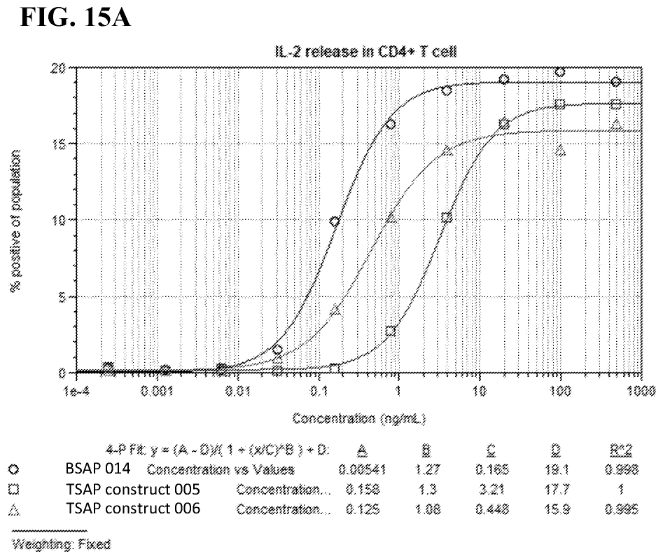

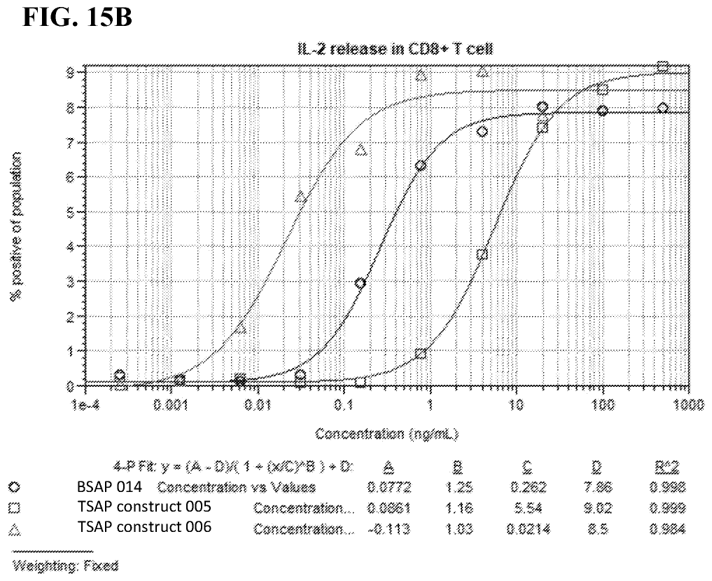

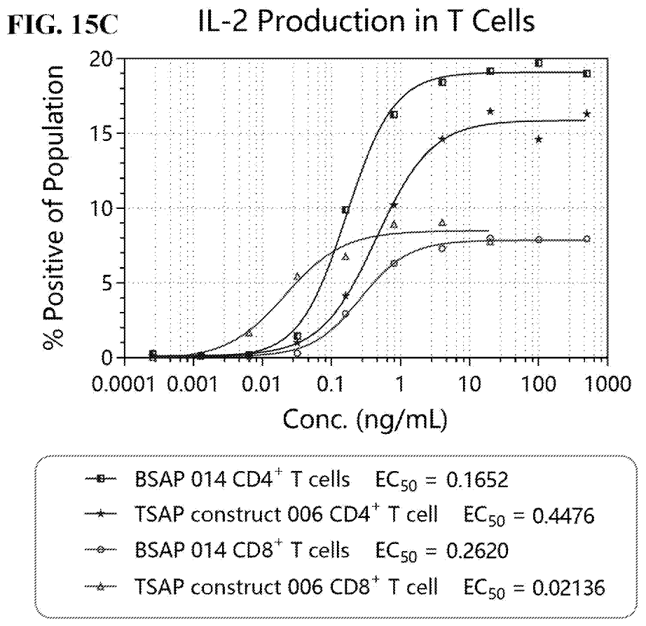

The level of intracellular cytokines was assessed by calculating the percentage of cytokine-positive cells (i.e., TNFα, IL-2, and IFNγ) and using GraphPad Prism software to fit the data with a 4-parameter equation and calculate the EC₅₀ values were calculated (Table 7). Compared with the BSAP control construct 014, the exemplary TSAP construct 006 showed CD8+ T cell cytokine release selectivity (e.g., TNFα in FIGS. 10A and 10B; IFNγ in FIGS. 11A and 11B; and IL-2 in FIGS. 12A and 12B), wherein the EC₅₀ of CD8+ T cells releasing cytokines was significantly lower than that of CD4+ T cells (see FIGS. 13A-13C for TNFα; FIGS. 14A-14C for IFNγ; and FIGS. 15A-15C for IL-2). CD8+ T cells are known to produce IFNγ for cytolytic activity and target cell killing, whereas CD4+ T cells produce more IL-2 than CD8+ T cells. TNFα is pro-inflammatory cytokine that, at high circulating levels, can induce cytokine release syndrome and immune toxicity. The superior effects of TSAP construct 006 could come from its superior CD8+ T cell binding activity compared to TSAP construct 005 and BSAP control 014. See EC₅₀ values for T cell binding in Table 4.

TABLE 7
EC₅₀ values for T cell cytokine release of exemplary CD19
× CD8 × CD3 TSAPs and BSAP control.

| | BSAP construct 014 | | TSAP construct 005 | | TSAP construct 006 | |
|---|---|---|---|---|---|---|
| ng/mL | CD4 | CD8 | CD4 | CD8 | CD4 | CD8 |
| TNFα | 0.133 | 0.219 | 2.2 | 2.33 | 0.439 | 0.00712 |
| IFNγ | 0.192 | 0.162 | 3.24 | 1.92 | 0.337 | 0.00528 |
| IL-2 | 0.165 | 0.262 | 3.21 | 5.54 | 0.448 | 0.0214 |

### Example 6: The Effect of CD19×CD8×CD3 TSAPs on Tumor Burden in a Humanized Mouse Xenograft Model

Experiment 1: The human Burkitt lymphoma cell line, Raji, was cultured and expanded in vitro, and once in the logarithmic growth phase, the tumor cells were collected, resuspended in sterile PBS, and inoculated into NSG mice by subcutaneous injection at a dose of 3×10⁶ cells on day 0. On day 4 after inoculation, human PBMC cells were collected, resuspended in sterile PBS, and administered to mice by intraperitoneal injection (i.p.) at a dose of 5×10⁶ cells per mouse. When the tumors grew to approximately 100 mm³, the mice were randomly divided into 3 groups, with 6 mice per group. The vehicle control group was administered vehicle (PBS) by intraperitoneal injection (i.p.) every other day starting on day 8 post-inoculation. The BSAP control construct 014 (CD19 Fab×CD3) was administered (i.p.) at a dose of 30 μg/kg every other day starting on day 8 post-inoculation. The exemplary TSAP construct 015 (CD19×CD8×CD3 Fab) was administered (i.p.) starting on day 8 at a dose of 100 μg/kg every other day for 6 doses before escalating to 3000 μg/kg daily for 10 injections.

The tumor volumes and mouse weight changes were measured and recorded twice a week, and the inhibitory effect of the exemplary TSAP constructs compared to vehicle control on tumor growth was observed. At the end of the study, the mice are euthanized, and the tumor tissues are harvested, weighed, and photographed. The spleen also is collected at the end of the study, and CD8+ T cells are analyzed by flow cytometry for activation markers and cytokine production. Mice administered the TSAP construct 015 showed a dose-dependent anti-tumor response (data not shown).

Experiment 2:5 days before tumor inoculation (Day-5), hPBMCs (purchased from OriBiotech) were resuspended in sterile PBS to adjust to cell density of 2.5×10⁷/mL, and administered to mice by intraperitoneal injection (i.p.) at a dose of 5×10⁶ cells per mouse, 0.2 mL per mouse. The human Burkitt lymphoma cell line, Raji, was cultured and expanded in vitro, and once in the logarithmic growth phase, the tumor cells were collected, resuspended in sterile PBS to adjust to cell density of 3×10⁷/mL, and inoculated into NSG mice by subcutaneous injection at a dose of 3×10⁶ cells per mouse, 0.2 mL per mouse, on Day 0. On Day 7, when the tumors grew to an average size of approximately 50-100 mm³, the mice with tumor size within normal distribution were randomly divided into 3 groups, with N=5 mice per group. Each group received subcutaneous injection of either vehicle (PBS) or test TSAP every other day starting on Day 7 post-inoculation, for a total of 3 weeks. The study ended on Day 28. The vehicle control group was administered vehicle (PBS) by subcutaneous injection (s.c.) for each administration. The exemplary TSAP construct 020 (CD3×CD8×CD19 Fab) was administered (s.c.) starting on Day 7 at a dose of 100 μg/kg (N=5) or a dose of 300 μg/kg (N=5) per administration.

The tumor long (L) and short(S) diameters were measured using vernier calipers and mouse weight changes were measured and recorded twice to three times per week, and the tumor volume (TV) and tumor growth inhibition rate (TGI %) were calculated according to the equations below. avT₀ represents the average tumor volume on Day 7 when TSAP construct 020 was administered, and avT₁ represents the average tumor volume on Day 7+i when TSAP construct 020 was administered. avC₀ represents the average tumor volume on Day 7 when PBS control was administered, and avCᵢ represents the average tumor volume on Day 7+i when PBS control was administered.

$$Tumor Volume ( TV ) = ( L \times {W}^{2} ) / 2$$

$$Tumor Growth Inhibition Rate ( TGI \% ) = ( 1 - \frac{{avT}_{0} - {avT}_{i}}{{avC}_{0} - {avC}_{i}} ) \times 100 \%$$

At the end of the study (Day 28), the mice were euthanized, and the tumor tissues were harvested, weighed, and photographed. The spleen also was collected at the end of the study, weighed, and the cells were processed into a single-cell suspension, and then stained using anti-human CD4 antibody (ThermoFisher, 69-0049-42) and anti-human CD8 antibody (ThermoFisher, 62-5273-42). The number of CD8+ T cells and CD4+ T cells per mg splenic tissue were analyzed by flow cytometry. Calculation is as follows: CD4+ T cells or CD8+ T cells per splenic tissue (mg)=(number of positive cells per unit volume)× cell suspension volume/tissue weight.

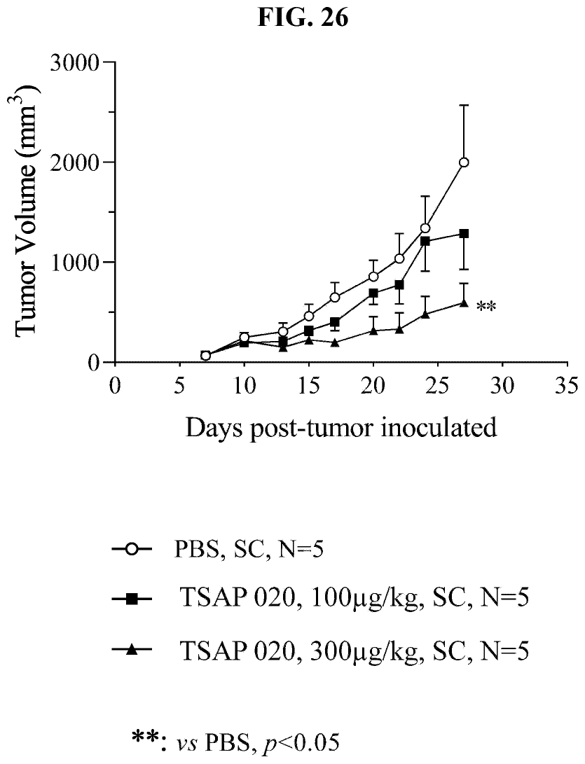

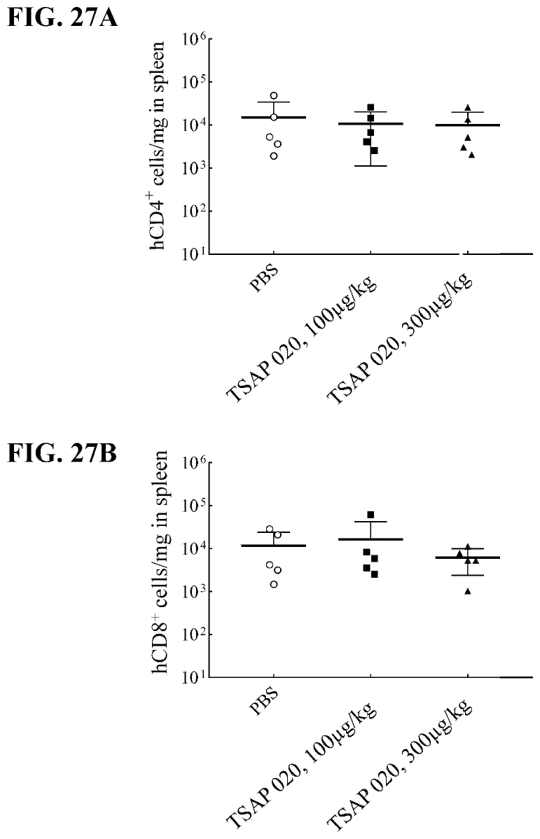

Mice administered the TSAP construct 020 showed a dose-dependent anti-tumor effect (FIG. 26), wherein the TGI % on Day 27 were 37.1% and 73.0% for tumors in mice treated with 100 μg/kg and 300 μg/kg TSAP construct 020, respectively (p-value of 0.1003 and 0.0064, respectively, compared to PBS control group). Further, there was no statistical difference in the number of CD4+ T cells (FIG. 27A) and CD8+ T cells (FIG. 27B) in the spleen tissue of the mice across the control group, 100 μg/kg TSAP construct 020 treatment group, and 300 μg/kg TSAP construct 020 treatment group. These results demonstrate that exemplary TSAPs described herein (e.g., TSAP construct 020) are not only effective in treating target tumor in vivo, but also safe-they do not lead to the death of CD8+ T cells in vivo, which corroborates the above in vitro data.

### Example 7: The Effect of CD19×CD8×CD3 TSAPs on Human PBMC Cytokine Production

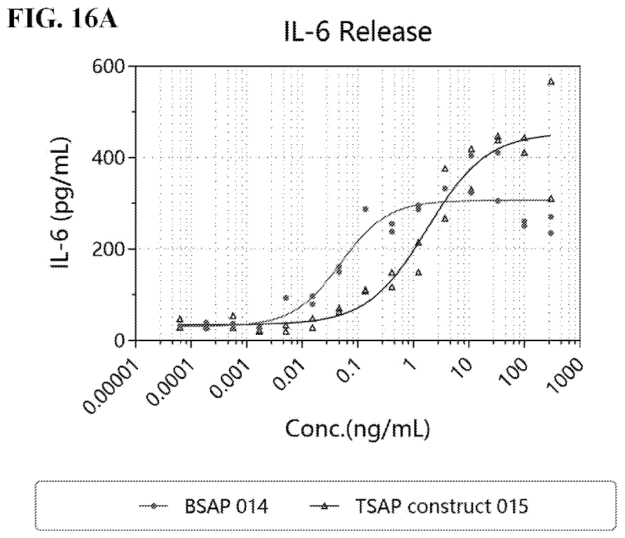

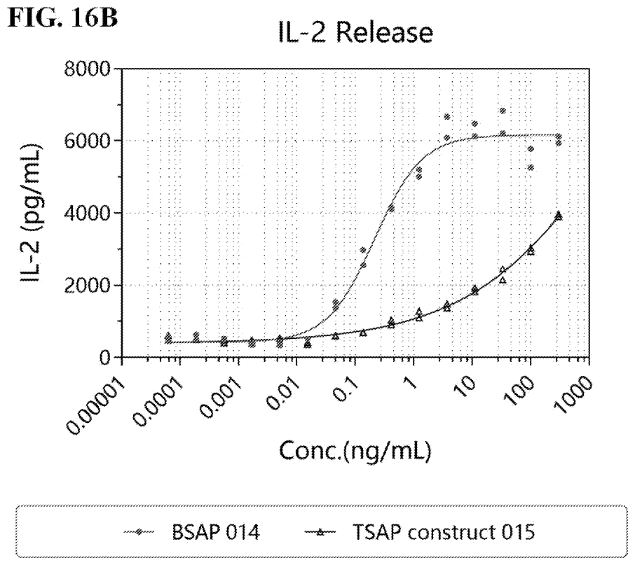

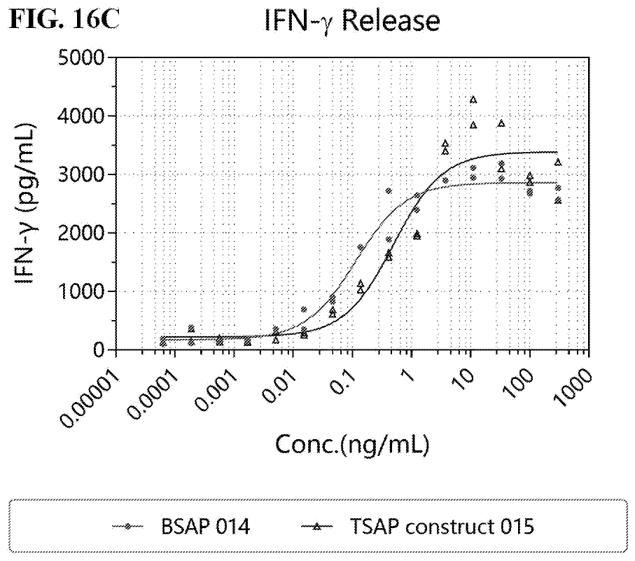

Human PBMCs were isolated from two individuals (i.e., Individual A and Individual B) and cultured as described above, then incubated with either BSAP control construct 014 (CD19 Fab×CD3) or exemplary TSAP construct 015 (CD19×CD8×CD3 Fab) for 48 hrs using a 3-fold serial dilution ranging from 300 ng/mL to 0.063 μg/mL. Media was harvested to assess pro-inflammatory cytokine production and secretion into the media. Cytokine levels were analyzed by ELISA. Concentration of each cytokine (pg/mL) was plotted against the concentration of construct 014 or 015 (FIGS. 16A-16C), and GraphPad Prism software was used to fit the data with a 4-parameter equation and calculate the EC₅₀ values were calculated (Table 8 below). TSAP construct 015 incubation increased IFNγ production as reflected by the approximately 40-fold decrease in EC₅₀. (FIG. 16C) and greatly reduced IL-6 production (FIG. 16A) and IL-2 production (FIG. 16B) by hPBMCs from healthy donors compared to BSAP 014. CD8+ T cells produce IFNγ for cytolysis, whereas CD4+ T cells produce more IL-2 than CD8+ T cells. IL-6 and TNFα are pro-inflammatory cytokines that, at high circulating levels, can lead to cytokine release syndrome and immune toxicity.

TABLE 8
EC₅₀ values for human PBMC cytokine release in the
presence of exemplary CD19 × CD8 × CD3
Fab TSAP and corresponding CD19 Fab × CD3 BSAP control.

| | BSAP 014 | | TSAP 015 | |
|---|---|---|---|---|
| | Individual A | Individual B | Individual A | Individual B |
| IL-2 | 0.217 | 0.045 | >100 | >100 |
| IL-6 | 0.05 | 0.031 | 1.77 | 0.476 |
| IFN-γ | 0.113 | 0.363 | 0.496 | 0.206 |
| TNF-α | N/A | 0.036 | N/A | 2.19 |

### Example 8: The Effect of CD19×CD8×CD3 TSAPs on a Cynomolgus Monkey Model

Non-naïve cynomolgus monkeys were administered the exemplary TSAP construct 015 (CD19×CD8×CD3 Fab) or BSAP control 014 (CD19 Fab×CD3) via either subcutaneous injection (S.C.) or intravenous infusion given over 2 hrs (I.V.). For I.V. infusion, cynomolgus monkeys were administered a single dose of TSAP 015 at a dose of 40 μg/kg or BSAP 014 at a dose of 30 μg/kg over a 2 hr infusion period. For S.C. injection, cynomolgus monkeys were administered with multiple doses of TSAP 015 at a dose of 400 μg/kg or BSAP 014 a dose of 300 μg/kg at 0 hr, 48 hrs, and 96 hrs. Serum concentration of TSAP 015 was measured, the levels of circulating B cells (represented by CD45+CD20+) and T cells (represented by either CD3+CD4+ or CD3+CD8+) were assessed, and the serum levels of IL-2, IL-6, TNFα, and IFNγ were measured by ELISA.

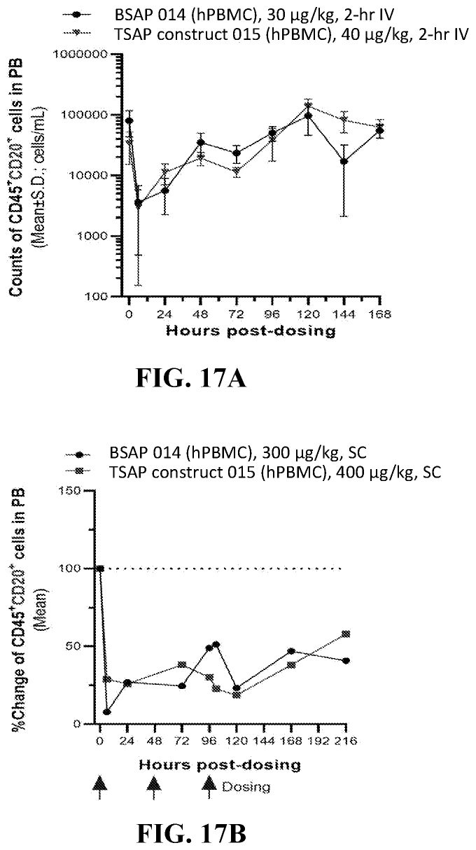

After I.V. infusion of TSAP 015, the levels of peripheral B cells showed a rapid decrease 6 hours after dosing and a sustained depletion between days 0-3 followed by recovery of B cell levels starting on day 4 (FIG. 17A), which tracked closely with peripheral B cell levels after I.V. administration of BSAP control construct 014. On the other hand, peripheral B cell depletion occurred 6 hours after dosing initiated and remained at a steady depletion level through day 7 in cynomolgus monkeys administered TSAP 015 or BSAP 014 by S.C. injection (FIG. 17B).

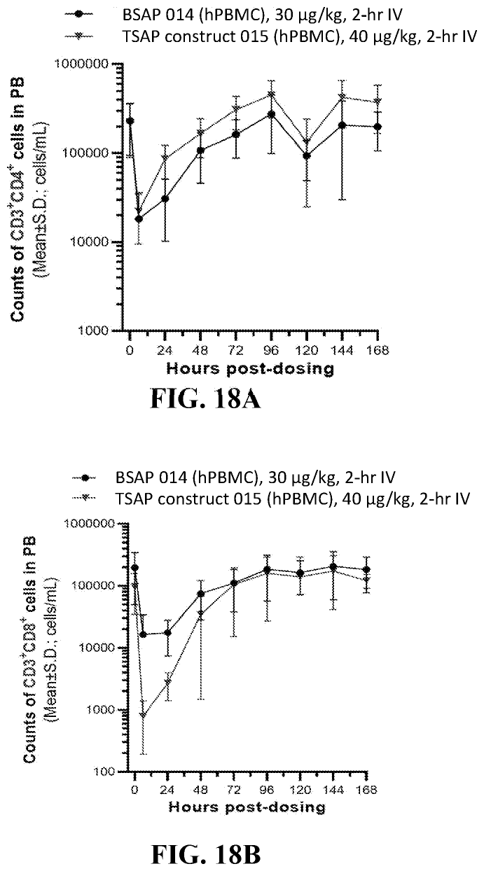

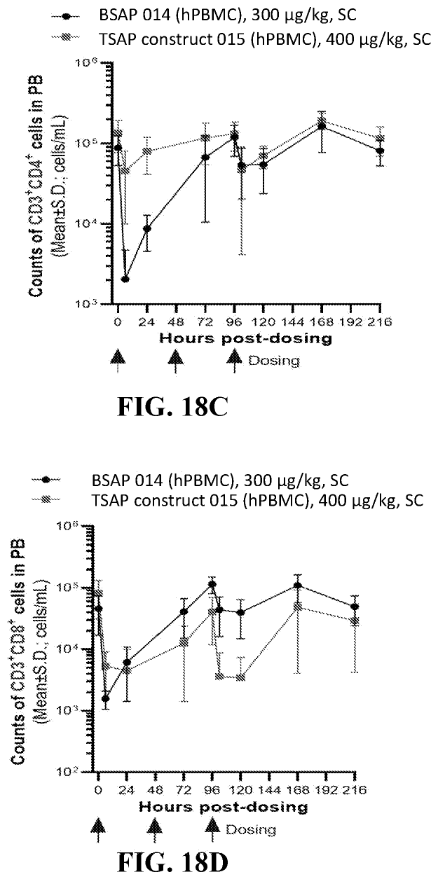

Further, cynomolgus monkeys administered TSAP 015 showed rapid T cell redistribution shortly after administration (i.e., before hour 12 after I.V. and S.C. administration, and by approximately hour 100 after the third S.C. dosing) and then T cell recovery with no evidence of peripheral T cell depletion by 216 hrs post-administration (FIGS. 18A-18D). When T cells are activated, there is a rapid decrease of T cell counts in the circulation as the T cells migrate into tissues or other organs before returning to circulation after 24 hrs. Importantly, S.C. administration of TSAP 015 showed rapid CD8+ T cell redistribution but only minimally impacted peripheral CD4+ T cells (FIGS. 18C-18D) compared to BSAP control construct 014, which significantly impacted CD4+ T cell redistribution after the first dose but showed lower impact after the administration of later doses. Thes results demonstrate the in vivo selectivity of TSAP 015 for CD8+ T cells compared to CD4+ T cells, and the effectiveness of targeting target cells (e.g., CD45+CD20+ B cells).

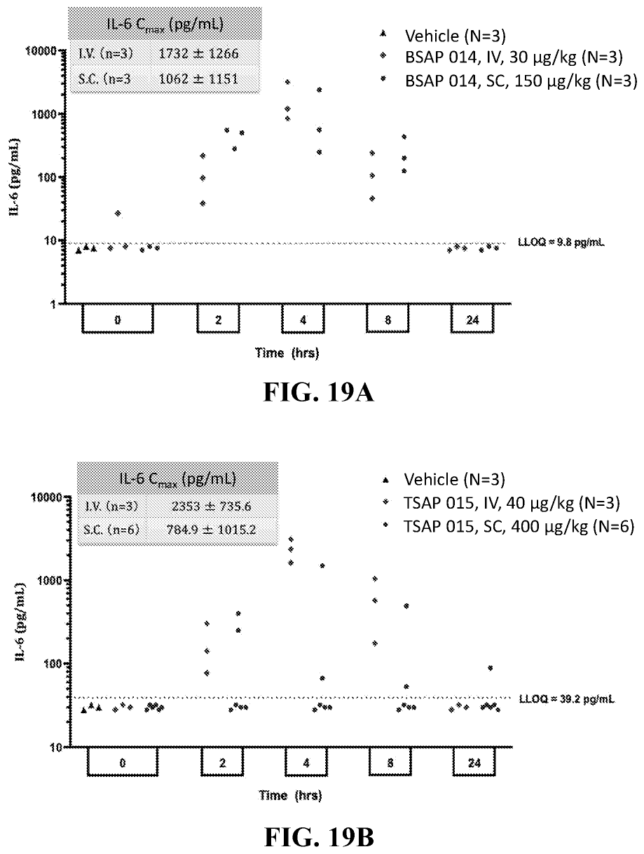

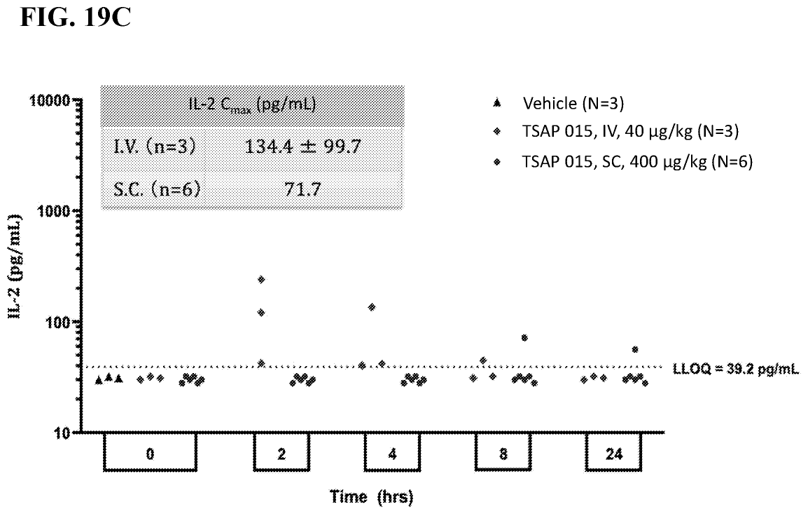

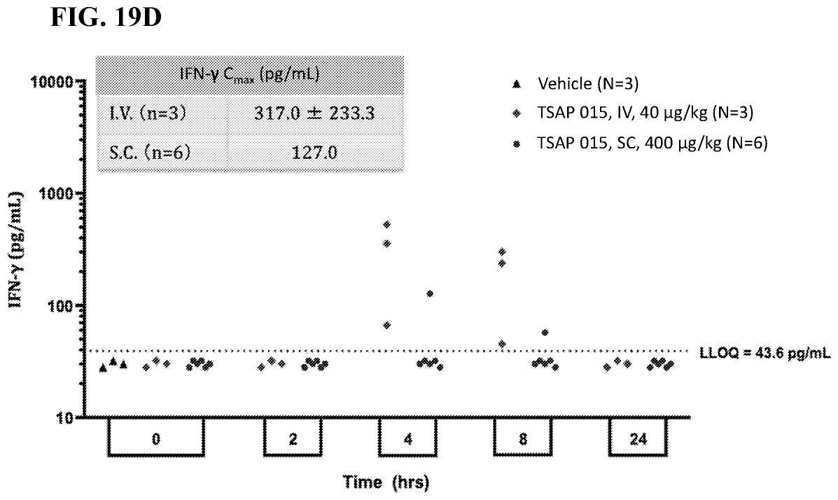

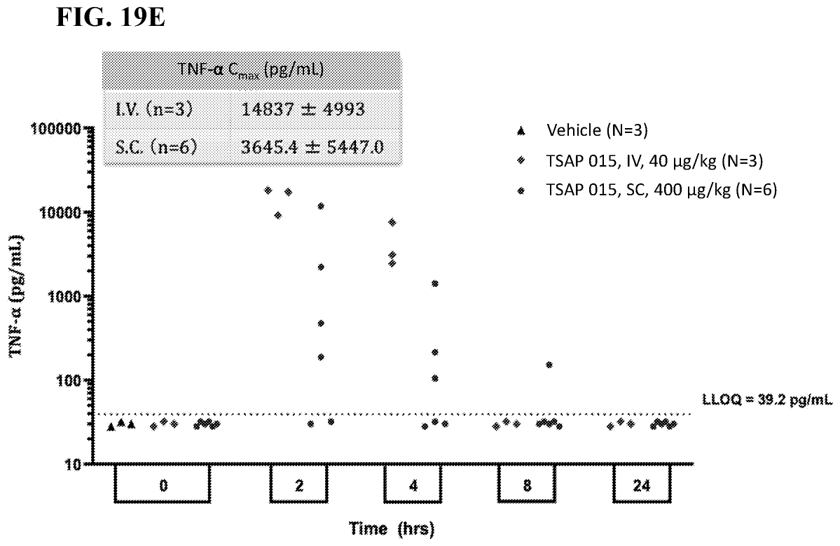

Serum levels of IL-6, IL-2, TNFα, and IFNγ consistently remained lower over 24 hrs in cynomolgus monkeys administered one dose of TSAP 015 by S.C. compared to I.V. (FIGS. 19A-19E). Comparison of circulating IL-6 levels in cynomolgus monkeys treated with TSAP 015 (FIG. 19B) or BSAP control construct 014 (FIG. 19A) showed lower IL-6 levels after S.C. administration of TSAP 015.

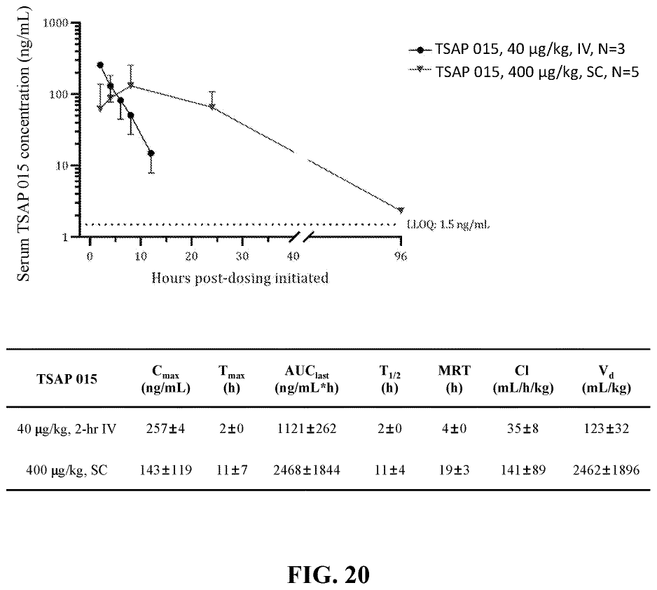

Pharmacokinetic properties of TSAP 015 were assessed based on serum concentration over time after I.V. or S.C. administration (FIG. 20). Results showed a steady decline hour-over-hour, with clearance of TSAP 015 occurring more slowly following S.C. administration compared to I.V. administration. The T_{1/2} of S.C. administration was about 11 hours, while the T_{1/2} of I.V. administration was about 2 hours.

These results demonstrate the pharmacokinetic and pharmacodynamic properties of the exemplary TSAP 015, including TSAP 015 stability, target B cell clearance, and safety profile after administration via either I.V. infusion or S.C. injection. Surprisingly, results for S.C. treatment with TSAP 015 showed comparable efficacy but better safety than I.V. treatment as compared to BSAP control. Cynomolgus monkeys showed significant peripheral blood B cell depletion after treatment with TSAP 015, and rapid T cell redistribution and recovery with no evidence of significant peripheral T cell depletion in blood, particularly following S.C. administration. Compared to BSAP control construct 014, TSAP 015 also induced lower levels of circulating IL-6 in cynomolgus monkeys over time, indicating a reduced risk of triggering cytokine release syndrome and demonstrating improved safety profile over the BSAP control when administered subcutaneously. These in vivo results show that the in vitro results observed above can be applicable to in vivo administration and therapeutic efficacy.

All references mentioned in the present invention are incorporated herein by reference as if each of those references has been incorporated by reference individually. Although the description referred to particular embodiments, it will be clear to a person skilled in the art that the present invention may be practiced with variation of these specific details. Hence this invention should not be construed as limited to the embodiments set forth herein.

1. A multispecific antigen binding protein comprising:
i) an anti-CD3 moiety that specifically binds to CD3;
ii) an anti-CD8 moiety that specifically binds to CD8; and
iii) an anti-target epitope moiety that specifically binds to a target epitope,
wherein a first moiety of the three moieties is a Fab fragment, wherein the Fab fragment comprises a first polypeptide comprising a heavy chain variable region (VH) and a heavy chain constant region (CH1), and a second polypeptide comprising a light chain variable region (VL) and a light chain constant region (CL);
wherein a second moiety of the three moieties is a first antigen binding fragment;
wherein a third moiety of the three moieties is a second antigen binding fragment; and
wherein:
(a) the first antigen binding fragment is fused to the N-terminus of the VH of the Fab fragment, and the second antigen binding fragment is fused to the N-terminus of the VL of the Fab fragment;
(b) the first antigen binding fragment is fused to the C-terminus of the CH1 of the Fab fragment, and the second antigen binding fragment is fused to the N-terminus of the VL of the Fab fragment;
(c) the first antigen binding fragment is fused to the N-terminus of the VH of the Fab fragment, and the second antigen binding fragment is fused to the C-terminus of the CL of the Fab fragment;
(d) the first antigen binding fragment is fused to the N-terminus of the VH of the Fab fragment, and the second antigen binding fragment is fused to the C-terminus of the CH1 of the Fab fragment; or
(e) the first antigen binding fragment is fused to the N-terminus of the VL of the Fab fragment, and the second antigen binding fragment is fused to the C-terminus of the CL of the Fab fragment.

2. The multispecific antigen binding protein of claim 1, wherein
(a) the anti-CD3 moiety is the Fab fragment (“anti-CD3 Fab fragment”), the anti-CD8 moiety is the first antigen binding fragment (“anti-CD8 first antigen binding fragment”), and the anti-target epitope moiety is the second antigen binding fragment (“anti-target epitope second antigen binding fragment”);
(b) the anti-CD3 moiety is the Fab fragment, the anti-target epitope moiety is the first antigen binding fragment (“anti-target epitope first antigen binding fragment”), and the anti-CD8 moiety is the second antigen binding fragment (“anti-CD8 second antigen binding fragment”);
(c) the anti-CD8 moiety is the Fab fragment (“anti-CD8 Fab fragment”), the anti-CD3 moiety is the first antigen binding fragment (“anti-CD3 first antigen binding fragment”), and the anti-target epitope moiety is the second antigen binding fragment;
(d) the anti-CD8 moiety is the Fab fragment, the anti-target epitope moiety is the first antigen binding fragment, and the anti-CD3 moiety is the second antigen binding fragment (“anti-CD3 second antigen binding fragment”);
(e) the anti-target epitope moiety is the Fab fragment (“anti-target epitope Fab fragment”), the anti-CD3 moiety is the first antigen binding fragment, and the anti-CD8 moiety is the second antigen binding fragment; or
(f) the anti-target epitope moiety is the Fab fragment, the anti-CD8 moiety is the first antigen binding fragment, and the anti-CD3 moiety is the second antigen binding fragment.

3. (canceled)

4. The multispecific antigen binding protein of claim 1, wherein the first antigen binding fragment and the second antigen binding fragment are both scFv.

5. The multispecific antigen binding protein of claim 1, wherein the anti-CD8 moiety specifically binds to CD8α, and wherein;
(I) the anti-CD8 moiety comprises:
an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23; and/or
(II) the anti-CD8 moiety comprises a VH comprising the amino acid sequence of SEQ ID NO: 34, and a VL comprising the amino acid sequence of SEQ ID NO: 35; and/or
(III) the anti-CD8 moiety is an anti-CD8 scFv, and wherein the anti-CD8 scFv comprises the amino acid sequence of SEQ ID NO: 49 or 55.

6-7. (canceled)

8. The multispecific antigen binding protein of claim 1, wherein:
(I) the anti-CD3 moiety comprises:
an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; and/or
(II) the anti-CD3 moiety comprises a VH comprising the amino acid sequence of SEQ ID NO: 167, and a VL comprising the amino acid sequence of SEQ ID NO: 43; and/or
(III) the anti-CD3 moiety is an anti-CD3 scFv, and wherein the anti-CD3 scFv comprises the amino acid sequence of SEQ ID NO: 183 or 184.

9-11. (canceled)

12. The multispecific antigen binding protein of claim 1, wherein the anti-target epitope moiety specifically recognizes CD19 (anti-CD19 moiety), and wherein:
(i) the anti-CD19 moiety comprises an HVR-H1 comprising the amino acid sequence of SEQ ID NO: 57, an HVR-H2 comprising the amino acid sequence of SEQ ID NO: 58, an HVR-H3 comprising the amino acid sequence of SEQ ID NO: 59, an HVR-L1 comprising the amino acid sequence of SEQ ID NO: 60, an HVR-L2 comprising the amino acid sequence of SEQ ID NO: 61, and an HVR-L3 comprising the amino acid sequence of SEQ ID NO: 62;
(ii) the anti-CD19 moiety comprises a VH comprising the amino acid sequence of SEQ ID NO: 63, and a VL comprising the amino acid sequence of SEQ ID NO: 64; and/or
(iii) the anti-CD19 moiety is an anti-CD19 Fab, and wherein the anti-CD19 Fab fragment comprises a first polypeptide comprising the amino acid sequence of SEQ ID NO: 137, and a second polypeptide comprising the amino acid sequence of SEQ ID NO: 139 or 169.

13-26. (canceled)

27. The multispecific antigen binding protein of claim 1, wherein the multispecific antigen binding protein comprises:
i) an anti-CD19 Fab fragment, an anti-CD8 first scFv, and an anti-CD3 second scFv; or
ii) an anti-CD19 Fab fragment, an anti-CD3 first scFv, and an anti-CD8 second scFv.

28. (canceled)

29. The multispecific antigen binding protein of claim 27, wherein the multispecific antigen binding protein comprises a first fusion polypeptide comprising an anti-CD8 first scFv fused to the C-terminus of the CH1 of the anti-CD19 Fab fragment via a first linker, and a second fusion polypeptide comprising an anti-CD3 second scFv fused to the N-terminus of the VL of the anti-CD19 Fab fragment via a second linker; and wherein
the first fusion polypeptide comprises the amino acid sequence of SEQ ID NO: 172, and the second fusion polypeptide comprises the amino acid sequence of SEQ ID NO: 176.

30-38. (canceled)

39. A pharmaceutical composition comprising the multispecific antigen binding protein of claim 1, and a pharmaceutically acceptable carrier.

40. A method of treating a disease associated with a target epitope in an individual, comprising administering to the individual an effective amount of the pharmaceutical composition of claim 39.

41-44. (canceled)

45. An anti-CD8 antibody or antigen binding fragment thereof that specifically binds to CD8, wherein the anti-CD8 antibody or antigen binding fragment thereof comprises:
i) an HVR-H1 comprising the amino acid sequence of SEQ ID NO: 13, an HVR-H2 comprising the amino acid sequence of SEQ ID NO: 14, an HVR-H3 comprising the amino acid sequence of SEQ ID NO: 15, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6;
ii) an HVR-H1 comprising the amino acid sequence of SEQ ID NO: 1, an HVR-H2 comprising the amino acid sequence of SEQ ID NO: 16, an HVR-H3 comprising the amino acid sequence of SEQ ID NO: 3, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6;
iii) an HVR-H1 comprising the amino acid sequence of SEQ ID NO: 1, an HVR-H2 comprising the amino acid sequence of SEQ ID NO: 2, an HVR-H3 comprising the amino acid sequence of SEQ ID NO: 17, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6;
iv) an HVR-H1 comprising the amino acid sequence of SEQ ID NO: 1, an HVR-H2 comprising the amino acid sequence of SEQ ID NO: 18, an HVR-H3 comprising the amino acid sequence of SEQ ID NO: 19, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6;
v) an HVR-H1 comprising the amino acid sequence of SEQ ID NO: 7, an HVR-H2 comprising the amino acid sequence of SEQ ID NO: 8, an HVR-H3 comprising the amino acid sequence of SEQ ID NO: 9, an HVR-L1 comprising the amino acid sequence of SEQ ID NO: 10, an HVR-L2 comprising the amino acid sequence of SEQ ID NO: 11, and an HVR-L3 comprising the amino acid sequence of SEQ ID NO: 12; or
vi) an HVR-H1 comprising the amino acid sequence of SEQ ID NO: 20, an HVR-H2 comprising the amino acid sequence of SEQ ID NO: 2, an HVR-H3 comprising the amino acid sequence of SEQ ID NO: 22, an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 23.

46-50. (canceled)

51. An anti-CD3 antibody or antigen binding fragment thereof that specifically binds to CD3, wherein the anti-CD3 antibody or antigen binding fragment thereof comprises:
(a) an HVR-H1 comprising the amino acid sequence of SEQ ID NO: 36, an HVR-H2 comprising the amino acid sequence of SEQ ID NO: 37, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41;
(b) an HVR-H1 comprising the amino acid sequence of SEQ ID NO: 36, an HVR-H2 comprising the amino acid sequence of SEQ ID NO: 157, an HVR-H3 comprising the amino acid sequence of SEQ ID NO: 158, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41; or
(c) an HVR-H1 comprising the amino acid sequence of SEQ ID NO: 36, an HVR-H2 comprising the amino acid sequence of SEQ ID NO: 166, an HVR-H3 comprising the amino acid sequence of SEQ ID NO: 38, an HVR-L1 comprising the amino acid sequence of SEQ ID NO: 39, an HVR-L2 comprising the amino acid sequence of SEQ ID NO: 40, and an HVR-L3 comprising the amino acid sequence of SEQ ID NO: 41.

52-55. (canceled)

56. A multispecific antigen binding protein comprising the anti-CD8 antibody or antigen binding fragment thereof of claim 45.

57. An isolated nucleic acid or a vector encoding the multispecific antigen binding protein of claim 1.

58. (canceled)

59. A host cell comprising the isolated nucleic acid or the vector of claim 57.

60. A method of making a multispecific antigen binding protein, comprising:
i) culturing a host cell comprising the isolated nucleic acid or vector of claim 57 under a condition suitable for the expression of the multispecific antigen binding protein; and
ii) obtaining the expressed multispecific antigen binding protein from said host cell.

61. A pharmaceutical composition comprising:
i) the anti-CD3 antibody or antigen binding fragment thereof of claim 51, or a multispecific antigen binding protein comprising thereof, and ii) a pharmaceutically acceptable carrier.

62. A multispecific antigen binding protein comprising the anti-CD3 antibody or antigen binding fragment thereof of claim 51.

63. An isolated nucleic acid or a vector encoding the anti-CD8 antibody or antigen binding fragment thereof of claim 45.

64. An isolated nucleic acid or a vector encoding the anti-CD3 antibody or antigen binding fragment thereof of claim 51.

65. A method of making an anti-CD8 antibody or antigen binding fragment thereof, or a multispecific antigen binding protein comprising thereof, comprising:
i) culturing a host cell comprising the isolated nucleic acid or vector of claim 63 under a condition suitable for the expression of the anti-CD8 antibody or antigen binding fragment thereof, or the multispecific antigen binding protein comprising thereof; and
ii) obtaining the expressed anti-CD8 antibody or antigen binding fragment thereof, or the expressed multispecific antigen binding protein comprising thereof, from said host cell.

66. A method of making an anti-CD3 antibody or antigen binding fragment thereof, or a multispecific antigen binding protein comprising thereof, comprising:
i) culturing a host cell comprising the isolated nucleic acid or vector of claim 64 under a condition suitable for the expression of the anti-CD3 antibody or antigen binding fragment thereof, or the multispecific antigen binding protein comprising thereof; and
ii) obtaining the expressed anti-CD3 antibody or antigen binding fragment thereof, or the expressed multispecific antigen binding protein comprising thereof, from said host cell.

67. A pharmaceutical composition comprising i) the anti-CD8 antibody or antigen binding fragment thereof of claim 45, or a multispecific antigen binding protein comprising thereof, and ii) a pharmaceutically acceptable carrier.


SEQUENCE LISTING (184 sequences)

SEQ ID NO: 1 - protein, 6 aa, synthetic construct
SSNWWS

SEQ ID NO: 2 - protein, 16 aa, synthetic construct
EINHSGSTNYNPSLKS

SEQ ID NO: 3 - protein, 9 aa, synthetic construct
QGASDAFDI

SEQ ID NO: 4 - protein, 11 aa, synthetic construct
RASQDISSWLA

SEQ ID NO: 5 - protein, 7 aa, synthetic construct
SASRLQG

SEQ ID NO: 6 - protein, 9 aa, synthetic construct
QQALSVPFT

SEQ ID NO: 7 - protein, 5 aa, synthetic construct
SYYMH

SEQ ID NO: 8 - protein, 17 aa, synthetic construct
IINPSGGSTSYAQKFQG

SEQ ID NO: 9 - protein, 12 aa, synthetic construct
DFYDSSELTMDV

SEQ ID NO: 10 - protein, 11 aa, synthetic construct
SADKLGHKYTS

SEQ ID NO: 11 - protein, 7 aa, synthetic construct
QDDKRPS

SEQ ID NO: 12 - protein, 9 aa, synthetic construct
QAWDSTSYV

SEQ ID NO: 13 - protein, 6 aa, synthetic construct
SRNWWS

SEQ ID NO: 14 - protein, 16 aa, synthetic construct
EVYHAGNTNYNPSLKS

SEQ ID NO: 15 - protein, 12 aa, synthetic construct
SLQQSGTDAFEI

SEQ ID NO: 16 - protein, 16 aa, synthetic construct
EIYHRGDTNYNPSLKS

SEQ ID NO: 17 - protein, 15 aa, synthetic construct
GYCSSTSCVIDAFDI

SEQ ID NO: 18 - protein, 16 aa, synthetic construct
EIYHSGSTNYNPSLKS

SEQ ID NO: 19 - protein, 10 aa, synthetic construct
AKAAFDAFDV

SEQ ID NO: 20 - protein, 5 aa, synthetic construct
GYYWS

SEQ ID NO: 21 - protein, 243 aa, synthetic construct
NIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKGGGGSGGGGSGGGGSRLQLQESGPGLVKPSGTLSLTCAVSGGSISSRNWWSWVRQPPGKGLEWIGEVYHAGNTNYNPSLKSRVTMSVDKSKNHFSLKLTSVTAADTAVYYCASSLQQSGTDAFEIWGQGTLVTVSA

SEQ ID NO: 22 - protein, 11 aa, synthetic construct
SGSYLADAFDI

SEQ ID NO: 23 - protein, 9 aa, synthetic construct
QQSLSVPFT

SEQ ID NO: 24 - protein, 121 aa, synthetic construct
RLQLQESGPGLVKPSGTLSLTCAVSGGSISSRNWWSWVRQPPGKGLEWIGEVYHAGNTNYNPSLKSRVTMSVDKSKNHFSLKLTSVTAADTAVYYCASSLQQSGTDAFEIWGQGTLVTVSA

SEQ ID NO: 25 - protein, 107 aa, synthetic construct
NIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFK

SEQ ID NO: 26 - protein, 118 aa, synthetic construct
QLQLQESGPGLVKPSGTLSLTCAVSGGSISSSNWWSWVRQPPGKGLEWIGEIYHRGDTNYNPSLKSRVTISVDTSKNQFSLKLSSVTAADTAVYYCARQGASDAFDIWGQGTMVTVSA

SEQ ID NO: 27 - protein, 107 aa, synthetic construct
DIQMTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFK

SEQ ID NO: 28 - protein, 124 aa, synthetic construct
RLQLQESGPGLVKPSGTLSLTCAVSGGSISSSNWWSWVRQPPGKGLEWIGEINHSGSTNYNPSLKSRVTISVDTSKNQFSLKLSSVTAADTAVYYCAIGYCSSTSCVIDAFDIWGQGTMVTVSA

SEQ ID NO: 29 - protein, 107 aa, synthetic construct
DIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFK

SEQ ID NO: 30 - protein, 119 aa, synthetic construct
RLQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARAKAAFDAFDVWGQGTMVTVSA

SEQ ID NO: 31 - skipped

SEQ ID NO: 32 - protein, 121 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDFYDSSELTMDVWGQGTTVTVSA

SEQ ID NO: 33 - protein, 106 aa, synthetic construct
SYELTQPPSVSVSPGQTARITCSADKLGHKYTSWYQQKPGQSPVLVIYQDDKRPSGIPERFSGSNSGNTATLTISGAQAVDEADYYCQAWDSTSYVFGTGTTVTVL

SEQ ID NO: 34 - protein, 119 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSGSYLADAFDIWGQGTMVTVSA

SEQ ID NO: 35 - protein, 107 aa, synthetic construct
DIQMTQSPSSVSASVGDRVTITCRASQDISSWLAWYQEKPGKPPHLLIHSASRLQGGVPSRFSGSGSGTDFTLTISSLQPEDFAIYYCQQSLSVPFTFGPGTKVDFK

SEQ ID NO: 36 - protein, 5 aa, synthetic construct
TYAMN

SEQ ID NO: 37 - protein, 19 aa, synthetic construct
RIRSKYNNYATYYADSVKD

SEQ ID NO: 38 - protein, 14 aa, synthetic construct
HGNFGNSYVSWFAY

SEQ ID NO: 39 - protein, 14 aa, synthetic construct
RSSTGAVTTSNYAN

SEQ ID NO: 40 - protein, 7 aa, synthetic construct
GTNKRAP

SEQ ID NO: 41 - protein, 9 aa, synthetic construct
ALWYSNLWV

SEQ ID NO: 42 - protein, 125 aa, synthetic construct
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSS

SEQ ID NO: 43 - protein, 109 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 44 - protein, 243 aa, synthetic construct
RLQLQESGPGLVKPSGTLSLTCAVSGGSISSRNWWSWVRQPPGKGLEWIGEVYHAGNTNYNPSLKSRVTMSVDKSKNHFSLKLTSVTAADTAVYYCASSLQQSGTDAFEIWGQGTLVTVSAGGGGSGGGGSGGGGSNIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFK

SEQ ID NO: 45 - protein, 240 aa, synthetic construct
QLQLQESGPGLVKPSGTLSLTCAVSGGSISSSNWWSWVRQPPGKGLEWIGEIYHRGDTNYNPSLKSRVTISVDTSKNQFSLKLSSVTAADTAVYYCARQGASDAFDIWGQGTMVTVSAGGGGSGGGGSGGGGSDIQMTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFK

SEQ ID NO: 46 - protein, 246 aa, synthetic construct
RLQLQESGPGLVKPSGTLSLTCAVSGGSISSSNWWSWVRQPPGKGLEWIGEINHSGSTNYNPSLKSRVTISVDTSKNQFSLKLSSVTAADTAVYYCAIGYCSSTSCVIDAFDIWGQGTMVTVSAGGGGSGGGGSGGGGSDIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFK

SEQ ID NO: 47 - protein, 241 aa, synthetic construct
RLQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARAKAAFDAFDVWGQGTMVTVSAGGGGSGGGGSGGGGSNIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFK

SEQ ID NO: 48 - protein, 242 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDFYDSSELTMDVWGQGTTVTVSAGGGGSGGGGSGGGGSSYELTQPPSVSVSPGQTARITCSADKLGHKYTSWYQQKPGQSPVLVIYQDDKRPSGIPERFSGSNSGNTATLTISGAQAVDEADYYCQAWDSTSYVFGTGTTVTVL

SEQ ID NO: 49 - protein, 241 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSGSYLADAFDIWGQGTMVTVSAGGGGSGGGGSGGGGSDIQMTQSPSSVSASVGDRVTITCRASQDISSWLAWYQEKPGKPPHLLIHSASRLQGGVPSRFSGSGSGTDFTLTISSLQPEDFAIYYCQQSLSVPFTFGPGTKVDFK

SEQ ID NO: 50 - protein, 249 aa, synthetic construct
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 51 - protein, 240 aa, synthetic construct
DIQMTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKGGGGSGGGGSGGGGSQLQLQESGPGLVKPSGTLSLTCAVSGGSISSSNWWSWVRQPPGKGLEWIGEIYHRGDTNYNPSLKSRVTISVDTSKNQFSLKLSSVTAADTAVYYCARQGASDAFDIWGQGTMVTVSA

SEQ ID NO: 52 - protein, 246 aa, synthetic construct
DIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKGGGGSGGGGSGGGGSRLQLQESGPGLVKPSGTLSLTCAVSGGSISSSNWWSWVRQPPGKGLEWIGEINHSGSTNYNPSLKSRVTISVDTSKNQFSLKLSSVTAADTAVYYCAIGYCSSTSCVIDAFDIWGQGTMVTVSA

SEQ ID NO: 53 - protein, 241 aa, synthetic construct
NIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKGGGGSGGGGSGGGGSRLQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARAKAAFDAFDVWGQGTMVTVSA

SEQ ID NO: 54 - protein, 242 aa, synthetic construct
SYELTQPPSVSVSPGQTARITCSADKLGHKYTSWYQQKPGQSPVLVIYQDDKRPSGIPERFSGSNSGNTATLTISGAQAVDEADYYCQAWDSTSYVFGTGTTVTVLGGGGSGGGGSGGGGSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDFYDSSELTMDVWGQGTTVTVSA

SEQ ID NO: 55 - protein, 241 aa, synthetic construct
DIQMTQSPSSVSASVGDRVTITCRASQDISSWLAWYQEKPGKPPHLLIHSASRLQGGVPSRFSGSGSGTDFTLTISSLQPEDFAIYYCQQSLSVPFTFGPGTKVDFKGGGGSGGGGSGGGGSQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSGSYLADAFDIWGQGTMVTVSA

SEQ ID NO: 56 - protein, 249 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSS

SEQ ID NO: 57 - protein, 5 aa, synthetic construct
SYVMH

SEQ ID NO: 58 - protein, 14 aa, synthetic construct
WIGYINPYNDGTKY

SEQ ID NO: 59 - protein, 12 aa, synthetic construct
GTYYYGSRVFDY

SEQ ID NO: 60 - protein, 16 aa, synthetic construct
RSSKSLQNVNGNTYLY

SEQ ID NO: 61 - protein, 7 aa, synthetic construct
RMSNLNS

SEQ ID NO: 62 - protein, 9 aa, synthetic construct
MQHLEYPIT

SEQ ID NO: 63 - protein, 121 aa, synthetic construct
QVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSS

SEQ ID NO: 64 - protein, 112 aa, synthetic construct
DVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIK

SEQ ID NO: 65 - protein, 248 aa, synthetic construct
DVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIKGGGGSGGGGSGGGGSQVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSS

SEQ ID NO: 66 - protein, 248 aa, synthetic construct
QVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSSGGGGSGGGGSGGGGSDVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIK

SEQ ID NO: 67 - protein, 5 aa, synthetic construct
NYWMS

SEQ ID NO: 68 - protein, 17 aa, synthetic construct
NIKQDGSEKFYADSVKG

SEQ ID NO: 69 - protein, 9 aa, synthetic construct
VGPSWEQDY

SEQ ID NO: 70 - protein, 14 aa, synthetic construct
TGSSSNIGSYYGVH

SEQ ID NO: 71 - protein, 7 aa, synthetic construct
SDTNRPS

SEQ ID NO: 72 - protein, 11 aa, synthetic construct
QSYDKGFGHRV

SEQ ID NO: 73 - protein, 118 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSA

SEQ ID NO: 74 - protein, 113 aa, synthetic construct
GAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVL

SEQ ID NO: 75 - protein, 246 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVL

SEQ ID NO: 76 - protein, 225 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 77 - protein, 222 aa, synthetic construct
GAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 78 - protein, 246 aa, synthetic construct
GAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGGGGSGGGGSGGGGSEVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSA

SEQ ID NO: 79 - protein, 5 aa, synthetic construct
SYAIS

SEQ ID NO: 80 - protein, 17 aa, synthetic construct
GIIPIFGTANYAQKFQG

SEQ ID NO: 81 - protein, 11 aa, synthetic construct
SIELWWGGFDY

SEQ ID NO: 82 - protein, 11 aa, synthetic construct
RASQSISSWLA

SEQ ID NO: 83 - protein, 7 aa, synthetic construct
DASSLES

SEQ ID NO: 84 - protein, 8 aa, synthetic construct
QQYEDYTT

SEQ ID NO: 85 - protein, 120 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSS

SEQ ID NO: 86 - protein, 106 aa, synthetic construct
DIQMTQSPSTLSASVGDRVTITCRASQSISSWLAWYQQKPGKAPKLLIYDASSLESGVPSRFSGSGSGTEFTLTIGSLQPDDFATYYCQQYEDYTTFGQGTKVEIK

SEQ ID NO: 87 - protein, 241 aa, synthetic construct
DIQMTQSPSTLSASVGDRVTITCRASQSISSWLAWYQQKPGKAPKLLIYDASSLESGVPSRFSGSGSGTEFTLTIGSLQPDDFATYYCQQYEDYTTFGQGTKVEIKGGGGSGGGGSGGGGSQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSS

SEQ ID NO: 88 - protein, 241 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSSGGGGSGGGGSGGGGSDIQMTQSPSTLSASVGDRVTITCRASQSISSWLAWYQQKPGKAPKLLIYDASSLESGVPSRFSGSGSGTEFTLTIGSLQPDDFATYYCQQYEDYTTFGQGTKVEIK

SEQ ID NO: 89 - protein, 232 aa, synthetic construct
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 90 - protein, 218 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 91 - protein, 231 aa, synthetic construct
RLQLQESGPGLVKPSGTLSLTCAVSGGSISSSNWWSWVRQPPGKGLEWIGEINHSGSTNYNPSLKSRVTISVDTSKNQFSLKLSSVTAADTAVYYCAIGYCSSTSCVIDAFDIWGQGTMVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 92 - protein, 216 aa, synthetic construct
DIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 93 - protein, 107 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 94 - protein, 109 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 95 - protein, 478 aa, synthetic construct
DIQMTQSPSTLSASVGDRVTITCRASQSISSWLAWYQQKPGKAPKLLIYDASSLESGVPSRFSGSGSGTEFTLTIGSLQPDDFATYYCQQYEDYTTFGQGTKVEIKGGGGSGGGGSGGGGSQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 96 - protein, 470 aa, synthetic construct
RLQLQESGPGLVKPSGTLSLTCAVSGGSISSSNWWSWVRQPPGKGLEWIGEINHSGSTNYNPSLKSRVTISVDTSKNQFSLKLSSVTAADTAVYYCAIGYCSSTSCVIDAFDIWGQGTMVTVSAGGGGSGGGGSGGGGSDIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 97 - protein, 480 aa, synthetic construct
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLSGGGGSEVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 98 - protein, 474 aa, synthetic construct
RLQLQESGPGLVKPSGTLSLTCAVSGGSISSSNWWSWVRQPPGKGLEWIGEINHSGSTNYNPSLKSRVTISVDTSKNQFSLKLSSVTAADTAVYYCAIGYCSSTSCVIDAFDIWGQGTMVTVSAGGGGSGGGGSGGGGSDIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 99 - protein, 483 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLSGGGGSRLQLQESGPGLVKPSGTLSLTCAVSGGSISSSNWWSWVRQPPGKGLEWIGEINHSGSTNYNPSLKSRVTISVDTSKNQFSLKLSSVTAADTAVYYCAIGYCSSTSCVIDAFDIWGQGTMVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 100 - protein, 471 aa, synthetic construct
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLSGGGGSDIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 101 - protein, 480 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGGEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 102 - protein, 470 aa, synthetic construct; identical to SEQ ID NO: 96

SEQ ID NO: 103 - protein, 485 aa, synthetic construct
DVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIKGGGGSGGGGSGGGGSQVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 104 - protein, 470 aa, synthetic construct; identical to SEQ ID NO: 96

SEQ ID NO: 105 - skipped

SEQ ID NO: 106 - protein, 465 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSGSYLADAFDIWGQGTMVTVSAGGGGSGGGGSGGGGSDIQMTQSPSSVSASVGDRVTITCRASQDISSWLAWYQEKPGKPPHLLIHSASRLQGGVPSRFSGSGSGTDFTLTISSLQPEDFAIYYCQQSLSVPFTFGPGTKVDFKSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 107 - protein, 5 aa, synthetic construct
CPPCS

SEQ ID NO: 108 - protein, 15 aa, synthetic construct
GGGGSGGGGSGGGGS

SEQ ID NO: 109 - protein, 5 aa, synthetic construct
GGGGS

SEQ ID NO: 110 - protein, 6 aa, synthetic construct
SGGGGS

SEQ ID NO: 111 - protein, 16 aa, synthetic construct
GGGGSGGGGSGGGGSG

SEQ ID NO: 112 - protein, 6 aa, synthetic construct
GGSGGS

SEQ ID NO: 113 - protein, 5 aa, synthetic construct
GGGGS

SEQ ID NO: 114 - protein, 12 aa, synthetic construct
GRAGGGGAGGGG

SEQ ID NO: 115 - protein, 6 aa, synthetic construct
GRAGGG

SEQ ID NO: 116 - protein, 5 aa, synthetic construct
  VARIANT 1..5: note = can be present in repeat of at least 1
GSGGS

SEQ ID NO: 117 - protein, 4 aa, synthetic construct
  VARIANT 1..4: note = can be present in repeat of at least 1
GGGS

SEQ ID NO: 118 - protein, 4 aa, synthetic construct
GGSG

SEQ ID NO: 119 - protein, 5 aa, synthetic construct
GGSGG

SEQ ID NO: 120 - protein, 5 aa, synthetic construct
GSGSG

SEQ ID NO: 121 - protein, 5 aa, synthetic construct
GSGGG

SEQ ID NO: 122 - protein, 5 aa, synthetic construct
GGGSG

SEQ ID NO: 123 - protein, 5 aa, synthetic construct
GSSSG

SEQ ID NO: 124 - protein, 476 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGGRLQLQESGPGLVKPSGTLSLTCAVSGGSISSRNWWSWVRQPPGKGLEWIGEVYHAGNTNYNPSLKSRVTMSVDKSKNHFSLKLTSVTAADTAVYYCASSLQQSGTDAFEIWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 125 - protein, 464 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGGNIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 126 - protein, 473 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGGQLQLQESGPGLVKPSGTLSLTCAVSGGSISSSNWWSWVRQPPGKGLEWIGEIYHRGDTNYNPSLKSRVTISVDTSKNQFSLKLSSVTAADTAVYYCARQGASDAFDIWGQGTMVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 127 - protein, 464 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGGDIQMTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 128 - protein, 479 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGGRLQLQESGPGLVKPSGTLSLTCAVSGGSISSSNWWSWVRQPPGKGLEWIGEINHSGSTNYNPSLKSRVTISVDTSKNQFSLKLSSVTAADTAVYYCAIGYCSSTSCVIDAFDIWGQGTMVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 129 - protein, 464 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGGDIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 130 - protein, 474 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGGRLQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARAKAAFDAFDVWGQGTMVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 131 - protein, 464 aa, synthetic construct; identical to SEQ ID NO: 125

SEQ ID NO: 132 - protein, 476 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGGQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDFYDSSELTMDVWGQGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 133 - protein, 463 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGGSYELTQPPSVSVSPGQTARITCSADKLGHKYTSWYQQKPGQSPVLVIYQDDKRPSGIPERFSGSNSGNTATLTISGAQAVDEADYYCQAWDSTSYVFGTGTTVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 134 - protein, 474 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGGQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSGSYLADAFDIWGQGTMVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 135 - protein, 464 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSAGGGGSGGGGSGGGGSGAQSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVLGGDIQMTQSPSSVSASVGDRVTITCRASQDISSWLAWYQEKPGKPPHLLIHSASRLQGGVPSRFSGSGSGTDFTLTISSLQPEDFAIYYCQQSLSVPFTFGPGTKVDFKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 136 - skipped

SEQ ID NO: 137 - protein, 228 aa, synthetic construct
QVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 138 - protein, 483 aa, synthetic construct
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLSGGGGSQVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 139 - protein, 223 aa, synthetic construct
DVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCS

SEQ ID NO: 140 - protein, 6 aa, synthetic construct
PLGLAG

SEQ ID NO: 141 - protein, 19 aa, synthetic construct
MEWSWVFLFFLSVTTGVHS

SEQ ID NO: 142 - DNA, 57 bp, synthetic construct
  source 1..57: mol_type = other DNA
atggaatggagctgggtctttctcttcttcctgtcagtaacgactggtgtccactcc

SEQ ID NO: 143 - protein, 4 aa, synthetic construct
CPPC

SEQ ID NO: 144 - DNA, 15 bp, synthetic construct
  source 1..15: mol_type = other DNA
tgtccaccgtgctca

SEQ ID NO: 145 - protein, 227 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 146 - protein, 215 aa, synthetic construct
DIQMTQSPSTLSASVGDRVTITCRASQSISSWLAWYQQKPGKAPKLLIYDASSLESGVPSRFSGSGSGTEFTLTIGSLQPDDFATYYCQQYEDYTTFGQGTKVEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 147 - protein, 228 aa, synthetic construct
RLQLQESGPGLVKPSGTLSLTCAVSGGSISSRNWWSWVRQPPGKGLEWIGEVYHAGNTNYNPSLKSRVTMSVDKSKNHFSLKLTSVTAADTAVYYCASSLQQSGTDAFEIWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 148 - protein, 216 aa, synthetic construct
NIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 149 - protein, 225 aa, synthetic construct
QLQLQESGPGLVKPSGTLSLTCAVSGGSISSSNWWSWVRQPPGKGLEWIGEIYHRGDTNYNPSLKSRVTISVDTSKNQFSLKLSSVTAADTAVYYCARQGASDAFDIWGQGTMVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 150 - protein, 216 aa, synthetic construct
DIQMTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 151 - protein, 226 aa, synthetic construct
RLQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARAKAAFDAFDVWGQGTMVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 152 - skipped

SEQ ID NO: 153 - protein, 228 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDFYDSSELTMDVWGQGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 154 - protein, 215 aa, synthetic construct
SYELTQPPSVSVSPGQTARITCSADKLGHKYTSWYQQKPGQSPVLVIYQDDKRPSGIPERFSGSNSGNTATLTISGAQAVDEADYYCQAWDSTSYVFGTGTTVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 155 - protein, 226 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSGSYLADAFDIWGQGTMVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 156 - protein, 216 aa, synthetic construct
DIQMTQSPSSVSASVGDRVTITCRASQDISSWLAWYQEKPGKPPHLLIHSASRLQGGVPSRFSGSGSGTDFTLTISSLQPEDFAIYYCQQSLSVPFTFGPGTKVDFKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 157 - protein, 19 aa, synthetic construct
RIRSKYNNYATAYADSVKD

SEQ ID NO: 158 - protein, 14 aa, synthetic construct
HGNFGNSGVSWFAY

SEQ ID NO: 159 - protein, 125 aa, synthetic construct
EVQLVESGGGLVQPGGSLRLSCAASGFTFNTYAMNWVRQAPGKGLEWVARIRSKYNNYATAYADSVKDRFTISRDDSKNTLYLQMNSLRAEDTAVYYCVRHGNFGNSGVSWFAYWGQGTMVTVSS

SEQ ID NO: 160 - protein, 232 aa, synthetic construct
EVQLVESGGGLVQPGGSLRLSCAASGFTFNTYAMNWVRQAPGKGLEWVARIRSKYNNYATAYADSVKDRFTISRDDSKNTLYLQMNSLRAEDTAVYYCVRHGNFGNSGVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 161 - protein, 485 aa, synthetic construct
DVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIKGGGGSGGGGSGGGGSQVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSSGGGGSEVQLVESGGGLVQPGGSLRLSCAASGFTFNTYAMNWVRQAPGKGLEWVARIRSKYNNYATAYADSVKDRFTISRDDSKNTLYLQMNSLRAEDTAVYYCVRHGNFGNSGVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 162 - protein, 480 aa, synthetic construct
RLQLQESGPGLVKPSGTLSLTCAVSGGSISSSNWWSWVRQPPGKGLEWIGEINHSGSTNYNPSLKSRVTISVDTSKNQFSLKLSSVTAADTAVYYCAIGYCSSTSCVIDAFDIWGQGTMVTVSAGGGGSGGGGSGGGGSDIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFKSGGGGSQVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 163 - protein, 249 aa, synthetic construct
EVQLVESGGGLVQPGGSLRLSCAASGFTFNTYAMNWVRQAPGKGLEWVARIRSKYNNYATAYADSVKDRFTISRDDSKNTLYLQMNSLRAEDTAVYYCVRHGNFGNSGVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 164 - protein, 249 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLRLSCAASGFTFNTYAMNWVRQAPGKGLEWVARIRSKYNNYATAYADSVKDRFTISRDDSKNTLYLQMNSLRAEDTAVYYCVRHGNFGNSGVSWFAYWGQGTMVTVSS

SEQ ID NO: 165 - protein, 111 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCS

SEQ ID NO: 166 - protein, 19 aa, synthetic construct
RIRSKYNNYGTAYADSVKD

SEQ ID NO: 167 - protein, 125 aa, synthetic construct
EVQLVESGGGLVQPGGSLRLSCAASGFTFNTYAMNWVRQAPGKGLEWVARIRSKYNNYGTAYADSVKDRFTISRDDSKNTLYLQMNSLRAEDTAVYYCVRHGNFGNSYVSWFAYWGQGTMVTVSS

SEQ ID NO: 168 - protein, 232 aa, synthetic construct
EVQLVESGGGLVQPGGSLRLSCAASGFTFNTYAMNWVRQAPGKGLEWVARIRSKYNNYGTAYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 169 - protein, 221 aa, synthetic construct
DVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 170 - protein, 479 aa, synthetic construct
QVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCSGGEVQLVESGGGLVQPGGSLRLSCAASGFTFNTYAMNWVRQAPGKGLEWVARIRSKYNNYATAYADSVKDRFTISRDDSKNTLYLQMNSLRAEDTAVYYCVRHGNFGNSGVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 171 - protein, 468 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSGSYLADAFDIWGQGTMVTVSAGGGGSGGGGSGGGGSDIQMTQSPSSVSASVGDRVTITCRASQDISSWLAWYQEKPGKPPHLLIHSASRLQGGVPSRFSGSGSGTDFTLTISSLQPEDFAIYYCQQSLSVPFTFGPGTKVDFKSGGGGSDVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 172 - protein, 471 aa, synthetic construct
QVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCSGGQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSGSYLADAFDIWGQGTMVTVSAGGGGSGGGGSGGGGSDIQMTQSPSSVSASVGDRVTITCRASQDISSWLAWYQEKPGKPPHLLIHSASRLQGGVPSRFSGSGSGTDFTLTISSLQPEDFAIYYCQQSLSVPFTFGPGTKVDFK

SEQ ID NO: 173 - protein, 476 aa, synthetic construct
EVQLVESGGGLVQPGGSLRLSCAASGFTFNTYAMNWVRQAPGKGLEWVARIRSKYNNYATAYADSVKDRFTISRDDSKNTLYLQMNSLRAEDTAVYYCVRHGNFGNSGVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLSGGGGSDVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 174 - protein, 476 aa, synthetic construct
QVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCSGGRLQLQESGPGLVKPSGTLSLTCAVSGGSISSSNWWSWVRQPPGKGLEWIGEINHSGSTNYNPSLKSRVTISVDTSKNQFSLKLSSVTAADTAVYYCAIGYCSSTSCVIDAFDIWGQGTMVTVSAGGGGSGGGGSGGGGSDIQLTQSPSSVSASVGDRVTITCRASQDISSWLAWYQEKPGKPPHLLIHSASRLQGGVPSRFSGSGSGTDFTLTISSLQPEDFAIYYCQQALSVPFTFGPGTKVDFK

SEQ ID NO: 175 - protein, 476 aa, synthetic construct
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLSGGGGSDVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 176 - protein, 476 aa, synthetic construct
EVQLVESGGGLVQPGGSLRLSCAASGFTFNTYAMNWVRQAPGKGLEWVARIRSKYNNYGTAYADSVKDRFTISRDDSKNTLYLQMNSLRAEDTAVYYCVRHGNFGNSYVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLSGGGGSDVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCS

SEQ ID NO: 177 - protein, 483 aa, synthetic construct
EVQLVESGGGLVQPGGSLRLSCAASGFTFNTYAMNWVRQAPGKGLEWVARIRSKYNNYGTAYADSVKDRFTISRDDSKNTLYLQMNSLRAEDTAVYYCVRHGNFGNSYVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLSGGGGSQVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCS

SEQ ID NO: 178 - protein, 465 aa, synthetic construct
DVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCSSGGQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSGSYLADAFDIWGQGTMVTVSAGGGGSGGGGSGGGGSDIQMTQSPSSVSASVGDRVTITCRASQDISSWLAWYQEKPGKPPHLLIHSASRLQGGVPSRFSGSGSGTDFTLTISSLQPEDFAIYYCQQSLSVPFTFGPGTKVDFK

SEQ ID NO: 179 - protein, 220 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCS

SEQ ID NO: 180 - protein, 726 aa, synthetic construct
EVQLVESGGGLVQPGGSLRLSCAASGFTFNTYAMNWVRQAPGKGLEWVARIRSKYNNYGTAYADSVKDRFTISRDDSKNTLYLQMNSLRAEDTAVYYCVRHGNFGNSYVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLSGGGGSQVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCSGGQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSGSYLADAFDIWGQGTMVTVSAGGGGSGGGGSGGGGSDIQMTQSPSSVSASVGDRVTITCRASQDISSWLAWYQEKPGKPPHLLIHSASRLQGGVPSRFSGSGSGTDFTLTISSLQPEDFAIYYCQQSLSVPFTFGPGTKVDFK

SEQ ID NO: 181 - protein, 719 aa, synthetic construct
EVQLVESGGGLVQPGGSLRLSCAASGFTFNTYAMNWVRQAPGKGLEWVARIRSKYNNYGTAYADSVKDRFTISRDDSKNTLYLQMNSLRAEDTAVYYCVRHGNFGNSYVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLSGGGGSDVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECPPCSGGQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSGSYLADAFDIWGQGTMVTVSAGGGGSGGGGSGGGGSDIQMTQSPSSVSASVGDRVTITCRASQDISSWLAWYQEKPGKPPHLLIHSASRLQGGVPSRFSGSGSGTDFTLTISSLQPEDFAIYYCQQSLSVPFTFGPGTKVDFK

SEQ ID NO: 182 - protein, 478 aa, synthetic construct
EVQLVESGGGLVQPGGSLRLSCAASGFTFNTYAMNWVRQAPGKGLEWVARIRSKYNNYGTAYADSVKDRFTISRDDSKNTLYLQMNSLRAEDTAVYYCVRHGNFGNSYVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLSGGGGSDVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCS

SEQ ID NO: 183 - protein, 249 aa, synthetic construct
EVQLVESGGGLVQPGGSLRLSCAASGFTFNTYAMNWVRQAPGKGLEWVARIRSKYNNYGTAYADSVKDRFTISRDDSKNTLYLQMNSLRAEDTAVYYCVRHGNFGNSYVSWFAYWGQGTMVTVSSGGGGSGGGGSGGGGSQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 184 - protein, 249 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLRLSCAASGFTFNTYAMNWVRQAPGKGLEWVARIRSKYNNYGTAYADSVKDRFTISRDDSKNTLYLQMNSLRAEDTAVYYCVRHGNFGNSYVSWFAYWGQGTMVTVSS